(19) United States
(12) Patent Application Publication
(10) Pub. No.: US 2025/0108124 A1
(43) Pub. Date: Apr. 3, 2025

(54) ANTIBODY DRUG CONJUGATES USING MATES TECHNOLOGY FOR DELIVERING CYTOTOXIC AGENTS

(71) Applicant: Biohaven Therapeutics Ltd., New Haven, CT (US)

(72) Inventors: Gene M. DUBOWCHIK, Middlefield, CT (US); Wieslaw KAZMIERSKI, Raleigh, NC (US); Richard PRACITTO, Wallingford, CT (US); Reese M. CALDWELL, Cambridge, CT (US)

(21) Appl. No.: 18/555,858

(22) PCT Filed: May 19, 2022
(86) PCT No.: PCT/US2022/030070

§ 371 Date: Oct. 17, 2023

Related U.S. Application Data
(60) Provisional application No. 63/190,703, filed on May 19, 2021.

(51) Int. Cl. A61K 47/68

(52) U.S. Cl. CPC A61K 47/6889; A61K 47/68031; A61K 47/6855; A61K 47/6861

(57) ABSTRACT

Among other things, the present disclosure provides technologies for site-directed conjugation of various moieties of interest to target agents. In some embodiments, the present disclosure utilizes target binding moieties to provide high conjugation efficiency and selectivity. In some embodiments, provided technologies are useful for preparing antibody conjugates.

### CROSS REFERENCE TO RELATED APPLICATION

This application claims priority of U.S. Application No. 63/190,703 filed May 19, 2021, which is hereby incorporated by reference in its entirety.

### BACKGROUND

Antibody-drug conjugates are useful for various purposes, e.g., as diagnostic reagents, therapeutics (e.g., antigen targeted therapeutics), etc. Existing drug antibody conjugation technologies can suffer from various challenges. For example, reactions conjugating moieties of interest (e.g., detection moieties, drug moieties, etc.) to target molecules (e.g., antibodies for antibody-drug conjugates) can be of low efficiency and/or have low selectivity (e.g., conjugation at various locations (e.g., various amino acid residues of antibodies) of target molecules), and product conjugate compositions are often highly heterogeneous, comprising a number of individual conjugate types each independently having its own copy number of moieties of interest, conjugation locations (e.g., different amino acid residues of proteins), etc.

Approved antibody drug conjugates for delivering for delivering cytotoxic agents to cancer cells include PADCEV (enfortumab vedotin) are ADCETRIS (brentuximab vedotin), both useful for delivering monomethyl auristatin E (MMAE). Current drug antibody conjugation technologies include conjugation through lysine residues, conjugation through reduced interchain disulfide bonds, and conjugation through engineered cysteine residues. FIG. 1. Each of these techniques has shortcomings. Conjugation through lysine produces a broad range of drug anti-body ratios (DAR), with each lysine labeled at is statistically probability. The result being millions of possible drug antibody conjugates. High DAR specifies are prone to CMC issues such as aggregation. Some species may easily release their conjugated drug, leading to toxicity. Conjugation through reduced inter-chain disulfide bonds also produces a variety of antibody conjugate species. The drug linkage can reverse over time, releasing free drug. Existing techniques for conjugation through engineered cysteines involved extensive antibody manipulation or engineering.

There exists a need for drug an antibody conjugates with predictable DAR and conjugation sites that do not “leak” the conjugated drug, and without the need for extensive antibody engineering. This disclosure fulfills that need and has additional advantages.

### SUMMARY

This disclosure provides bifunctional molecules comprising monomethyl auristatin E (MMAE) and capable of forming antibody drug conjugates, in which conjugation occurs at finite and predictable sites on the antibody.

In some embodiments, manufacturing of conjugates involves multiple steps and includes various reactions, such as reduction, oxidation, hydrolysis, etc., and such reactions may cause undesired transformations, e.g., at one or more locations of target agent moieties (e.g., at one or more residues, and/or one or more modifications (e.g., glycans) of antibody moieties). Such undesired transformations may further lower efficiency and/or increase heterogeneity of product conjugate compositions, complicate characterization, assessment and/or purification processes and increase product cost.

In some embodiments, the present disclosure provides conjugation technologies for conjugating various moieties of interest to targets (e.g., proteins). In some embodiments, provided technologies provide directed conjugation in that moieties of interest are selectively conjugated at certain locations of targets (e.g., proteins such as antibodies). In some embodiments, provided technologies utilizes fewer steps. In some embodiments, provided technologies utilizes mild reaction conditions. In some embodiments, provided technologies include no reaction conditions such as reduction, oxidation, and/or hydrolysis. In some embodiments, provided technologies include substantially no cleavage from conjugate molecules comprising target agent moieties and moieties of interest (e.g., no cleavage of a group from target agent moieties, moieties of interest and/or linker moieties). In some embodiments, moieties of interest are detectable moieties (e.g., FITC). In some embodiments, moieties of interest are drug moieties (e.g., various drug moieties utilized in antibody-drug conjugates). In some embodiments, moieties of interest are protein moieties (e.g., antibody agents conjugated to other antibody agents (as target agent moieties)). In some embodiments, moieties of interest are or comprise reaction groups. In some embodiments, moieties of interest are or comprise reaction groups so that other moieties of interest can be further incorporated through reactions at the reaction groups.

Technologies of the present disclosure may provide various advantages. In some embodiments, the present disclosure provides improved efficiency and/or selectivity, reduced levels of heterogeneity, and/or reduced undesired transformations (e.g., through fewer steps of reactions (in some embodiments, only one), avoidance of certain reaction conditions (e.g., reduction, oxidation, hydrolysis, etc.).

In some embodiments, the present disclosure provides agents comprising moieties of interest are conjugated at certain locations of target agent moieties. In some embodiments, the present disclosure provides compositions of increased homogeneity compared to compositions from a reference technology (e.g., a technology without using target binding moieties (e.g., LG) as described in provided methods).

In some embodiments, the present disclosure provides technologies, e.g., mAb therapy enhancer (MATE™) technologies that can provide efficient site-directed chemical conjugation to “off-the-shelf” therapeutic antibody agents, e.g. various mAbs, and allow development of various bispecific therapeutic agents. Among other things, technologies of the present disclosure, e.g., MATE technologies, provide chemical engineering of antibody agents, e.g., various existing antibodies, without the need to create new DNA vectors or genetic engineering of master cell lines. In some embodiments, advantages of provided technologies include 1) site-directed conjugation specificity, and/or 2) no requirement of genetic engineering, compared to certain existing methods that 1) lack site-directed conjugation specificity by indiscriminately binding/conjugating to available amino acid residues, and/or 2) require genetic engineering to create conjugate tags. Schematics of the MATES technology are shown in FIGS. 2 and 3.

### BRIEF DESCRIPTION OF THE DRAWINGS

FIG. 1. Diagrams of existing drug antibody technologies. A. Conjugation through lysine residues. B. Conjugation through reduced inter-chain disulfide bonds. C. Conjugation through engineered cysteine residues.

FIG. 2. Schematic of MATES technology. Reaction partner bearing a reactive target binding moiety, a reactive group connected via a linker to a MOI specifically binds to the target (antibody). The reactive group attaches at an antibody lysine residue, releasing the target binding moiety.

FIG. 3. Chemical diagram of reactive target moiety specifically binding to an antibody following reaction of its reactive group with an antibody heavy chain lysine. The reactive target binding moiety comprise a cyclic peptide target binding moiety, a fluorophenyl reactive moiety, a PEG linker, and a peptide MOI.

FIG. 4. Standard curves for residual payload analysis (FIG. 4A) for Cmp. 1101, residual reagent analysis (FIG. 4B) for Cmp. 1101, and residual uABT analysis (FIG. 4C).

FIG. 5. HPLC traces and peak areas for Cmp. 1101 payload analysis. Successive traces are shown for payloads from 10 μM (largest peak), 5 μM, 2 μM, and 1 μM.

FIG. 6. HPLC traces and peak areas for Cmp. 1101 residual reagent analysis. Successive traces are shown for payloads from 10 μM (largest peak), 5 μM, 2 μM, and 1 μM.

FIG. 7. HPLC traces for uABT analysis. Successive traces are shown for payloads from 14 μM (largest peak), 7 μM, 3.5 μM, 1.75 μM, and 0.7 μM.

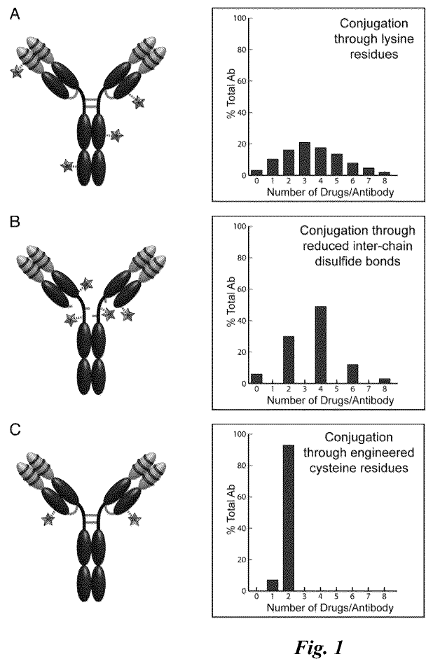

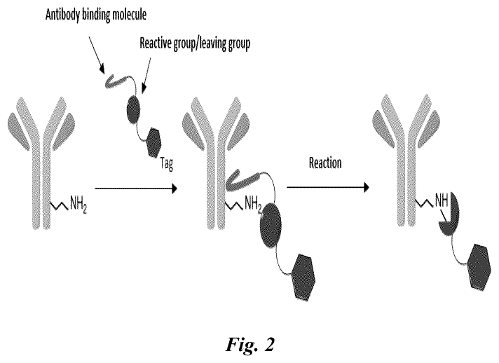

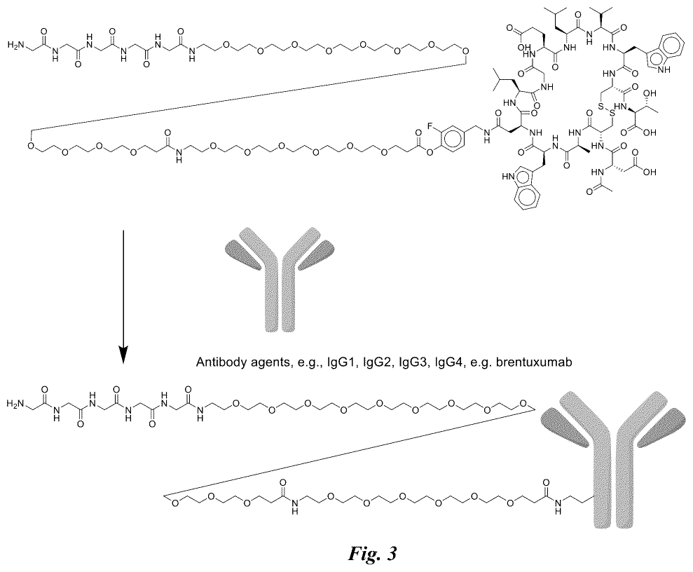

### DETAILED DESCRIPTION OF CERTAIN EMBODIMENTS

### 1. Definitions

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001.

As used herein, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the open ended transitional phrase “comprising” (and other opened ended transitional phrases such as “comprise,” “including,” and “include”) encompass and include the intermediate and closed ended phrases “consisting essentially of” and “consisting of” and unless indicated otherwise by the context a claim with an open ended phrase can be amended to have an intermediate or closed transitional phrase (v) the term “another” may be understood to mean at least an additional/second one or more; (v) the terms “about” and “approximately” may be understood to permit standard variation as would be understood by those of ordinary skill in the art; and (vi) where ranges are provided, endpoints are included. Unless otherwise specified, compounds described herein may be provided and/or utilized in a salt form, particularly a pharmaceutically acceptable salt form.

Agent: The term “agent” may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively, or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety. In some embodiments, an agent is a compound (e.g., a small molecule, a protein, a nucleic acid, etc.). In some embodiments, an agent is a mono-, bi- or polyvalent moiety of a compound (e.g., by removing one (for a monovalent moiety) or more (for a bi- or polyvalent moiety) hydrogen atoms and/or other monovalent groups from a compound).

Aliphatic: “Aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: “Alkenyl” means an aliphatic group, as defined herein, having one or more double bonds.

Alkyl: “Alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, an alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkynyl: “Alkynyl” is an aliphatic group, as defined herein, having one or more triple bonds.

Aryl: “Aryl,” used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Antibody: The term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally occurring antibodies binds to elements of the complement system, and to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present disclosure is in a format selected from, but not limited to, intact IgA, IgG, IgE or IgM antibodies; bi- or multi-specific antibodies (e.g., Zybodies®, additional bi- or multi-specific antibodies described in Ulrich Brinkmann & Roland E. Kontermann (2017) The making of bispecific antibodies, mAbs, 9:2, 182-212, doi: 10.1080/19420862.2016.1268307, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; KALBITOR®s; CovX-Bodies; and CrossMabs. In some embodiments, antibodies may have enhanced Fc domains. In some embodiments, antibodies may comprise one or more unnatural amino acid residues. In some embodiments, an antibody may lack a covalent modification (e.g., attachment of a glycan) that it would have if produced naturally. In some embodiments, an antibody is an afucosylated antibody. In some embodiments, an antibody is conjugated with another entity. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.]).

Comparable: The term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Cycloaliphatic: The term “cycloaliphatic,” “carbocycle,” “carbocyclyl,” “carbocyclic radical,” and “carbocyclic ring,” are used interchangeably, and refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Haloalkyl and Haloalkoxy: The term “haloalkyl” refers to a C₁₋₄ straight or branched alkyl group that is substituted with one or more halogen atoms, examples are trifluoromethyl, difluoromethyl, and dichloromethyl. The term “haloalkoxy” is a haloalkyl group attached to the group it substitutes via a an —O— linkage. Examples include trifluoromethoxy and difluoromethoxy.

Heteroaliphatic: The term “heteroaliphatic” is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroalkyl: The term “heteroalkyl” is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The term “heteroaryl” as used herein, also includes groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom” means an atom that is not carbon or hydrogen. In some embodiments, a heteroatom is boron, oxygen, sulfur, nitrogen, phosphorus, or silicon (including various forms of such atoms, such as oxidized forms (e.g., of nitrogen, sulfur, phosphorus, or silicon), quaternized form of a basic nitrogen or a substitutable nitrogen of a heterocyclic ring (for example, N as in 3,4-dihydro-2H-pyrrolyl), NH (as in pyrrolidinyl) or NR⁺ (as in N-substituted pyrrolidinyl) etc.). In some embodiments, a heteroatom is oxygen, sulfur or nitrogen.

Heterocycle: As used herein, the terms “heterocycle,” “heterocyclyl,” “heterocyclic radical,” and “heterocyclic ring” are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Optionally Substituted: As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents on a substitutable atom, e.g., a suitable carbon atom, are independently halogen; —(CH₂)₀₋₄Rº; —(CH₂)₀₋₄ORº; —O(CH₂)₀₋₄Rº, —O—(CH₂)₀₋₄C(O)ORº; —(CH₂)₀₋₄CH(ORº)₂; —(CH₂)₀₋₄Ph, which may be substituted with Rº; —(CH₂)₀₋₄O(CH₂)₀₋₁Ph which may be substituted with Rº; —CH═CHPh, which may be substituted with Rº; —(CH₂)₀₋₄O(CH₂)₀₋₁-pyridyl which may be substituted with Rº; —NO₂; —CN; —N₃; —(CH₂)₀₋₄N(Rº)₂; —(CH₂)₀₋₄N(Rº)C(O)Rº; —N(Rº)C(S)Rº; —(CH₂)₀₋₄N(Rº)C(O)NRº₂; —N(Rº)C(S)NRº₂; —(CH₂)₀₋₄N(Rº)C(O)ORº; —N(Rº)N(Rº)C(O)Rº; —N(Rº)N(Rº)C(O)NRº₂; —N(Rº)N(Rº)C(O)ORº; —(CH₂)₀₋₄C(O)Rº; —C(S)Rº; —(CH₂)₀₋₄C(O)ORº; —(CH₂)₀₋₄C(O)SRº; —(CH₂)₀₋₄C(O)OSiRº₃; —(CH₂)₀₋₄OC(O)Rº; —OC(O)(CH₂)₀₋₄SRº, —SC(S)SRº; —(CH₂)₀₋₄SC(O)Rº; —(CH₂)₀₋₄C(O)NRº₂; —C(S)NRº₂; —C(S)SRº; —(CH₂)₀₋₄OC(O)NRº₂; —C(O)N(ORº)Rº; —C(O)C(O)Rº; —C(O)CH₂C(O)Rº; —C(NORº)Rº; —(CH₂)₀₋₄SSRº; —(CH₂)₀₋₄S(O)₂Rº; —(CH₂)₀₋₄S(O)₂ORº; —(CH₂)₀₋₄OS(O)₂Rº; —S(O)₂NRº₂; —(CH₂)₀₋₄S(O)Rº; —N(Rº)S(O)₂NRº₂; —N(Rº)S(O)₂Rº; —N(ORº)Rº; —C(NH)NRº₂; —Si(Rº)₃; —OSi(Rº)₃; —B(Rº)₂; —OB(Rº)₂; —OB(ORº)₂; —P(Rº)₂; —P(ORº)₂; —P(Rº)(ORº); —OP(Rº)₂; —OP(ORº)₂; —OP(Rº)(ORº); —P(O)(Rº)₂; —P(O)(ORº)₂; —OP(O)(Rº)₂; —OP(O)(ORº)₂; —OP(O)(ORº)(SRº); —SP(O)(Rº)₂; —SP(O)(ORº)₂; —N(Rº)P(O)(Rº)₂; —N(Rº)P(O)(ORº)₂; —P(Rº)₂[B(Rº)₃]; —P(ORº)₂[B(Rº)₃]; —OP(Rº)₂[B(Rº)₃]; —OP(ORº)₂[B(Rº)₃]; —(C₁₋₄ straight or branched alkylene)O—N(Rº)₂; or —(C₁₋₄ straight or branched alkylene)C(O)O—N(Rº)₂, wherein each Rº may be substituted as defined herein and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of Rº, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on Rº (or the ring formed by taking two independent occurrences of Rº together with their intervening atoms), are independently halogen, —(CH₂)₀₋₂R^{●}, -(haloR^{●}), —(CH₂)₀₋₂O, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂ NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of Rº include ═O and ═S.

Suitable divalent substituents, e.g., on a suitable carbon atom, are independently the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, and aryl ring having 0-4 heteroatoms independently selected from nitrogen, oxygen, and sulfur.

Suitable substituents on the aliphatic group of R* are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen are independently —R^{†}, —NR^{†}₂, —C(O)R^{†}, —C(O)OR^{†}, —C(O)C(O)R^{†}, —C(O)CH₂C(O)R^{t}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{t}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, lists of pharmaceutically acceptable salts may be found in G. Steffen Paulekuhn, et al., Journal of Medicinal Chemistry 2007, 50, 6665 and Handbook of Pharmaceutical Salts: Properties, Selection and Use, P. Heinrich Stahl and Camille G. Wermuth Editors, Wiley-VCH, 2002.

In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups. In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations.

Protecting group: The term “protecting group” is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (lpaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), i-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (phenylxanthen-9-yl (pixyl) or 9-(p-methoxyphenyl)xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some Dbmb), 2-(isopropylthiomethoxymethyl)benzoyl (Ptmt), 9-embodiments, a phosphorous linkage protecting group is a group attached to the phosphorous linkage (e.g., an internucleotidic linkage) throughout oligonucleotide synthesis. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Subject: As used herein, the term “subject” refers to any organism to which a compound or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Therapeutic agent: The term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a compound described herein.

Therapeutically effective amount: The term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: The term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unsaturated: The term “unsaturated,” as used herein, means that a moiety has one or more units of unsaturation.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms of the compounds are within the scope of the present disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of the present disclosure. Such compounds are useful, for example, as analytical tools, as probes in biological assays, or as therapeutic agents in accordance with the present disclosure.

### 2. DESCRIPTION OF EXEMPLARY EMBODIMENTS

As described herein, in some embodiments, the present disclosure provides technologies that can conjugate moieties of interest to targets with high efficiency, high selectivity, and/or reduced side transformations (e.g., due to numbers of chemical reactions and/or conditions/types of chemical reactions). In some embodiments, the present disclosure provides useful reagents and methods for conjugation, and provide product compositions with enhanced homogeneity (e.g., 10%, 20%, 30%, 40%, 50%, 60%, 70%, 80%, 90%, 100%, or 2, 3, 4, 5, 6, 7, 8, 9, 10, 11, 12, 13, 14, 15, 16, 17, 18, 19, 20 or more fold, increase of modification/conjugation at one or more desired sites of target agents, and/or 10%, 20%, 30%. 40%, 50%, 60%, 70%, 80%, 90%, 100%, or 2, 3, 4, 5, 6, 7, 8, 9, 10, 11, 12, 13, 14, 15, 16, 17, 18, 19, 20 or more fold, decrease of modification/conjugation at one or more undesired sites of target agents), purity and/or reduced undesired modifications (e.g., to certain protein residues as results of side reactions). In some embodiments, the present disclosure provides a compound of formula R—I or a salt thereof as described herein. In some embodiments, a compound of formula R—I or a salt thereof is useful for introducing a moiety of interest to a target in one step of reaction. In some embodiments, the present disclosure provides agents of formula P—I or P-II, or a salt thereof. In some embodiments, a product composition comprise a plurality of agents having the structure of formula P—I or P-II, or a salt thereof, wherein the product composition has a higher level of homogeneity of said agents compared to a reference product composition (e.g., a product composition from a method in which a compound of formula R—I or a salt thereof is replaced with a compound which has the same structure as the compound of formula R—I or a salt thereof except that each target binding moiety is replaced with —H).

In some embodiments, the present disclosure provides a method, comprising steps of:
1) contacting a target agent, such as an antibody, with a reaction partner comprising:
   a first group comprising a target binding moiety that binds to a target agent,
   a reactive group;
   a moiety of interest that is or comprises MMAE; and
   optionally one or more linker moieties;
2) forming an agent comprising:
   a target agent moiety;
   a moiety of interest that is or comprises MMAE; and
   optionally one or more linker moieties.
      Monomethyl auristatin E (MMAE), CAS Reg. No. 474645-27-7 is a compound having the chemical formula:

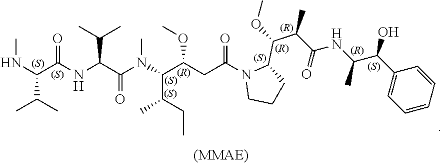

In the method of this disclosure the target binding moiety binds specifically to the target agent and the reactive group reacts with specific sites of the target agent, such as specific lysine residues of a target agent antibody, such that the agent formed by the method comprises the target agent with MMAE attached, optionally via a linker, to the specific sites.

In some embodiments, a reaction group is located between a first group and a moiety of interest, and is connected to a first group and a moiety of interest independently and optionally through a linker moiety. In some embodiments, a reaction partner is a compound of formula R—I or a salt thereof. In some embodiments, a first group is or comprises a LG group as described herein. In some embodiments, a first group is or comprises a LG group as described herein.

In some embodiments the disclosure provides a compound having the structure of formula R-l:

LG-RG-L^{RM}-MOI (R-I)

or a salt thereof, wherein:
LG is a group comprising a target binding moiety that binds to a target agent,
RG is a reactive group;
L^{RM} is a linker; and
MOI is a moiety of interest comprising monomethyl auristatin E (MMAE).

In some embodiments the disclosure provides a compound having the structure of formula R-l:

LG-RG-L^{RM}-MOI (R-I)

or a salt thereof, wherein:
LG is R^{LG}-L^{LG};
R^{LG} is

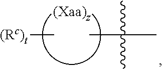

R^{c}-(Xaa)z-, a nucleic acid moiety, or a small molecule moiety;
each Xaa is independently a residue of an amino acid or an amino acid analog;
t is 0-50;
z is 1-50;
each R^{c} is independently -L^{a}-R′;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
L^{LG} is -L^{LG1}-, L^{LG1}-L^{LG2}-, -L^{LG1}-L^{LG2}-L^{LG3}- or -L^{LG1}-L^{LG2}-L^{LG3}L^{LG4}-;
RG is -L^{RG1}-L^{RG2}-, -L^{LG4}-L^{RG1}-L^{RG2}-, -L^{LG3}-L^{LG4}-L^{RG1}-L^{RG2}-, -L^{LG2}-L^{LG3}-L^{LG4}-L^{RG1}-L^{RG2}-.
each of L^{LG1}, L^{LG2}, L^{LG3}, L^{LG4}, L^{RG1}, L^{RG2}, and L^{RM} is independently L;
each L is independently a covalent bond, or a bivalent optionally substituted, linear or branched C₁₋₁₀₀ group comprising one or more aliphatic moieties, aryl moieties, heteroaliphatic moieties each independently having 1-20 heteroatoms, heteroaromatic moieties each independently having 1-20 heteroatoms, or any combinations of any one or more of such moieties, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or —[(—O—C(R′)₂—C(R′)₂—)ₙ]—, wherein n is 1-20;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms; and
MOI is a moiety of interest comprising monomethyl auristatin E (MMAE).

In some embodiments, the present disclosure provides a method comprising steps of:
1) contacting a target agent with a reaction partner having the structure of formula R—I:

LG-RG-L^{RM}-MOI (R-I)

or a salt thereof, wherein:
LG is a group comprising a target binding domain that binds to a target agent,
RG is a reactive group;
L^{RM} is a linker; and
MOI is a moiety of interest that is or comprises MMAE; and
2) forming an agent having the structure of formula P—I:

P-L^{PM}-MOI (P—I)

or a salt thereof, wherein:
P is a target agent moiety;
L^{PM} is a linker; and
MOI is a moiety of interest that is or comprises MMAE.

In some embodiments, a target agent is an antibody. In some embodiments, a target agent is an IgG antibody. For example, the antibody can be an anti-CD30 monoclonal antibody such as brentuximab, or an anti nectin-4 antibody such as enfortumab. In some embodiments, a target is a protein, and the moiety of interest is conjugated at one or more lysine residues. In some embodiments, an agent of formula P—I or a salt thereof is an agent of formula P-II or a salt thereof.

In some embodiments, the present disclosure provides a method of manufacturing an agent having the structure of P-II:

P—N-L^{PM}-MOI (P-II)
wherein:
   P—N is a protein agent moiety comprising a lysine residue;
   L^{PM} is a linker; and
   MOI is a moiety of interest;
the method comprising:
contacting P—N with a reaction partner having a structure of formula R—I:

LG-RG-L^{RM}-MOI (R-I)
or a salt thereof, wherein:
   LG is a group comprising a protein-binding domain that binds to P—N,
   RG is a reactive group;
   L^{RM} is a linker; and
   MOI is a moiety of interest that is or comprises MMAE.

In some embodiments, as exemplified herein, contacting is performed under conditions and for a time sufficient for the lysine residue N to react and form a bond with an atom of RG and release LG.

### Targets

Those skilled in the art after reading the present disclosure will appreciate that provided technologies herein are useful for conjugating various target agents to many types of moieties of interest. In some embodiments, provided technologies are particularly useful for conjugating protein agents with various moieties of interest. In some embodiments, target agents are or comprise nucleic acids.

In some embodiments, a target agent is or comprises a protein agent. In some embodiments, a target agent is a protein agent. In some embodiments, a target agent is a natural protein in a cell, tissue, organ or organism. In some embodiments, a target agent is an endogenous protein. In some embodiments, a target agent is an exogenous protein. In some embodiments, a target agent is a manufactured protein, e.g., a protein produced using various biotechnologies. In some embodiments, a target agent is an antibody agent. In some embodiments, a target agent is an antibody useful as therapeutics. Various such antibodies are known in the art and can be utilized as target agents. In some embodiments, an antibody is a monoclonal antibody. In some embodiments, an antibody is a polyclonal antibody. In some embodiments, an antibody is an IgG antibody. In some embodiments, an antibody is IVIG (in some embodiments, pooled from healthy donors). In some embodiments, a protein comprises a Fc region. In some embodiments, an antibody comprises a Fc region. In some embodiments, a Fc region comprises a single heavy chain or a fragment thereof. In some embodiments, a Fc region comprises two heavy chains or fragments thereof. In some embodiments, an antibody is a human antibody. In some embodiments, an antibody is a chimeric antibody. In some embodiments, an antibody is a humanized antibody. In some embodiments, an antibody is a mouse antibody.

In some embodiments, when characterizing polyclonal antibody agents or IVIG agents, either before, during or after conjugation, digestions are performed, e.g., enzyme digestions using IdeZ, IdeS, etc., so that certain regions of antibodies (e.g., Fab) are removed to provide compositions with improved homogeneity for characterization (e.g., by MS).

In some embodiments, an antibody is a therapeutic antibody, e.g., a FDA-approved antibody for therapeutic uses. In some embodiments, a therapeutic antibody is useful for treating cancer. In some embodiments, an antibody is adalimumab, alemtuzumab, atezolizumab, avelumab, basiliximab, brentuximab, enfortumab, ipilimumab, cetuximab, daratumumab, dinutuximab, elotuzumab, ibritumomab tiuxetan, imgatuzumab, infliximab, necitumumab, obinutuzumab, ofatumumab, pertuzumab, reslizumab, rituximab, trastuzumab, mogamulizumab, AMP-224, FS-102, GSK-2857916, ARGX-111, ARGX-110, AFM-13, APN-301, BI-836826, BI-836858, enoblituzumab, otlertuzumab, veltuzumab, KHK-4083, BIW-8962, ALT-803, carotuximab, epratuzumab, inebilizumab, isatuximab, margetuximab, MOR-208, ocaratuzumab, talacotuzumab, tremelimumab, benralizumab, lumiliximab, MOR-208, Ifibatuzumab, GSK2831781, SEA-CD40, KHK-2823, or B1836858. In some embodiments, an antibody is siltuximab, daclizumab, palivizumab, omalizumab, efalizumab, bevacizumab, natalizumab, tocilizumab, eculizumab, vedolizumab, pembrolizumab, mepolizumab, ixekizumab, panitumumab, golimumab, ustekinumab, canakinumab, denosumab, belimumab, raxibacumab, ramucirumab, nivolumab, secukinumab, evolocumab, alirocumab, brodalumab, or olaratumab. In some embodiments, an antibody is brentuximab or enfortumab. In some embodiments, an antibody is cetuximab. In some embodiments, a provided compound or agent comprising an antibody agent moiety is useful for treating a condition, disorder or disease that may be treated by the antibody agent.

Antibodies may be prepared in a number of technologies in accordance with the present disclosure. In some embodiments, antibodies may have engineered structures compared to natural immunoglobulins. In some embodiments, antibodies may comprise certain tags for purification, identification, assessment, etc. In some embodiments, antibodies may contain fragments (e.g., CDR and/or Fc, etc.) and not full immunoglobulins. Those skilled in the art appreciate that when a site of an antibody is recited in the present disclosure (e.g., K246, K248, K288, K290, K317, etc.; unless indicated otherwise, human antibody per EU numbering), an amino acid residue may not be at the exact numbered site but may be at a site that corresponds to that numbered site per, e.g., EU numbering and/or sequence homology (e.g., homologues of the same or different species).

As those skilled in the art will appreciate, provided technologies among other things can provide directed conjugation with native targets, e.g., native antibodies. In some embodiments, target agents are or comprise native antibody agents. In some embodiments, target agents are or comprise engineered antibody agents. In some embodiments, target agents, e.g., antibodies, comprise no engineered unnatural amino acid residues.

### Partner Compounds

In some embodiments, the present disclosure provides compounds each independently comprising a first group comprising a target binding moiety that binds to a target agent, a reactive group, a moiety of interest, and optionally one or more linker moieties linking such groups/moieties. In some embodiments, such a compound is useful as reaction partners for conjugating moieties of interest to targets. In some embodiments, the present disclosure provides compounds for conjugating moieties of interest to targets, e.g., various proteins. In some embodiments, provided compounds each comprise a moiety of interest, a reactive group, a target binding moiety, and optionally one or more linker moieties (linkers) linking such moieties. In some embodiments, a target binding moiety is part of a leaving group that is released upon contacting such a compound with a target and reacting a reactive group of the compound with a reactive group of a target (e.g., —NH₂ of a Lys residue of a target protein). As demonstrated herein, provided compounds among other things can provide improved conjugation efficiency, high selectivity, and fewer steps (in some cases, single step) to conjugation products. In some embodiments, a provided compound has the structure of formula R—I or a salt thereof:

LG-RG-L^{RM}-MOI (R-I)

or a salt thereof, wherein:
LG is a group comprising a target binding moiety that binds to a target agent,
RG is a reactive group;
L^{RM} is a linker; and
MOI is a moiety of interest that is or comprises MMAE.

In some embodiments, a first group is LG.

In some embodiments, LG is or comprises a target binding moiety that can bind to a target agent, and optionally a linker moiety.

As used in the present disclosure, a moiety generally refers to a part of a molecule, e.g., in an ester RCOOR′, the alcohol moiety is RO—. In some embodiments, a moiety of a compound (e.g., a target agent, a protein agent, an antibody agent, etc.) retains one or more or all desirable structural features, properties, functions, and/or activities of a compound. For example, in some embodiments, a target binding moiety can bind to a target, optionally in a comparable fashion, as its corresponding target binding compound; in some embodiments, a target agent moiety maintains one or more desired structural features, properties, functions, and/or properties comparable to its corresponding target agent compound; in some embodiments, an antibody agent moiety maintains one or more desired structural features, properties, functions, and/or properties (e.g., 3-dimension structure, antigen specificity, antigen-binding capacity, and/or immunological functions, etc.) comparable to its corresponding antibody agent compound. In some embodiments, a moiety of a compound, e.g., a target agent moiety, a protein agent moiety, an antibody agent moiety, etc. is a monovalent (for a monovalent moiety), bivalent (for a bivalent moiety), or polyvalent (for a polyvalent moiety) radical of a compound, e.g., a target agent compound (for a target agent moiety), a protein agent compound (for a protein agent moiety), an antibody agent compound (for an antibody agent moiety), etc. In some embodiments, a monovalent radical is formed by removing a monovalent part (e.g., hydrogen, halogen, another monovalent group like alkyl, aryl, etc.) from a compound. In some embodiments, a bivalent or polyvalent radical is formed by removing one or more monovalent (e.g., hydrogen, halogen, monovalent groups like alkyl, aryl, etc.), bivalent and/or polyvalent parts from a compound. In some embodiments, radicals are formed by removing hydrogen atoms. In some embodiments, a moiety is monovalent. In some embodiments, a moiety is bivalent. In some embodiments, a moiety is polyvalent.

In some embodiments, LG is or comprises R^{LG}-L^{LG}-, wherein R^{LG} is or comprises a target binding moiety, and L^{LG} is L^{LG1} as described herein. In some embodiments, L^{LG} is -L^{LG1}-L^{LG2}-, wherein each of L^{LG1} and L^{LG2} is independently as described herein. In some embodiments, L^{LG} is -L^{LG1}-L^{LG2}-L^{LG3}-, wherein each of L^{LG1}, L^{LG2} and L^{LG3} is independently as described herein. In some embodiments, L^{LG} is -L^{LG1}-L^{LG2}-L^{LG3}-L^{LG4}-, wherein each of L^{LG1}, L^{LG2}L^{LG3} and L^{LG4} is independently as described herein. In some embodiments, L^{LG1} is bonded to R^{LG}In some embodiments, L^{LG1} is bonded to moiety of interest. In some embodiments, L^{LG} is -L^{LG1}-, and a reactive group comprises L^{LG2}, L^{LG3} and L^{LG4}. In some embodiments, L^{LG} is -L^{LG1}-L^{LG2}-, and a reactive group comprises L^{LG3} and L^{LG4}. In some embodiments, L^{LG} is -L^{LG1}L^{LG2}L^{LG3}- and a reactive group comprises L^{LG4}.

In some embodiments, target binding moieties, first groups, and/or LG are released after reactions, e.g., after partner compounds react with target agents. In some embodiments, a first group is released after a reaction. In some embodiments, a target binding moiety is released after a reaction. In some embodiments, LG is released after a reaction. In some embodiments, a first group is released as part of a compound having the structure of LG-H or a salt thereof. In some embodiments, a target binding moiety is released as part of a compound having the structure of LG-H or a salt thereof. In some embodiments, LG is released as part of a compound having the structure of LG-H or a salt thereof. In some embodiments, a first group is released as part of a compound having the structure of R^{LG}-L^{LG1}-L^{LG2}-L^{LG3}-L^{LG4}-H or a salt thereof. In some embodiments, a target binding moiety is released as part of a compound having the structure of R^{LG}-L^{LG1}-L^{LG2}-L^{LG3}-L^{LG4}-H or a salt thereof. In some embodiments, a target binding moiety is released as part of a compound having the structure of R^{LG}-L^{LG1}-L^{LG2}-L^{LG3}-L^{LG4}-H or a salt thereof, wherein R^{LG} is or comprises the target binding moiety. In some embodiments, LG is released as part of a compound having the structure of R^{LG}-L^{LG1}-L^{LG2}-L^{LG3}-L^{LG4}-H or a salt thereof, wherein LG is R^{LG}-L^{LG}, and L^{LG} is -L^{LG1}-, -L^{LG1}-L^{LG2}-, -L^{LG1}-L^{LG2}-L^{LG3}-, or -L^{LG1}-L^{LG2}-L^{LG3}-L^{LG4}-. In some embodiments, LG is released as part of a compound having the structure of RG-L^{LG1}-L^{LG2}-L^{LG3}-L^{LG4}-H or a salt thereof, wherein LG is R^{LG}-L^{LG1}-. In some embodiments, LG is released as part of a compound having the structure of R^{LG}-L^{LG1}-L^{LG2}-L^{LG3}-L^{LG4}-H or a salt thereof, wherein LG is R^{LG}-L^{LG1}-L^{LG2}. In some embodiments, LG is released as part of a compound having the structure of R^{LG}-L^{LG1}-L^{LG2}-L^{LG3}-L^{LG4}-H or a salt thereof, wherein LG is R^{LG}-L^{LG1}-L^{LG2}-L^{LG3}. In some embodiments, LG is released as part of a compound having the structure of RG-L^{LG1}-L^{LG2}-L^{LG3}-L^{LG4}-H or a salt thereof, wherein LG is R^{LG}-L^{LG1}-L^{LG2}-L^{LG3}-L^{LG4}.

In some embodiments, L is a covalent bond, or a bivalent optionally substituted, linear or branched C₁₋₁₀₀ group comprising one or more aliphatic moieties, aryl moieties, heteroaliphatic moieties each independently having 1-20 heteroatoms, heteroaromatic moieties each independently having 1-20 heteroatoms, or any combinations of any one or more of such moieties, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or —[(—O—C(R′)₂—C(R′)₂—)ₙ]—, wherein n is 1-20. In some embodiments, L is a covalent bond, or a bivalent optionally substituted, linear or branched C₁₋₁₀₀ aliphatic or heteroaliphatic group 1-20 heteroatoms, wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, or —[(—O—C(R′)₂—C(R′)₂—)ₙ]—, wherein n is 1-20. In some embodiments, L is a covalent bond, or a bivalent optionally substituted, linear or branched C₁, C₂, C₃, C₄, C₅, C₁₀, C₁₅, C₂₀, C₂₅, C₃₀, C₄₀, C₅₀, C₆₀, C₁₋₂, C₁₋₅, C₁₋₁₀, C₁₋₁₅, C₁₋₂₀, C₁₋₃₀, C₁₋₄₀, C₁₋₅₀, C₁₋₆₀, C₁₋₇₀, C₁₋₈₀, or C₁₋₉₀ aliphatic or heteroaliphatic group 1-10 heteroatoms, wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, amino acid residues, or —[(—O—C(R′)₂—C(R′)₂—)ₙ]—, wherein n is 1-20. In some embodiments, L is a covalent bond, or a bivalent optionally substituted, linear or branched C₁, C₂, C₃, C₄, C₅, C₁₀, C₁₅, C₂₀, C₂₅, C₃₀, C₄₀, C₅₀, C₆₀, C₁₋₂, C₁₋₅, C₁₋₁₀, C₁₋₁₅, C₁₋₂₀, C₁₋₃₀, C_{1- 40}, C₁₋₅₀, C₁₋₆₀, C₁₋₇₀, C₁₋₈₀, or C₁₋₉₀ aliphatic or heteroaliphatic group 1-10 heteroatoms, wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, amino acid residues, or —[(—O—C(R′)₂—C(R′)₂—)ₙ]—, wherein n is 1-10. In some embodiments, L is a covalent bond, or a bivalent optionally substituted, linear or branched C₁, C₂, C₃, C₄, C₅, C₁₀, C₁₅, C₂₀, C₂₅, C₃₀, C₄₀, C₅₀, C₆₀, C₁₋₂, C₁-s, C₁₋₁₀, C₁₋₁₅, C₁₋₂₀, C₁₋₃₀, C₁₋₄₀, C₁₋₅₀, C₁₋₆₀, C₁₋₇₀, C₁₋₈₀, or C₁₋₉₀ aliphatic group, wherein one or more methylene units of the group are optionally and independently replaced with —O—, —N(R′)—, —C(O)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, amino acid residues, or —[(—O—C(R′)₂—C(R′)₂—)ₙ]—, wherein n is 1-10. In some embodiments, L is a covalent bond, or a bivalent optionally substituted, linear or branched C₁, C₂, C₃, C₄, C₅, C₁₀, C₁₅, C₂₀, C₂₅, C₃₀, C₄₀, C₅₀, C₆₀, C₁₋₂, C₁₋₅, C₁₋₁₀, C₁₋₁₅, C₁₋₂₀, C₁₋₃₀, C₁₋₄₀, C₁₋₅₀, C₁₋₆₀, C₁₋₇₀, C₁₋₈₀, or C₁₋₉₀ aliphatic group, wherein one or more methylene units of the group are optionally and independently replaced with —O—, —N(R′)—, —C(O)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, or —[(—O—C(R′)₂—C(R′)₂—)ₙ]—, wherein n is 1-10. In some embodiments, L is a covalent bond, or a bivalent optionally substituted, linear or branched C₁₋₁₀ aliphatic group, wherein one or more methylene units of the group are optionally and independently replaced with —O—, —N(R′)—, —C(O)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, -Cy-, or —[(—O—C(R′)₂—C(R′)₂—)ₙ]—, wherein n is 1-10. In some embodiments, L is a covalent bond, or a bivalent optionally substituted, linear or branched C₁₋₁₀ aliphatic group, wherein one or more methylene units of the group are optionally and independently replaced with —O—, —N(R′)—, —C(O)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, or —[(—O—C(R′)₂—C(R′)₂—)ₙ]—, wherein n is 1-10. In some embodiments, L comprises no —C(O)O—. In some embodiments, L comprises no —C(O)—N(R′)—. In some embodiments, L comprises no —S—. In some embodiments, L comprises no —S-Cy-. In some embodiments, L comprises no —S—S—. In some embodiments, L does not contain one or more or any of —C(O)O—, —C(O)—N(R′)—, —S—, and —S—S—. In some embodiments, L does not contain one or more or any of —C(O)O—, —C(O)—N(R′)—, —S-Cy-, and —S—S—. In some embodiments, L does not contain one or more or any of —C(O)O—, —S—, and —S—S—. In some embodiments, L does not contain one or more or any of —C(O)O—, —S-Cy-, and —S—S—. In some embodiments, L contains none of —C(O)O—, —S—, and —S—S—. In some embodiments, L contains none of —C(O)O—, —S-Cy-, and —S—S—. In some embodiments, L contains none of —C(O)O— and —S—S—.

In some embodiments, each amino acid residue is independently a residue of an amino acid having the structure of formula A-I or a salt thereof. In some embodiments, each amino acid residue independently has the structure of —N(R^{a1})-L^{a1}-C(R^{a2})(R^{a3})-L^{a2}-CO— or a salt form thereof. In some embodiments, each amino acid residue independently has the structure of —N(R^{a1})—C(R^{a2})(R^{a3})—CO— or a salt form thereof.

In some embodiments, L is a covalent bond. In some embodiments, L is not a covalent bond.

In some embodiments, L^{LG1} is a covalent bond. In some embodiments, L^{LG1} is not a covalent bond. In some embodiments, L^{LG1} is or comprises —(CH₂CH₂O)n-. In some embodiments, L^{LG1} is or comprises —(CH₂)n-O—(CH₂CH₂O)n-(CH₂)n-, wherein each n is independently as described herein, and each —CH₂— is independently optionally substituted. In some embodiments, L^{LG1} is —(CH₂)n-O—(CH₂CH₂O)n-(CH₂)n-, wherein each n is independently as described herein, and each —CH₂— is independently optionally substituted. In some embodiments, L^{LG1} is —(CH₂)₂—O—(CH₂CH₂O)n-(CH₂)₂—, wherein n is as described herein, and each —CH₂— is independently optionally substituted. In some embodiments, L^{LG1} is —(CH₂)₂—O—(CH₂CH₂O)n-(CH₂)₂—, wherein n is as described herein.

In some embodiments, L^{LG1} is —CH₂—. In some embodiments, L^{LG1} is —(CH₂)₂—. In some embodiments, L^{LG1} is —(CH₂)₂—C(O)—. In some embodiments, L^{LG1} is —(CH₂)₂—C(O)—NH—. In some embodiments, L^{LG1} is —(CH₂)₃—. In some embodiments, L^{LG1} is —(CH₂)₃NH—. In some embodiments, L^{LG1} is —(CH₂)₃NH—C(O)—. In some embodiments, L^{LG1} is —C(O)—(CH₂)₃NH—C(O)—. In some embodiments, L^{LG1} is —C(O)—(CH₂)₃—. In some embodiments, L^{LG1} is —NH—C(O)—(CH₂)₃—. In some embodiments, L^{LG1} is —NHC(O)—(CH₂)₃NH—C(O)—. In some embodiments, a —CH₂— is bonded to a target binding moiety.

In some embodiments, L^{LG1} is —CH₂CH₂—O—CH₂CH₂—O—CH₂CH₂—. In some embodiments, L^{LG1} is —CH₂CH₂—O—CH₂CH₂—O—CH₂CH₂—C(O)—. In some embodiments, L^{LG1} is —CH₂CH₂—O—CH₂CH₂—O—CH₂CH₂—C(O)NH—. In some embodiments, L^{LG1} is —CH₂CH₂—O—CH₂CH₂—O—CH₂CH₂—C(O)NH—CH₂—. In some embodiments, —CH₂CH₂— is bonded to a target binding moiety.

In some embodiments, L^{LG1} is —(CH₂CH₂O)n-. In some embodiments, L^{LG1} is —(CH₂CH₂O)n-CH₂—CH₂—. In some embodiments, L^{LG1} is —(CH₂CH₂O)n-CH₂—CH₂—C(O)—. In some embodiments, L^{LG1} is —(CH₂CH₂O)₂—CH₂—CH₂—C(O)—. In some embodiments, L^{LG1} is —(CH₂CH₂O)₄—CH₂—CH₂—C(O)—. In some embodiments, L^{LG1} is —(CH₂CH₂O)₈—CH₂—CH₂—C(O)—. In some embodiments, —C(O)— is bonded to a target binding moiety.

In some embodiments, L^{LG1} is —N(R′)—. In some embodiments, L^{LG1} is —NH—. In some embodiments, L^{LG1} is —NH—[(—CH₂CH₂—O—)]n-. In some embodiments, L^{LG1} is —NH—[(—CH₂CH₂—O-)]n-CH₂CH₂—. In some embodiments, L^{LG1} is —NH—[(—CH₂CH₂—O-)]n-CH₂CH₂—NH—. In some embodiments, L^{LG1} is —NH—[(—CH₂CH₂—O-)]n-CH₂CH₂—NH—C(O)—. In some embodiments, n is 1. In some embodiments, n is 2. In some embodiments, n is 3. In some embodiments, n is 4. In some embodiments, n is 5. In some embodiments, L^{LG1} is —NH—CH₂CH₂—O—. In some embodiments, L^{LG1} is —NH—CH₂CH₂—O—CH₂CH₂—. In some embodiments, L^{LG1} is —NH—CH₂CH₂—O—CH₂CH₂—NH—. In some embodiments, L^{LG1} is —NH—CH₂CH₂—O—CH₂CH₂—NH—C(O)—.

In some embodiments, L^{LG1} is —NH—[(—CH₂CH₂—O—)]₂—. In some embodiments, L^{LG1} is —NH—[(—CH₂CH₂—O—)]₂—CH₂CH₂—. In some embodiments, L^{LG1} is —NH—[(—CH₂CH₂—O—)]₂—CH₂CH₂—NH—. In some embodiments, L^{LG1} is —NH—[(—CH₂CH₂—O—)]₂—CH₂CH₂—NH—C(O)—.

In some embodiments, L^{LG1} is —NH—[(—CH₂CH₂—O—)]₃—. In some embodiments, L^{LG1} is —NH—[(—CH₂CH₂—O—)]₃—CH₂CH₂—. In some embodiments, L^{LG1} is —NH—[(—CH₂CH₂—O—)]₃—CH₂CH₂—NH—. In some embodiments, L^{LG1} is —NH—[(—CH₂CH₂—O—)]₃—CH₂CH₂—NH—C(O)—. In some embodiments, L^{LG1} is —NH—[(—CH₂CH₂—O—)]₄—. In some embodiments, L^{LG1} is —NH—[(—CH₂CH₂—O—)]₄—CH₂CH₂—. In some embodiments, L^{LG1} is —NH—[(—CH₂CH₂—O—)]₄—CH₂CH₂—NH—. In some embodiments, L^{LG1} is —NH—[(—CH₂CH₂—O—)]₄—CH₂CH₂—NH—C(O)—. In some embodiments, L^{LG1} is —NH—[(—CH₂CH₂—O—)]₅—. In some embodiments, L^{LG1} is —NH—[(—CH₂CH₂—O—)]₅—CH₂CH₂—. In some embodiments, L^{LG1} is —NH—[(—CH₂CH₂—O—)]₅—CH₂CH₂—NH—. In some embodiments, L^{LG1} is —NH—[(—CH₂CH₂—O—)]₅—CH₂CH₂—NH—C(O)—. In some embodiments, —NH— is bonded to a target binding moiety.

In some embodiments, L^{LG1} is —CH₂—. In some embodiments, L^{LG1} is —CH₂CH₂—. In some embodiments, L^{LG1} is —CH₂CH₂NH—. In some embodiments, L^{LG1} is —CH₂CH₂NH—(CO)—. In some embodiments, —CH₂— is bonded to a target binding moiety.

In some embodiments, L^{LG1} is —CH₂—. In some embodiments, L^{LG1} is —CH₂C(O)—. In some embodiments, L^{LG1} is —CH₂C(O)NH—. In some embodiments, L^{LG1} is —CH₂(CO)NHCH₂—. In some embodiments, —CH₂—C(O)— is bonded to a target binding moiety at —CH₂—.

In some embodiments, L^{LG2} is a covalent bond. In some embodiments, L^{LG2} is not a covalent bond. In some embodiments, L^{LG2} is —N(R′)C(O)—. In some embodiments, L^{LG2} is —NHC(O)—. In some embodiments, L^{LG2} is —(CH₂)n-N(R′)C(O)—, wherein —(CH₂)n- is optionally substituted. In some embodiments, L^{LG2} is —(CH₂)n-OC(O)—, wherein —(CH₂)n- is optionally substituted. In some embodiments, L^{LG2} is —(CH₂)n-OC(O)N(R′)—, wherein —(CH₂)n- is optionally substituted. In some embodiments, L^{LG2} is —(CH₂)n-OC(O)NH—, wherein —(CH₂)n- is optionally substituted. In some embodiments, n is 1-10, e.g., 1, 2, 3, 4, 5, 6, 7, 8, 9, or 10. In some embodiments, n is 1. In some embodiments, n is 2. In some embodiments, n is 3. In some embodiments, —(CH₂)n- is substituted. In some embodiments, —(CH₂)n- is unsubstituted. In some embodiments, L^{LG2} is —CH₂N(CH₂CH₂CH₂S(O)₂OH)—C(O)—. In some embodiments, L^{LG2} is —C(O)—NHCH₂—. In some embodiments, L^{LG2} is —C(O)—NHCH₂CH₂—. In some embodiments, L^{LG2} is —C(O)O—CH₂—. In some embodiments, L^{LG2} is —NH—C(O)O—CH₂—. In some embodiments, —C(O)— is bonded to L^{LG3}. In some embodiments, —N(R′)—, —NH—, or an optionally substituted —CH₂— unit (of optionally substituted —(CH₂)n-) is bonded to L^{LG3}.

In some embodiments, L^{LG2} is —N(R′)—. In some embodiments, L^{LG2} is —N(R)—. In some embodiments, L^{LG2} is —NH—.

In some embodiments, L^{LG2} is optionally substituted bivalent C₁₋₆ aliphatic. In some embodiments, L^{LG2} is —CH₂—. In some embodiments, L^{LG2} is —CH₂NH—. In some embodiments, L^{LG2} is —CH₂NH—C(O)—. In some embodiments, L^{LG2} is —CH₂NH—C(O)—CH₂—.

In some embodiments, L^{LG3} is or comprises an optionally substituted aryl ring. In some embodiments, L^{LG3} is or comprises an optionally substituted phenyl ring. In some embodiments, L^{LG3} is a phenyl ring substituted with one or more electron-withdrawing groups. As appreciated by those skilled in the art, various electron-withdrawing groups are known in the art and may be utilized in accordance with the present disclosure. In some embodiments, an electron-withdrawing group is halogen. In some embodiments, an electron-withdrawing group is —F. In some embodiments, it is —C₁. In some embodiments, it is —Br. In some embodiments, it is —I. In some embodiments, an electron-withdrawing group comprises an X═Y double bond, wherein X is bonded to the group to which the electron-withdrawing group is a substituent, and at least one of X and Y is a heteroatom. In some embodiments, X is a heteroatom. In some embodiments, Y is a heteroatom. In some embodiments, each of X and Y is independently a heteroatom. In some embodiments, Y is O. In some embodiments, Y is S. In some embodiments, X is C. In some embodiments, X is N. In some embodiments, X is P. In some embodiments, X is S. In some embodiments, X═Y is C═O. In some embodiments, X═Y is N═O. In some embodiments, X═Y is S═O. In some embodiments, X═Y is P═O. In some embodiments, an electron-withdrawing group is —C(O)-L-R′. In some embodiments, an electron-withdrawing group is —C(O)—R′. In some embodiments, it is —NO₂. In some embodiments, it is —S(O)-L-R′. In some embodiments, it is —S(O)—R′. In some embodiments, it is —S(O)₂-L-R′. In some embodiments, it is —S(O)₂—O—R′. In some embodiments, it is —S(O)₂—N(R′)₂. In some embodiments, it is —P(O)(-L-R′)₂. In some embodiments, it is —P(O)(R′)₂. In some embodiments, it is —P(O)(OR′)₂. In some embodiments, it is —P(O)[N(R′)₂]2.

In some embodiments, L^{LG3} is -L^{LG3a}-L^{LG3b}-, wherein L^{LG3a} is a covalent bond or —C(O)O—CH₂—, wherein —CH₂— is optionally substituted, and L^{LG3b} is an optionally substituted aryl ring. In some embodiments, L^{LG3a} is bonded to L^{LG2}, and L^{LG3b} is bonded to L^{LG4}.

In some embodiments, L^{LG3a} is a covalent bond. In some embodiments, L^{LG3a} is —C(O)O—CH₂—, wherein —CH₂— is optionally substituted. In some embodiments, L^{LG3a} is —C(O)O—CH₂—, wherein —CH₂— is substituted. In some embodiments, L^{LG3a} is —C(O)O—CH₂—, wherein —CH₂— is unsubstituted.

In some embodiments, a first group, a target binding moiety, and/or LG is released as part of a compound having the structure of R^{LG}-L^{LG1}-L^{LG2}-H or a salt thereof.

In some embodiments, L^{LG3b} is an optionally substituted phenyl ring. In some embodiments, at least one substituent is an electron-withdrawing group as described herein.

In some embodiments, L^{LG3} is

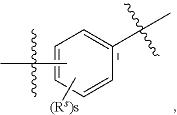

wherein s is 0-4, each R^{s} is independently halogen, —NO₂, -L-R′—C(O)-L-R′—S(O)-L-R′, —S(O)₂-L-R′, or —P(O)(-L-R′)₂. In some embodiments, C1 is bonded to L^{LG4}. In some embodiments, L^{LG3} is

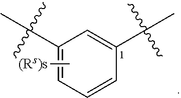

In some embodiments, L^{LG3} is

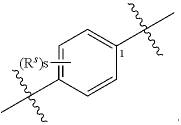

In some embodiments, L^{LG3} is

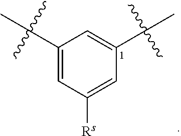

In some embodiments, L^{LG3} is

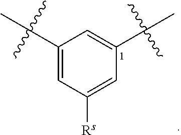

In some embodiments, L^{LG3} is

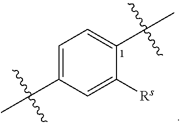

In some embodiments, L^{LG3} is

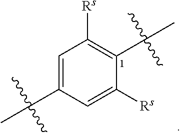

In some embodiments, L^{LG3b} is

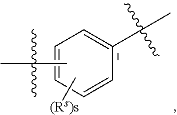

wherein s is 0-4, each R^{s} is independently halogen, —NO₂, -L-R′—C(O)-L-R′—S(O)-L-R′—S(O)₂-L-R′ or —P(O)(-L-R′)₂. In some embodiments, C1 is bonded to L^{LG4}. In some embodiments, L^{LG3b} is

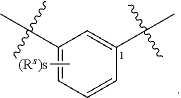

In some embodiments, L^{LG3b} is

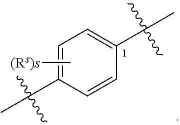

In some embodiments, L^{LG3b} is

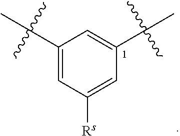

In some embodiments, L^{LG3b} is

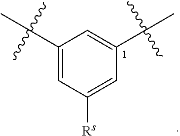

In some embodiments, L^{LG3b} is

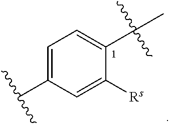

In some embodiments, L^{LG3b} is

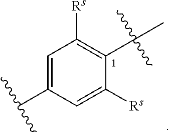

In some embodiments, s is 0. In some embodiments, s is 1-4. In some embodiments, s is 1. In some embodiments, s is 2. In some embodiments, s is 3. In some embodiments, s is 4.

In some embodiments, s is 1-4, and at least one R^{s} is an electron-withdrawing group, e.g., an electron-withdrawing group described above. In some embodiments, at least one R^{s} is —NO₂. In some embodiments, at least one R^{s} is —F. In some embodiments, each R^{s} is independently an electron-withdrawing group. In some embodiments, each R^{s} is —NO₂. In some embodiments, each R^{s} is —F.

In some embodiments, an electron-withdrawing group or R^{s} is at C₂. In some embodiments, an electron-withdrawing group or R^{s} is at C₃. In some embodiments, an electron-withdrawing group or R^{s} is at C₄. In some embodiments, an electron-withdrawing group or R^{s} is at C₂ and C₅.

In some embodiments, L^{LG3} is

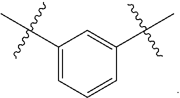 [SMILES: *c1cccc(*)c1]

In some embodiments, L^{LG3} is

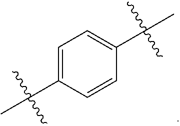 [SMILES: *c1ccc(*)cc1]

In some embodiments, L^{LG3} is

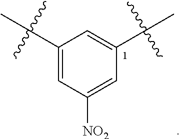 [SMILES: *c1cc(*)cc([N+](=O)[O-])c1]

In some embodiments, L^{LG3} is

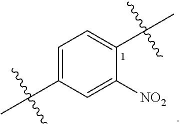 [SMILES: *c1ccc(*)c([N+](=O)[O-])c1]

In some embodiments, L^{LG3} is

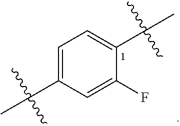 [SMILES: *c1ccc(*)c(F)c1]

In some embodiments, L^{LG3} is

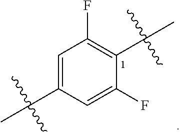 [SMILES: *c1cc(F)c(*)c(F)c1]

In some embodiments, L^{LG3} is

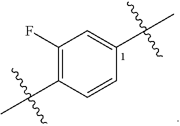 [SMILES: *c1ccc(*)c(F)c1]

In some embodiments, L^{LG3} is

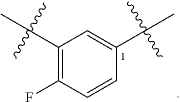 [SMILES: *c1ccc(F)c(*)c1]

In some embodiments, L^{LG3b} is

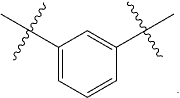 [SMILES: *c1cccc(*)c1]

In some embodiments, L^{LG3b} is

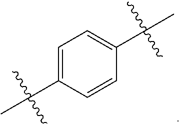 [SMILES: *c1ccc(*)cc1]

In some embodiments, L^{LG3b} is

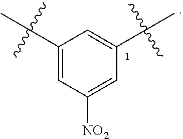 [SMILES: *c1cc(*)cc([N+](=O)[O-])c1]

In some embodiments, L^{LG3b} is

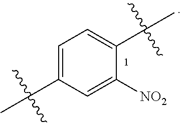 [SMILES: *c1ccc(*)c([N+](=O)[O-])c1]

In some embodiments, L^{LG3b} is

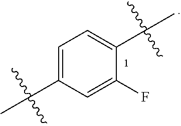 [SMILES: *c1ccc(*)c(F)c1]

In some embodiments, L^{LG3b} is

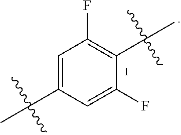 [SMILES: *c1cc(F)c(*)c(F)c1]

In some embodiments, L^{LG3b} is

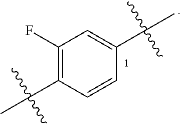 [SMILES: *c1ccc(*)c(F)c1]

In some embodiments, L^{LG3b} is

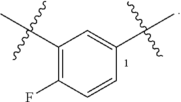 [SMILES: *c1ccc(F)c(*)c1]

In some embodiments, L^{LG3b} is optionally substituted

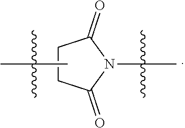

In some embodiments, the nitrogen atom is boned to L^{LG4} which is —O—. In some embodiments, the nitrogen atom is boned to L^{LG4} which is —O—, and -L^{RG1}-L^{RG2}- is —C(O)—.

In some embodiments, -L^{LG4}-L^{RG1}-L^{RG2}- is —O—C(O)—. In some embodiments, -L^{LG4}-L^{RG1}-L^{RG2}- is —S—C(O)—. In some embodiments, -L^{LG4}-L^{RG1}-L^{RG2}- is —S—C(O)—.

In some embodiments, L^{LG4} is a covalent bond. In some embodiments, L^{LG4} is not a covalent bond. In some embodiments, L^{LG4} is —O—. In some embodiments, L^{LG4} is —N(R′)—. In some embodiments, L^{LG4} is —NH—. In some embodiments, L^{LG4} is —N(CH₃)—. In some embodiments, L^{LG4} is —N(R′)—, and L^{LG3} is —O—. In some embodiments, R′ is optionally substituted C₁₋₆ alkyl. In some embodiments, L^{LG4} is —S—.

### Target Binding Moieties

As appreciated by those skilled in the art, various target binding moieties can be utilized in accordance with the present disclosure. Various technologies are also available in the art for developing and assessing target binding moieties and can be utilized in accordance with the present disclosure.

In some embodiments, a target binding moiety is or comprises a small molecule moiety. In some embodiments, a target binding moiety is or comprises a polymeric moiety. In some embodiments, a target binding moiety is or comprises nucleic acid or fragments thereof. In some embodiments, a target binding moiety is or comprises a peptide moiety. In some embodiments, a target binding moiety is a polypeptide moiety.

In some embodiments, provided technologies comprise one and no more than one target binding moiety. In some embodiments, provided technologies comprise two or more target binding moieties. For example, in some embodiments, provided compounds may comprise two or more target binding moieties that can bind to target antibody agents.

### A. Small Molecules

In some embodiments, a target binding moiety is or comprises a small molecule moiety that can selectively bind to a target agent. Small molecule binders to target agents including various protein agents are widely known in the art and can be utilized in accordance with the present disclosure. In some embodiments, a small molecule binder is or is a moiety of a therapeutic agent, e.g., a drug, an antibody-drug conjugate, etc.

In some embodiments, a target binding moiety is a small molecule moiety. In some embodiments, a small molecule moiety has a molecular weight no more than 8000, 7000, 6000, 5000, 4000, 3000, 2000, 1500, 1000, 900, 800, 700, or 600. In some embodiments, a small molecule moiety has a molecular weight no more than 8000. In some embodiments, a small molecule moiety has a molecular weight no more than 7000. In some embodiments, a small molecule moiety has a molecular weight no more than 6000. In some embodiments, a small molecule moiety has a molecular weight no more than 5000. In some embodiments, a small molecule moiety has a molecular weight no more than 4000. In some embodiments, a small molecule moiety has a molecular weight no more than 3000. In some embodiments, a small molecule moiety has a molecular weight no more than 2000. In some embodiments, a small molecule moiety has a molecular weight no more than 1500. In some embodiments, a small molecule moiety has a molecular weight no more than 1000. In some embodiments, a small molecule moiety has a molecular weight no more than 900.

b. Peptide Agents

In some embodiments, a target binding moiety is or comprises a peptide agent. In some embodiments, a target binding moiety is a peptide moiety. In some embodiments, a peptide moiety can either be linier or cyclic. In some embodiments, a target binding moiety is or comprises a cyclic peptide moiety. Various peptide target binding moieties are known in the art and can be utilized in accordance with the present disclosure.

In some embodiments, a target binding moiety is or comprises a peptide aptamer agent.

As described herein, in some embodiments, R^{LG} is or comprises a target binding moiety. In some embodiments, R^{LG} is or comprises a protein binding moiety. In some embodiments, R^{LG} is or comprises an antibody binding moiety. In some embodiments, R^{LG} is a target binding moiety. In some embodiments, R^{LG} is a protein binding moiety. In some embodiments, R^{LG} is an antibody binding moiety.

c. Aptamer Agents

In some embodiments, a target binding moiety is or comprises a nucleic acid agent. In some embodiments, a target binding moiety is or comprises an oligonucleotide moiety. In some embodiments, a target binding moiety is or comprises an aptamer agent. Various aptamer agents are known in the art or can be readily developed using common technologies, and can be utilized in provided technologies in accordance with the present disclosure.

In some embodiments, a target binding moiety is an antibody binding moiety. Such target binding moieties are, among other things, for conjugating moieties of interest to antibody agents.

### Antibody Binding Moieties

In some embodiments, targets are antibody agents. In some embodiments, target binding moieties are antibody binding moieties. In some embodiments, provided compounds and/or agents comprise antibody binding moieties. Various antibody binding moieties can be utilized in accordance with the present disclosure. In some embodiments, antibody binding moieties are universal antibody binding moieties which can bind to antibodies having different Fab regions and different specificity. Among other things, compounds comprising such antibody binding moieties may be utilized for conjugation with antibodies having different specificity. In some embodiments, antibody binding moieties of the present disclosure, e.g., universal antibody binding moieties, bind to Fc regions. In some embodiments, binding of antibody binding moieties to Fc regions can happen at the same time as binding of Fc receptors, e.g., CD16a, to the same Fc regions (e.g., may at different locations/amino acid residues of the same Fc regions). In some embodiments, upon binding of antibody binding moieties, e.g., those in provided agents, compounds, methods, etc., an Fc region can still interact with Fc receptors and perform one or more or all of its immune activities, including recruitment of immune cells (e.g., effector cells such as NK cells), and/or triggering, generating, encouraging, and/or enhancing immune system activities toward target cells, tissues, objects and/or entities, for example, antibody-dependent cell-mediated cytotoxicity (ADCC) and/or ADCP.

Various antibody binding moieties including universal antibody binding moieties can be utilized in accordance with the present disclosure. Certain antibody binding moieties and technologies for identifying and/or assessing antibody binding moieties are described in WO/2019/023501 and WO/2019/136442, and are incorporated herein by reference. Those skilled in the art appreciates that additional technologies in the art may be suitable for identifying and/or assessing antibody binding moieties in accordance with the present disclosure. In some embodiments, an antibody binding moiety comprises one or more amino acid residues, each independently natural or unnatural.

In some embodiments, a target binding moiety, e.g., a protein binding moiety (e.g., an antibody binding moiety (e.g., a universal antibody binding moiety)), has the structure of

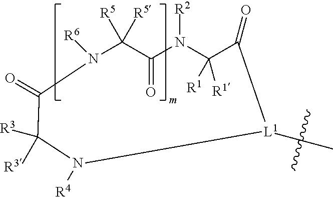

or a salt form thereof, wherein:
each of R¹, R³ and R⁵ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   R¹ and R^{1′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
   R³ and R^{3′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
   an R⁵ group and the R^{5′} group attached to the same carbon atom are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur; or two R⁵ groups are optionally taken together with their intervening atoms to form a C₁₋₁₀ optionally substituted bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂—, or -Cy¹-, wherein each -Cy¹- is independently a 5-6 membered heteroarylenyl with 1-4 heteroatoms independently selected from nitrogen, oxygen or sulfur;
each of R^{1′}, R^{3′} and R^{5′} is independently hydrogen or optionally substituted C₁₋₃ aliphatic;
each of R², R⁴ and R⁶ is independently hydrogen, or optionally substituted C₁₋₄ aliphatic, or:
   R² and R¹ are optionally taken together with their intervening atoms to form a 4-8 membered, optionally substituted saturated or partially unsaturated monocyclic heterocyclic ring having 1-2 heteroatoms independently selected from nitrogen, oxygen, or sulfur;
   R⁴ and R³ are optionally taken together with their intervening atoms to form a 4-8 membered optionally substituted saturated or partially unsaturated monocyclic heterocyclic ring having 1-2 heteroatoms independently selected from nitrogen, oxygen, or sulfur; or
   an R⁶ group and its adjacent R⁵ group are optionally taken together with their intervening atoms to form a 4-8 membered optionally substituted saturated or partially unsaturated monocyclic heterocyclic ring having 1-2 heteroatoms independently selected from nitrogen, oxygen, or sulfur;
L¹ is a trivalent linker moiety; and
each of m and n is independently 1, 2, 3, 4, 5, 6, 7, 8, 9, 10, 11, 12, 13, 14, 15, 16, 17, 18, 19, or 20.

In some embodiments, L¹ is an optionally substituted trivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, a target binding moiety, e.g. a protein binding moiety (e.g., an antibody binding moiety (e.g., a universal antibody binding moiety)), has the structure of

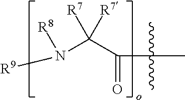

or a salt form thereof, wherein:
each of R⁷ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   an R⁷ group and the R^{7′} group attached to the same carbon atom are optionally taken together with their intervening carbon atom to form a 3-8 membered optionally substituted saturated or partially unsaturated spirocyclic carbocyclic ring or a 3-8 membered optionally substituted saturated or partially unsaturated spirocyclic heterocyclic ring having 1-2 heteroatoms independently selected from nitrogen, oxygen, or sulfur;
each of R^{7′} is independently hydrogen or optionally substituted C₁₋₃ aliphatic;
each of R¹ is independently hydrogen, or optionally substituted C₁₋₄ aliphatic, or:
   an R⁸ group and its adjacent R⁷ group are optionally taken together with their intervening atoms to form a 4-8 membered optionally substituted saturated or partially unsaturated monocyclic heterocyclic ring having 1-2 heteroatoms independently selected from nitrogen, oxygen, or sulfur; and
R⁹ is hydrogen, optionally substituted C₁₋₃ aliphatic, or —C(O)—.

In some embodiments, an antibody binding moiety, e.g., a universal antibody binding moiety is or comprises a peptide moiety, e.g., a moiety having the structure of R^{c}-(Xaa)z- or a salt form thereof, wherein each of R^{c}, z and Xaa is independently as described herein. In some embodiments, one or more Xaa are independently an unnatural amino acid residue. In some embodiments, side chains of two or more amino acid residues may be linked together to form bridges. For example, in some embodiments, side chains of two cysteine residues may form a disulfide bridge comprising —S—S— (which, as in many proteins, can be formed by two —SH groups).

In some embodiments, a target binding moiety, e.g. a protein binding moiety (e.g., an antibody binding moiety (e.g., a universal antibody binding moiety)), is or comprises a cyclic peptide moiety, e.g., a moiety having the structure of

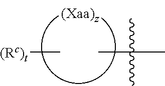

or a salt form thereof, wherein:
each Xaa is independently a residue of an amino acid or an amino acid analog;
t is 0-50;
z is 1-50;
L is a linker moiety;
each R^{c} is independently -L^{a}-R′;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆-20 aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, a heteroatom is independently selected from oxygen, nitrogen, sulfur, phosphorus and silicon.

In some embodiments, a target binding moiety is or comprises R^{c}-(Xaa)z- or a salt form thereof, wherein each variable is as described herein. In some embodiments, a protein binding moiety is or comprises R^{c}-(Xaa)z- or a salt form thereof, wherein each variable is as described herein. In some embodiments, an antibody binding moiety, e.g., a universal antibody binding moiety, is or comprises R^{c}-(Xaa)z- or a salt form thereof, wherein each variable is as described herein. In some embodiments, a target binding moiety is or comprises

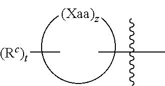

or a salt form thereof, wherein each variable is as described herein. In some embodiments, a protein binding moiety is or comprises

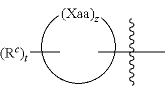

or a salt form thereof, wherein each variable is as described herein. In some embodiments, an antibody binding moiety, e.g., a universal antibody binding moiety, is or comprises

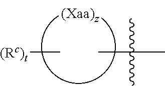

or a salt form thereof, wherein each variable is as described herein. In some embodiments, an antibody binding moiety, e.g., a universal antibody binding moiety is R^{c}-(Xaa)z- or

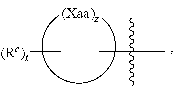

or a salt form thereof, and is or comprises a peptide unit. In some embodiments, -(Xaa)z- is or comprises a peptide unit. In some embodiments, amino acid residues may form bridges, e.g., connections formed by side chains optionally through linker moieties (e.g., L); for example, as in many polypeptides, cysteine residues may form disulfide bridges. In some embodiments, a peptide unit comprises an amino acid residue (e.g., at physiological pH about 7.4, “positively charged amino acid residue”, Xaa^{P}), e.g., a residue of an amino acid of formula A-I that has a positively charged side chain. In some embodiments, a peptide unit comprises R. In some embodiments, at least one Xaa is R. In some embodiments, a peptide unit is or comprises APAR. In some embodiments, a peptide unit is or comprises RAPA. In some embodiments, a peptide unit comprises an amino acid residue, e.g., a residue of an amino acid of formula A-I, that has a side chain comprising an aromatic group (“aromatic amino acid residue”, Xaa^{A}). In some embodiments, a peptide unit comprises a positively charged amino acid residue and an aromatic amino acid residue. In some embodiments, a peptide unit comprises W. In some embodiments, a peptide unit comprises a positively charged amino acid residue and an aromatic amino acid residue. In some embodiments, a peptide unit is or comprises Xaa^{A}XaaXaa^{P}Xaa^{P}. In some embodiments, a peptide unit is or comprises Xaa^{P}Xaa^{P}XaaXaa^{A}. In some embodiments, a peptide unit is or comprises Xaa^{P}Xaa^{A}Xaa^{P}. In some embodiments, a peptide unit is or comprises two or more Xaa^{P}Xaa^{A}Xaa^{P}. In some embodiments, a peptide unit is or comprises Xaa^{P}Xaa^{A}Xaa^{P}XaaXaa^{P}Xaa^{A}Xaa^{P}. In some embodiments, a peptide unit is or comprises Xaa^{P}Xaa^{P}Xaa^{A}Xaa^{A}Xaa^{P}. In some embodiments, a peptide unit is or comprises Xaa^{P}Xaa^{P}Xaa^{P}Xaa^{A}. In some embodiments, a peptide unit is or comprises two or more XaaAXaa^{A}Xaa^{P}. In some embodiments, a peptide residue comprises one or more proline residues. In some embodiments, a peptide unit is or comprises HWRGWA (SEQ ID NO:1). In some embodiments, a peptide unit is or comprises WGRR (SEQ ID NO:2). In some embodiments, a peptide unit is or comprises RRGW (SEQ ID NO:3). In some embodiments, a peptide unit is or comprises NKFRGKYK (SEQ ID NO:4). In some embodiments, a peptide unit is or comprises NRFRGKYK (SEQ ID NO:5). In some embodiments, a peptide unit is or comprises NARKFYK (SEQ ID NO:6). In some embodiments, a peptide unit is or comprises NARKFYKG (SEQ ID NO:7). In some embodiments, a peptide unit is or comprises HWRGWV (SEQ ID NO:8). In some embodiments, a peptide unit is or comprises KHFRNKD (SEQ ID NO:9). In some embodiments, a peptide unit comprises a positively charged amino acid residue, an aromatic amino acid residue, and an amino acid residue, e.g., a residue of an amino acid of formula A-I, that has a negatively charged side chain (e.g., at physiological pH about 7.4, “negatively charged amino acid residue”, Xaa^{N}). In some embodiments, a peptide unit comprises RHRFNKD (SEQ ID NO:10). In some embodiments, a peptide unit is RHRFNKD (SEQ ID NO:10). In some embodiments, a peptide unit comprises TY. In some embodiments, a peptide unit is TY. In some embodiments, a peptide unit comprises TYK. In some embodiments, a peptide unit is TYK. In some embodiments, a peptide unit comprises RTY. In some embodiments, a peptide unit is RTY. In some embodiments, a peptide unit comprises RTYK (SEQ ID NO:11). In some embodiments, a peptide unit is RTYK (SEQ ID NO:11). In some embodiments, a peptide unit is or comprises a sequence selected from PAM. In some embodiments, a peptide unit comprises WHL. In some embodiments, a peptide unit is WHL. In some embodiments, a peptide unit is or comprises WXL, wherein X is an amino acid residue as described herein, e.g., one suitable for connection with another moiety (e.g., an amino acid residue comprising —COOH or a salt or activated form thereof such as D, E, etc.). In some embodiments, a peptide unit comprises WDL. In some embodiments, a peptide unit is WDL. In some embodiments, a peptide unit comprises ELVW (SEQ ID NO:12). In some embodiments, a peptide unit is ELVW (SEQ ID NO:12). In some embodiments, a peptide unit comprises GELVW (SEQ ID NO:13). In some embodiments, a peptide unit is GELVW (SEQ ID NO:13). In some embodiments, a peptide unit is or comprises a sequence selected from AWHLGELVW (SEQ ID NO:14). In some embodiments, a peptide unit is or comprises AWHLGELVW (SEQ ID NO:14). In some embodiments, a peptide unit is or comprises a sequence selected from AWDLGELVW (SEQ ID NO:15). In some embodiments, a peptide unit is or comprises AWDLGELVW (SEQ ID NO:15). In some embodiments, a peptide unit is or comprises AWXLGELVW (SEQ ID NO:16), wherein X is an amino acid residue as described herein, e.g., one suitable for connection with another moiety (e.g., an amino acid residue comprising —COOH or a salt or activated form thereof such as D, E, etc.). In some embodiments, a peptide unit is or comprises a sequence selected from DCAWHLGELVWCT (SEQ ID NO:17), wherein the two cysteine residues can form a disulfide bond as found in natural proteins. In some embodiments, a peptide unit is or comprises DCAWHLGELVWCT (SEQ ID NO:17), wherein the two cysteine residues can form a disulfide bond as found in natural proteins. In some embodiments, a peptide unit is or comprises a sequence selected from DCAWXLGELVWCT (SEQ ID NO:18), wherein the two cysteine residues can form a disulfide bond as found in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises DCAWXLGELVWCT (SEQ ID NO:18), wherein the two cysteine residues can form a disulfide bond as found in natural proteins, and X is an amino acid residue as described herein, e.g., one suitable for connection with another moiety (e.g., an amino acid residue comprising —COOH or a salt or activated form thereof such as D, E, etc.). In some embodiments, X comprises —COOH or a salt or activated form thereof in its side chain. In some embodiments, a peptide unit is or comprises a sequence selected from DCAWDLGELVWCT (SEQ ID NO:19), wherein the two cysteine residues can form a disulfide bond as found in natural proteins. In some embodiments, a peptide unit is or comprises DCAWDLGELVWCT (SEQ ID NO:19), wherein the two cysteine residues can form a disulfide bond as found in natural proteins. In some embodiments, a peptide unit is or comprises a sequence selected from Fc-III. In some embodiments, a peptide unit is or comprises Fc-III. In some embodiments, a peptide unit is or comprises a sequence selected from DpLpAWXLGELVW (SEQ ID NO:20), wherein X is an amino acid residue as described herein, e.g., one suitable for connection with another moiety (e.g., an amino acid residue comprising —COOH or a salt or activated form thereof such as D, E, etc.). In some embodiments, a peptide unit is or comprises DpLpAWXLGELVW (SEQ ID NO:20), wherein X is an amino acid residue as described herein, e.g., one suitable for connection with another moiety (e.g., an amino acid residue comprising —COOH or a salt or activated form thereof such as D, E, etc.). In some embodiments, a peptide unit is or comprises a sequence selected from DpLpAWDLGELVW (SEQ ID NO:21). In some embodiments, a peptide unit is or comprises DpLpAWDLGELVW (SEQ ID NO:21). In some embodiments, a peptide unit is or comprises a sequence selected from DpLpAWHLGELVW (SEQ ID NO:22), wherein the two cysteine residues can form a disulfide bond as found in natural proteins. In some embodiments, a peptide unit is or comprises DpLpAWHLGELVW (SEQ ID NO:22) (e.g., FcBP-1), wherein the two cysteine residues can form a disulfide bond as found in natural proteins. In some embodiments, a peptide unit is or comprises a sequence selected from FcBP-1. In some embodiments, a peptide unit is or comprises a sequence selected from DpLpDCAWXLGELVWCT (SEQ ID NO:23), wherein the two cysteine residues can form a disulfide bond as found in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises DpLpDCAWXLGELVWCT (SEQ ID NO:23), wherein the two cysteine residues can form a disulfide bond as found in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises a sequence selected from DpLpDCAWHLGELVWCT (SEQ ID NO:24), wherein the two cysteine residues can form a disulfide bond as found in natural proteins. In some embodiments, a peptide unit is or comprises DpLpDCAWHLGELVWCT (SEQ ID NO:24) (e.g., FcBP-2), wherein the two cysteine residues can form a disulfide bond as found in natural proteins. In some embodiments, a peptide unit is or comprises a sequence selected from DpLpDCAWDLGELVWCT (SEQ ID NO:25), wherein the two cysteine residues can form a disulfide bond as found in natural proteins. In some embodiments, a peptide unit is or comprises DpLpDCAWDLGELVWCT (SEQ ID NO:25), wherein the two cysteine residues can form a disulfide bond as found in natural proteins. In some embodiments, a peptide unit is or comprises a sequence selected from FcBP-2. In some embodiments, a peptide unit is or comprises a sequence selected from CDCAWXLGELVWCTC (SEQ ID NO:26), wherein the first and the last cysteines, and the two cysteines in the middle of the sequence, can each independently form a disulfide bond as in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises CDCAWXLGELVWCTC (SEQ ID NO:26), wherein the first and the last cysteines, and the two cysteines in the middle of the sequence, can each independently form a disulfide bond as in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises a sequence selected from CDCAWHLGELVWCTC (SEQ ID NO:27), wherein the first and the last cysteines, and the two cysteines in the middle of the sequence, can each independently form a disulfide bond as in natural proteins. In some embodiments, a peptide unit is or comprises CDCAWHLGELVWCTC (SEQ ID NO:27), wherein the first and the last cysteines, and the two cysteines in the middle of the sequence, can each independently form a disulfide bond as in natural proteins. In some embodiments, a peptide unit is or comprises a sequence selected from CDCAWDLGELVWCTC (SEQ ID NO:28), wherein the first and the last cysteines, and the two cysteines in the middle of the sequence, can each independently form a disulfide bond as in natural proteins. In some embodiments, a peptide unit is or comprises CDCAWDLGELVWCTC (SEQ ID NO:28), wherein the first and the last cysteines, and the two cysteines in the middle of the sequence, can each independently form a disulfide bond as in natural proteins. In some embodiments, a peptide unit is or comprises a sequence selected from Fc-III-4c. In some embodiments, a peptide unit is or comprises a sequence selected from FcRM. In some embodiments, a peptide unit is or comprises a cyclic peptide unit. In some embodiments, a cyclic peptide unit comprises amide group formed by an amino group of a side chain and the C-terminus —COOH. It is appreciated by those skilled in the art that in various embodiments, when a peptide unit is connected to another moiety, an amino acid residue of a peptide unit may be connected through various positions, e.g., its backbone, its side chain, etc. In some embodiments, an amino acid residue is modified for connection. In some embodiments, an amino acid residue is replaced with another suitable residue for connection while maintaining one or more properties and/or activities a peptide unit (e.g., binding to an antibody as described herein). For example, in some embodiments, an amino acid residue is replaced with an amino acid residue with a side chain comprising —COOH or a salt or activated form thereof (e.g., side chain being —CH₂—COOH or a salt or activated form thereof). As exemplified herein, in various sequences H may be replaced with D (e.g., in various peptide units comprising WHL). In some embodiments, a peptide unit is connected to another moiety through —COOH or a salt or activated form thereof, e.g., through formation of e.g., —CON(R′)—. In some embodiments, R′ is —H. In some embodiments, —COOH is in a side chain of an amino acid residue. In some embodiments, in a sequence described herein (e.g., DCAWHLGELVWCT) SEQ ID NO:17), 1-5 (e.g., 1, 2, 3, 4, or 5) amino acid residues may be independently and optionally replaced with another amino acid residue, 1-5 (e.g., 1, 2, 3, 4, or 5) amino acid residues may be independently and optionally deleted, and/or 1-5 (e.g., 1, 2, 3, 4, or 5) amino acid residues may be independently and optionally inserted. In some embodiments, a peptide moiety is connected to the rest of a molecule through its N-terminus. In some embodiments, it is connected to the rest of a molecule through its C-terminus. In some embodiments, it is connected to the rest of a molecule through a side chain of an amino acid residue (e.g., various X residues as described in the present disclosure). In some embodiments, two cysteine residues may independently and optionally form a disulfide bond. In some embodiments, the total number of replacement, deletion and insertion is no more than 10 (e.g., 0, or no more than 1, 2, 3, 4, 5, 6, 7, 8, 9, or 10). In some embodiments, the total number is 0. In some embodiments, the total number is no more than 1. In some embodiments, the total number is no more than 2. In some embodiments, the total number is no more than 3. In some embodiments, the total number is no more than 4. In some embodiments, the total number is no more than 5. In some embodiments, the total number is no more than 6. In some embodiments, the total number is no more than 7. In some embodiments, the total number is no more than 8. In some embodiments, the total number is no more than 9. In some embodiments, the total number is no more than 10. In some embodiments, there are no insertions. In some embodiments, there are no deletions.

In some embodiments, -(Xaa)z- is or comprises [X¹]ₚ₁[X²]ₚ₂—X³X⁴X⁵X⁶X⁷X⁸X⁹X¹⁰X¹¹X¹²—[X¹³]ₚ₁₃—[X¹⁴]ₚ₁₄[X¹⁵]ₚ₁₅[X¹⁶]ₚ₁₆, wherein each of X¹, X², X³, X⁴, X⁵, X⁶, X⁷, X⁸, X⁹, X¹⁰, X¹¹, X¹², and X¹³ is independently an amino acid residue, e.g., of an amino acid of formula A-I, and each of p1, p2, p13, p14, p15 and p16 is independently 0, 1, 2, 3, 4, 5, 6, 7, 8, 9, or 10. In some embodiments, each of X¹, X², X³, X⁴, X⁵, X⁶, X⁷, X⁸, X⁹, X¹⁰, X¹¹, X¹², and X¹³ is independently an amino acid residue of an amino acid of formula A-I. In some embodiments, each of X¹, X², X³, X⁴, X⁵, X⁶, X⁷, X⁸, X⁹, X¹⁰, X¹¹, X¹², and X¹³ is independently a natural amino acid residue. In some embodiments, one or more of X¹, X², X³, X⁴, X⁵, X⁶, X⁷, X⁸, X⁹, X¹⁰, X¹¹, X¹², and X¹³ are independently an unnatural amino acid residue as described in the present disclosure.

In some embodiments, a peptide unit comprises a functional group in an amino acid residue that can react with a functional group of another amino acid residue. In some embodiments, a peptide unit comprises an amino acid residue with a side chain which comprises a functional group that can react with another functional group of the side chain of another amino acid residue to form a linkage (e.g., see moieties described in Table A-1, example 8, etc.). In some embodiments, one functional group of one amino acid residue is connected to a functional group of another amino acid residue to form a linkage (or bridge). Linkages are bonded to backbone atoms of peptide units and comprise no backbone atoms. In some embodiments, a peptide unit comprises a linkage formed by two side chains of non-neighboring amino acid residues. In some embodiments, a linkage is bonded to two backbone atoms of two non-neighboring amino acid residues. In some embodiments, both backbone atoms bonded to a linkage are carbon atoms. In some embodiments, a linkage has the structure of L^{b}, wherein L^{b} is L^{a} as described in the present disclosure, wherein L^{a} is not a covalent bond. In some embodiments, L^{a} comprises -Cy-. In some embodiments, L^{a} comprises -Cy-, wherein -Cy- is optionally substituted heteroaryl. In some embodiments, -Cy- is

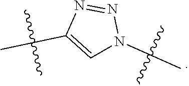 [SMILES: *c1cn(*)nn1]

In some embodiments, L^{a} is

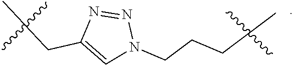 [SMILES: *CCCn1cc(C*)nn1]

In some embodiments, such an L^{a} can be formed by a —N₃ group of the side chain of one amino acid residue, and the -≡- of the side chain of another amino acid residue. In some embodiments, a linkage is formed through connection of two thiol groups, e.g., of two cysteine residues. In some embodiments, L^{a} comprises —S—S—. In some embodiments, L^{a} is —CH₂—S—S—CH₂—. In some embodiments, a linkage is formed through connection of an amino group (e.g., —NH₂ in the side chain of a lysine residue) and a carboxylic acid group (e.g., —COOH in the side chain of an aspartic acid or glutamic acid residue). In some embodiments, L^{a} comprises —C(O)—N(R′)—. In some embodiments, L^{a} comprise —C(O)—NH—. In some embodiments, L^{a} is —CH₂CONH—(CH₂)₃—. In some embodiments, L^{a} comprises —C(O)—N(R′)—, wherein R′ is R, and is taken together with an R group on the peptide backbone to form a ring (e.g., in A-34). In some embodiments, L^{a} is —(CH₂)₂—N(R′)—CO—(CH₂)₂—. In some embodiments, -Cy- is optionally substituted phenylene. In some embodiments, -Cy- is optionally substituted 1,2-phenylene. In some embodiments, L^{a} is

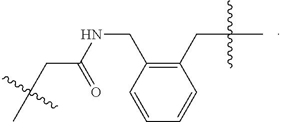 [SMILES: *CC(=O)NCc1ccccc1C*]

In some embodiments, L^{a} is

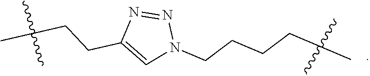 [SMILES: *CCCCn1cc(CC*)nn1]

In some embodiments, L^{a} is optionally substituted bivalent C₂₋₂₀ bivalent aliphatic. In some embodiments, L^{a} is optionally substituted —(CH₂)₉—CH═CH—(CH₂)₉—. In some embodiments, L^{a} is —(CH₂)₃—CH═CH—(CH₂)₃—.

In some embodiments, two amino acid residues bonded to a linkage are separated by 1, 2, 3, 4, 5, 6, 7, 8, 9, 10, 11, 12, 13, 14, 15, or more than 15 amino acid residues between them (excluding the two amino acid residues bonded to the linkage). In some embodiments, the number is 1. In some embodiments, the number is 2. In some embodiments, the number is 3. In some embodiments, the number is 4. In some embodiments, the number is 5. In some embodiments, the number is 6. In some embodiments, the number is 7. In some embodiments, the number is 8. In some embodiments, the number is 9. In some embodiments, the number is 10. In some embodiments, the number is 11. In some embodiments, the number is 12. In some embodiments, the number is 13. In some embodiments, the number is 14. In some embodiments, the number is 15.

In some embodiments, each of p1, p2, p13, p14, p15 and p16 is 0. In some embodiments, -(Xaa)z- is or comprises —X³X⁴X⁵X⁶X⁷X⁸X⁹X¹⁰X¹¹X¹²—, wherein:
each of X³, X⁴, X⁵, X⁶, X⁷, X⁸, X⁹, X¹⁰, X¹¹, and X¹² is independently an amino acid residue;
X⁶ is Xaa^{A} or Xaa^{P};
X⁹ is Xaa^{N}; and
X¹² is Xaa^{A} or Xaa^{P}.

In some embodiments, each of X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, X⁵ is Xaa^{A} or Xaa^{P}. In some embodiments, X⁵ is Xaa^{A}. In some embodiments, X⁵ is Xaa^{P}. In some embodiments, X⁵ is an amino acid residue whose side chain comprises an optionally substituted saturated, partially saturated or aromatic ring. In some embodiments, X⁵ is

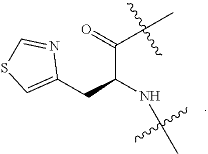 [SMILES: *N[C@@H](Cc1cscn1)C(*)=O]

In some embodiments, X⁵ is

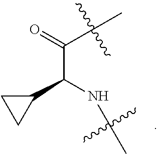 [SMILES: *N[C@H](C(*)=O)C1CC1]

In some embodiments, X⁶ is Xaa^{A}. In some embodiments, X⁶ is Xaa^{P}. In some embodiments, X⁶ is His. In some embodiments, X¹² is Xaa^{A}. In some embodiments, X¹² is Xaa^{P}. In some embodiments, X⁹ is Asp. In some embodiments, X⁹ is Glu. In some embodiments, X¹² is

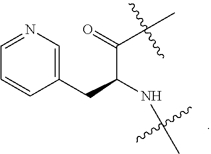 [SMILES: *N[C@@H](Cc1cccnc1)C(*)=O]

In some embodiments, X¹² is

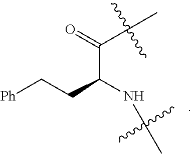 [SMILES: *N[C@@H](CCc1ccccc1)C(*)=O]

In some embodiments, each of X⁷, X¹⁰, and X¹¹ is independently an amino acid residue with a hydrophobic side chain (“hydrophobic amino acid residue”, Xaa^{H}). In some embodiments, X⁷ is Xaa^{H}. In some embodiments, X⁷ is

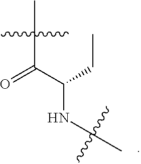 [SMILES: *N[C@@H](CC)C(*)=O]

In some embodiments, X⁷ is Val. In some embodiments, X¹⁰ is Xaa^{H}. In some embodiments, X¹⁰ is Met. In some embodiments, X¹⁰ is

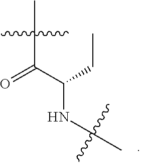 [SMILES: *N[C@@H](CC)C(*)=O]

In some embodiments, X¹¹ is Xaa^{H}. In some embodiments, X¹¹ is

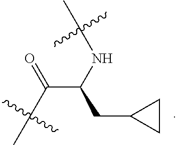 [SMILES: *N[C@@H](CC1CC1)C(*)=O]

In some embodiments, X⁸ is Gly. In some embodiments, X⁴ is Pro. In some embodiments, X³ is Lys. In some embodiments, the —COOH of X¹² forms an amide bond with the side chain amino group of Lys (X³), and the other amino group of the Lys (X³) is connected to a linker moiety and then a target binding moiety.

In some embodiments, -(Xaa)z- is or comprises —X³X⁴X⁵X⁶X⁷X⁸X⁹X¹X¹¹X¹²—, wherein:
each of X³, X⁴, X⁵, X⁶, X⁷, X⁸, X⁹, X¹⁰, X¹¹, and X¹² is independently an amino acid residue;
at least two amino acid residues are connected through one or more linkages L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁶ is Xaa^{A} or Xaa^{P};
X⁹ is Xaa^{N}; and
X¹² is Xaa^{A} or Xaa^{P}.

In some embodiments, each of X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, X⁵ and X¹⁰ are connected by L^{b}. In some embodiments, there is one linkage L^{b}. In some embodiments, X⁶ is Xaa^{A}. In some embodiments, X⁶ is Xaa^{P}. In some embodiments, X⁶ is His. In some embodiments, X⁹ is Asp. In some embodiments, X⁹ is Glu. In some embodiments, X¹² is Xaa^{A}. In some embodiments, X¹² is

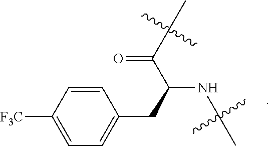 [SMILES: *N[C@@H](Cc1ccc(C(F)(F)F)cc1)C(*)=O]

In some embodiments, X¹² is

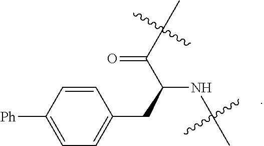 [SMILES: *N[C@@H](Cc1ccc(-c2ccccc2)cc1)C(*)=O]

In some embodiments, X¹² is

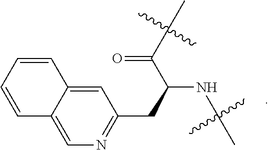 [SMILES: *N[C@@H](Cc1cc2ccccc2cn1)C(*)=O]

In some embodiments, each of X⁴, X⁷, and X¹¹ is independently Xaa^{H}. In some embodiments, X⁴ is Xaa^{H}. In some embodiments, X⁴ is Ala. In some embodiments, X⁷ is Xaa^{H}. In some embodiments, X⁷ is

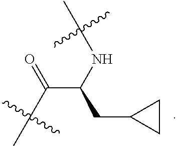 [SMILES: *N[C@@H](CC1CC1)C(*)=O]

In some embodiments, X¹¹ is Xaa^{H}. In some embodiments, X¹¹ is

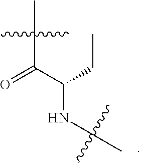 [SMILES: *N[C@@H](CC)C(*)=O]

In some embodiments, X⁸ is Gly. In some embodiments, X³ is Lys. In some embodiments, the —COOH of X¹² forms an amide bond with the side chain amino group of Lys (X³), and the other amino group of the Lys (X³) is connected to a linker moiety and then a target binding moiety. In some embodiments, L^{b} is

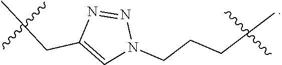 [SMILES: *CCCn1cc(C*)nn1]

In some embodiments, L^{b} is

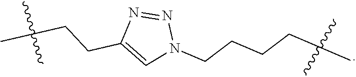 [SMILES: *CCCCn1cc(CC*)nn1]

In some embodiments, L^{b} connects two alpha-carbon atoms of two different amino acid residues. In some embodiments, both X⁵ and X¹⁰ are Cys, and the two —SH groups of their side chains form —S—S-(L^{b} is —CH₂—S—S—CH₂—).

In some embodiments, -(Xaa)z- is or comprises —X²X³X⁴X⁵X⁶X⁷X⁸X⁹X¹X¹¹X¹²—, wherein:
each of X², X³, X⁴, X⁵, X⁶, X⁷, X⁸, X⁹, X¹⁰, X¹¹, and X¹² is independently an amino acid residue;
at least two amino acid residues are connected through one or more linkages L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁴ is Xaa^{A};
X⁵ is Xaa^{A} or Xaa^{P};
X⁸ is Xaa^{N}; and
X¹¹ is Xaa^{A}.

In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, X² and X¹² are connected by L^{b}. In some embodiments, L^{b} is —CH₂—S—S—CH₂—. In some embodiments, L^{b} is —CH₂—CH₂—S—CH₂—. In some embodiments, L^{b} is

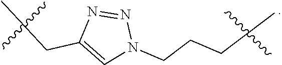 [SMILES: *CCCn1cc(C*)nn1]

In some embodiments, L^{b} is

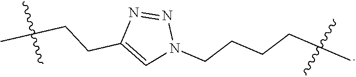 [SMILES: *CCCCn1cc(CC*)nn1]

In some embodiments, L^{b} is —CH₂CH₂CO—N(R′)—CH₂CH₂—. In some embodiments, R′ are taken together with an R group on the backbone atom that —N(R′)—CH₂CH₂— is bonded to form a ring, e.g., as in A-34. In some embodiments, a formed ring is 3-, 4-, 5-, 6-, 7- or 8-membered. In some embodiments, a formed ring is monocyclic. In some embodiments, a formed ring is saturated. In some embodiments, L^{b} is

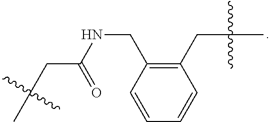 [SMILES: *CC(=O)NCc1ccccc1C*]

In some embodiments, L^{b} connects two alpha-carbon atoms of two different amino acid residues. In some embodiments, X⁴ is Xaa^{A}. In some embodiments, X⁴ is Tyr. In some embodiments, X⁵ is Xaa^{A}. In some embodiments, X⁵ is Xaa^{P}. In some embodiments, X⁵ is His. In some embodiments, X⁸ is Asp. In some embodiments, X⁸ is Glu. X¹¹ is Tyr. In some embodiments, both X² and X¹² are Cys, and the two —SH groups of their side chains form —S—S-(L^{b} is —CH₂—S—S—CH₂—). In some embodiments, each of X³, X⁶, X⁹, and X¹⁰ is independently Xaa^{H}. In some embodiments, X³ is Xaa^{H}. In some embodiments, X³ is Ala. In some embodiments, X⁶ is Xaa^{H}. In some embodiments, X⁶ is Leu. In some embodiments, X⁹ is Xaa^{H}. In some embodiments, X⁹ is Leu. In some embodiments, X⁹ is

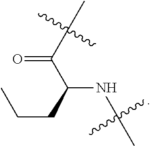 [SMILES: *N[C@@H](CCC)C(*)=O]

In some embodiments, X¹⁰ is Xaa^{H}. In some embodiments, X¹⁰ is Val. In some embodiments, X¹⁰ is

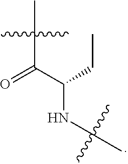 [SMILES: *N[C@@H](CC)C(*)=O]

In some embodiments, X⁷ is Gly. In some embodiments, p1 is 1. In some embodiments, X¹ is Asp. In some embodiments, p13 is 1. In some embodiments, p14, p15 and p16 are 0. In some embodiments, X¹³ is an amino acid residue comprising a polar uncharged side chain (e.g., at physiological pH, “polar uncharged amino acid residue”, Xaa^{L}). In some embodiments, X¹³ is Thr. In some embodiments, X¹³ is Val. In some embodiments, p13 is 0. In some embodiments, R^{c} is —NHCH₂CH(OH)CH₃. In some embodiments, R^{c} is (R)—NHCH₂CH(OH)CH₃. In some embodiments, R^{c} is (S)—NHCH₂CH(OH)CH₃.

In some embodiments, -(Xaa)z- is or comprises —X²X³X⁴X⁵X⁶X⁷X⁸X⁹X¹X¹¹X¹²—, wherein:
each of X², X³, X⁴, X⁵, X⁶, X⁷, X⁸, X⁹, X¹⁰, X¹¹, and X¹² is independently an amino acid residue;
at least two amino acid residues are connected through one or more linkages L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁵ is Xaa^{A} or Xaa^{P};
X⁸ is Xaa^{N}; and
X¹¹ is Xaa^{A}.

In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, there are two or more linkages L^{b}. In some embodiments, there are two linkages L^{b}. In some embodiments, X² and X¹² are connected by L^{b}. In some embodiments, X⁴ and X⁹ are connected by L^{b}. In some embodiments, X⁴ and X¹⁰ are connected by L^{b}. In some embodiments, L^{b} is —CH₂—S—S—CH₂—. In some embodiments, L^{b} is

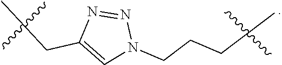 [SMILES: *CCCn1cc(C*)nn1]

In some embodiments, L^{b} is

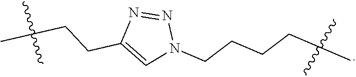 [SMILES: *CCCCn1cc(CC*)nn1]

In some embodiments, both X² and X¹² are Cys, and the two —SH groups of their side chains form —S—S-(L^{b} is —CH₂—S—S—CH₂—). In some embodiments, both X⁴ and X¹⁰ are Cys, and the two —SH groups of their side chains form —S—S-(L^{b} is —CH₂—S—S—CH₂—). In some embodiments, X⁴ and X⁹ are connected by L^{b}, wherein L^{b} is

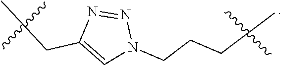 [SMILES: *CCCn1cc(C*)nn1]

In some embodiments, X⁴ and X⁹ are connected by L^{b}, wherein L^{b} is

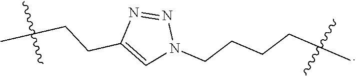 [SMILES: *CCCCn1cc(CC*)nn1]

In some embodiments, X⁵ is Xaa^{A}. In some embodiments, X⁵ is Xaa^{P}. In some embodiments, X⁵ is His. In some embodiments, X⁸ is Asp. In some embodiments, X⁸ is Glu. In some embodiments, X¹¹ is Tyr. In some embodiments, X¹¹ is

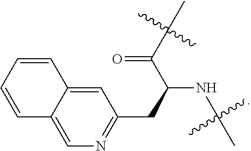 [SMILES: *N[C@@H](Cc1cc2ccccc2cn1)C(*)=O]

In some embodiments, X² and X¹² are connected by L^{b}, wherein L^{b} is —CH₂—S—CH₂CH₂—. In some embodiments, L^{b} connects two alpha-carbon atoms of two different amino acid residues. In some embodiments, each of X³, X⁶, and X⁹ is independently Xaa^{H}. In some embodiments, X³ is Xaa^{H}. In some embodiments, X³ is Ala. In some embodiments, X⁶ is Xaa^{H}. In some embodiments, X⁶ is Leu. In some embodiments, X⁶ is

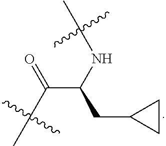 [SMILES: *N[C@@H](CC1CC1)C(*)=O]

In some embodiments, X⁹ is Xaa^{H}. In some embodiments, X⁹ is Leu. In some embodiments, X⁹ is

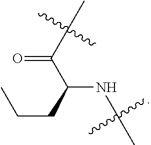 [SMILES: *N[C@@H](CCC)C(*)=O]

In some embodiments, X¹⁰ is Xaa^{H}. In some embodiments, X¹⁰ is Val. In some embodiments, X⁷ is Gly. In some embodiments, p1 is 1. In some embodiments, X¹ is Xaa^{N}. In some embodiments, X¹ is Asp. In some embodiments, X¹ is Glu. In some embodiments, p13 is 1. In some embodiments, p14, p15 and p16 are 0. In some embodiments, X¹³ is Xaa^{L}. In some embodiments, X¹³ is Thr. In some embodiments, X¹³ is Val.

In some embodiments, -(Xaa)z- is or comprises —X²X³X⁴X⁵X⁶X⁷X⁸X⁹X¹⁰X¹¹X¹²X¹³X¹⁴X¹⁵X¹⁶—, wherein:
each of X², X³, X⁴, X⁵, X⁶, X⁷, X⁸, X⁹, X¹⁰, X¹¹, X¹², X¹³, X¹⁴, X¹⁵, and X¹⁶ is independently an amino acid residue;
at least two amino acid residues are connected through a linkage L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X³ is Xaa^{N};
X⁶ is Xaa^{A};
X⁷ is Xaa^{A} or Xaa^{P};
X⁹ is Xaa^{N}; and
X¹³ is Xaa^{A}.

In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. As appreciated by those skilled in the art, an amino acid residue may be replaced by another amino acid residue having similar properties, e.g., one Xaa^{H} (e.g., Val, Leu, etc.) may be replaced with another Xaa^{H} (e.g., Leu, lie, Ala, etc.), one Xaa^{A} may be replaced with another Xaa^{A}, one Xaa^{P} may be replaced with another Xaa^{P}, one Xaa^{N} may be replaced with another Xaa^{N}, one Xaa^{L} may be replaced with another Xaa^{L}, etc.

In some embodiments, a target binding moiety is or comprises optionally substituted moiety of Table A-1. In some embodiments, a protein binding moiety is or comprises optionally substituted moiety of Table A-1. In some embodiments, an antibody binding moiety, e.g., a universal antibody binding moiety, is or comprises optionally substituted moiety of Table A-1. In some embodiments, a target binding moiety is selected from able A-1. In some embodiments, a protein binding moiety is selected from able A-1. In some embodiments, an antibody binding moiety, e.g., a universal antibody binding moiety, is selected from able A-1. In some embodiments, C-terminus and/or N-terminus are optionally capped (e.g., for C-terminus, by converting —COOH into —C(O)N(R′)₂ like —C(O)NH₂; for N-terminus, by adding R′ C(O)— like CH₃C(O)— to an amino group).

TABLE A-1
Exemplary antibody binding moieties.

| | |
|---|---|
| 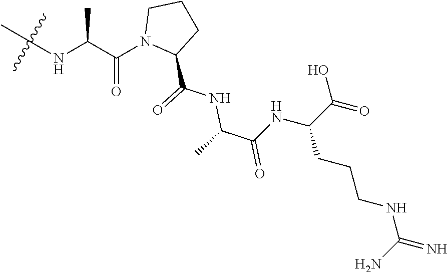 [SMILES: *N[C@@H](C)C(=O)N1CCC[C@H]1C(=O)N[C@@H](C)C(=O)N[C@@H](CCCNC(=N)N)C(=O)O] | A-1 |
| 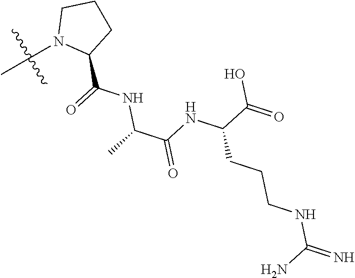 [SMILES: *N1CCC[C@H]1C(=O)N[C@@H](C)C(=O)N[C@@H](CCCNC(=N)N)C(=O)O] | A-2 |
| 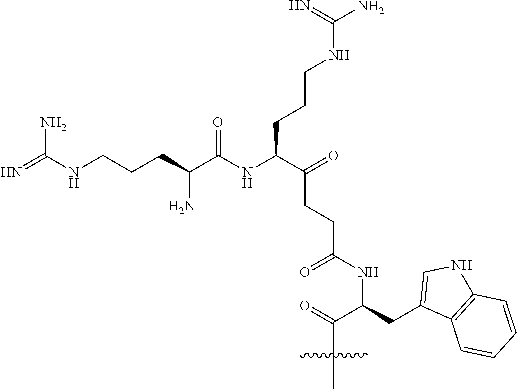 [SMILES: *C(=O)[C@H](Cc1c[nH]c2ccccc12)NC(=O)CCC(=O)[C@H](CCCNC(=N)N)NC(=O)[C@@H](N)CCCNC(=N)N] | A-3 |
| 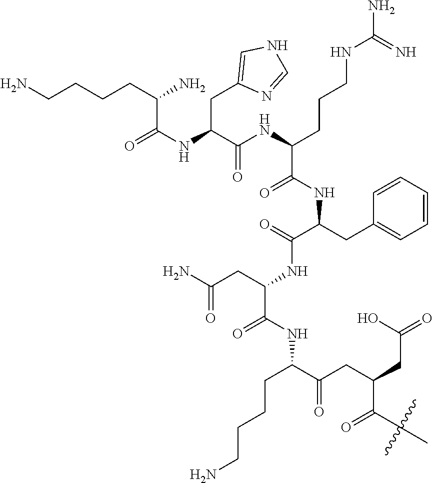 [SMILES: *C(=O)[C@H](CC(=O)O)CC(=O)[C@H](CCCCN)NC(=O)[C@H](CC(N)=O)NC(=O)[C@H](Cc1ccccc1)NC(=O)[C@H](CCCNC(=N)N)NC(=O)[C@H](Cc1c[nH]cn1)NC(=O)[C@@H](N)CCCCN] | A-4 |
| 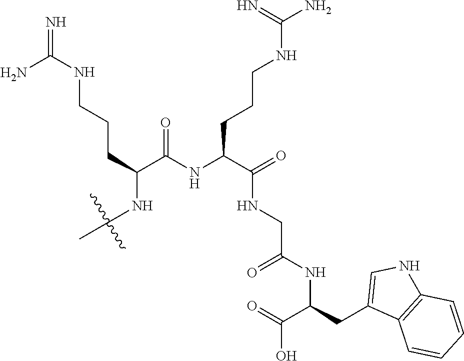 [SMILES: *N[C@@H](CCCNC(=N)N)C(=O)N[C@@H](CCCNC(=N)N)C(=O)NCC(=O)N[C@@H](Cc1c[nH]c2ccccc12)C(=O)O] | A-5 |
| 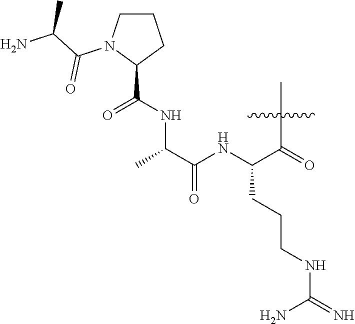 [SMILES: *C(=O)[C@H](CCCNC(=N)N)NC(=O)[C@H](C)NC(=O)[C@@H]1CCCN1C(=O)[C@H](C)N] | A-6 |
| 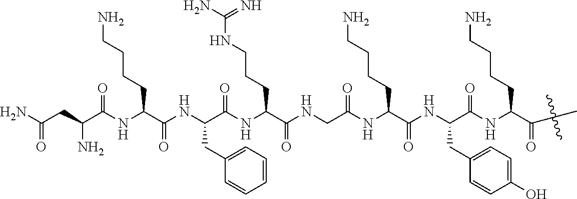 [SMILES: *C(=O)[C@H](CCCCN)NC(=O)[C@H](Cc1ccc(O)cc1)NC(=O)[C@H](CCCCN)NC(=O)CNC(=O)[C@H](CCCNC(=N)N)NC(=O)[C@H](Cc1ccccc1)NC(=O)[C@H](CCCCN)NC(=O)[C@@H](N)CC(N)=O] | A-7 |
| 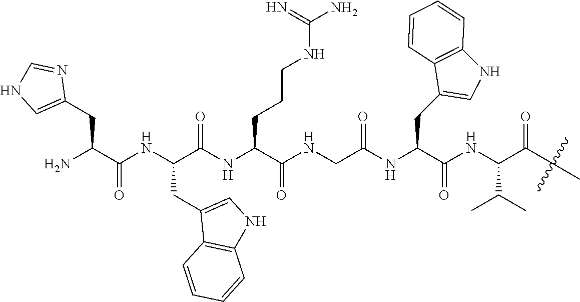 [SMILES: *C(=O)[C@@H](NC(=O)[C@H](Cc1c[nH]c2ccccc12)NC(=O)CNC(=O)[C@H](CCCNC(=N)N)NC(=O)[C@H](Cc1c[nH]c2ccccc12)NC(=O)[C@@H](N)Cc1c[nH]cn1)C(C)C] | A-8 |
| 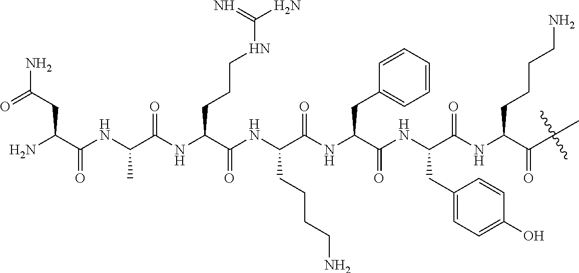 [SMILES: *C(=O)[C@H](CCCCN)NC(=O)[C@H](Cc1ccc(O)cc1)NC(=O)[C@H](Cc1ccccc1)NC(=O)[C@H](CCCCN)NC(=O)[C@H](CCCNC(=N)N)NC(=O)[C@H](C)NC(=O)[C@@H](N)CC(N)=O] | A-9 |
| 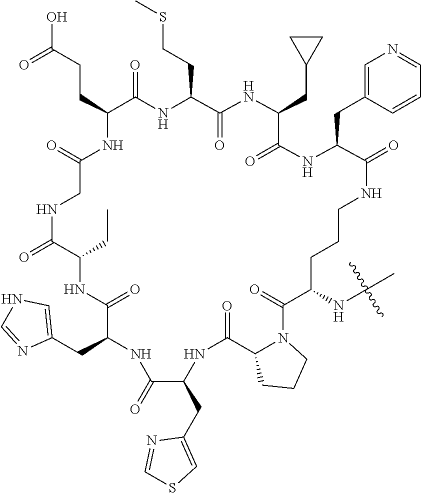 [SMILES: *N[C@H]1CCCNC(=O)[C@H](Cc2cccnc2)NC(=O)[C@H](CC2CC2)NC(=O)[C@H](CCSC)NC(=O)[C@H](CCC(=O)O)NC(=O)CNC(=O)[C@H](CC)NC(=O)[C@H](Cc2c[nH]cn2)NC(=O)[C@H](Cc2cscn2)NC(=O)[C@H]2CCCN2C1=O] | A-10 |
| 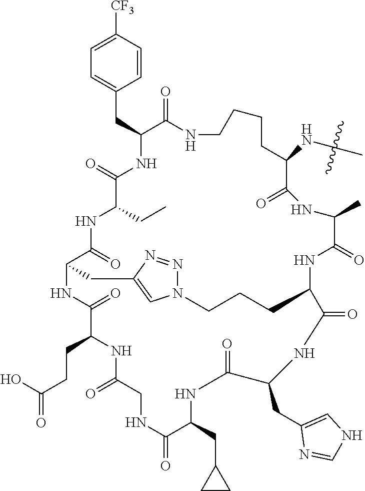 [SMILES: *N[C@@H]1CCCCNC(=O)[C@H](Cc2ccc(C(F)(F)F)cc2)NC(=O)[C@H](CC)NC(=O)[C@H]2Cc3cn(nn3)CCC[C@@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-11 |
| 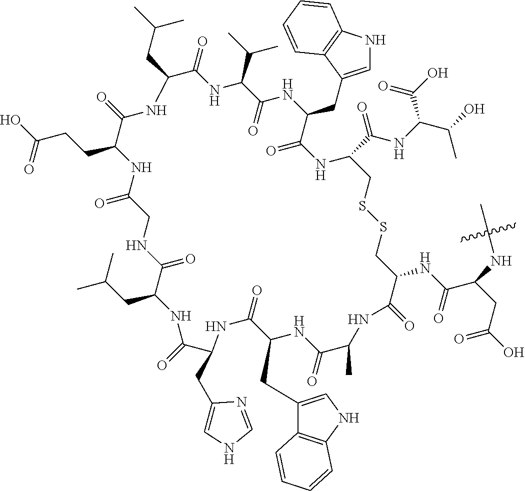 [SMILES: *N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-12 |
| 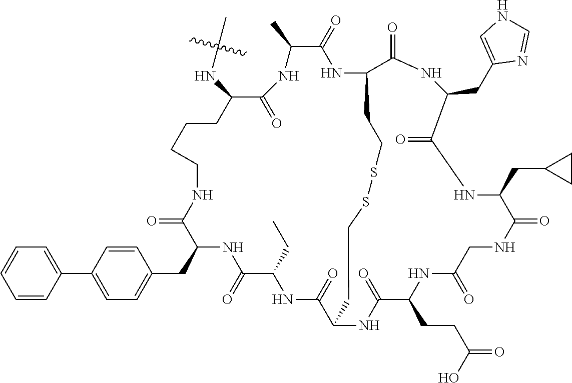 [SMILES: *N[C@@H]1CCCCNC(=O)[C@H](Cc2ccc(-c3ccccc3)cc2)NC(=O)[C@H](CC)NC(=O)[C@H]2CCSSCC[C@@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-13 |
| 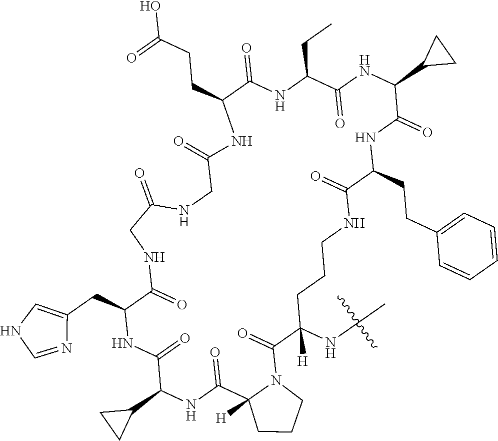 [SMILES: *N[C@H]1CCCNC(=O)[C@H](CCc2ccccc2)NC(=O)[C@H](C2CC2)NC(=O)[C@H](CC)NC(=O)[C@H](CCC(=O)O)NC(=O)CNC(=O)CNC(=O)[C@H](Cc2c[nH]cn2)NC(=O)[C@H](C2CC2)NC(=O)[C@H]2CCCN2C1=O] | A-14 |
| 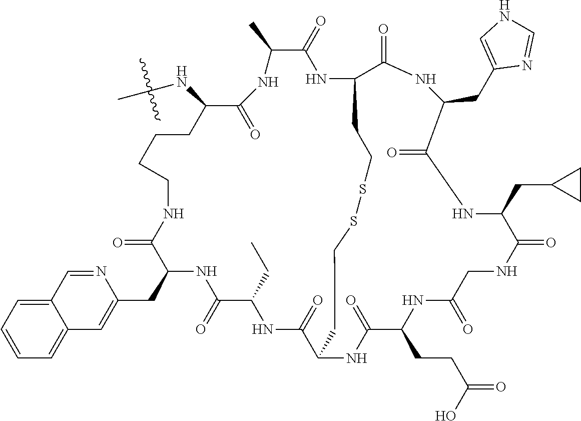 [SMILES: *N[C@@H]1CCCCNC(=O)[C@H](Cc2cc3ccccc3cn2)NC(=O)[C@H](CC)NC(=O)[C@H]2CCSSCC[C@@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-15 |
| 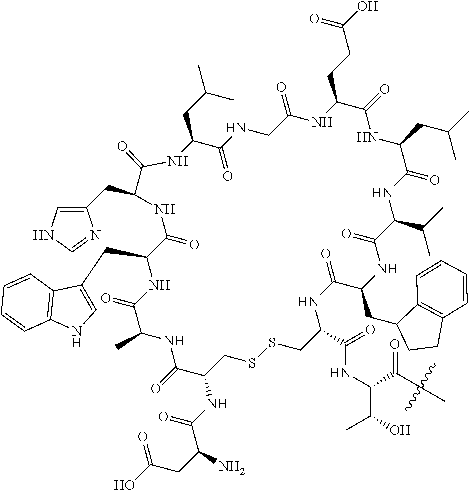 [SMILES: *C(=O)[C@@H](NC(=O)[C@@H]1CSSC[C@H](NC(=O)[C@@H](N)CC(=O)O)C(=O)N[C@@H](C)C(=O)N[C@@H](Cc2c[nH]c3ccccc23)C(=O)N[C@@H](Cc2c[nH]cn2)C(=O)N[C@@H](CC(C)C)C(=O)NCC(=O)N[C@@H](CCC(=O)O)C(=O)N[C@@H](CC(C)C)C(=O)N[C@@H](C(C)C)C(=O)N[C@@H](CC2CCc3ccccc32)C(=O)N1)[C@@H](C)O] | A-16 |
| 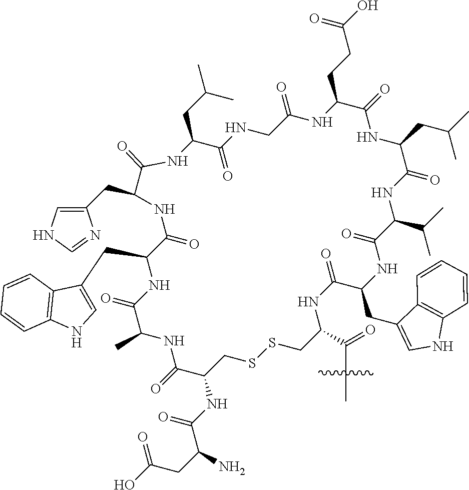 [SMILES: *C(=O)[C@@H]1CSSC[C@H](NC(=O)[C@@H](N)CC(=O)O)C(=O)N[C@@H](C)C(=O)N[C@@H](Cc2c[nH]c3ccccc23)C(=O)N[C@@H](Cc2c[nH]cn2)C(=O)N[C@@H](CC(C)C)C(=O)NCC(=O)N[C@@H](CCC(=O)O)C(=O)N[C@@H](CC(C)C)C(=O)N[C@@H](C(C)C)C(=O)N[C@@H](Cc2c[nH]c3ccccc23)C(=O)N1] | A-17 |
| 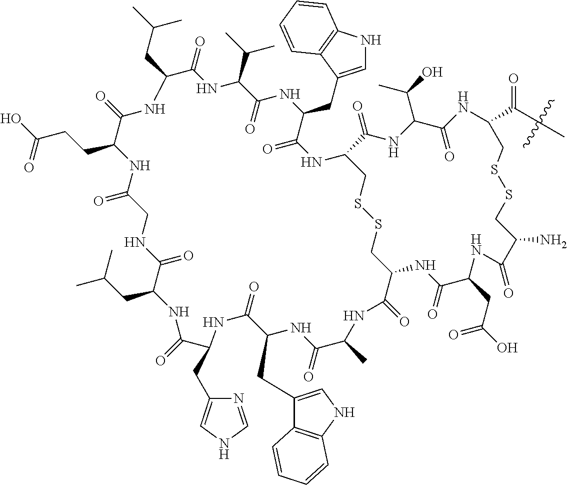 [SMILES: *C(=O)[C@@H]1CSSC[C@H](N)C(=O)N[C@@H](CC(=O)O)C(=O)N[C@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc3c[nH]cn3)NC(=O)[C@H](Cc3c[nH]c4ccccc34)NC(=O)[C@H](C)NC2=O)C(=O)NC([C@@H](C)O)C(=O)N1] | A-18 |
| 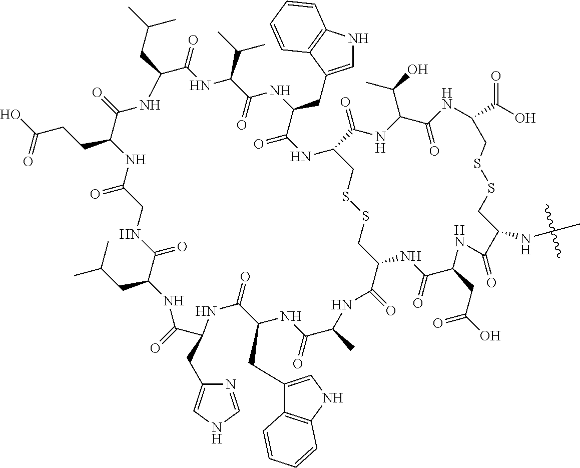 [SMILES: *N[C@H]1CSSC[C@@H](C(=O)O)NC(=O)C([C@@H](C)O)NC(=O)[C@@H]2CSSC[C@H](NC(=O)[C@H](CC(=O)O)NC1=O)C(=O)N[C@@H](C)C(=O)N[C@@H](Cc1c[nH]c3ccccc13)C(=O)N[C@@H](Cc1c[nH]cn1)C(=O)N[C@@H](CC(C)C)C(=O)NCC(=O)N[C@@H](CCC(=O)O)C(=O)N[C@@H](CC(C)C)C(=O)N[C@@H](C(C)C)C(=O)N[C@@H](Cc1c[nH]c3ccccc13)C(=O)N2] | A-19 |
| 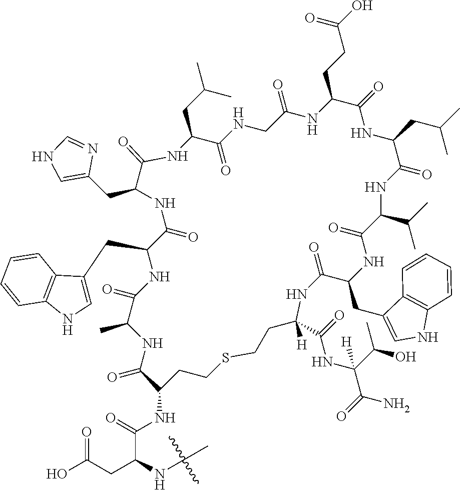 | A-20 |
| 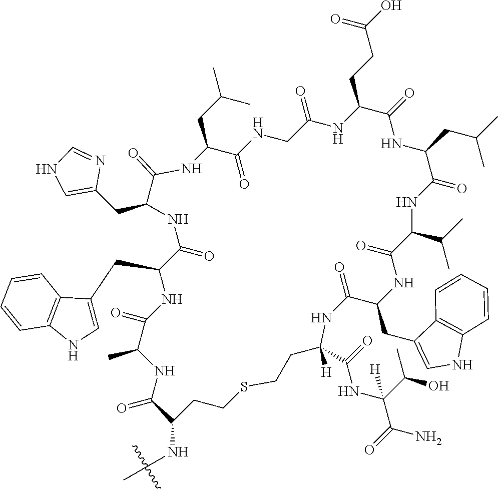 | A-21 |
| 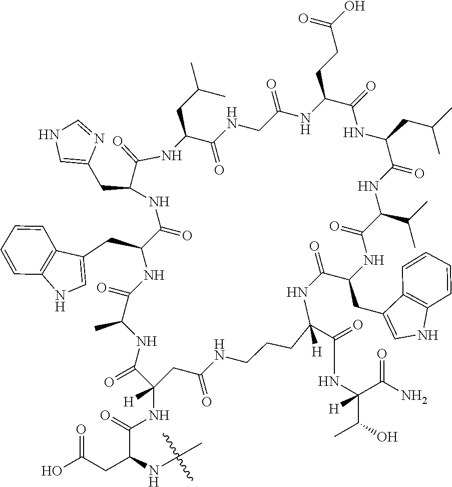 [SMILES: *N[C@@H](CC(=O)O)C(=O)N[C@H]1CC(=O)NCCC[C@@H](C(=O)N[C@H](C(N)=O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-22 |
| 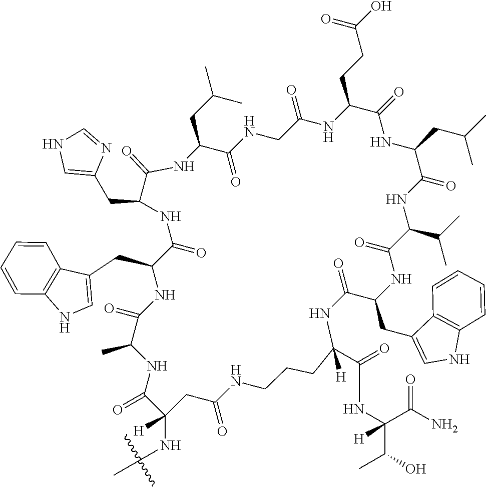 [SMILES: *N[C@H]1CC(=O)NCCC[C@@H](C(=O)N[C@H](C(N)=O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-23 |
| 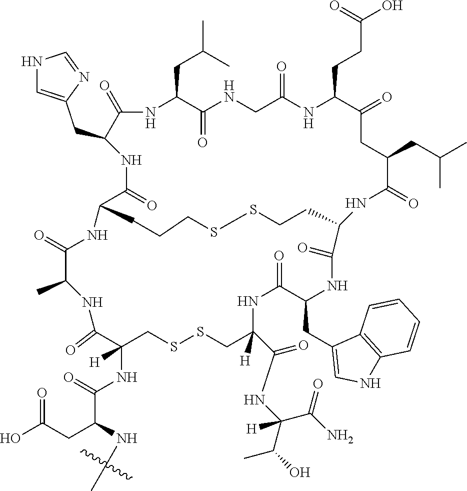 [SMILES: *N[C@@H](CC(=O)O)C(=O)N[C@H]1CSSC[C@@H](C(=O)N[C@H](C(N)=O)[C@@H](C)O)NC(=O)[C@H](Cc2c[nH]c3ccccc23)NC(=O)[C@@H]2CCSSCCC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)C[C@@H](CC(C)C)C(=O)N2] | A-24 |
| 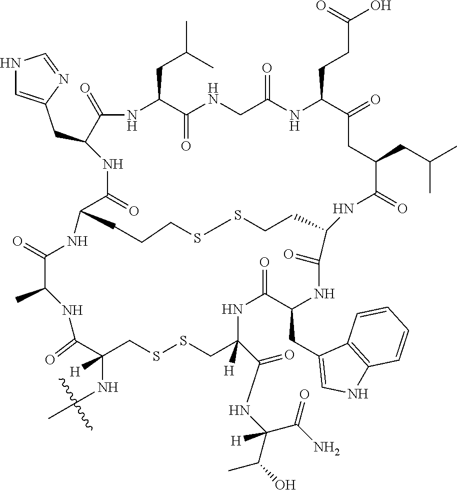 [SMILES: *N[C@H]1CSSC[C@@H](C(=O)N[C@H](C(N)=O)[C@@H](C)O)NC(=O)[C@H](Cc2c[nH]c3ccccc23)NC(=O)[C@@H]2CCSSCCC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)C[C@@H](CC(C)C)C(=O)N2] | A-25 |
| 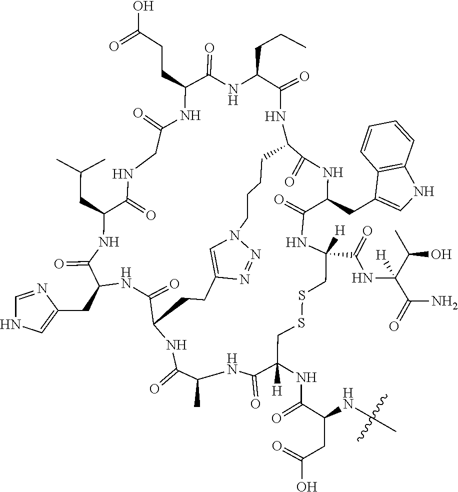 [SMILES: *N[C@@H](CC(=O)O)C(=O)N[C@H]1CSSC[C@@H](C(=O)N[C@H](C(N)=O)[C@@H](C)O)NC(=O)[C@H](Cc2c[nH]c3ccccc23)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CCC)C(=O)N2] | A-26 |
| 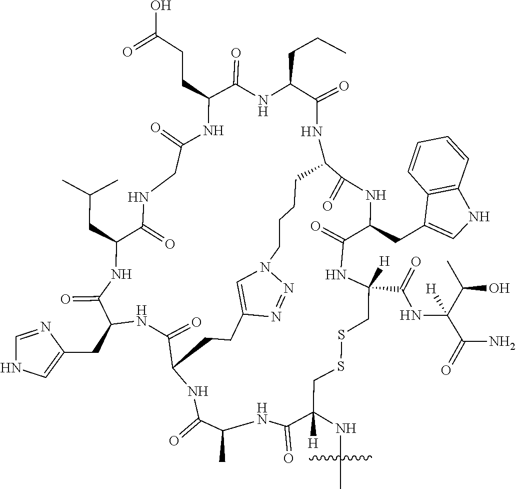 [SMILES: *N[C@H]1CSSC[C@@H](C(=O)N[C@H](C(N)=O)[C@@H](C)O)NC(=O)[C@H](Cc2c[nH]c3ccccc23)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CCC)C(=O)N2] | A-27 |
| 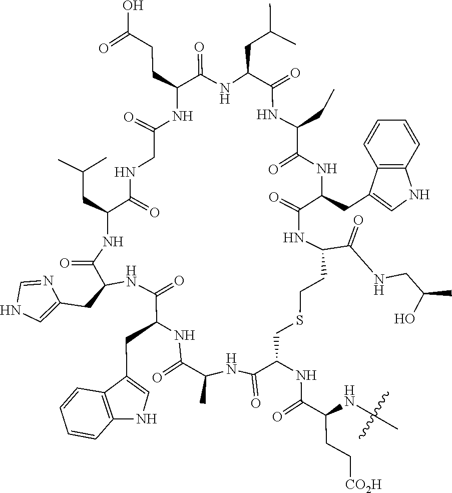 [SMILES: *N[C@@H](CCC(=O)O)C(=O)N[C@H]1CSCC[C@@H](C(=O)NC[C@@H](C)O)NC(=O)[C@H](Cc2c[nH]c3ccccc23)NC(=O)[C@H](CC)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-28 |
| 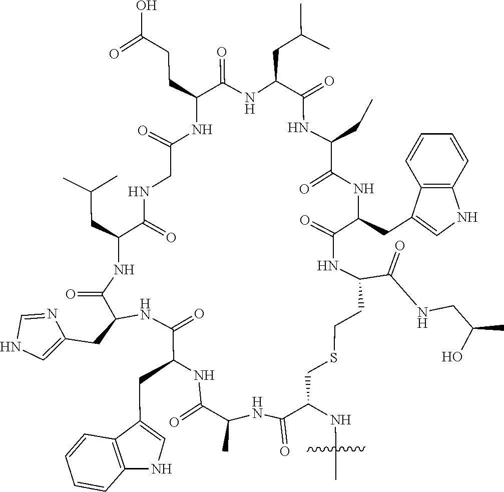 [SMILES: *N[C@H]1CSCC[C@@H](C(=O)NC[C@@H](C)O)NC(=O)[C@H](Cc2c[nH]c3ccccc23)NC(=O)[C@H](CC)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-29 |
| 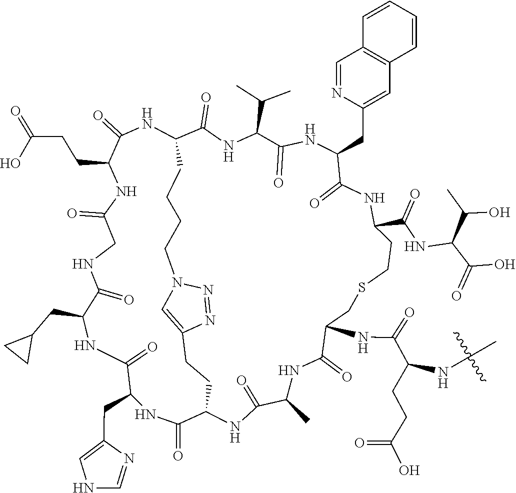 [SMILES: *N[C@@H](CCC(=O)O)C(=O)N[C@H]1CSCC[C@@H](C(=O)N[C@H](C(=O)O)C(C)O)NC(=O)[C@H](Cc2cc3ccccc3cn2)NC(=O)[C@H](C(C)C)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-30 |
| 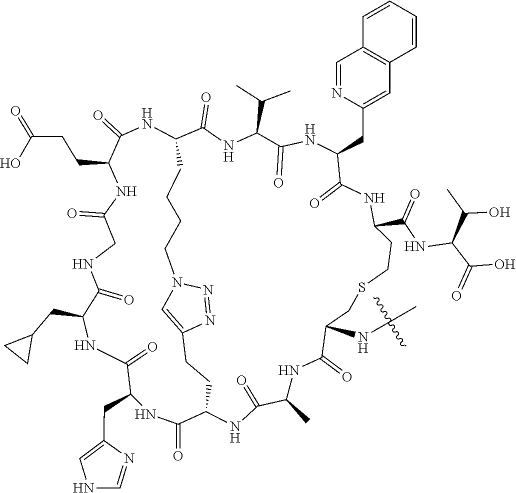 [SMILES: *N[C@H]1CSCC[C@@H](C(=O)N[C@H](C(=O)O)C(C)O)NC(=O)[C@H](Cc2cc3ccccc3cn2)NC(=O)[C@H](C(C)C)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-31 |
| 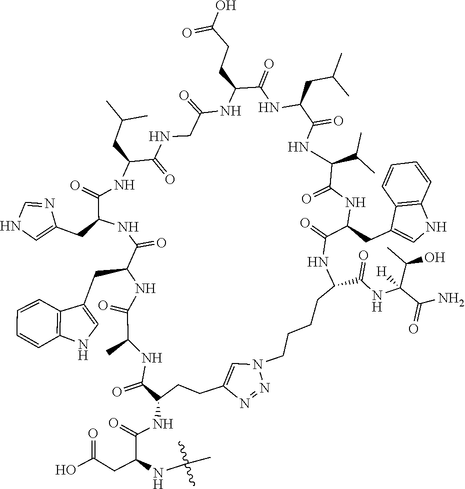 | A-32 |
| 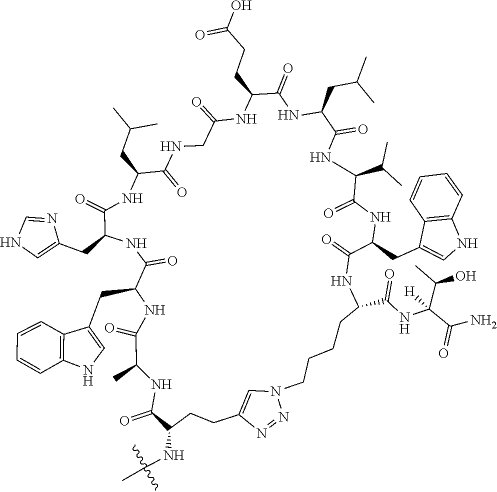 | A-33 |
| 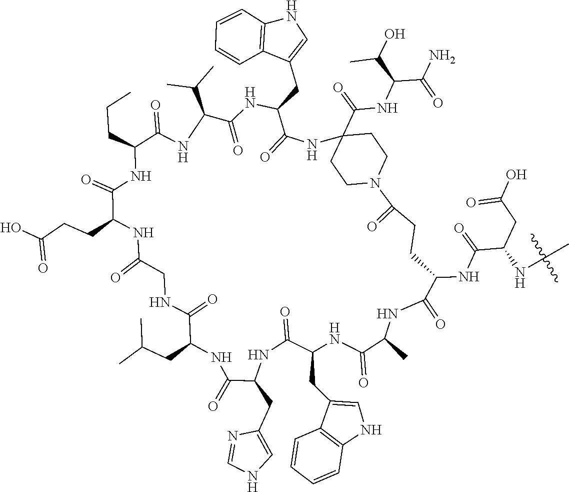 [SMILES: *N[C@@H](CC(=O)O)C(=O)N[C@H]1CCC(=O)N2CCC(C(=O)N[C@H](C(N)=O)C(C)O)(CC2)NC(=O)[C@H](Cc2c[nH]c3ccccc23)NC(=O)[C@H](C(C)C)NC(=O)[C@H](CCC)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-34 |
| 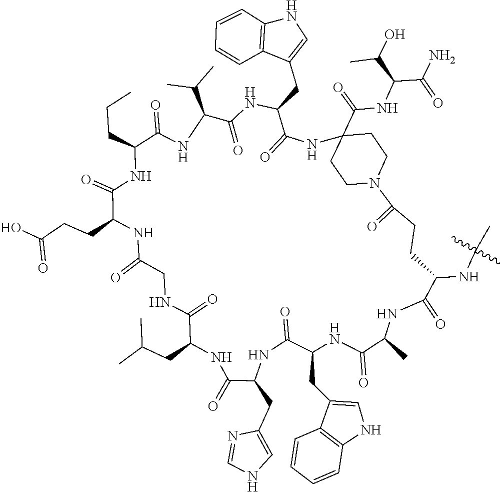 [SMILES: *N[C@H]1CCC(=O)N2CCC(C(=O)N[C@H](C(N)=O)C(C)O)(CC2)NC(=O)[C@H](Cc2c[nH]c3ccccc23)NC(=O)[C@H](C(C)C)NC(=O)[C@H](CCC)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-35 |
| 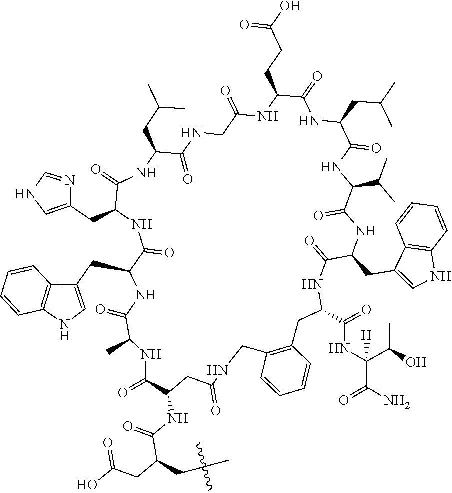 | A-36 |
| 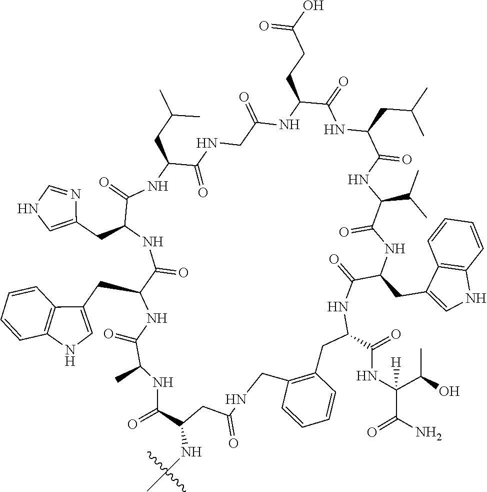 | A-37 |
| 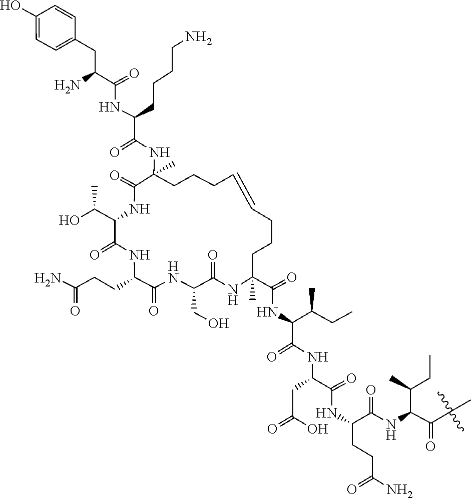 [SMILES: *C(=O)[C@@H](NC(=O)[C@H](CCC(N)=O)NC(=O)[C@H](CC(=O)O)NC(=O)[C@@H](NC(=O)[C@]1(C)CCC/C=C/CCC[C@](C)(NC(=O)[C@H](CCCCN)NC(=O)[C@@H](N)Cc2ccc(O)cc2)C(=O)N[C@@H]([C@@H](C)O)C(=O)N[C@@H](CCC(N)=O)C(=O)N[C@@H](CO)C(=O)N1)[C@@H](C)CC)[C@@H](C)CC] | A-38 |
| 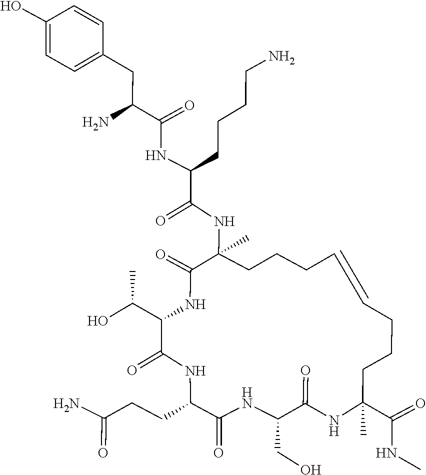 [SMILES: CNC(=O)[C@]1(C)CCC/C=C/CCC[C@](C)(NC(=O)[C@H](CCCCN)NC(=O)[C@@H](N)Cc2ccc(O)cc2)C(=O)N[C@@H]([C@@H](C)O)C(=O)N[C@@H](CCC(N)=O)C(=O)N[C@@H](CO)C(=O)N1] | A-39 |
| 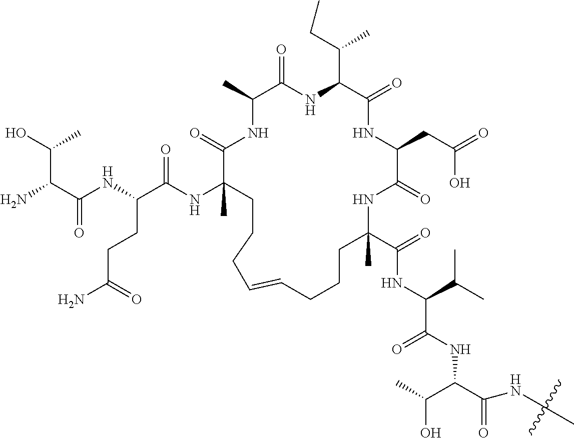 [SMILES: *NC(=O)[C@@H](NC(=O)[C@@H](NC(=O)[C@]1(C)CCC/C=C/CCC[C@](C)(NC(=O)[C@H](CCC(N)=O)NC(=O)[C@H](N)[C@@H](C)O)C(=O)N[C@@H](C)C(=O)N[C@@H]([C@@H](C)CC)C(=O)N[C@@H](CC(=O)O)C(=O)N1)C(C)C)[C@@H](C)O] | A-40 |
| 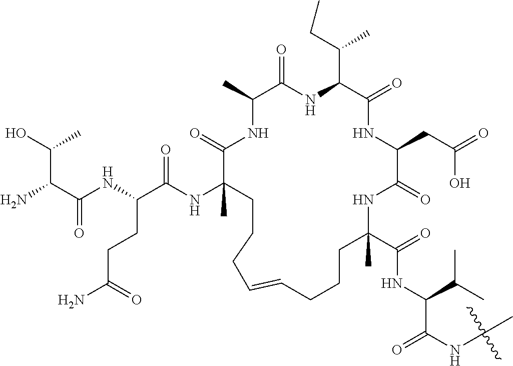 [SMILES: *NC(=O)[C@@H](NC(=O)[C@]1(C)CCC/C=C/CCC[C@](C)(NC(=O)[C@H](CCC(N)=O)NC(=O)[C@H](N)[C@@H](C)O)C(=O)N[C@@H](C)C(=O)N[C@@H]([C@@H](C)CC)C(=O)N[C@@H](CC(=O)O)C(=O)N1)C(C)C] | A-41 |
| 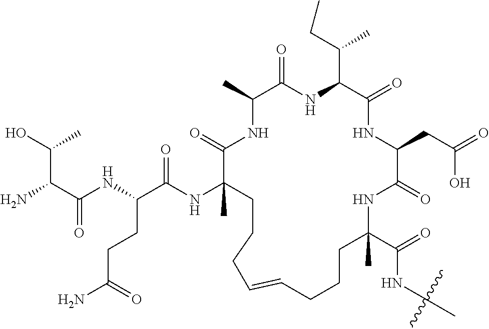 [SMILES: *NC(=O)[C@]1(C)CCC/C=C/CCC[C@](C)(NC(=O)[C@H](CCC(N)=O)NC(=O)[C@H](N)[C@@H](C)O)C(=O)N[C@@H](C)C(=O)N[C@@H]([C@@H](C)CC)C(=O)N[C@@H](CC(=O)O)C(=O)N1] | A-42 |
| 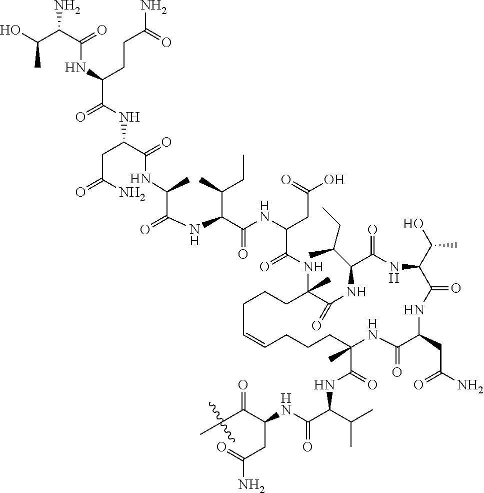 [SMILES: *C(=O)[C@H](CC(N)=O)NC(=O)[C@@H](NC(=O)[C@]1(C)CCC/C=C\CCC[C@](C)(NC(=O)C(CC(=O)O)NC(=O)[C@@H](NC(=O)[C@H](C)NC(=O)[C@H](CC(N)=O)NC(=O)[C@H](CCC(N)=O)NC(=O)[C@@H](N)[C@@H](C)O)[C@@H](C)CC)C(=O)N[C@@H]([C@@H](C)CC)C(=O)N[C@@H]([C@@H](C)O)C(=O)N[C@@H](CC(N)=O)C(=O)N1)C(C)C] | A-43 |
| 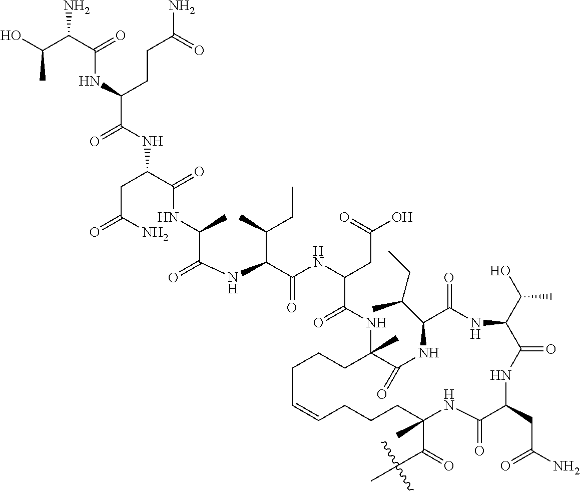 [SMILES: *C(=O)[C@]1(C)CCC/C=C\CCC[C@](C)(NC(=O)C(CC(=O)O)NC(=O)[C@@H](NC(=O)[C@H](C)NC(=O)[C@H](CC(N)=O)NC(=O)[C@H](CCC(N)=O)NC(=O)[C@@H](N)[C@@H](C)O)[C@@H](C)CC)C(=O)N[C@@H]([C@@H](C)CC)C(=O)N[C@@H]([C@@H](C)O)C(=O)N[C@@H](CC(N)=O)C(=O)N1] | A-44 |
| 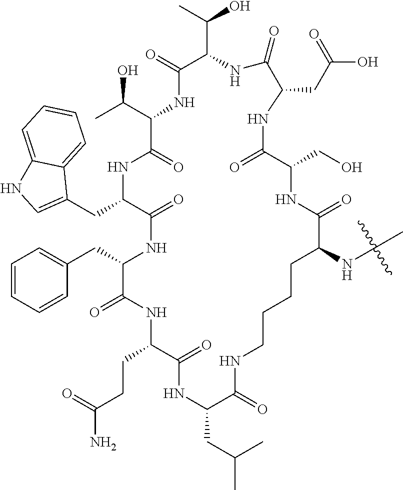 [SMILES: *N[C@H]1CCCCNC(=O)[C@H](CC(C)C)NC(=O)[C@H](CCC(N)=O)NC(=O)[C@H](Cc2ccccc2)NC(=O)[C@H](Cc2c[nH]c3ccccc23)NC(=O)[C@H]([C@@H](C)O)NC(=O)[C@H]([C@@H](C)O)NC(=O)[C@H](CC(=O)O)NC(=O)[C@H](CO)NC1=O] | A-45 |
| 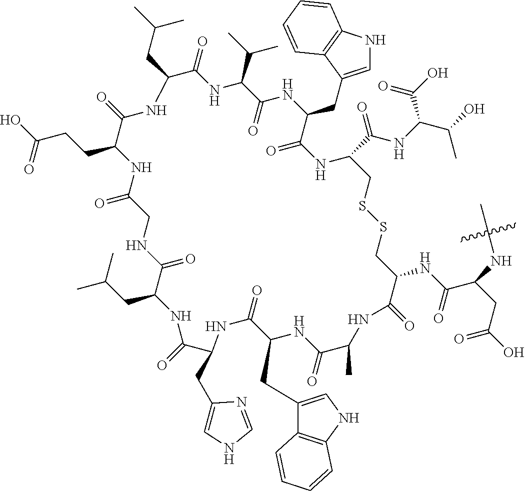 [SMILES: *N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-46 |
| 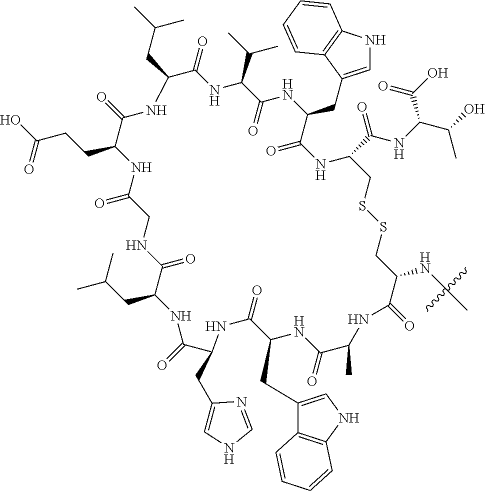 [SMILES: *N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-47 |
| 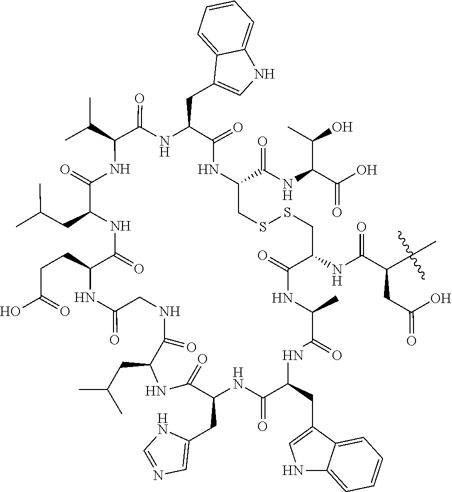 [SMILES: *[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC1=O] | A-48 |
| 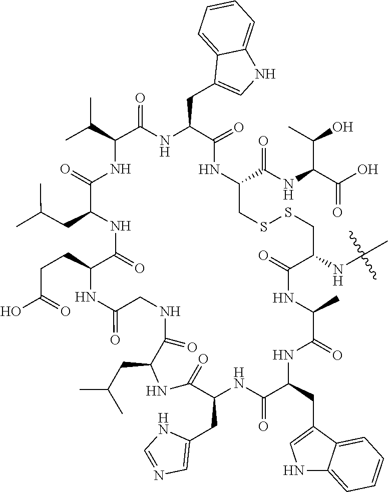 [SMILES: *N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC1=O] | A-49 |
| 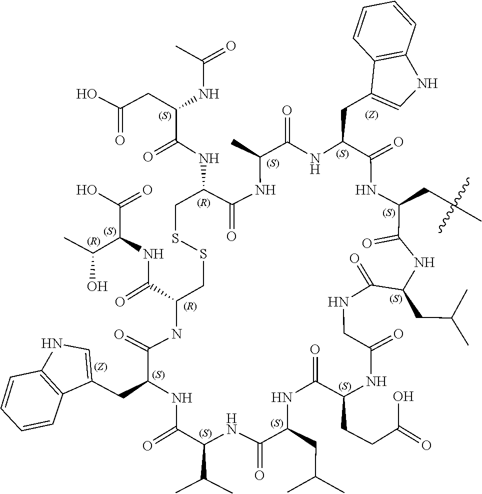 | A-50 |

In some embodiments, a target binding moiety is an antibody binding moiety described herein. In some embodiments, a protein binding moiety is an antibody binding moiety described herein. In some embodiments, —COOH and/or amino groups of amino acid residues, e.g., those at the C-terminus or N-terminus, is optionally capped. For example, in some embodiments, a —COOH group (e.g., a C-terminus —COOH) is amidated (e.g., converted into —CON(R′)₂, e.g., —C(O)NHR (e.g., —C(O)NH₂)), and in some embodiments, an amino group, e.g. —NH₂ (e.g., a N-terminus —NH₂) is capped with R′— or R′C(O)— (e.g., in some embodiments, by conversion —NH₂ into —NHR′ (e.g., —NHC(O)R, (e.g., —NHC(O)CH₃))).

In some embodiments, a target binding moiety is or comprises optionally substituted A-1, A-2, A-3, A-4, A-5, A-6, A-7, A-8, A-9, A-10, A-11, A-12, A-13, A-14, A-15, A-16, A-17, A-18, A-19, A-20, A-21, A-22, A-23, A-24, A-25, A-26, A-27, A-28, A-29, A-30, A-31, A-32, A-33, A-34, A-35, A-36, A-37, A-38, A-39, A-40, A-41, A-42, A-43, A-44, A-45, A-46, A-47, A-48, A-49, or A-50, each of which is optionally substituted. In some embodiments, such a target binding moiety is an antibody binding moiety. In some embodiments, such a target binding moiety is a universal antibody binding moiety.

In some embodiments, a target binding moiety, e.g., a protein binding moiety (e.g., an antibody binding moiety (e.g., a universal antibody binding moiety)) comprises a peptide unit, and is connected to a linker moiety through the C-terminus of the peptide unit. In some embodiments, it is connected to a linker moiety through the N-terminus of the peptide unit. In some embodiments, it is connected to a linker through a side chain group of the peptide unit. In some embodiments, an antibody binding moiety, e.g., a universal antibody binding moiety comprises a peptide unit, and is connected to a target binding moiety optionally through a linker moiety through the C-terminus of the peptide unit. In some embodiments, a target binding moiety, e.g., a protein binding moiety (e.g., an antibody binding moiety (e.g., a universal antibody binding moiety)) comprises a peptide unit, and is connected to a target binding moiety optionally through a linker moiety through the N-terminus of the peptide unit. In some embodiments, a target binding moiety, e.g., a protein binding moiety (e.g., an antibody binding moiety (e.g., a universal antibody binding moiety)) comprises a peptide unit, and is connected to a target binding moiety optionally through a linker moiety through a side chain of the peptide unit.

In some embodiments, a target binding moiety is or comprises (DCAWHLGELVWCT, (SEQ ID NO:17))-, wherein 1-5 (e.g., 1, 2, 3, 4, or 5) amino acid residues may be independently and optionally replaced with another amino acid residue, 1-5 (e.g., 1, 2, 3, 4, or 5) amino acid residues may be independently and optionally deleted, and/or 1-5 (e.g., 1, 2, 3, 4, or 5) amino acid residues may be independently and optionally inserted. In some embodiments, it is connected to the rest of a molecule through its N-terminus. In some embodiments, it is connected to the rest of a molecule through its C-terminus. In some embodiments, it is connected to the rest of a molecule through a side chain of an amino acid residue (e.g., various X residues as described in the present disclosure). In some embodiments, two cysteine residues form a disulfide bond. In some embodiments, a target binding moiety is or comprises

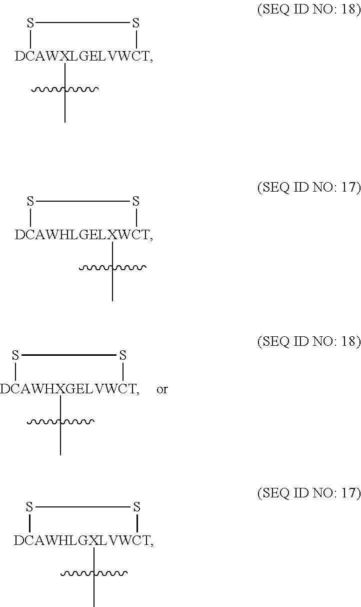

wherein X is an amino acid residue bonded to the rest of a compound or agent, and wherein 1-5 (e.g., 1, 2, 3, 4, or 5) amino acid residues may be independently and optionally replaced with another amino acid residue, 1-5 (e.g., 1, 2, 3, 4, or 5) amino acid residues may be independently and optionally deleted, and/or 1-5 (e.g., 1, 2, 3, 4, or 5) amino acid residues may be independently and optionally inserted. In some embodiments, the total number of replacements, deletions, and insertions is no more than 10 (e.g., 0, or no more than 1, 2, 3, 4, 5, 6, 7, 8, 9, or 10). In some embodiments, the total number is 0. In some embodiments, the total number is no more than 1. In some embodiments, the total number is no more than 2. In some embodiments, the total number is no more than 3. In some embodiments, the total number is no more than 4. In some embodiments, the total number is no more than 5. In some embodiments, the total number is no more than 6. In some embodiments, the total number is no more than 7. In some embodiments, the total number is no more than 8. In some embodiments, the total number is no more than 9. In some embodiments, the total number is no more than 10. In some embodiments, there are no insertions. In some embodiments, there are no deletions. In some embodiments, there are no replacements. In some embodiments X is X is an amino acid residue bonded to the rest of a compound or agent. In some embodiments, X is —N(R′)—CH(−)—C(O)—. In some embodiments, X is —N(R′)—CH(-L^{G}-)—C(O)—. In some embodiments, X is —N(R′)—CH(-L^{LG1}-L^{LG2}-)—C(O)—. In some embodiments, X is —N(R′)—CH(-L^{LG1}-L^{LG2}-L^{LG3}-)—C(O)—. In some embodiments, X is —N(R′)—CH(-L^{LG1}-L^{LG2}-L^{LG3}-L^{LG4}-)—C(O)—.

In some embodiments, X is a residue of any of the following:

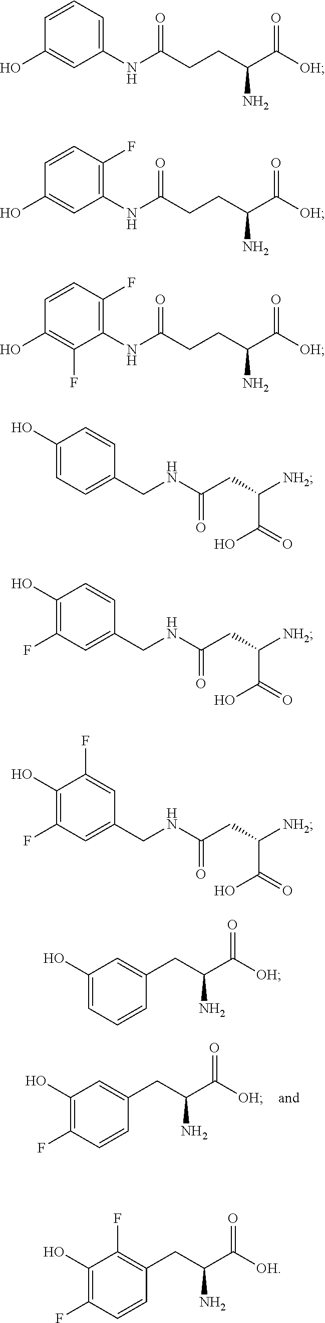

In some embodiments, X is K. In some embodiments, X is D. In some embodiments, X is a residue of Dab. In some embodiments, X is E.

In some embodiments, an antibody binding moiety, e.g., a universal antibody binding moiety, is or comprises a small molecule entity, with a molecular weight of, e.g., less than 10000, 9000, 8000, 7000, 6000, 5000, 4000, 3000, 2000, 1500, 1000, etc. Suitable such antibody binding moieties include small molecule Fc binder moieties, e.g., those described in U.S. Pat. No. 9,745,339, US 201/30131321, etc. In some embodiments, an antibody binding moiety is of such a structure that its corresponding compound is a compound described in U.S. Pat. No. 9,745,339 or US 2013/0131321, the compounds of each of which are independently incorporated herein by reference. In some embodiments, an antibody binding moiety ABT is of such a structure that H-ABT is a compound described in U.S. Pat. No. 9,745,339 or US 2013/0131321, the compounds of each of which are independently incorporated herein by reference. In some embodiments, such a compound can bind to an antibody. In some embodiments, such a compound can bind to Fc region of an antibody.

In some embodiments, a target binding moiety is or comprises any of the following, each of which is optionally substituted:

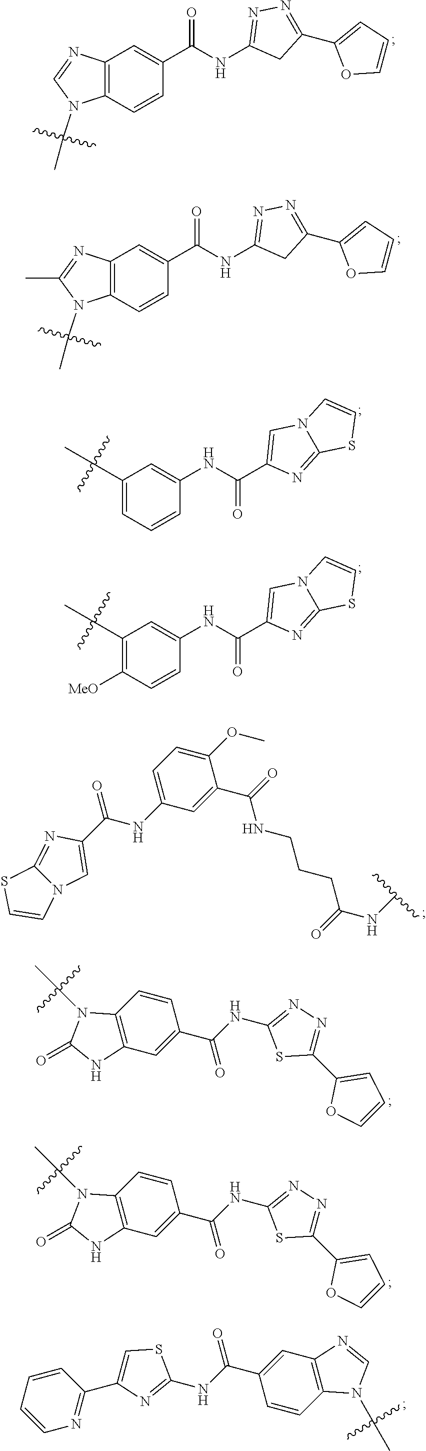

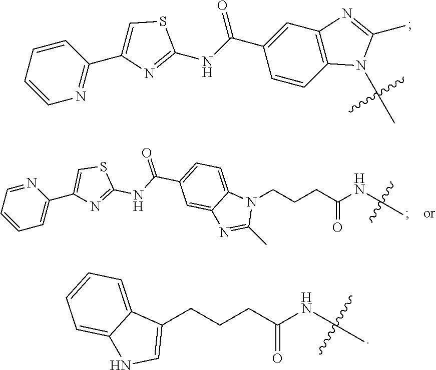 [SMILES: *n1c(C)nc2cc(C(=O)Nc3nc(-c4ccccn4)cs3)ccc21 ; *NC(=O)CCCn1c(C)nc2cc(C(=O)Nc3nc(-c4ccccn4)cs3)ccc21 ; *NC(=O)CCCc1c[nH]c2ccccc12]

In some embodiments, target binding moiety is or comprises any of the following, each of which is optionally substituted:

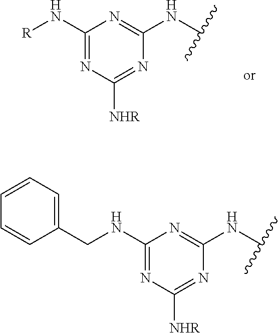

where R can be, for example, hydrogen, C₁-C₄alkyl, or C₃-C₆cycloalkyl.

In some embodiments, a target binding moiety is or comprises any of the following:

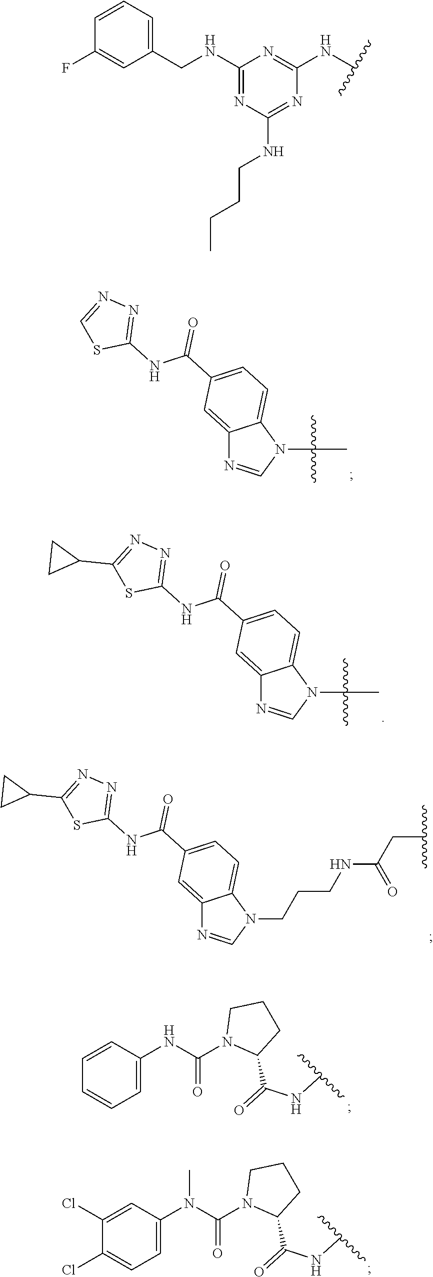 [SMILES: *Nc1nc(NCCCC)nc(NCc2cccc(F)c2)n1 ; *n1cnc2cc(C(=O)Nc3nncs3)ccc21 ; *n1cnc2cc(C(=O)Nc3nnc(C4CC4)s3)ccc21 ; *CC(=O)NCCCn1cnc2cc(C(=O)Nc3nnc(C4CC4)s3)ccc21 ; *NC(=O)[C@H]1CCCN1C(=O)Nc1ccccc1 ; *NC(=O)[C@H]1CCCN1C(=O)N(C)c1ccc(Cl)c(Cl)c1]

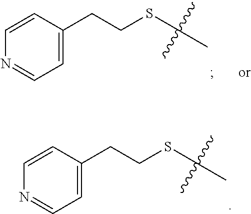 [SMILES: *SCCc1ccncc1 ; *SCCc1ccncc1]

In some embodiments, target binding moiety is or comprises

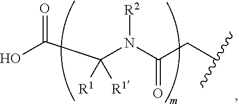

wherein each variable is independently as described herein. In some embodiments, m is 4 to 13.

In some embodiments, a target binding moiety is or comprises

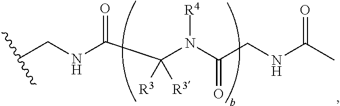

wherein b is 1-20, and each other variable is independently as described herein.

In some embodiments, b is 4-13. In some embodiments, a target binding moiety, e.g., R^{c}-(Xaa)z-, is or comprises any of the following:

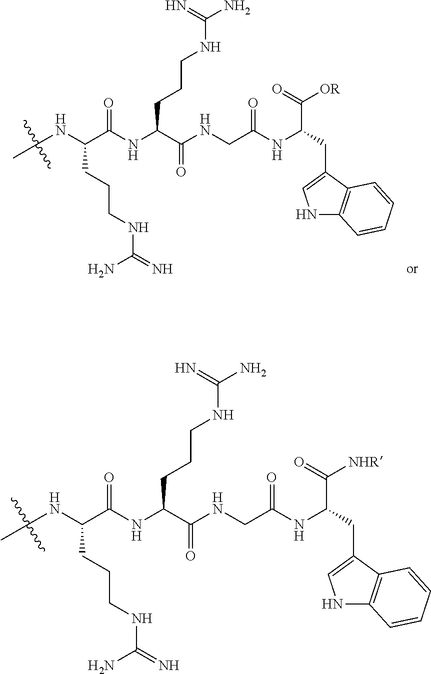

is, e.g., H or C₁-C₄ alkyl and R′ is e.g., H or C₁-C₄alkyl.

In some embodiments, a target binding moiety, e.g., R^{c}-(Xaa)z-, is or comprises

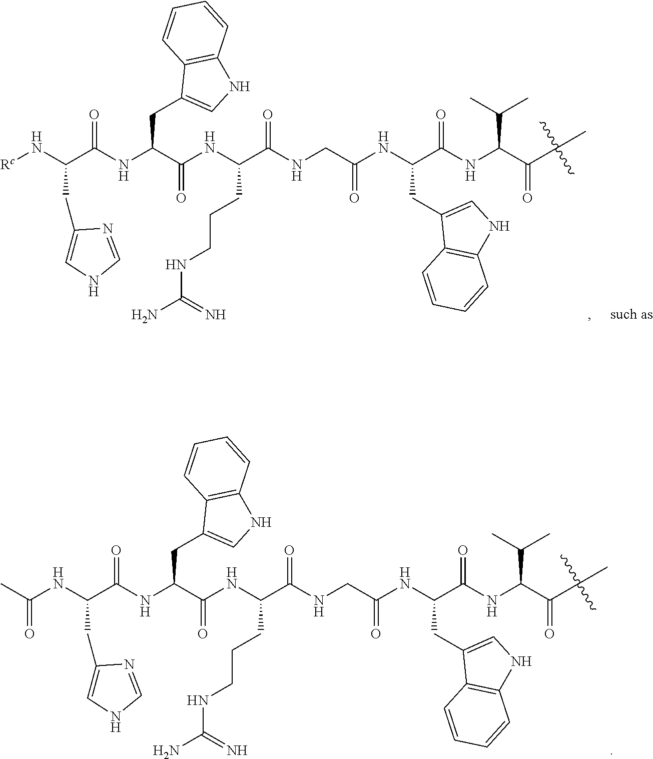

In some embodiments, a target binding moiety, e.g., R^{c}-(Xaa)z-, is or comprises

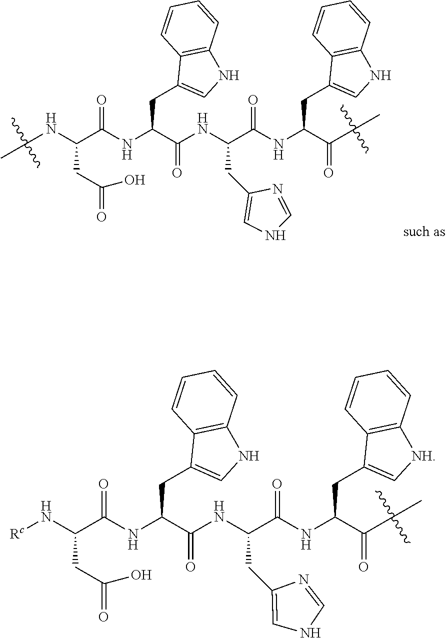

In some embodiments, a target binding moiety, e.g., R^{c}-(Xaa)z-, is or comprises

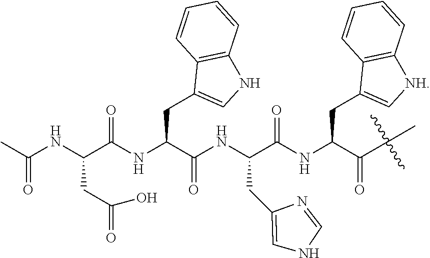 [SMILES: *C(=O)[C@H](Cc1c[nH]c2ccccc12)NC(=O)[C@H](Cc1c[nH]cn1)NC(=O)[C@H](Cc1c[nH]c2ccccc12)NC(=O)[C@H](CC(=O)O)NC(C)=O]

In some embodiments, a target binding moiety, e.g., R^{c}-(Xaa)z-, is or comprises any one of the following:

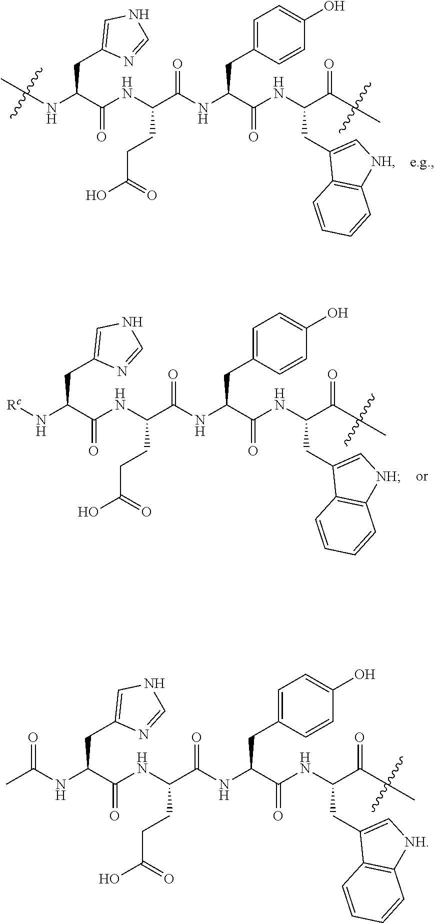

In some embodiments, a target binding moiety, e.g., R^{c}-(Xaa)z-, is or comprises

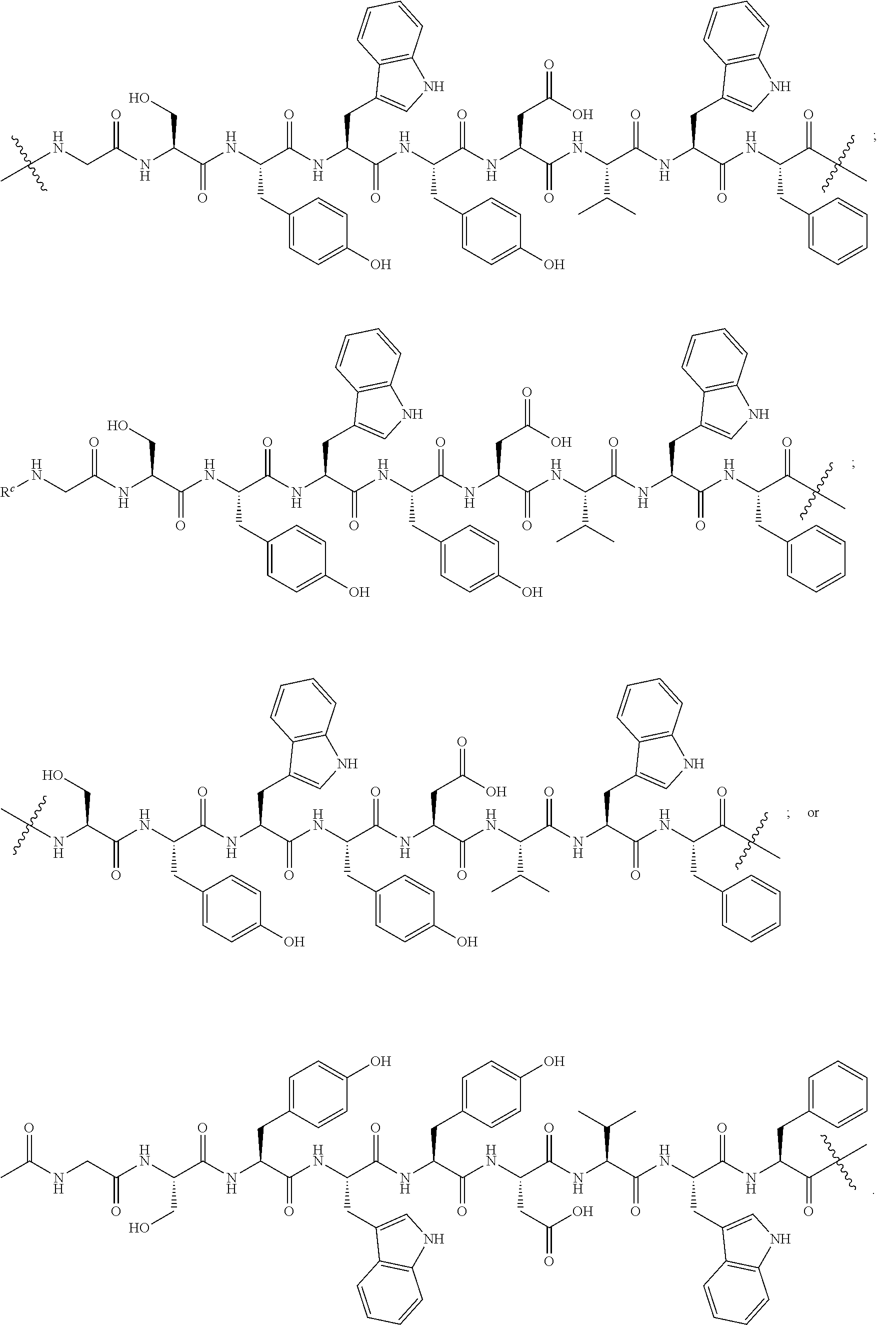

In some embodiments, a target binding moiety, e.g., R^{c}-(Xaa)z-, is or comprises

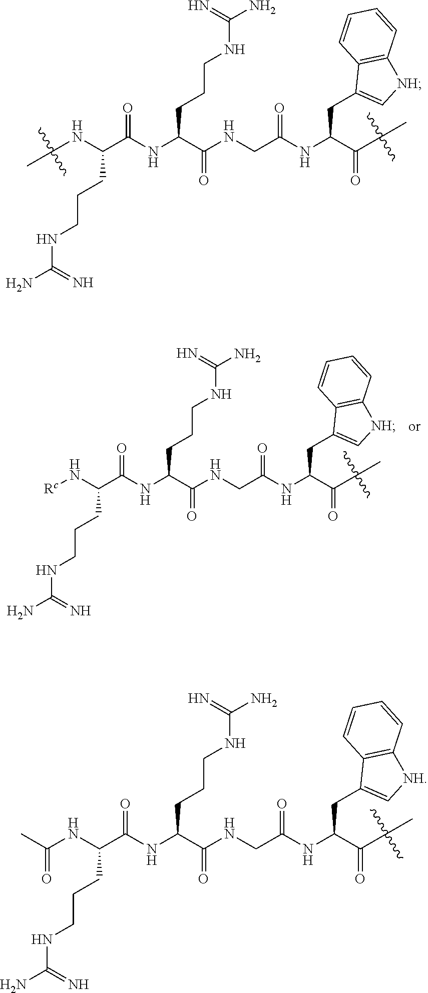

In some embodiments, —NH— is bonded to a R^{c} group. In some embodiments, R^{c} is R—C(O)—. In some embodiments, R^{c} is CH₃C(O)—. In some embodiments, such target binding moieties are antibody binding moieties.

In some embodiments, a target binding moiety, e.g.,

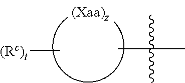

or R^{c}-(Xaa)z-, is or comprises any one of the following:

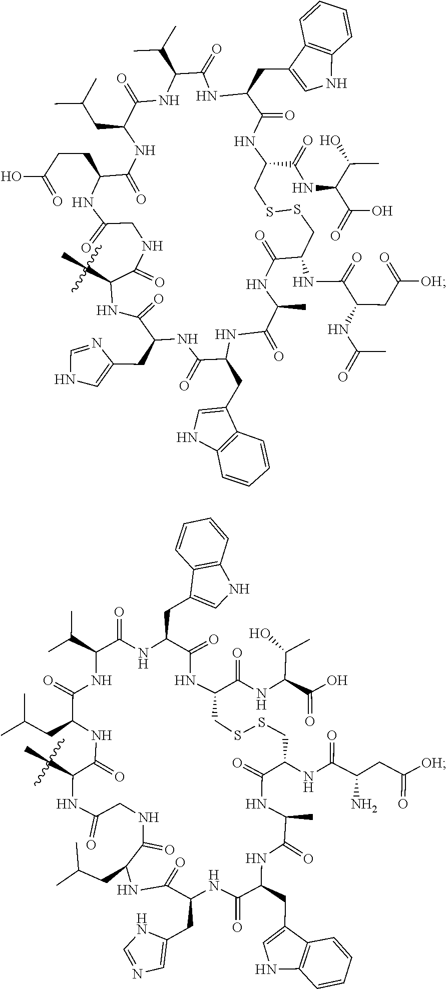

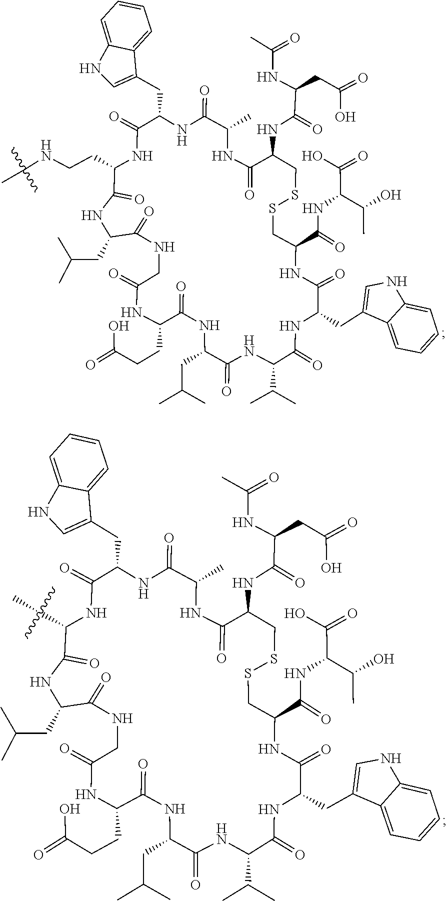

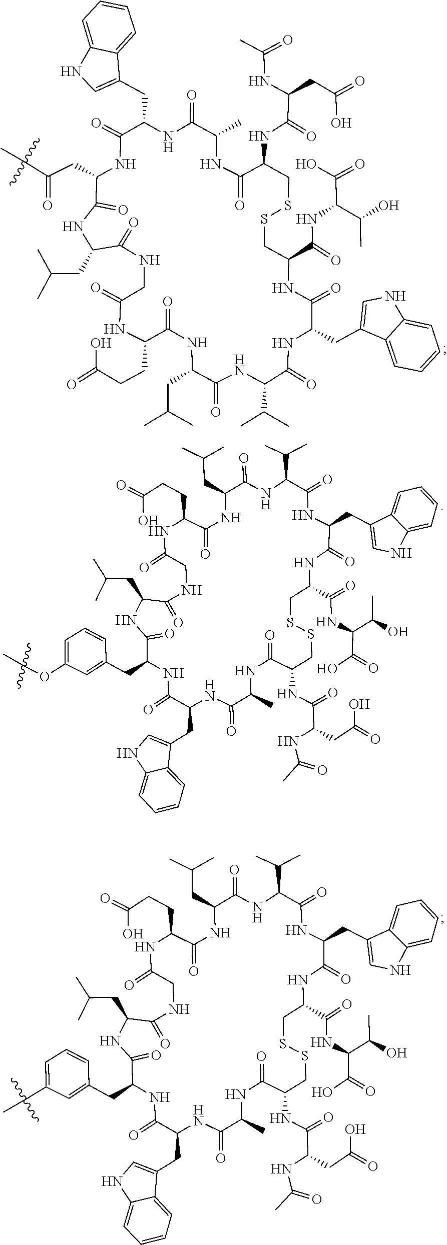

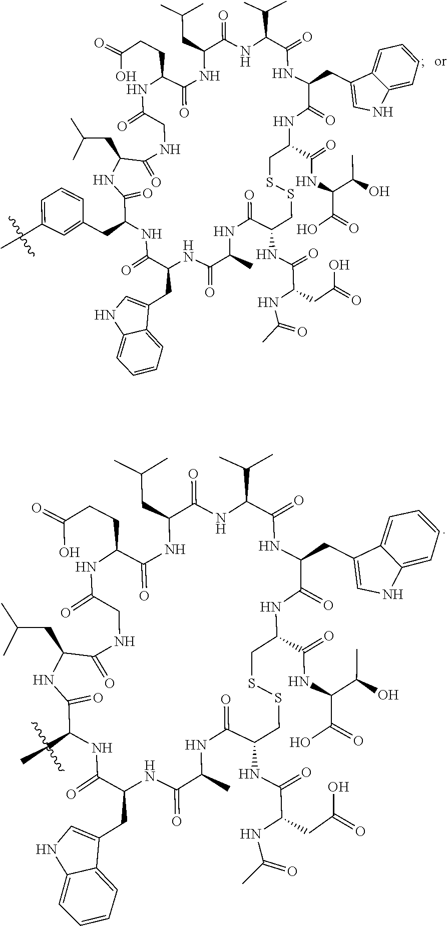

In some embodiments, a target binding moiety, e.g., R^{c}-(Xaa)z-, is or comprises a Z33 peptide moiety. In some embodiments, a target binding moiety, e.g., R^{c}-(Xaa)z-, is or comprises -FNMQQQRRFYEALHDPNLNEEQRNAKIKSIRDD-NH₂ (SEQ ID NO: 29) or a fragment thereof. In some embodiments, a target binding moiety, e.g., R^{c}-(Xaa)z-, is or comprises FNMQCQRRFYEALHDPNLNEEQRNAKIKSIRDDC (SEQ ID NO:30) or a fragment thereof. In some embodiments, a target binding moiety, e.g.,

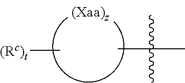

or R^{c}-(Xaa)z-, is or comprises a moiety of a peptide such as FNMQCQRRFYEALHDPNLNEEQRNAKIKSIRDDC (SEQ ID NO:30), RGNCAYHRGQLVWCTYH (SEQ ID NO:31), RGNCAYHKGQLVWCTYH, RGNCKYHRGQLVWCTYH (SEQ ID NO:32), RGNCAWHRGKLVWCTYH(SEQ ID NO:33), RGNCAWHRGKLVWCTYH (SEQ ID NO:34), RGNCKWHRGELVWCTYH (SEQ ID NO:35), RGNCKWHRGQLVWCTYH (SEQ ID NO:36), RGNCKYHLGELVWCTYH (SEQ ID NO:37), RGNCKYHLGQLVWCTYH (SEQ ID NO:38), DCKWHLGELVWCT (SEQ ID NO:39), DCKYHLGELVWCT (SEQ ID NO:40), DCKWHRGELVWCT (SEQ ID NO:41), DCKWHLGQLVWCT (SEQ ID NO:42), DCKYHRGELVWCT (SEQ ID NO:43), DCKYHLGQLVWCT (SEQ ID NO:44), DCKWHRGQLVWCT (SEQ ID NO:45), DCKYHRGQLVWCT (SEQ ID NO:46), FNKQCQRRFYEALHDPNLNEEQRNARIRSIRDDC (SEQ ID NO:47), FNMQCQRRFYEALHDPNLNEEQRNARIRSIKDDC (SEQ ID NO:48), FNMQCQRRFYEALHDPNLNKEQRNARIRSIRDDC (SEQ ID NO:49), FNMQCQRRFYEALHDPNLNEEQRNARIRSIRDDC (SEQ ID NO:50), RGNCAWHLGQLVWCKYH (SEQ ID NO:51), RGNCAWHLGELVWCKYH (SEQ ID NO:52), RGNCAYHLGQLVWCTKH (SEQ ID NO:53), RGNCAYHLGQLVWCTYK (SEQ ID NO:54), RGNCAYHRGQLVWCTKH (SEQ ID NO:55), KNMQCQRRFYEALHDPNLNEEQRNARIRSIRDDC (SEQ ID NO:56), FNMQCQKRFYEALHDPNLNEEQRNARIRSIRDDC (SEQ ID NO:57), FNMQCQRRFYEAKHDPNLNEEQRNARIRSIRDDC (SEQ ID NO:58), FNMQCQRRFYEALHDPNLNEEQRKARIRSIRDDC (SEQ ID NO:59), FNMQCQRRFYEALHDPNLNKEQRNARIRSIRDDC (SEQ ID NO:49), FNMQCQRRFYEALHDPNLNEEQRNARIRSIKDDC (SEQ ID NO:48), FNKQCQRRFYEALHDPNLNEEQRNARIRSIRDDC (SEQ ID NO:47), FNMQCKRRFYEALHDPNLNEEQRNARIRSIRDDC (SEQ ID NO:60), FNMQCQRRFYEALHDPNLNEEQRNARIRSIRKDC (SEQ ID NO:61), Fc-III, FcBP-2, Fc-III-4C,

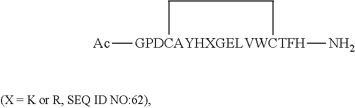

etc., wherein two cysteine residues may optionally form a disulfide bond. In some embodiments, in a peptide described herein, two cysteine residues form a disulfide bond. In some embodiments, a peptide, such as Z33, FNMQCQRRFYEALHDPNLNEEQRNAKIKSIRDDC (SEQ ID NO:30), RGNCAYHRGQLVWCTYH (SEQ ID NO:31), RGNCKYHRGQLVWCTYH (SEQ ID NO:33), RGNCAYHKGQLVWCTYH (SEQ ID NO:32), RGNCAWHRGKLVWCTYH (SEQ ID NO:34), RGNCKWHRGQLVWCTYH (SEQ ID NO:36), RGNCKWHRGELVWCTYH (SEQ ID NO:34), RGNCKYHLGELVWCTYH (SEQ ID NO:37), RGNCKYHLGQLVWCTYH (SEQ ID NO:38), DCKWHLGELVWCT (SEQ ID NO:39), DCKYHLGELVWCT (SEQ ID NO:40), DCKWHRGELVWCT (SEQ ID NO:41), DCKWHLGQLVWCT (SEQ ID NO:42), DCKYHRGELVWCT (SEQ ID NO:43), DCKYHLGQLVWCT (SEQ ID NO:44), DCKWHRGQLVWCT (SEQ ID NO:45), DCKYHRGQLVWCT (SEQ ID NO:46), FNKQCQRRFYEALHDPNLNEEQRNARIRSIRDDC (SEQ ID NO:47), FNMQCQRRFYEALHDPNLNEEQRNARIRSIKDDC (SEQ ID NO:48), FNMQCQRRFYEALHDPNLNKEQRNARIRSIRDDC (SEQ ID NO:49), FNMQCQRRFYEALHDPNLNEEQRNARIRSIRDDC (SEQ ID NO:50), RGNCAWHLGQLVWCKYH (SEQ ID NO:51), RGNCAWHLGELVWCKYH (SEQ ID NO:52), RGNCAYHLGQLVWCTKH (SEQ ID NO:53), RGNCAYHLGQLVWCTYK (SEQ ID NO:54), RGNCAYHRGQLVWCTKH (SEQ ID NO:55), KNMQCQRRFYEALHDPNLNEEQRNARIRSIRDDC (SEQ ID NO:56), FNMQCQKRFYEALHDPNLNEEQRNARIRSIRDDC (SEQ ID NO:57), FNMQCQRRFYEAKHDPNLNEEQRNARIRSIRDDC (SEQ ID NO:58), FNMQCQRRFYEALHDPNLNEEQRKARIRSIRDDC (SEQ ID NO:59), FNMQCQRRFYEALHDPNLNKEQRNARIRSIRDDC (SEQ ID NO:49), FNMQCQRRFYEALHDPNLNEEQRNARIRSIKDDC (SEQ ID NO:48), FNKQCQRRFYEALHDPNLNEEQRNARIRSIRDDC (SEQ ID NO:47), FNMQCKRRFYEALHDPNLNEEQRNARIRSIRDDC (SEQ ID NO:60), FNMQCQRRFYEALHDPNLNEEQRNARIRSIRKDC (SEQ ID NO:61), Fc-III, FcBP-2, Fc-III-4C,

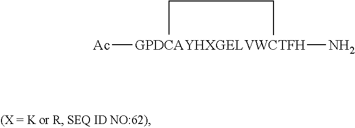

etc., is connected through its N-terminus, C-terminus, or a side chain (e.g., of K (e.g., of underlined K residues in RGNCAYHKGQLVWCTYH (SEQ ID NO:32), RGNCKYHRGQLVWCTYH (SEQ ID NO:33), RGNCAWHRGKLVWCTYH (SEQ ID NO:34), RGNCKWHRGELVWCTYH (SEQ ID NO:35), RGNCKWHRGQLVWCTYH (SEQ ID NO:36), RGNCKYHLGELVWCTYH (SEQ ID NO:37), RGNCKYHLGQLVWCTYH (SEQ ID NO:38), DCKWHLGELVWCT (SEQ ID NO:39), DCKYHLGELVWCT (SEQ ID NO:40), DCKWHRGELVWCT (SEQ ID NO:41), DCKWHLGQLVWCT (SEQ ID NO:42), DCKYHRGELVWCT (SEQ ID NO:43), DCKYHLGQLVWCT (SEQ ID NO:44), DCKWHRGQLVWCT (SEQ ID NO:45), DCKYHRGQLVWCT (SEQ ID NO:46), RGNCAWHLGQLVWCKYH (SEQ ID NO:51), FNKQCQRRFYEALHDPNLNEEQRNARIRSIRDDC (SEQ ID NO:47), FNMQCQRRFYEALHDPNLNEEQRNARIRSIKDDC (SEQ ID NO:48), FNMQCQRRFYEALHDPNLNKEQRNARIRSIRDDC (SEQ ID NO:49), RGNCAWHLGELVWCKYH (SEQ ID NO:52), RGNCAYHLGQLVWCTKH (SEQ ID NO:53), RGNCAYHLGQLVWCTYK (SEQ ID NO:54), RGNCAYHRGQLVWCTKH (SEQ ID NO:55), KNMQCQRRFYEALHDPNLNEEQRNARIRSIRDDC (SEQ ID NO:56), FNMQCQKRFYEALHDPNLNEEQRNARIRSIRDDC (SEQ ID NO:57), FNMQCQRRFYEAKHDPNLNEEQRNARIRSIRDDC (SEQ ID NO:58), FNMQCQRRFYEALHDPNLNEEQRKARIRSIRDDC (SEQ ID NO:59), FNMQCQRRFYEALHDPNLNKEQRNARIRSIRDDC (SEQ ID NO:49), FNMQCQRRFYEALHDPNLNEEQRNARIRSIKDDC (SEQ ID NO:48), FNKQCQRRFYEALHDPNLNEEQRNARIRSIRDDC (SEQ ID NO:47), FNMQCKRRFYEALHDPNLNEEQRNARIRSIRDDC (SEQ ID NO:60), FNMQCQRRFYEALHDPNLNEEQRNARIRSIRKDC (SEQ ID NO:61), etc.)). In some embodiments, one or more amino acid residues of a sequence may be independently and optionally replaced (e.g., 1-5), deleted (e.g., 1-5) and/or inserted (e.g., 1-5) as described herein. In some embodiments, a target binding moiety, e.g.

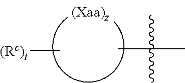

or R^{c}-(Xaa)z-, is or comprises -CXYHXXXLVWC- (SEQ ID NO:63), -XCXYHXXXLVWC- (SEQ ID NO:64), -CXYHXXXLVWCX- (SEQ ID NO:65), —X₀₋₃CXYHXXXLVWCX₀₋₃- (SEQ ID NO:66), -XCXYHXXXLVWCXXX (SEQ ID NO:67), -XXXCXYHXXXLVWCXXX (SEQ ID NO:66)-, wherein each X is independently an amino acid residue, and the two C residues optionally form a disulfide bond. In some embodiments, X⁸ (the X after H) is Orn. In some embodiments, X⁸ is Dab. In some embodiments, X⁸ is Lys(Ac). In some embodiments, X⁸ is Orn(Ac). In some embodiments, X⁸ is Dab(Ac). In some embodiments, X⁸ is Arg. In some embodiments, X⁸ is Nle. In some embodiments, X⁸ is Nva. In some embodiments, X⁸ is Val. In some embodiments, X⁸ is Tle. In some embodiments, X⁸ is Leu. In some embodiments, X⁸ is Ala(tBu). In some embodiments, X⁸ is Cha. In some embodiments, X⁸ is Phe. In some embodiments, a target binding moiety, e.g.,

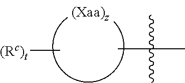

or R^{c}-(Xaa)z-, is or comprises DCAWHLGELVWCT (SEQ ID NO:17). In some embodiments, a C-terminus and/or a N-terminus of a protein agent/peptide agent moiety are independently capped (e.g., R^{c}(O)— such as CH₃C(O)— for N-terminus, —N(R′)₂ such as —NH₂ for C-terminus, etc.). In some embodiments, such target binding moieties are antibody binding moieties. In some embodiments, as described herein, a residue may be modified or replaced for connection with another moiety, e.g., in some embodiments, H may be replaced with an amino acid residue comprises a side chain that contain —COOH or a salt or activated form thereof (e.g., D).

In some embodiments, a target binding moiety, e.g.,

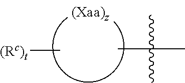

or R^{c}-(Xaa)z-, is or comprises (X₁₋₃)—C—(X₂)—H-(Xaa1)-G-(Xaa2)-L-V—W—C—(X₁₋₃) (SEQ ID NO:68), wherein each of X and Xaa is independently an amino acid residue and optionally not a cysteine residue. In some embodiments, Xaa^{L} is R, L, L, D, E, a 2-amino suberic acid residue, or a diaminopropionic acid residue. In some embodiments, Xaa2 is L, D, E, N, or Q. In some embodiments, Xaa^{L} is a lysine residue, a cysteine residue, an aspartic acid residue, a glutamic acid residue, a 2-amino suberic acid residue, or a diaminopropionic acid residue. In some embodiments, Xaa2 is a glutamic acid residue or an aspartic acid residue. In some embodiments, Xaa1 is an arginine residue or a leucine residue. In some embodiments, Xaa2 is a lysine residue, a glutamine residue, or an aspartic acid residue. In some embodiments, such target binding moieties are antibody binding moieties.

In some embodiments, a target binding moiety, e.g.,

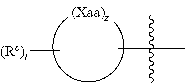

or R^{c}-(Xaa)z-, is or comprises (X1-3)-C-(Xaa3)-(xaa4)-H-(Xaa1)-G-(Xaa2)-L-V—W—C-(Xaa5)-(Xaa6)-(Xaa7) (SEQ ID NO:68), wherein each of X and Xaa is independently an amino acid residue and optionally not a cysteine residue. In some embodiments, Xaa3 is an alanine residue or a lysine residue. In some embodiments, Xaa4 is a tryptophan residue or a tyrosine residue. In some embodiments, Xaa1 is an arginine residue, a leucine residue, a lysine residue, an aspartic acid residue, a glutamic acid residue, a 2-amino suberic acid residue, or a diaminopropionic acid residue. In some embodiments, Xaa2 is a lysine residue, a glutamine residue, a glutamic acid residue, an asparagine residue, or an aspartic acid residue. In some embodiments, Xaa5 is a threonine residue or a lysine residue. In some embodiments, Xaa6 is a tyrosine residue, a lysine residue, or absent. In some embodiments, Xaa7 is a histidine residue, a lysine residue, or absent. In some embodiments, such target binding moieties are antibody binding moieties.

In some embodiments, a target binding moiety, e.g.,

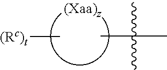

or R^{c}-(Xaa)z-, is or comprises D-C-(Xaa3)-(Xaa4)-H-(Xaa1)-G-(Xaa2)-L-V—W—C-(Xaa5)-(Xaa6)-(Xaa7) (SEQ ID NO:69), wherein each of X and Xaa is independently an amino acid residue and optionally not a cysteine residue. In some embodiments, Xaa3 is an alanine residue or a lysine residue. In some embodiments, Xaa4 is a tryptophan residue or a tyrosine residue. In some embodiments, Xaa1 is an arginine residue, a leucine residue, a lysine residue, an aspartic acid residue, a glutamic acid residue, a 2-amino suberic acid residue, or a diaminopropionic acid residue. In some embodiments, Xaa2 is a lysine residue, a glutamine residue, a glutamic acid residue, an asparagine residue, or an aspartic acid residue. In some embodiments, Xaa5 is a threonine residue or a lysine residue. In some embodiments, Xaa6 is a tyrosine residue, a lysine residue, or absent. In some embodiments, Xaa7 is a histidine residue, a lysine residue, or absent. In some embodiments, such target binding moieties are antibody binding moieties.

In some embodiments, a target binding moiety, e.g.,

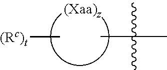

or R^{c}-(Xaa)z-, is or comprises D-C-(Xaa3)-(Xaa4)-H-(Xaa1)-G-(Xaa2)-L-V—W—C-T (SEQ ID NO:70), wherein each of X and Xaa is independently an amino acid residue and optionally not a cysteine residue. In some embodiments, Xaa3 is an alanine residue or a lysine residue. In some embodiments, Xaa4 is a tryptophan residue or a tyrosine residue. In some embodiments, Xaa1 is an arginine residue, a leucine residue, a lysine residue, an aspartic acid residue, a glutamic acid residue, a 2-amino suberic acid residue, or a diaminopropionic acid residue. In some embodiments, Xaa2 is a lysine residue, a glutamine residue, a glutamic acid residue, an asparagine residue, or an aspartic acid residue. In some embodiments, such target binding moieties are antibody binding moieties.

In some embodiments, a target binding moiety, e.g.,

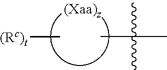

or R^{c}-(Xaa)z-, is or comprises R-G-N—C-(Xaa3)-(Xaa4)-H-(Xaa1)-G-(Xaa2)-L-V—W—C-(Xaa5)-(Xaa6)-(Xaa7) (SEQ ID NO:71), wherein each of X and Xaa is independently an amino acid residue and optionally not a cysteine residue. In some embodiments, Xaa3 is an alanine residue or a lysine residue. In some embodiments, Xaa4 is a tryptophan residue or a tyrosine residue. In some embodiments, Xaa1 is an arginine residue, a leucine residue, a lysine residue, an aspartic acid residue, a glutamic acid residue, a 2-amino suberic acid residue, or a diaminopropionic acid residue. In some embodiments, Xaa2 is a lysine residue, a glutamine residue, a glutamic acid residue, an asparagine residue, or an aspartic acid residue. In some embodiments, Xaa5 is a threonine residue or a lysine residue. In some embodiments, Xaa6 is a tyrosine residue, a lysine residue, or absent. In some embodiments, Xaa7 is a histidine residue, a lysine residue, or absent. In some embodiments, such target binding moieties are antibody binding moieties.

In some embodiments, target binding moieties, e.g., various target binding moieties described above, are protein binding moieties. In some embodiments, target binding moieties are antibody binding moieties. In some embodiments, LG is or comprises such a target binding moiety. In some embodiments, LG is or comprises a protein binding moiety. In some embodiments, LG is or comprises an antibody binding moiety.

In some embodiments, a target binding moiety, e.g., an antibody binding moiety, is or comprises an adapter protein agent, e.g., as described in Hui, et al., Bioconjugate Chem. 2015, 26, 1456-1460, doi: 10.1021/acs.bioconjchem.5b00275. In some embodiments, when utilized in accordance with the present disclosure, adapter proteins do not require reactive residues (e.g., BPA) to achieve one or more or all advantages.

In some embodiments, target binding moiety, e.g., an antibody binding moiety is or comprises a triazine moiety, e.g., one is described in US 2009/0286693. In some embodiments, a target binding moiety, e.g., an antibody binding moiety is of such a structure that its corresponding compound is a compound described in US 2009/0286693, the compounds of which are independently incorporated herein by reference. In some embodiments, a target binding moiety, e.g., an antibody binding moiety, is ABT. In some embodiments, ABT is of such a structure that H-ABT is a compound described in US 2009/0286693, the compounds of which are independently incorporated herein by reference. In some embodiments, such a compound can bind to an antibody. In some embodiments, such a compound can bind to Fc region of an antibody.

In some embodiments, a target binding moiety, e.g., an antibody binding moiety is or comprises a triazine moiety, e.g., one described in Teng, et al., A strategy for the generation of biomimetic ligands for affinity chromatography. Combinatorial synthesis and biological evaluation of an IgG binding ligand, J. Mol. Recognit. 1999; 12:67-75 (“Teng”). In some embodiments, a target binding moiety, e.g., an antibody binding moiety is of such a structure that its corresponding compound is a compound described in Teng, the compounds of which are independently incorporated herein by reference. In some embodiments, a target binding moiety, e.g., an antibody binding moiety, ABT is of such a structure that H-ABT is a compound described in Teng, the compounds of which are independently incorporated herein by reference. In some embodiments, such a compound can bind to an antibody. In some embodiments, such a compound can bind to Fc region of an antibody.

In some embodiments, v target binding moiety, e.g., an antibody binding moiety is a triazine moiety, e.g., one described in Uttamchandani, et al., Microarrays of Tagged Combinatorial Triazine Libraries in the Discovery of Small-Molecule Ligands of Human IgG, J Comb Chem. 2004 November-December; 6(6):862-8 (“Uttamchandani”). In some embodiments, a target binding moiety, e.g., an antibody binding moiety is of such a structure that its corresponding compound is a compound described in Uttamchandani, the compounds of which are independently incorporated herein by reference. In some embodiments, a target binding moiety, e.g., an antibody binding moiety, ABT is of such a structure that H-ABT is a compound described in Uttamchandani, the compounds of which are independently incorporated herein by reference. In some embodiments, such a compound can bind to an antibody. In some embodiments, such a compound can bind to Fc region of an antibody.

In some embodiments, an antibody binding moiety binds to one or more binding sites of protein A. In some embodiments, an antibody binding moiety binds to one or more binding sites of protein G. In some embodiments, an antibody binding moiety binds to one or more binding sites of protein L. In some embodiments, an antibody binding moiety binds to one or more binding sites of protein Z. In some embodiments, an antibody binding moiety binds to one or more binding sites of protein LG. In some embodiments, an antibody binding moiety binds to one or more binding sites of protein LA. In some embodiments, an antibody binding moiety binds to one or more binding sites of protein AG.

In some embodiments, a target binding moiety, e.g., an antibody binding moiety can bind to a nucleotide-binding site. In some embodiments, a target binding moiety, e.g., an antibody binding moiety is a small molecule moiety that can bind to a nucleotide-binding site. In some embodiments, a small molecule is tryptamine. In some embodiments, a target binding moiety, e.g., an antibody binding moiety, ABT is of such a structure that H-ABT is tryptamine.

In some embodiments, an antibody binding moiety is a moiety (e.g., small molecule moiety, peptide moiety, nucleic acid moiety, etc.) that can selectively bind to IgG, and when used in provided technologies can provide and/or stimulate ADCC and/or ADCP. In some embodiments, peptide display technologies (e.g., phase display, non-cellular display, etc.) can be utilized to identify antibody binding moieties. In some embodiments, an antibody binding moiety is a moiety (e.g., small molecule moiety, peptide moiety, nucleic acid moiety, etc.) that can bind to IgG and optionally can compete with known antibody binders, e.g., protein A, protein G, protein L, etc.

As appreciated by those skilled in the art, antibodies of various properties and activities (e.g., antibodies recognizing different antigens, having optional modifications, etc.) may be targeted by antibody binding moieties described in the present disclosure. In some embodiments, such antibodies include antibodies administered to a subject, e.g., for therapeutic purposes. In some embodiments, antibody binding moieties described herein may bind antibodies toward different antigens and are useful for conjugating moieties of interest with various antibodies.

In some embodiments, a target binding moiety, e.g., an antibody binding moiety, is or comprises a meditope agent moiety. In some embodiments, a meditope agent is described in, e.g., US 2019/0111149.

In some embodiments, a target binding moiety, e.g., an antibody binding moiety, can bind to human IgG. In some embodiments, a target binding moiety, e.g., an antibody binding moiety, can bind to rabbit IgG. In some embodiments, a target binding moiety, e.g., an antibody binding moiety, binds to IgG1. In some embodiments, a target binding moiety, e.g., an antibody binding moiety, binds to IgG2. In some embodiments, a target binding moiety, e.g., an antibody binding moiety, binds to IgG3. In some embodiments, a target binding moiety, e.g., an antibody binding moiety, binds to IgG4. In some embodiments, a target binding moiety, e.g., an antibody binding moiety, binds to IgG1, IgG2 and/or IgG4. In some embodiments, a target binding moiety, e.g., an antibody binding moiety, binds to IgG1, IgG2 and IgG4.

In some embodiments, target binding moieties (e.g., antibody binding moieties) bind to targets (e.g., antibody agents for antibody binding moieties) with a Kd that is about 1 mM-1 μM or less. In some embodiments, a Kd is about 1 mM, 0.5 mM, 0.2 mM, 0.1 mM, 0.05 mM, 0.02 mM, 0.01 mM, 0.005 mM, 0.002 mM, 0.001 mM, 500 nM, 200 nM, 100 nM, 50 nM, 20 nM, 10 nM, 5 nM, 2 nM, 1 nM, 0.5 nM, 0.2 nM, 0.1 nM, or less. In some embodiments, Kd is about 1 mM or less. In some embodiments, Kd is about 0.5 mM or less. In some embodiments, Kd is about 0.1 mM or less. In some embodiments, Kd is about 0.05 mM or less. In some embodiments, Kd is about 0.01 mM or less. In some embodiments, Kd is about 0.005 mM or less. In some embodiments, Kd is about 0.001 mM or less. In some embodiments, Kd is about 500 nM or less. In some embodiments, Kd is about 200 nM or less. In some embodiments, Kd is about 100 nM or less. In some embodiments, Kd is about 50 nM or less. In some embodiments, Kd is about 20 nM or less. In some embodiments, Kd is about 10 nM or less. In some embodiments, Kd is about 5 nM or less. In some embodiments, Kd is about 2 nM or less. In some embodiments, Kd is about 1 nM or less. For example, in some embodiments, antibody binding moieties bind to IgG antibody agents with Kd described herein.

### Amino Acids

In some embodiments, provided compounds and agents may comprise one or more amino acid moieties, e.g., in antibody binding moieties, linker moieties, etc. Amino acid moieties can either be those of natural amino acids or unnatural amino acids. In some embodiments, an amino acid has the structure of formula A-I:

NH(R^{a1})-L^{a1}-C(R^{a2})(R^{a3})-L^{a2}-COOH A-I

or a salt thereof, wherein:
each of R^{a1}, R^{a2} and R^{a3} is independently -L^{a}-R′ or an amino acid side chain;
each of L^{a1} and L^{a2} is independently L^{a};
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—; each -Cy- is independently an optionally substituted bivalent monocyclic, bicyclic or polycyclic group wherein each monocyclic ring is independently selected from a C₃₋₂₀ cycloaliphatic ring, a C₆-20 aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon. In some embodiments, an amino acid residue, e.g., of an amino acid having the structure of formula A-I, has the structure of —N(R^{a1})-L^{a1}-C(R^{a2})(R^{a3})-L^{a2}-CO—. In some embodiments, each amino acid residue in a peptide independently has the structure of

—N(R^{a1})-L^{a1}-C(R^{a2})(R^{a3})-L^{a2}-CO—.

In some embodiments, the present disclosure provides a derivative of an amino acid of formula A-I or a salt thereof. In some embodiments, a derivative is an ester. In some embodiments, the present disclosure provides a compound of formula NH(R^{a1})-L^{a1}-C(R^{a2})(R^{a3})-L^{a2}-COOR^{CT} or salt thereof, wherein R^{CT} is R′ and each other variable is independently as described herein. In some embodiments, R^{CT} is R. In some embodiments, R^{CT} is optionally substituted aliphatic. In some embodiments, R^{CT} is t-butyl.

In some embodiments, L^{a1} is a covalent bond. In some embodiments, a compound of formula A-I is of the structure NH(R^{a1})—C(R^{a2})(R^{a3})-L^{a2}-COOH. In some embodiments, L^{a2} is —CH₂SCH₂—.

In some embodiments, L^{a2} is a covalent bond. In some embodiments, a compound of formula A-I is of the structure NH(R^{a1})-L^{a1}-C(R^{a2})(R^{a3})—COOH. In some embodiments, an amino acid residue has the structure of —N(R^{a1})-L^{a1}-C(R^{a2})(R^{a3})—CO—. In some embodiments, L^{a1} is —CH₂CH₂S—. In some embodiments, L^{a1} is —CH₂CH₂S—, wherein the CH₂ is bonded to NH(R^{a1}).

In some embodiments, L^{a1} is a covalent bond and L^{a2} is a covalent bond. In some embodiments, a compound of formula A-I is of the structure NH(R^{a1})—C(R^{a2})(R^{a3})—COOH; NH(R^{a1})—CH(R^{a2})—COOH; NH(R^{a1})—CH(R^{a3})—COOH; NH₂—CH(R^{a2})—COOH; NH₂—CH(R^{a3})—COOH; —N(R^{a1})—C(R^{a2})(R^{a3})—CO—; N(R^{a1})—CH(R^{a2})—CO—; —N(R^{a1})—CH(R^{a3})—CO—; —NH—CH(R^{a2})—CO—; or —NH—CH(R^{a3})—CO—.

In some embodiments, L^{a} is a covalent bond; L^{a} is optionally substituted C₁₋₆ bivalent aliphatic; L^{a} is optionally substituted C₁₋₆ alkylene; L^{a} is —CH₂—; L^{a} is —CH₂CH₂—; or L^{a} is —CH₂CH₂CH₂—.

In some embodiments, L^{a} is bivalent optionally substituted C₁₋₂₀ aliphatic, wherein one or more methylene units are independently replaced with —C(O)—, —N(R′)—, -Cy-, and/or —O—. In some embodiments, L^{a} is bivalent optionally substituted C₁₋₂₀ aliphatic, wherein one or more methylene units are independently replaced with —C(O)N(R′)—, -Cy-, and —O—. In some embodiments, L^{a} is bivalent optionally substituted C₁₋₂₀ aliphatic, wherein two or more methylene units are independently replaced with —C(O)N(R′)—, and -Cy- in addition to other optional replacements. In some embodiments, -Cy- is optionally substituted. In some embodiments, -Cy- is optionally substituted with an electron-withdrawing group as described herein. In some embodiments, -Cy- is substituted with one or more —F. In some embodiments, -Cy- is optionally substituted 1,3-phenylene. In some embodiments, -Cy- is optionally substituted 1,4-phenylene. In some embodiments, L^{a} is or comprises

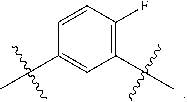 [SMILES: *c1ccc(F)c(*)c1]

In some embodiments, L^{a} is or comprises

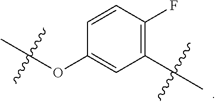 [SMILES: *Oc1ccc(F)c(*)c1]

In some embodiments, L^{a} is or comprises

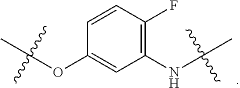 [SMILES: *Nc1cc(O*)ccc1F]

In some embodiments, L^{a} is or comprises

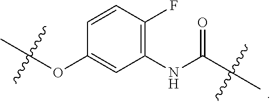 [SMILES: *Oc1ccc(F)c(NC(*)=O)c1]

In some embodiments, L^{a} is or comprises

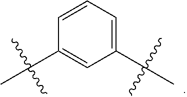 [SMILES: *c1cccc(*)c1]

In some embodiments, L^{a} is or comprises

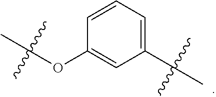 [SMILES: *Oc1cccc(*)c1]

In some embodiments, L^{a} is or comprises

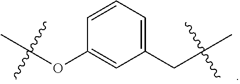 [SMILES: *Cc1cccc(O*)c1]

In some embodiments, L^{a} is or comprises

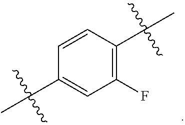 [SMILES: *c1ccc(*)c(F)c1]

In some embodiments, L^{a} is or comprises

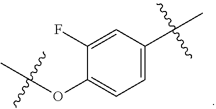 [SMILES: *Oc1ccc(*)cc1F]

In some embodiments, L^{a} is or comprises

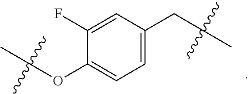 [SMILES: *Cc1ccc(O*)c(F)c1]

In some embodiments, L^{a} is or comprises

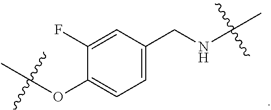 [SMILES: *NCc1ccc(O*)c(F)c1]

In some embodiments, L^{a} is or comprises

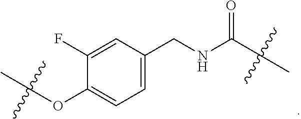 [SMILES: *Oc1ccc(CNC(*)=O)cc1F]

In some embodiments, L^{a} is or comprises

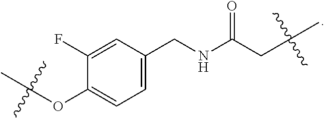 [SMILES: *CC(=O)NCc1ccc(O*)c(F)c1]

In some embodiments, L^{a} is or comprises

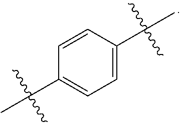 [SMILES: *c1ccc(*)cc1]

In some embodiments, L^{a} is or comprises

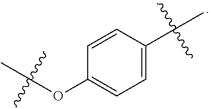 [SMILES: *Oc1ccc(*)cc1]

In some embodiments, L^{a} is or comprises

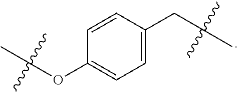 [SMILES: *Cc1ccc(O*)cc1]

In some embodiments, L^{a} is or comprises

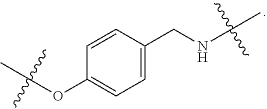 [SMILES: *NCc1ccc(O*)cc1]

In some embodiments, L^{a} is or comprises

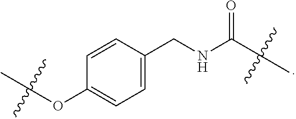 [SMILES: *Oc1ccc(CNC(*)=O)cc1]

In some embodiments, L^{a} is or comprises

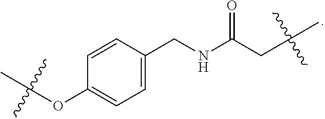 [SMILES: *CC(=O)NCc1ccc(O*)cc1]

In some embodiments, R′ is R. In some embodiments, R^{a1} is R, wherein R is as described in the present disclosure. In some embodiments, R^{a1} is R, wherein R methyl. In some embodiments, R^{a2} is R, wherein R is as described in the present disclosure. In some embodiments, R^{a3} is R, wherein R is as described in the present disclosure. In some embodiments, each of R^{a1}, R^{a2}, and R^{a3} is independently R, wherein R is as described in the present disclosure.

In some embodiments, R^{a1} is hydrogen. In some embodiments, R^{a1} is a protective group. In some embodiments, R^{a1} is -Fmoc. In some embodiments, R^{a1} is -Dde.

In some embodiments, each of R^{a1}, R^{a2} and R^{a3} is independently -L^{a}-R′.

In some embodiments, R^{a2} is hydrogen. In some embodiments, R^{a3} is hydrogen. In some embodiments, R^{a1} is hydrogen, and at least one of R^{a2} and R^{a3} is hydrogen. In some embodiments, R^{a1} is hydrogen, one of R^{a2} and R^{a3} is hydrogen, and the other is not hydrogen. In some embodiments, R^{a2} is -L^{a}-R and R^{a3} is —H. In some embodiments, R^{a3} is -L^{a}-R and R^{a2} is —H. In some embodiments, R^{a2} is —CH₂—R and R^{a3} is —H. In some embodiments, R^{a3} is —CH₂—R and R^{a2} is —H. In some embodiments, R^{a2} is R and R^{a3} is —H. In some embodiments, R^{a3} is R and R^{a2} is —H.

In some embodiments, R^{a2} is -L^{a}-R, wherein R is as described in the present disclosure. In some embodiments, R^{a2} is -L^{a}-R, wherein R is an optionally substituted group selected from C₃₋₃₀ cycloaliphatic, C₅₋₃₀ aryl, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, R^{a2} is -L^{a}-R, wherein R is an optionally substituted group selected from C₆₋₃₀ aryl and 5-30 membered heteroaryl having 1-10 heteroatoms independently selected from oxygen, nitrogen, sulfur, phosphorus and silicon. In some embodiments, R^{a2} is a side chain of an amino acid. In some embodiments, R^{a2} is a side chain of a standard amino acid.

In some embodiments, R^{a3} is -L^{a}-R, wherein R is as described in the present disclosure. In some embodiments, R^{a3} is -L^{a}-R, wherein R is an optionally substituted group selected from C₃₋₃₀ cycloaliphatic, C₅₋₃₀ aryl, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, R^{a3} is -L^{a}-R, wherein R is an optionally substituted group selected from C₆₋₃₀ aryl and 5-30 membered heteroaryl having 1-10 heteroatoms independently selected from oxygen, nitrogen, sulfur, phosphorus and silicon. In some embodiments, R^{a3} is a side chain of an amino acid. In some embodiments, R^{a3} is a side chain of a standard amino acid.

In some embodiments, one or R^{a2} and R^{a3} is —H. In some embodiments, one or R^{a2} and R^{a3} is -L^{a}-R, wherein L^{a} is as described herein. In some embodiments, L^{a} is not a covalent bond. In some embodiments, one or more methylene units of L^{a} are independently and optionally replaced as described herein, e.g., with —C(O)—, —N(R′)—, —O—, —C(O)—N(R′)— and/or -Cy-, etc. In some embodiments, L^{a} is or comprises —C(O)—, —N(R′)— and -Cy-. In some embodiments, L^{a} is or comprises —C(O)N(R′)— and -Cy-. In some embodiments, as described herein, -Cy- is substituted and one or more substituents are independently an electron-withdrawing group.

In some embodiments, an amino acid side chain is R^{a2} or R^{a3}. In some embodiments, an amino acid side chain is or comprises -L^{LG1}-L^{LG2}-L^{LG3}-L^{LG4}-H. In some embodiments, an amino acid side chain is or comprises -L^{LG2}-L^{LG3}-L^{LG4}-H. In some embodiments, an amino acid side chain is or comprises -L^{LG3}-L^{LG4}-H. In some embodiments, an amino acid side chain is or comprises -L^{LG4}-H. In some embodiments, such a side chain is

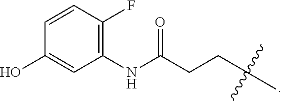 [SMILES: *CCC(=O)Nc1cc(O)ccc1F]

In some embodiments, such a side chain is

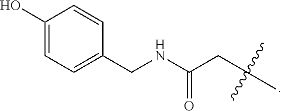 [SMILES: *CC(=O)NCc1ccc(O)cc1]

In some embodiments, such a side chain is

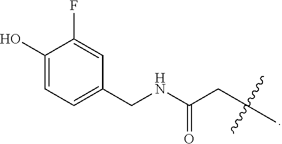 [SMILES: *CC(=O)NCc1ccc(O)c(F)c1]

In some embodiments, such a side chain is

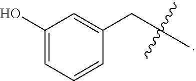 [SMILES: *Cc1cccc(O)c1]

In some embodiments, R is an optionally substituted C₁₋₆ aliphatic. In some embodiments, R is an optionally substituted C₁₋₆ alkyl. In some embodiments, R is —CH₃. In some embodiments, R is optionally substituted pentyl. In some embodiments, R is n-pentyl.

In some embodiments, R is a cyclic group. In some embodiments, R is an optionally substituted C₃₋₃₀ cycloaliphatic group. In some embodiments, R is cyclopropyl.

In some embodiments, R is an optionally substituted aromatic group, and an amino acid residue of an amino acid of formula A-I is a Xaa^{A}. In some embodiments, R^{a2} or R^{a3} is —CH₂—R, wherein R is an optionally substituted aryl or heteroaryl group. In some embodiments, R is optionally substituted phenyl. In some embodiments, R is phenyl. In some embodiments, R is optionally substituted phenyl. In some embodiments, R is 4-trifluoromethylphenyl. In some embodiments, R is 4-phenylphenyl. In some embodiments, R is optionally substituted 5-30 membered heteroaryl having 1-10 heteroatoms independently selected from oxygen, nitrogen, sulfur, phosphorus and silicon. In some embodiments, R is optionally substituted 5-14 membered heteroaryl having 1-5 heteroatoms independently selected from oxygen, nitrogen, and sulfur. In some embodiments, R is

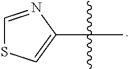 [SMILES: *c1cscn1]

In some embodiments, R is optionally substituted pyridinyl. In some embodiments, R is 1-pyridinyl. In some embodiments, R is 2-pyridinyl. In some embodiments, R is 3-pyridinyl. In some embodiments, R is

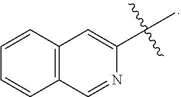 [SMILES: *c1cc2ccccc2cn1]

In some embodiments, R′ is-COOH. In some embodiments, a compound of and an amino acid residue of an amino acid of formula A-I is a Xaa^{N}.

In some embodiments, R′ is-NH₂. In some embodiments, a compound of an amino acid residue of an amino acid of formula A-I is a Xaa^{P}.

In some embodiments, R^{a2} or R^{a3} is R, wherein R is C₁₋₂₀ aliphatic as described in the present disclosure. In some embodiments, a compound of an amino acid residue of an amino acid of formula A-I is a Xaa^{H}. In some embodiments, R is —CH₃. In some embodiments, R is ethyl. In some embodiments, R is propyl. In some embodiments, R is n-propyl. In some embodiments, R is butyl. In some embodiments, R is n-butyl. In some embodiments, R is pentyl. In some embodiments, R is n-pentyl. In some embodiments, R is cyclopropyl.

In some embodiments, two or more of R^{a1}, R^{a2}, and R^{a3} are R and are taken together to form an optionally substituted ring as described in the present disclosure.

In some embodiments, R^{a1} and one of R^{a2} and R^{a3} are R and are taken together to form an optionally substituted 3-6 membered ring having no additional ring heteroatom other than the nitrogen atom to which R^{a1} is bonded to. In some embodiments, a formed ring is a 5-membered ring as in proline.

In some embodiments, R^{a2} and R^{a3} are R and are taken together to form an optionally substituted 3-6 membered ring as described in the present disclosure. In some embodiments, R^{a2} and R^{a3} are R and are taken together to form an optionally substituted 3-6 membered ring having one or more nitrogen ring atom. In some embodiments, R^{a2} and R^{a3} are R and are taken together to form an optionally substituted 3-6 membered ring having one and no more than one ring heteroatom which is a nitrogen atom. In some embodiments, a ring is a saturated ring.

In some embodiments, an amino acid is a natural amino acid. In some embodiments, an amino acid is an unnatural amino acid. In some embodiments, an amino acid is an alpha-amino acid. In some embodiments, an amino acid is a beta-amino acid. In some embodiments, a compound of formula A-I is a natural amino acid. In some embodiments, a compound of formula A-I is an unnatural amino acid.

In some embodiments, an amino acid comprises a hydrophobic side chain. In some embodiments, an amino acid with a hydrophobic side chain is A, V, I, L, M, F, Y or W. In some embodiments, an amino acid with a hydrophobic side chain is A, V, I, L, M, or F. In some embodiments, an amino acid with a hydrophobic side chain is A, V, I, L, or M. In some embodiments, an amino acid with a hydrophobic side chain is A, V, I, or L. In some embodiments, a hydrophobic side chain is R wherein R is C₁₋₁₀ aliphatic. In some embodiments, R is C₁₋₁₀ alkyl. In some embodiments, R is methyl. In some embodiments, R is ethyl. In some embodiments, R is propyl. In some embodiments, R is butyl. In some embodiments, R is pentyl. In some embodiments, R is n-pentyl. In some embodiments, an amino acid with a hydrophobic side chain is NH₂CH(CH₂CH₂CH₂CH₂CH₃)COOH. In some embodiments, an amino acid with a hydrophobic side chain is (S)—NH₂CH(CH₂CH₂CH₂CH₂CH₃)COOH. In some embodiments, an amino acid with a hydrophobic side chain is (R)—NH₂CH(CH₂CH₂CH₂CH₂CH₃)COOH. In some embodiments, a hydrophobic side chain is —CH₂R wherein R is optionally substituted phenyl. In some embodiments, R is phenyl. In some embodiments, R is phenyl substituted with one or more hydrocarbon group. In some embodiments, R is 4-phenylphenyl. In some embodiments, an amino acid with a hydrophobic side chain is NH₂CH(CH₂₋₄-phenylphenyl)COOH. In some embodiments, an amino acid with a hydrophobic side chain is (S)—NH₂CH(CH₂₋₄-phenylphenyl)COOH. In some embodiments, an amino acid with a hydrophobic side chain is (R)—NH₂CH(CH₂₋₄-phenylphenyl)COOH.

In some embodiments, an amino acid comprises a positively charged side chain (e.g., at physiological pH) as described herein. In some embodiments, such an amino acid comprises a basic nitrogen in its side chain. In some embodiments, such an amino acid is Arg, His or Lys. In some embodiments, such an amino acid is Arg. In some embodiments, such an amino acid is His. In some embodiments, such an amino acid is Lys.

In some embodiments, an amino acid comprises a negatively charged side chain (e.g., at physiological pH) as described herein. In some embodiments, such an amino acid comprises a —COOH in its side chain. In some embodiments, such an amino acid is Asp. In some embodiments, such an amino acid is Glu.

In some embodiments, an amino acid comprises a side chain comprising an aromatic group as described herein. In some embodiments, such an amino acid is Phe, Tyr, Trp, or His. In some embodiments, such an amino acid is Phe. In some embodiments, such an amino acid is Tyr. In some embodiments, such an amino acid is Trp. In some embodiments, such an amino acid is His. In some embodiments, such an amino acid is NH₂—CH(CH₂₋₄-phenylphenyl)-COOH. In some embodiments, such an amino acid is (S)—NH₂—CH(CH₂₋₄-phenylphenyl)-COOH. In some embodiments, such an amino acid is (R)—NH₂—CH(CH₂₋₄-phenylphenyl)-COOH.

In some embodiments, an amino acid is

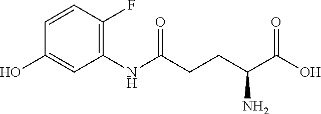 [SMILES: N[C@@H](CCC(=O)Nc1cc(O)ccc1F)C(=O)O]

or a salt thereof. In some embodiments, an amino acid is

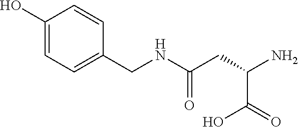 [SMILES: N[C@@H](CC(=O)NCc1ccc(O)cc1)C(=O)O]

or a salt thereof. In some embodiments, an amino acid is

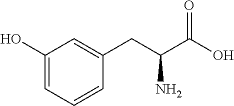 [SMILES: N[C@@H](Cc1cccc(O)c1)C(=O)O]

or a salt thereof. In some embodiments, an amino acid is

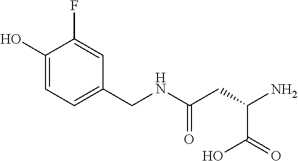 [SMILES: N[C@@H](CC(=O)NCc1ccc(O)c(F)c1)C(=O)O]

or a salt thereof. In some embodiments, an amino acid is

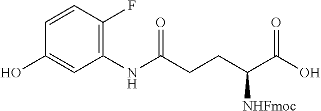

or a salt thereof. In some embodiments, an amino acid is

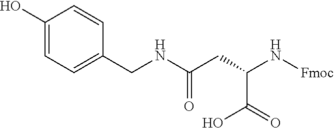

or a salt thereof. In some embodiments, an amino acid is

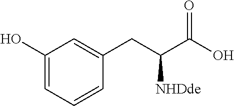

or a salt thereof. In some embodiments, an amino acid is

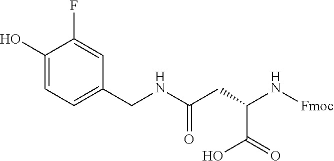

or a salt thereof. In some embodiments, a provided compound is

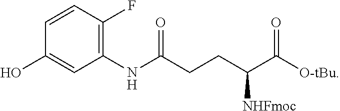

In some embodiments, the present disclosure provides polypeptide agents comprising one or more amino acid residues described in the present disclosure.

### Reactive Group

In some embodiments, provided compounds, e.g., those useful as reaction partners, comprise reactive groups (e.g., RG). As exemplified herein, in many embodiments, in provided compounds reactive groups (e.g., RG) are located between first groups (e.g., LG) and moieties of interest (e.g., MOI), and are optionally and independently linked to first groups and moieties of interest via linkers. In some embodiments, RG is a reaction group as described herein.

In some embodiments, as demonstrated herein, reactive groups when utilized in compounds that comprise no target binding moieties react slowly and provide low level of, in some embodiments, substantially no conjugation of moieties of interest with target agents. As demonstrated herein, combination of reactive groups with target binding moieties in the same compounds, e.g., as in compounds of formula R—I or salts thereof, can, among other things, promote reactions between reactive groups and target agents, enhance reaction efficiency, reduce side reactions, and/or improve reaction selectivity (e.g., in terms of target sites wherein conjugation of moieties of interest with target agents occurs).

Reactive groups in provided compounds can react with various types of groups in target agents. In some embodiments, reactive groups in provided compounds selectively react with amino groups of target agents, e.g., —NH₂ groups on side chains of lysine residues of proteins. In some embodiments, reactive groups when utilized in provided compounds, e.g., those of formula R—I or salts thereof, selectively react with particular sites of target agents, e.g., as shown in examples herein, one or more of K246, K248, K288, K290, K317, etc. of IgG1, K251, K 253, etc. for IgG2, K239, K241 for IgG4, etc. In some embodiments, a site is K246 or K248 of an antibody heavy chain. In some embodiments, sites are K246 and/or K248 of an antibody heavy chain. In some embodiments, a site is K246 of an antibody heavy chain. In some embodiments, a site is K248 of an antibody heavy chain. In some embodiments, a site is K288 or K290 of an antibody heavy chain. In some embodiments, a site is K288 of an antibody heavy chain. In some embodiments, a site is K290 of an antibody heavy chain. In some embodiments, a site is K317. In some embodiments, a site is K414 of an antibody heavy chain. In some embodiments, a site is K185 of an antibody light chain. In some embodiments, a site is K187 of an antibody light chain. In some embodiments, sites are K251 and/or K253 of an IgG2 heavy chain. In some embodiments, a site is K251 of an IgG2 heavy chain. In some embodiments, a site is K253 of an IgG2 heavy chain. In some embodiments, sites are K239 and/or K241 of an IgG4 heavy chain. In some embodiments, a site is K239 of an IgG4 heavy chain. In some embodiments, a site is K241 of an IgG4 heavy chain. In some embodiments, conjugation selectively occurs at one or more heavy chain sites over light chain sites. In some embodiments, for technologies without target binding moieties, conjugation occurs at light chain sites more than heavy chain sites (e.g., see FIG. 15).

In some embodiments, a reactive group, e.g., RG, is or comprises an ester group. In some embodiments, a reactive group, e.g., RG, is or comprises an electrophilic group, e.g., a Michael acceptor.

In some embodiments, a reactive group, e.g., RG, is or comprises -L^{RG1}-L^{RG2}-, wherein each of L^{RG1} and L^{RG2} is independently L as described herein. In some embodiments, a reactive group, e.g., RG, is or comprises -L^{LG4}-L^{RG1}-L^{RG2}-, wherein each variable is as described herein. In some embodiments, a reactive group, e.g., RG, is or comprises -L^{LG3}-L^{LG4}-L^{RG1}-L^{RG2}- wherein each variable is as described herein. In some embodiments, a reactive group, e.g., RG, is or comprises -L^{LG2}-L^{LG3}-L^{LG4}-L^{RG1}-L^{RG2} wherein each variable is as described herein. In some embodiments, a reactive group, e.g., RG, is or comprises -L^{LG4}-L^{RG2}-, wherein each variable is as described herein. In some embodiments, a reactive group, e.g., RG, is or comprises -L^{LG3}-L^{LG4}-L^{RG2}-, wherein each variable is as described herein. In some embodiments, a reactive group, e.g., RG, is or comprises -L^{LG2}-L^{LG3}-L^{LG4}-L^{RG2}-, wherein each variable is as described herein.

In some embodiments, as described herein, L^{LG4} is —O—. In some embodiments, L^{LG4} is —N(R)—. In some embodiments, L^{LG4} is —NH—.

In some embodiments, as described herein, L^{LG3} is or comprises an optionally substituted aryl ring. In some embodiments, L^{LG3} is or comprises a phenyl ring. In some embodiments, an aryl or phenyl ring is substituted. In some embodiments, a substituent is an electron-withdrawing group as described herein, e.g., —NO₂, —F, etc.

In some embodiments, L^{RG1} is a covalent bond. In some embodiments, L^{RG1} is not a covalent bond. In some embodiments, L^{RG1} is —S(O)₂—.

In some embodiments, L^{RG2} is —C(O)—. In some embodiments, a reactive group is or comprises -L^{LG4}-C(O)—, wherein each variable is as described herein. In some embodiments, a reactive group is or comprises -L^{LG3}-L^{LG4}-C(O)—, wherein each variable is as described herein. In some embodiments, a reactive group is or comprises -L^{LG2}-L^{LG3}-L^{LG4}-C(O)—, wherein each variable is as described herein.

In some embodiments, L^{RG2} is -L^{RG3}-C(═CR^{RG1}R^{RG2})—CR^{RG3}R^{RG4}—, wherein each of R^{RG1}, R^{RG2}, R^{RG3} and R^{RG4} is independently -L-R′ and L^{RG3} is —C(O)—, —C(O)O—, —C(O)N(R′)—, —S(O)—, —S(O)₂—, —P(O)(OR′)—, —P(O)(SR′)—, or —P(O)(N(R′)₂)—. In some embodiments, each of R^{RG1}, R^{RG2}, R^{RG3} and R^{RG4} is independently R′. In some embodiments, one or more of R^{RG1}, R^{RG2}, R^{RG3} and R^{RG4} is independently —H. In some embodiments, L^{RG3} is —C(O)—. In some embodiments, L^{RG3} is —C(O)O—. In some embodiments, —O—, —N(R′)—, etc. of L^{RG3} is bonded to L^{PM}.

In some embodiments, R^{RG1} is —H. In some embodiments, R^{RG3} is —H.

In some embodiments, L^{RG2} is optionally substituted -L^{RG3}-C(═CHR^{RG2})—CHR^{RG4}—, wherein each variable is as described herein.

In some embodiments, R^{RG2} and R^{RG4} are taken together with their intervening atoms to form an optionally substituted ring as described herein. In some embodiments, a formed ring is an optionally substituted 3-10 membered monocyclic or bicyclic ring having 0-5 heteroatoms. In some embodiments, a formed ring is an optionally substituted 3-10 membered cycloaliphatic ring. In some embodiments, a formed ring is an optionally substituted 3-8 membered cycloaliphatic ring. In some embodiments, a formed ring is an optionally substituted 5-8 membered cycloaliphatic ring. In some embodiments, a formed ring is an optionally substituted 5-membered cycloaliphatic ring. In some embodiments, a formed ring is an optionally substituted 6-membered cycloaliphatic ring. In some embodiments, a formed ring is an optionally substituted 7-membered cycloaliphatic ring. In some embodiments, a formed ring is substituted. In some embodiments, a formed ring is not substituted. In some embodiments, a formed ring contains no additional unsaturation in addition to the double bond in C(═CHR^{RG2}) or C(═CR^{RG1}R^{RG2}).

In some embodiments, —C(═CHR^{RG2})—CHR^{RG4} or —C(═CR^{RG1}R^{RG2})—CR^{RG3}R^{RG4} is optionally substituted

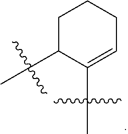 [SMILES: *C1=CCCCC1*]

In some embodiments, —C(═CHR^{RG2})—CHR^{RG4} or —C(═CR^{RG1}R^{RG2})—CR^{RG3}R^{RG4} is

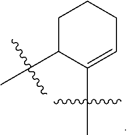 [SMILES: *C1=CCCCC1*]

In some embodiments, —C(═CHR^{RG2})—CHR^{RG4}-L^{RG3}- or —C(═CR^{RG1}R^{RG2})—CR^{RG3}R^{RG4}-L^{RG3}- is optionally substituted

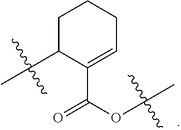 [SMILES: *OC(=O)C1=CCCCC1*]

In some embodiments, —C(═CHR^{RG2})—CHR^{RG4}-L^{RG3}- or —C(═CR^{RG1}R^{RG2})—CR^{RG3}R^{RG4}-L^{RG3}- is

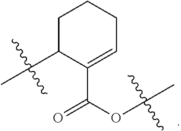 [SMILES: *OC(=O)C1=CCCCC1*]

In some embodiments, -L^{RG1}-C(═CHR^{RG2})—CHR^{RG4}-L^{RG3}- or -L^{RG1}-C(═CR^{RG1}R^{RG2})—CR^{RG3}R^{RG4}-L^{RG3}- is optionally substituted

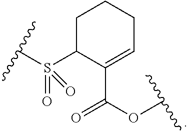 [SMILES: *OC(=O)C1=CCCCC1S(*)(=O)=O]

In some embodiments, -L^{RG1}-C(═CHR^{RG2})—CHR^{RG4}-L^{RG3}- or -L^{RG1}-C(═C^{RG1}R^{RG2})—CR^{RG3}R^{RG4}-L^{RG3}- is optionally substituted

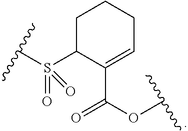 [SMILES: *OC(=O)C1=CCCCC1S(*)(=O)=O]

In some embodiments, a reactive group is a structure selected from the Table below. In some embodiments, -L^{LG2}-L^{LG3}-L^{LG4}-L^{RG1}-L^{RG2}- is a structure selected from the Table below. In some embodiments, -L^{LG2}-L^{LG3}-L^{LG4}-RG- is a structure selected from the Table below.

TABLE RG-1
Certain structures as examples.

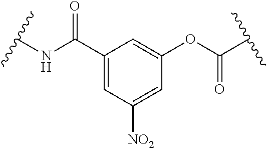 [SMILES: *NC(=O)c1cc(OC(*)=O)cc([N+](=O)[O-])c1]

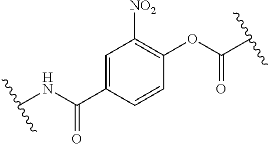 [SMILES: *NC(=O)c1ccc(OC(*)=O)c([N+](=O)[O-])c1]

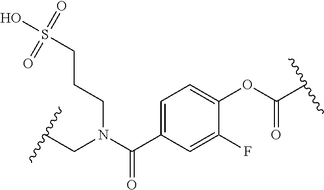 [SMILES: *CN(CCCS(=O)(=O)O)C(=O)c1ccc(OC(*)=O)c(F)c1]

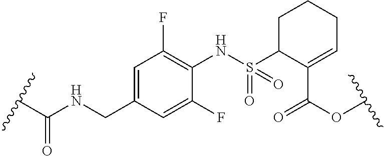 [SMILES: *OC(=O)C1=CCCCC1S(=O)(=O)Nc1c(F)cc(CNC(*)=O)cc1F]

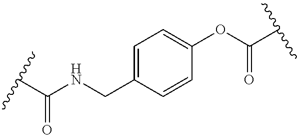 [SMILES: *C(=O)NCc1ccc(OC(*)=O)cc1]

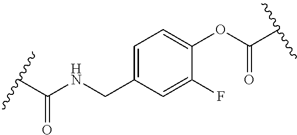 [SMILES: *C(=O)NCc1ccc(OC(*)=O)c(F)c1]

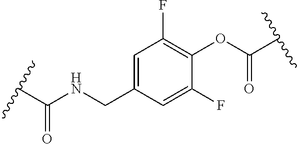 [SMILES: *C(=O)NCc1cc(F)c(OC(*)=O)c(F)c1]

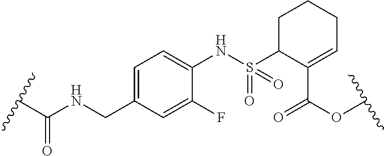 [SMILES: *OC(=O)C1=CCCCC1S(=O)(=O)Nc1ccc(CNC(*)=O)cc1F]

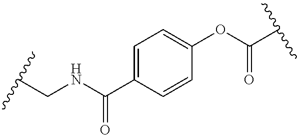 [SMILES: *CNC(=O)c1ccc(OC(*)=O)cc1]

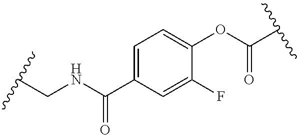 [SMILES: *CNC(=O)c1ccc(OC(*)=O)c(F)c1]

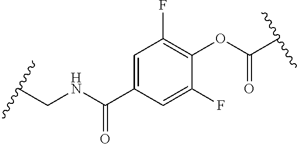 [SMILES: *CNC(=O)c1cc(F)c(OC(*)=O)c(F)c1]

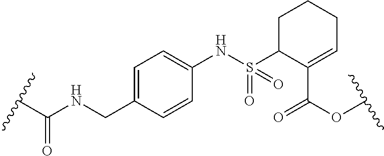 [SMILES: *OC(=O)C1=CCCCC1S(=O)(=O)Nc1ccc(CNC(*)=O)cc1]

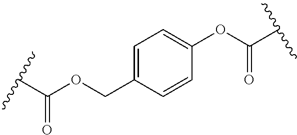 [SMILES: *C(=O)OCc1ccc(OC(*)=O)cc1]

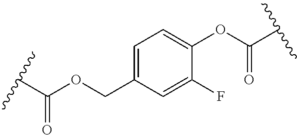 [SMILES: *C(=O)OCc1ccc(OC(*)=O)c(F)c1]

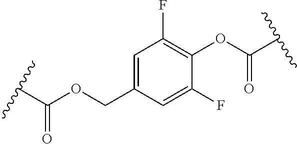 [SMILES: *C(=O)OCc1cc(F)c(OC(*)=O)c(F)c1]

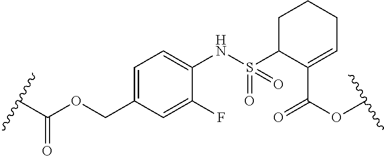 [SMILES: *OC(=O)C1=CCCCC1S(=O)(=O)Nc1ccc(COC(*)=O)cc1F]

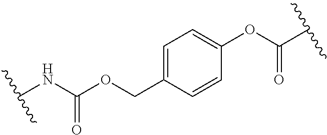 [SMILES: *NC(=O)OCc1ccc(OC(*)=O)cc1]

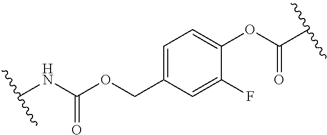 [SMILES: *NC(=O)OCc1ccc(OC(*)=O)c(F)c1]

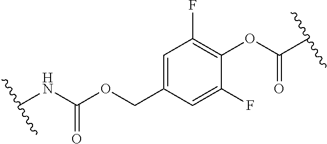 [SMILES: *NC(=O)OCc1cc(F)c(OC(*)=O)c(F)c1]

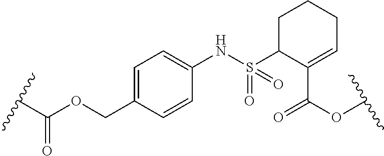 [SMILES: *OC(=O)C1=CCCCC1S(=O)(=O)Nc1ccc(COC(*)=O)cc1]

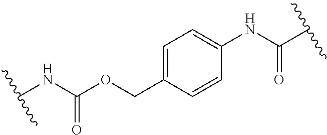 [SMILES: *NC(=O)OCc1ccc(NC(*)=O)cc1]

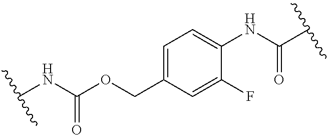 [SMILES: *NC(=O)OCc1ccc(NC(*)=O)c(F)c1]

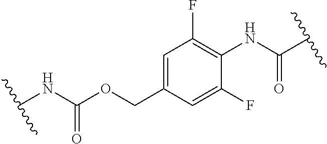 [SMILES: *NC(=O)OCc1cc(F)c(NC(*)=O)c(F)c1]

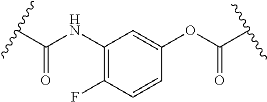 [SMILES: *C(=O)Nc1cc(OC(*)=O)ccc1F]

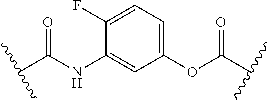 [SMILES: *C(=O)Nc1cc(OC(*)=O)ccc1F]

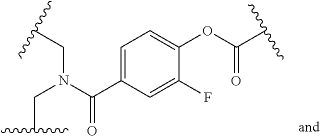 [SMILES: *CN(C*)C(=O)c1ccc(OC(*)=O)c(F)c1]

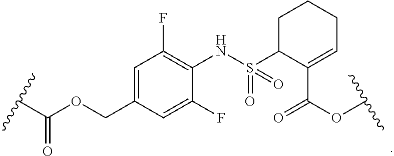 [SMILES: *OC(=O)C1=CCCCC1S(=O)(=O)Nc1c(F)cc(COC(*)=O)cc1F]

In some embodiments, -L^{LG4}-L^{RG2}- is —O—C(O)—. In some embodiments, -L^{LG4}-L^{RG2}- is —S—C(O)—. In some embodiments, L^{LG4}-L^{RG1}-L^{RG2}- is —S—C(O)—.

In some embodiments, -L^{LG4}-L^{RG2}- is —N(−)—C(O)—, wherein N is a ring atom of an optionally substituted heteroaryl ring. In some embodiments, -L^{LG4}-L^{RG2}- is —N(−)—C(O)—, wherein N is a ring atom of L^{LG4} which is or Comprises an optionally substituted heteroaryl ring. In some embodiments, -L^{LG4}-L^{RG2}- is —N(−)—C(O)—O—, wherein N is a ring atom of L^{LG4} which is or Comprises an optionally substituted heteroaryl ring.

In some embodiments, L^{RG2} is optionally substituted —CH₂—C(O)—, wherein —CH₂— is bonded to an electron-withdrawing group comprising or connected to a target binding moiety. In some embodiments, L^{RG2} is optionally substituted —CH₂— bonded to an electron-withdrawing group comprising or connected to a target binding moiety. In some embodiments, L^{RG1} is an electron-withdrawing group. In some embodiments, L^{RG1} is —C(O)—. In some embodiments, L^{RG1} is —S(O)—. In some embodiments, L^{RG1} is —S(O)₂—. In some embodiments, L^{RG1} is —P(O(OR)—. In some embodiments, L^{RG1} is —P(O(SR)—. In some embodiments, L^{RG1} is —P(O(N(R)₂)—. In some embodiments, L^{RG1} is —OP(O(OR)—. In some embodiments, L^{RG1} is —OP(O(SR)—. In some embodiments, L^{RG1} is —OP(O(N(R)₂)—.

In some embodiments, L^{RG2} is optionally substituted —CH₂—C(O)—, wherein —CH₂— is bonded to a leaving group comprising or connected to a target binding moiety. In some embodiments, L^{RG2} is optionally substituted —CH₂— bonded to a leaving group comprising or connected to a target binding moiety. In some embodiments, L^{RG1} is —O—C(O)—. In some embodiments, L^{RG1} is —OS(O)₂—. In some embodiments, L^{RG1} is —OP(O(OR)—. In some embodiments, L^{RG1} is —OP(O(SR)—. In some embodiments, L^{RG1} is —OP(O(N(R)₂)—.

In some embodiments, a reactive group reacts with an amino group of a target agent. In some embodiments, an amino group is —NH₂ of the side chain of a lysine residue.

In some embodiments, a target agent is a protein agent. In some embodiments, a target agent is an antibody agent. In some embodiments, a reactive group reacts with an amino acid residue of such protein or antibody agent. In some embodiments, an amino acid residue is a lysine residue. In some embodiments, a reactive group reacts with —NH₂ of the side chain of a lysine residue. In some embodiments, a reactive group is or comprises —C(O)—O—, it reacts with —NH₂ (e.g., of the side chain of a lysine residue), and forms an amide group —C(O)—O— with the —NH₂.

### Linker Moieties

In some embodiments, moieties are optionally connected to each other through linker moieties. For example, in some embodiments, a reactive group, e.g., RG, is connected to a moiety of interest, e.g., MOI, through a linker, e.g., L^{RM}. In some embodiments, a moiety, e.g., LG, may also comprise one or more linkers, e.g., L^{LG1}, L^{LG2}, L^{LG3}, L^{LG4}, etc., to link various portions. In some embodiments, L^{LG} is a linker moiety described herein. In some embodiments, L^{LG1} is a linker moiety described herein. In some embodiments, L^{LG2} is a linker moiety described herein. In some embodiments, L^{LG3} is a linker moiety described herein. In some embodiments, L^{LG4} is a linker moiety described herein. In some embodiments, L^{RM} is a linker moiety described herein. In some embodiments, L^{PM} is L as described herein. In some embodiments, L^{PM} is a linker moiety described herein. In some embodiments, L^{PM} is L as described herein.

Linker moieties of various types and/or for various purposes, e.g., those utilized in antibody-drug conjugates, etc., may be utilized in accordance with the present disclosure.

Linker moieties can be either bivalent or polyvalent depending on how they are used. In some embodiments, a linker moiety is bivalent. In some embodiments, a linker is polyvalent and connecting more than two moieties.

In some embodiments, a linker moiety, e.g., L^{z} (wherein z represents superscript text; e.g., L^{PM}, L^{RM}, L^{LG}, L^{LG1}, etc.), is or comprises L.

In some embodiments, L is a covalent bond, or a bivalent or polyvalent optionally substituted, linear or branched C₁₋₁₀₀ group comprising one or more aliphatic, aryl, heteroaliphatic having 1-20 heteroatoms, heteroaromatic having 1-20 heteroatoms, or any combinations thereof, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or —[(—O—C(R′)₂—C(R′)₂—)ₙ]—, wherein n is 1-20. In some embodiments, each amino acid residue is independently a residue of an amino acid having the structure of formula A-I or a salt thereof. In some embodiments, each amino acid residue independently has the structure of —N(R^{a1})-L^{a1}-C(R^{a2})(R^{a3})-L^{a2}-CO— or a salt form thereof.

In some embodiments, L is bivalent. In some embodiments, L is a covalent bond.

In some embodiments, L is a bivalent or optionally substituted, linear or branched group selected from C₁₋₁₀₀ aliphatic and C₁₋₁₀₀ heteroaliphatic having 1-50 heteroatoms, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue or —[(—O—C(R′)₂—C(R′)₂—)ₙ]—.

In some embodiments, L is a bivalent or optionally substituted, linear or branched group selected from C₁₋₂₀ aliphatic and C₁₋₂₀ heteroaliphatic having 1-10 heteroatoms, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue or —[(—O—C(R′)₂—C(R′)₂—)ₙ]—.

In some embodiments, L is a bivalent or optionally substituted, linear or branched group selected from C₁₋₂₀ aliphatic wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue or —[(—O—C(R′)₂—C(R′)₂—)ₙ]—.

In some embodiments, L is a bivalent or optionally substituted, linear or branched group selected from C₁₋₂₀ aliphatic wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, an amino acid residue or —[(—O—C(R′)₂—C(R′)₂—)ₙ]—.

In some embodiments, L is a bivalent or optionally substituted, linear or branched group selected from C₁₋₂₀ aliphatic wherein one or more methylene units of the group are optionally and independently replaced with —C≡C—, -Cy-, —C(R′)₂—, —O—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, an amino acid residue or —[(—O—C(R′)₂—C(R′)₂—)ₙ]—.

In some embodiments, a linker moiety, e.g., L, L^{PM}, L^{RM}, etc., comprises an acidic group, e.g., —S(O)₂OH.

In some embodiments, L is or comprises —[(—O—C(R′)₂—C(R′)₂—)ₙ]—. In some embodiments, L is or comprises —[(—O—CH₂—CH₂—)ₙ]—. In some embodiments, L is —[(—CH₂—CH₂₋₀)₆]—CH₂—CH₂—. In some embodiments, L is -[(—CH₂—CH₂₋₀)₈]—CH₂—CH₂—. In some embodiments, —CH₂—CH₂—O— is bonded to a target binding moiety at a —CH₂—. In some embodiments, —CH₂—CH₂—O— is bonded to a moiety of interest at a —CH₂—. In some embodiments, L^{PM} is such L as described herein. In some embodiments, L^{RM} is such L as described herein.

In some embodiments, a linker moiety is trivalent or polyvalent. For example, in some embodiments, a linker moiety is L as described herein and L is trivalent or polyvalent. In some embodiments, L is trivalent. For example, in some embodiments, L is —CH₂—N(—CH₂—)—C(O)—.

In some embodiments, L is or comprises a bioorthogonal or enzymatic reaction product moiety. In some embodiments, L is or comprise an optionally substituted triazole moiety (which is optionally part of a bi- or poly-cyclic ring system). In some embodiments, L is or comprises LPXTG. In some embodiments, L is or comprises LPETG. In some embodiments, L is or comprises LPXT(G)n, wherein n is 1, 2, 3, 4, 5, 6, 7, 8, 9, or 10. In some embodiments, L is or comprises LPET(G)n, wherein n is 1, 2, 3, 4, 5, 6, 7, 8, 9, or 10.

In some embodiments, provided compounds/agents (e.g., reaction partners, agents (e.g., products of provided methods and/or steps therein) comprise no cleavable groups (except one or more reactive groups and/or moieties therein) that could be cleaved under conditions that would not substantially damage or transform target agents and/or agents comprising target agent moieties (e.g., conjugation products comprising target agent moieties). In some embodiments, provided compounds/agents (e.g., reaction partners, agents (e.g., products of provided methods and/or steps therein) comprise no cleavable groups (except one or more reactive groups and/or moieties therein) that could be cleaved under conditions that would not render target agents and/or agents comprising target agent moieties (e.g., conjugation products comprising target agent moieties) ineffective for one or more uses (e.g., for use as diagnostic agents, therapeutic agents, etc.). In some embodiments, provided compounds/agents (e.g., reaction partners, agents (e.g., products of provided methods and/or steps therein) comprise no cleavable groups which can be cleaved under bioorthogonal conditions. In some embodiments, provided compounds/agents (e.g., reaction partners, agents (e.g., products of provided methods and/or steps therein) comprise no cleavable groups which can be cleaved without substantively damaging and/or transforming proteins. In some embodiments, a cleavable group is or comprises -5-, —S—S—, —S-Cy-, —C(O)—O—, —C(O)—S—, acetal moiety, —N═N—, imine moiety, —CH═N—, —P(O)(OR)O— moiety, —P(O)(OR)—N(R)— moiety, —C(O)—CH₂—C(COOH)═CHC(O)— moiety, —CHOH—CHOH— moiety, —Se— moiety, Si bonded to two oxygen atoms, —C(O)—CH₂— wherein the —CH₂— is bonded to a benzylic carbon wherein the phenyl ring of the benzyl group is substituted with —NO₂—, —C(O)—CH₂— wherein the —CH₂— is bonded to a benzylic carbon wherein the phenyl ring of the benzyl group is substituted with —NO₂— at o-position, or —C(O)—N(−)— moiety, wherein N is a ring atom of a heteroaryl ring. In some embodiments, a cleavable group is or comprises —S—S—, —S—CH₂—Cy-, —S-Cy-, —C(O)—O—, —C(O)—S—, acetal moiety, —N═N—, imine moiety, —CH═N—, —P(O)(OR)O— moiety, —P(O)(OR)—N(R)— moiety, —C(O)—CH₂—C(COOH)═CHC(O)— moiety, —CHOH—CHOH— moiety, —Se— moiety, Si bonded to two oxygen atoms, —C(O)—CH₂— wherein the —CH₂— is bonded to a benzylic carbon wherein the phenyl ring of the benzyl group is substituted with —NO₂—, —C(O)—CH₂— wherein the —CH₂— is bonded to a benzylic carbon wherein the phenyl ring of the benzyl group is substituted with —NO₂— at o-position, or —C(O)—N(−)— moiety, wherein N is a ring atom of a heteroaryl ring.

In some embodiments, a linker moiety does not contain a cleavage group above. In some embodiments, a linker moiety does not contain one or more or any of the following moieties: —S—, —S—S—, —S—CH₂—Cy-, —S-Cy-, —C(O)—O—, —C(O)—S—, acetal moiety, —N═N—, imine moiety, —CH═N—, —P(O)(OR)O— moiety, —P(O)(OR)—N(R)— moiety, —C(O)—CH₂—C(COOH)═CHC(O)— moiety, —CHOH—CHOH— moiety, —Se— moiety, Si bonded to two oxygen atoms, —C(O)—CH₂— wherein the —CH₂— is bonded to a benzylic carbon wherein the phenyl ring of the benzyl group is substituted with —NO₂—, —C(O)—CH₂— wherein the —CH₂— is bonded to a benzylic carbon wherein the phenyl ring of the benzyl group is substituted with —NO₂— at o-position, or —C(O)—N(−)— moiety, wherein N is a ring atom of a heteroaryl ring. In some embodiments, a linker moiety does not contain one or more or any of the following moieties: —S—S—, —S—CH₂—Cy-, —S-Cy-, —C(O)—O—, —C(O)—S—, acetal moiety, —N═N—, imine moiety, —CH═N—, —P(O)(OR)O— moiety, —P(O)(OR)—N(R)— moiety, —C(O)—CH₂—C(COOH)═CHC(O)— moiety, —CHOH—CHOH— moiety, —Se— moiety, Si bonded to two oxygen atoms, —C(O)—CH₂— wherein the —CH₂— is bonded to a benzylic carbon wherein the phenyl ring of the benzyl group is substituted with —NO₂—, —C(O)—CH₂— wherein the —CH₂— is bonded to a benzylic carbon wherein the phenyl ring of the benzyl group is substituted with —NO₂— at o-position, or —C(O)—N(−)— moiety, wherein N is a ring atom of a heteroaryl ring. In some embodiments, a linker moiety comprises no —S—. In some embodiments, a linker moiety comprises no —S—S— (optionally except a disulfide moiety formed by two amino acid residues, in some embodiments, optionally except a disulfide moiety formed by two cysteine residues). In some embodiments, a linker moiety comprises no —S-Cy-. In some embodiments, a linker moiety comprises no —S—CH₂—Cy-. In some embodiments, a linker moiety comprises no —C(O)—O—. In some embodiments, a linker moiety comprises no —C(O)—S—. In some embodiments, a linker moiety comprises no acetal moiety. In some embodiments, a linker moiety comprises no —N═N—. In some embodiments, a linker moiety comprises no imine moiety. In some embodiments, a linker moiety comprises no —CH═N— (optionally except in a ring, in some embodiments, optionally except in a heteroaryl ring). In some embodiments, a linker moiety comprises no —P(O)(OR)O— moiety. In some embodiments, a linker moiety comprises no —P(O)(OR)—N(R)— moiety. In some embodiments, a linker moiety comprises no —C(O)—CH₂—C(COOH)═CHC(O)— moiety. In some embodiments, a linker moiety comprises no —CHOH—CHOH— moiety. In some embodiments, a linker moiety comprises no —Se— moiety. In some embodiments, a linker moiety comprises no Si bonded to two oxygen atoms. In some embodiments, a linker moiety comprises no —C(O)—CH₂—, wherein the —CH₂— is bonded to a benzylic carbon, wherein the phenyl ring of the benzyl group is substituted with —NO₂—. In some embodiments, a linker moiety comprises no —C(O)—CH₂—, wherein the —CH₂— is bonded to a benzylic carbon, wherein the phenyl ring of the benzyl group is substituted with —NO₂— at o-position. In some embodiments, a linker moiety comprise no —C(O)—N(−)— moiety, wherein N is a ring atom of a heteroaryl ring. In some embodiments, a linker moiety does not contain any of these groups. In some embodiments, L^{RM} is such a linker moiety. In some embodiments, L^{PM} is such a linker moiety. In some embodiments, L^{LG} is such a linker moiety. In some embodiments, an agent of the present disclosure does not contain one or more or all of such moieties.

In some embodiments, L is a covalent bond. In some embodiments, L is a bivalent optionally substituted, linear or branched C₁₋₁₀₀ aliphatic group wherein one or more methylene units of the group are optionally and independently replaced. In some embodiments, L is a bivalent optionally substituted, linear or branched C₆₋₁₀₀ arylaliphatic group wherein one or more methylene units of the group are optionally and independently replaced. In some embodiments, L is a bivalent optionally substituted, linear or branched C₅100 heteroarylaliphatic group having 1-20 heteroatoms wherein one or more methylene units of the group are optionally and independently replaced. In some embodiments, L is a bivalent optionally substituted, linear or branched C₁₋₁₀₀ heteroaliphatic group having 1-20 heteroatoms wherein one or more methylene units of the group are optionally and independently replaced.

In some embodiments, a linker moiety (e.g., L) is or comprises one or more (e.g., 1, 2, 3, 4, 5, 6, 7, 8, 9, 10, 11, 12, 13, 14, 15, 16, 17, 18, 19, 20 or more) polyethylene glycol units. In some embodiments, a linker moiety is or comprises —(CH₂CH₂O)n-, wherein n is as described in the present disclosure. In some embodiments, one or more methylene units of L are independently replaced with —(CH₂CH₂O)n-.

As described herein, in some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12. In some embodiments, n is 13. In some embodiments, n is 14. In some embodiments, n is 15. In some embodiments, n is 16. In some embodiments, n is 17. In some embodiments, n is 18. In some embodiments, n is 19. In some embodiments, n is 20.

In some embodiments, a linker moiety (e.g., L) is or comprises one or more (e.g., 1, 2, 3, 4, 5, 6, 7, 8, 9, 10, 11, 12, 13, 14, 15, 16, 17, 18, 19, 20 or more) amino acid residues. As used in the present disclosure, “one or more” can be 1-100, 1-50, 1-40, 1-30, 1-20, 1-10, 1-5, 1, 2, 3, 4, 5, 6, 7, 8, 9, 10, 11, 12, 13, 14, 15, 16, 17, 18, 19, or 20 or more. In some embodiments, one or more methylene units of L are independently replaced with an amino acid residue. In some embodiments, one or more methylene units of L are independently replaced with an amino acid residue, wherein the amino acid residue is of an amino acid of formula A-I or a salt thereof. In some embodiments, one or more methylene units of L are independently replaced with an amino acid residue, wherein each amino acid residue independently has the structure of —N(R^{a1})-L^{a1}-C(R^{a2})(R^{a3})-L^{a2}-CO— or a salt form thereof.

In some embodiments, a linker moiety comprises one or more moieties, e.g., amino, carbonyl, etc., that can be utilized for connection with other moieties. In some embodiments, a linker moiety comprises one or more —NR′—, wherein R′ is as described in the present disclosure. In some embodiments, —NR′— improves solubility. In some embodiments, —NR′— serves as connection points to another moiety. In some embodiments, R′ is —H. In some embodiments, one or more methylene units of L are independently replaced with —NR′—, wherein R′ is as described in the present disclosure.

In some embodiments, a linker moiety, e.g., L, comprises a —C(O)— group, which can be utilized for connections with a moiety. In some embodiments, one or more methylene units of L are independently replaced with —C(O)—.

In some embodiments, a linker moiety, e.g., L, comprises a —NR′— group, which can be utilized for connections with a moiety. In some embodiments, one or more methylene units of L are independently replaced with —N(R′)—.

In some embodiments, a linker moiety, e.g., L, comprises a —C(O)NR′— group, which can be utilized for connections with a moiety. In some embodiments, one or more methylene units of L are independently replaced with —C(O)N(R′)—.

In some embodiments, a linker moiety, e.g., L, comprises a —C(R′)₂— group. In some embodiments, one or more methylene units of L are independently replaced with —C(R′)₂—. In some embodiments, —C(R′)₂— is —CHR′—. In some embodiments, R′ is —(CH₂)₂C(O)NH(CH₂)₁₁COOH. In some embodiments, R′ is —(CH₂)₂COOH. In some embodiments, R′ is —COOH.

In some embodiments, a linker moiety is or comprises one or more ring moieties, e.g., one or more methylene units of L are replaced with -Cy-. In some embodiments, a linker moiety, e.g., L, comprises an aryl ring. In some embodiments, a linker moiety, e.g., L, comprises an heteroaryl ring. In some embodiments, a linker moiety, e.g., L, comprises an aliphatic ring. In some embodiments, a linker moiety, e.g., L, comprises an heterocyclyl ring. In some embodiments, a linker moiety, e.g., L, comprises a polycyclic ring. In some embodiments, a ring in a linker moiety, e.g., L, is 3-20 membered. In some embodiments, a ring is 5-membered. In some embodiments, a ring is 6-membered. In some embodiments, a ring in a linker is product of a cycloaddition reaction (e.g., click chemistry, and variants thereof) utilized to link different moieties together.

In some embodiments, a linker moiety (e.g., L) is or comprises

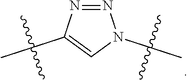 [SMILES: *c1cn(*)nn1]

In some embodiments, a methylene unit of L is replaced with

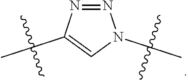 [SMILES: *c1cn(*)nn1]

In some embodiments, a methylene unit of L is replaced with -Cy-. In some embodiments, -Cy- is

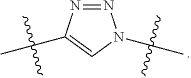 [SMILES: *c1cn(*)nn1]

In some embodiments, a linker moiety (e.g., L) is or comprises -Cy-. In some embodiments, a methylene unit of L is replaced with -Cy-. In some embodiments, -Cy- is

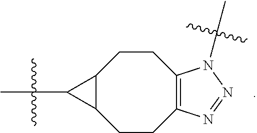 [SMILES: *C1C2CCc3nnn(*)c3CCC12]

In some embodiments, -Cy- is

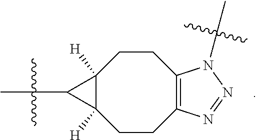 [SMILES: *C1[C@H]2CCc3c(nnn3*)CC[C@@H]12]

In some embodiments, -Cy- is

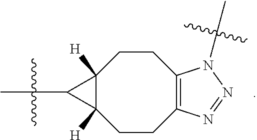 [SMILES: *C1[C@H]2CCc3nnn(*)c3CC[C@@H]12]

In some embodiments, a linker moiety, e.g., L, comprises

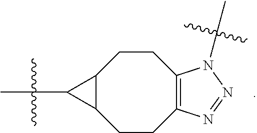 [SMILES: *C1C2CCc3nnn(*)c3CCC12]

In some embodiments, L^{RM} is a covalent bond. In some embodiments, L^{RM} is not a covalent bond. In some embodiments, L^{RM} is or comprises —(CH₂CH₂O)n-. In some embodiments, L^{RM} is or comprises —(CH₂)n-O—(CH₂CH₂O)n-(CH₂)n-, wherein each n is independently as described herein, and each —CH₂— is independently optionally substituted. In some embodiments, L^{RM} is —(CH₂)n-O—(CH₂CH₂O)n-(CH₂)n-, wherein each n is independently as described herein, and each —CH₂— is independently optionally substituted. In some embodiments, L^{RM} is —(CH₂)₂—O—(CH₂CH₂O)n-(CH₂)₂—, wherein n is as described herein, and each —CH₂— is independently optionally substituted. In some embodiments, L^{RM} is —(CH₂)₂—O—(CH₂CH₂O)n-(CH₂)₂—, wherein n is as described herein.

In some embodiments, L^{PM} is a covalent bond. In some embodiments, L^{PM} is not a covalent bond. In some embodiments, L^{PM} is or comprises —(CH₂CH₂O)n-. In some embodiments, L^{PM} is or comprises —(CH₂)n-O—(CH₂CH₂O)n-(CH₂)n-, wherein each n is independently as described herein, and each —CH₂— is independently optionally substituted. In some embodiments, L^{PM} is —(CH₂)n-O—(CH₂CH₂O)n-(CH₂)n-, wherein each n is independently as described herein, and each —CH₂— is independently optionally substituted. In some embodiments, L^{PM} is —(CH₂)₂—O—(CH₂CH₂O)n-(CH₂)₂—, wherein n is as described herein, and each —CH₂— is independently optionally substituted. In some embodiments, L^{PM} is —(CH₂)₂—O—(CH₂CH₂O)n-(CH₂)₂—, wherein n is as described herein.

In some embodiments, L^{PM} (e.g., in a product of a first and a second agents) is or comprises a reaction product moiety formed a first reactive moiety and a second reactive moiety.

In some embodiments, a linker moiety (e.g., L^{PM} in a product of a first and a second agents) is or comprises

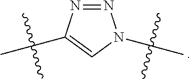 [SMILES: *c1cn(*)nn1]

In some embodiments, a methylene unit of a linker moiety, e.g., L or a linker moiety that can be L (e.g., L^{RM}, L^{PM}, etc.) is replaced with -Cy-. In some embodiments, -Cy- is optionally substituted

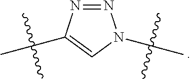 [SMILES: *c1cn(*)nn1]

In some embodiments, -Cy- is

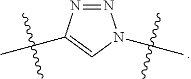 [SMILES: *c1cn(*)nn1]

In some embodiments, -Cy- is

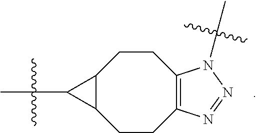 [SMILES: *C1C2CCc3nnn(*)c3CCC12]

In some embodiments, -Cy- is

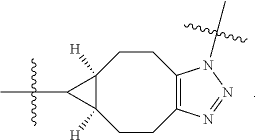 [SMILES: *C1[C@H]2CCc3c(nnn3*)CC[C@@H]12]

In some embodiments, -Cy- is

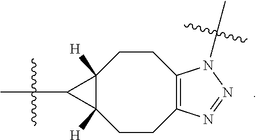 [SMILES: *C1[C@H]2CCc3nnn(*)c3CC[C@@H]12]

### Moieties of Interest

Those skilled in the art reading the present disclosure will appreciate that various types of moieties of interest can be utilized for various purposes in accordance with the present disclosure. For the present disclosure the moiety of interest is or comprises monomethyl auristatin E (MMAE), or a close analog of MMAE.

In some embodiments of this disclosure the moiety of interest is or comprises MMAE. MMAE is an anti-neoplastic agent that is used in drug-antibody conjugates, e.g. a MAB-MMAE conjugate. The MMAE is joined to a monoclonal antibody through a linking structure that can be cleaved once the drug-antibody conjugate has attached to the tumor cell. The linking structure includes a cathepsin-cleavable sequence (Valine-Citrulline) and a spacer. The spacer may be varied.

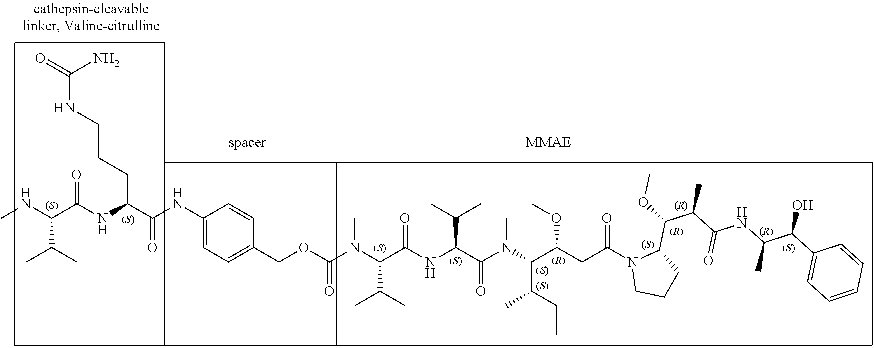

In some embodiments the moiety of interest is or comprises MMAD.

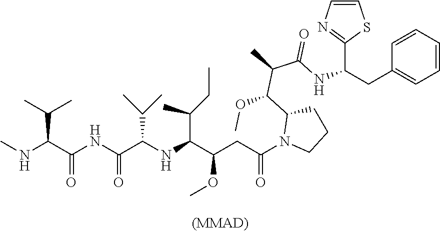

In some embodiment the moiety of interest is or comprises MMAF, that is monomethyl auristatin F or desmethyl-auristatin F, shown below with its linking structure.

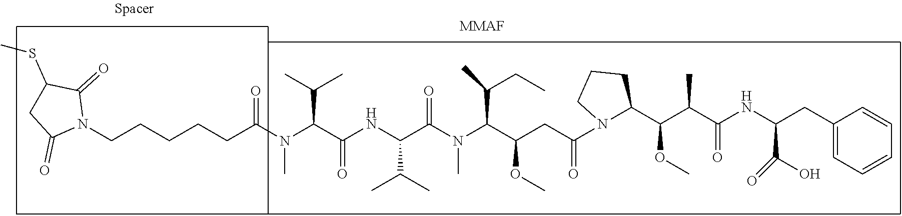

In some embodiments, a provided method further comprises: reacting a first agent comprising a first reactive moiety, e.g., in a first moiety of interest, with a second agent comprising a second reactive moiety. In various embodiments, a first reactive moiety is in a first moiety of interest, e.g., which can be incorporated through a method described herein (e.g., via contacting with a compound having the structure of formula R—I or a salt thereof).

In some embodiments, a second moiety of interest is in a compound which comprises no target binding moieties. In some embodiments, a second moiety of interest is in a compound of formula P—I or P-II, or a salt thereof. In some embodiments, a second moiety of interest is in a compound of R—I or a salt thereof. In some embodiments, a second agent has the structure of formula P—I or P-II, or a sat thereof. In some embodiments, a second reactive moiety is in a moiety of interest of a second agent. In some embodiments, a second agent comprises a target agent moiety as described herein. For example, in some embodiments, a target agent moiety in a second agent is or comprises a peptide moiety. In some embodiments, a target agent moiety in a second agent is or comprises an antibody agent moiety as described herein. In some embodiments, it comprises a scFv moiety. In some embodiments, a target agent moiety in a second agent provides different specificity compared to that of a first agent. In some embodiments, such first and second agents react with each other to provide various product agents comprising moieties having different specificities as described herein.

In some embodiments, a reaction between a first reactive moiety and a second reactive moiety is a bioorthogonal reaction. In some embodiments, a reaction is a cycloaddition reaction. In some embodiments, a reaction is a [3+2] reaction. Suitable such reactions and corresponding first and second reactive moieties are widely known in the art and can be utilized in accordance with the present disclosure. In some embodiments, a first reactive moiety is or comprises —N₃, and a second reactive moiety is or comprises -≡- (e.g., an alkyne moiety suitable for click chemistry, including those suitable for metal-free click chemistry). In some embodiments, a second reactive moiety is or comprises —N₃, and a first reactive moiety is or comprises -≡- (e.g., an alkyne moiety suitable for click chemistry, including those suitable for metal-free click chemistry).

As described herein, in some embodiments, a reaction between a first reactive moiety and a second reactive moiety is an enzymatic reaction. In some embodiments, a reaction is a sortase-mediated reaction. In some embodiments, each of the first and second reactive moiety independently is or comprises a substrate moiety for a reaction, e.g., an enzymatic reaction. For example, in some embodiments, for a sortase-mediated conjugation, a reactive moiety is or comprises (G)n (e.g., n is 3, 4, 5, etc.), and a reactive moiety is or comprises LPXTG (e.g., LPETG). In some embodiments, a reactive moiety is or comprises LPXTG-(X)n (e.g., LPETG-(X)n, LPETG-XX, etc.). Those skilled in the art reading the present disclosure will appreciate that various reactive moieties can be utilized in accordance with the present disclosure for conjugation, via either enzymatic and/or non-enzymatic pathways.

In some embodiments, a second agent is or comprises a second moiety of interest which is a moiety of interest as described herein. In some embodiments, a second reactive moiety and a second moiety of interest is connected through a linker (e.g., a linker as described herein (e.g., L^{PM}, L, etc. as described herein). In some embodiments, a second moiety of interest is as described herein (e.g., a detection moiety, a therapeutic moiety, a moiety of interest which can interact, recognize and/or bind proteins, nucleic acids, immune cells, disease cells, etc.). In some embodiments, a second moiety of interest is or comprises an antibody agent. In some embodiments, a second moiety of interest is or comprises a scFv antibody agent. In some embodiments, such an antibody agent has different specificity compared to the initial target antibody agent. Thus, in some embodiments, the present disclosure provides bispecific antibody agents, compositions, and methods thereof. In some embodiments, a target agent is or comprises a first antibody agent, and it is conjugated with a moiety of interest comprising a first reactive moiety. In some embodiments, an agent comprising a first antibody agent and a first reactive moiety is reacted with a second agent comprising a second reactive moiety and a second moiety of interest which is or comprises a second antibody agent to provide an agent comprising a first and a second antibody agents. In some embodiments, a first and a second antibody agents are different. In some embodiments, they are the same.

In some embodiments, an agent comprises two or more antibody agent moieties. In some embodiments, antibody agent moieties in a single agent molecule have different target specificity. In some embodiments, some or all antibody agent moieties in a single agent molecule have the same target specificity. In some embodiments, an agent as described herein is or comprises moieties having different target specificity (e.g., antibody moieties having different target specificity). In some embodiments, an agent is a bispecific antibody agent. In some embodiments, an agent comprises a first moiety (e.g., a first antibody agent moiety) and a second moiety (e.g., a second antibody agent moiety). In some embodiments, a first moiety (e.g., a first antibody agent moiety) is or comprises IgG or a fragment thereof. In some embodiments, a first moiety (e.g., a first antibody agent moiety) is or comprises an antibody agent moiety or a fragment thereof (e.g., Fc region or a fragment thereof) to which a target binding moiety may bind. In some embodiments, a second moiety (e.g., a second antibody agent moiety) is or comprises IgG or a fragment thereof. In some embodiments, a second moiety (e.g., a second antibody agent moiety) is or comprises an antibody agent moiety or a fragment thereof (e.g., Fc region or a fragment thereof) to which a target binding moiety may bind. In some embodiments, an antibody agent moiety, e.g., a second antibody agent moiety, comprises no moiety to which a target binding moiety may bind. In some embodiments, an antibody agent moiety, e.g., a second antibody agent moiety, comprises no Fc moiety to which a target binding moiety may bind. In some embodiments, an antibody agent moiety, e.g., a second antibody agent moiety, is or comprises scFv. In some embodiments, a first moiety is or comprises an agent moiety of a first agent. In some embodiments, a second moiety is or comprises a moiety of interest of a second agent. In some embodiments, a first agent, e.g., one comprising a first antibody agent moiety, is contacted with a second agent, e.g., one comprising a second antibody agent moiety, to provide an agent comprising two or more moieties having target specificity (e.g., antibody agent moieties).

In some embodiments, a moiety, e.g., a first moiety, is or comprises an antibody agent moiety that binds to a target (e.g., a protein, lipid, carbohydrate, object, etc.) associated with a condition, disorder or disease (e.g., cancer). In some embodiments, a moiety, e.g., a first moiety, is or comprises a moiety of an antibody agent suitable for preventing or treating a condition, disorder or disease, e.g., cancer. In some embodiments, a moiety, e.g., a first moiety, is or comprises a moiety of an antibody agent which targets cancer cells, tissues, organs, etc. For example, in some embodiments, a first moiety is or comprises a moiety of an anti-CD20 antibody or a fragment thereof. In some embodiments, a first moiety is or comprises rituximab or a fragment thereof. In some embodiments, a moiety, e.g., a second moiety, is a second moiety of interest. In some embodiments, a moiety, e.g., a second moiety, is or comprises an antibody agent moiety that can recruit and/or activate an immune activity, e.g. one or more immune cells. In some embodiments, a moiety, e.g., a second moiety, is or comprises an antibody agent moiety which can recruit and/or activate T cells. In some embodiments, a moiety, e.g., a second moiety is or comprises a moiety of an anti-CD3 antibody or fragment thereof. In some embodiments, an anti-CD3 antibody is a CD3-directed scFv. In some embodiments, a moiety, e.g., a first moiety, is a target agent moiety. In some embodiments, a provided agent comprises an anti-CD20 moiety and an anti-CD3 moiety. In some embodiments, a provided agent comprises an anti-CD20 moiety and an anti-CD3 moiety, wherein the two moieties are linked by a linker. In some embodiments, a linker comprises moieties that are not amino acid residues. In some embodiments, a linker comprises moieties that are not natural proteinogenic amino acid residues. In some embodiments, a linker is a linker moiety as described herein. Those skilled in the art will appreciate that agents comprising two or more target-specific moieties (e.g., antibody agent moieties) can be prepared with various benefits and characteristics according to the present disclosure, e.g., high site specificity, high homogeneity, low level of damages, low levels or substantially absence of reduction of desired properties and/or activities (e.g., target binding, recruitment and/or activation of immune activities, etc.), etc. Those skilled in the art will also appreciate that provided technologies can readily conjugate antibody agents, e.g., those readily available (e.g., “off-the-shelf” therapeutic antibodies) with other moieties, e.g., in some embodies other antibody agents, to, e.g., produce bispecific agents. In some embodiments, a first and a second moiety is linked by a linker as described herein.

In some embodiments, a provided product agent comprises a linker moiety connecting a target agent moiety and a second moiety of interest (e.g., two antibody agent moieties). In some embodiments, a linker is or comprises one or more of L^{RG2}, L^{PM} or fragments thereof, and one or more moiety formed by a first and second reactive moieties (e.g., for click chemistry, a triazole moiety). In some embodiments, a linker is or comprises a product linker moiety, e.g., one formed by a reaction between a first and a second reactive moiety. In some embodiments, a product linker moiety is or comprise LPXTG. In some embodiments, a product linker moiety is or comprise LPXT(G)n, wherein n is 1, 2, 3, 4, 5, 6, 7, 8, 9, or 10. In some embodiments, a product linker moiety is or comprises a bioorthogonal reaction product moiety, e.g., a click chemistry reaction product moiety.

In some embodiments, an agent comprising a second reactive moiety and a second moiety of interest is prepared using a technology provided herein. In some embodiments, a second moiety of interest is or comprises a protein agent moiety. In some embodiments, a second moiety of interest is or comprises an antibody agent moiety. In some embodiments, a second moiety of interest (e.g., a protein agent (e.g., an antibody agent)) can serve as a target agent moiety, and a second reactive moiety can serve as a moiety of interest (e.g., MOI in a compound of formula R—I or a salt thereof), for utilization of certain provided methods (e.g., comprising reacting target agents (e.g., protein agents (e.g., antibody agents, etc.)) with reaction partners comprising moieties of interest (e.g., those that are or comprise second reactive moieties), reactive groups and target binding moieties that can bind to target agents to provide second agents).

In some embodiments, each of a first agent and a second agent is independently and optionally an agent of formula P—I or P-II, or a salt thereof. In some embodiments, each of a first agent and a second agent is independently an agent of formula P—I or P-II, or a salt thereof. In some embodiments, at least one of a first agent and a second agent is prepared using a method of the present disclosure. In some embodiments, each of a first agent and a second agent is independently prepared using a method of the present disclosure. In some embodiments, a target agent moiety of a first agent is an antibody agent. In some embodiments, a moiety of interest of a first agent is or comprises a first reactive moiety. In some embodiments, a target agent moiety of a second agent is an antibody agent. In some embodiments, a moiety of interest of a second agent is or comprises a second reactive moiety. As described herein, in many embodiments, a first reactive moiety and a second reactive moiety can react with each to provide a product agent. In some embodiments, a reaction between a first and a second reactive moieties is or comprises a reaction compatible with target agents in the first and second agents, e.g., compatible with protein agents (e.g., antibody agents). In some embodiments, such a reaction is a bioorthogonal reaction. In some embodiments, such a reaction is a cycloaddition reaction. In some embodiments, such a reaction is a click reaction. In some embodiments, such a reaction is a metal free click reaction. In some embodiments, a product agent is of formula P—I or P-II, or a salt thereof. In some embodiments, in a product agent of formula P—I or P-II, or a salt thereof, a target agent moiety is a protein agent (e.g., an antibody agent), and in some embodiments, a target agent moiety of a first agent. In some embodiments, in a product agent of formula P—I or P-II, or a salt thereof, a moiety of interest is a protein agent (e.g., an antibody agent), and in some embodiments, a target agent moiety of a second agent. In some embodiments, a product agent comprises two or more antibody agents. In some embodiments, the two or more antibody agents have different antigen specificity. In some embodiments, the two or more antibody agents are toward different antigens. In some embodiments, a provided method comprises:
reacting a first agent which has the structure of formula P—I or P-II, or a salt thereof with a second agent which has the structure of formula P—I or P-II, or a salt thereof to provide a product agent.

### Methods and Products

In some embodiments, provided technologies comprise contacting a target agent (e.g., to which a moiety of interest is to be attached) with a reaction partner. In some embodiments, contact is performed under conditions and for a time so that a target agent react with a reaction partner to form an agent as a product. Many reaction conditions/reaction times in the art may be assessed and utilized if suitable for desired purposes in accordance with the present disclosure; certain such conditions, reaction times, assessment, etc. are described in the Examples.

In some embodiments, an agent formed comprises a target agent moiety, a moiety of interest and optionally a linker moiety connecting a target agent moiety and a moiety of interest. In some embodiments, a target agent moiety is derived from a target agent (e.g., by removing one or more —H from a target agent). In some embodiments, a target agent moiety maintains one or more, most, or substantially all structural features and/or biological functions of a target agent. For example, in some embodiments, a target agent is an antibody agent, and a target agent moiety in a formed agent is a corresponding antibody agent moiety and maintains major functions of the antibody agent, e.g., interacting with various receptors (e.g., Fc receptors such as FcRn), recognizing antigen with specificity, triggering, promoting, and/or enhancing immunological activities toward diseased cells, etc., as the antibody agent. In some embodiments, a formed agent provides one or more functions beyond those of a target agent, for example, from a moiety of interest and/or a formed agent as a whole.

In some embodiments, an agent formed has the structure of formula P—I or P-II, or a salt thereof. In some embodiments, a moiety of interest in a formed agent (e.g., MOI of formula P—I or P-II, or a salt thereof) is the same as a moiety of interest in a reaction partner (e.g., MOI of formula R—I or a salt thereof) utilized to prepare a formed agent. In some embodiments, P is a protein moiety. In some embodiments, P is an antibody moiety.

In some embodiments, linker moieties (or a part thereof) connected to moieties of interest may also be transferred from reaction partners (e.g., L^{RM} of formula R—I or a salt thereof). In some embodiments, a linker moiety in a formed agent (e.g., L^{P}M) is or comprises a linker moiety in a reaction partner (e.g., one between a reactive group and a moiety of interest, e.g., L^{RM}). In some embodiments, L^{PM} is or comprises L^{RM}. In some embodiments, L^{PM} is -L^{RM}-L^{RG2}-. In some embodiments, L^{RG2} is —C(O)—. In some embodiments, L^{RG2} is —C(O)—, and is bonded to —NH— of a target agent moiety, e.g., —NH— in a side chain of a lysine residue of a protein moiety, which in some embodiments, is an antibody moiety.

Reaction partners, e.g., compounds of formula R—I or salts thereof, typically do not contain moieties that can react with reactive groups under conditions under which reactive groups react with target agents. In some embodiments, to the extent that some moieties in reaction partners may react with reactive groups under conditions under which reactive groups react with target agents, reactions between such moieties and reactive groups are significantly slower and/or less efficient compared to reactions between reactive groups and target agents. In some embodiments, reactions between such moieties and reactive groups do not significantly reduce (e.g., no more than about 5%, 10%, 15%, 20%, 25%, 30%, 40%, 50%, 60%, 70%, 80%, 90%, etc. of reduction) efficiencies, yields, rates, and/or conversions, etc., of reactions between reactive groups and target agents. In some embodiments, reactive groups (e.g., ester groups, activated carboxylic acid derivatives, etc.) react with amino groups (e.g., —NH₂ groups) of target agents (e.g., protein agents such as antibody agents). In some embodiments, reaction partners, e.g., compounds of formula R—I or salts thereof, do not contain amine groups. In some embodiments, compounds of formula R—I or salts thereof (or portions thereof, such as R^{LG}, L^{LG}, L^{LG1}, L^{LG2}, L^{LG3}, L^{LG4}, L^{RG1}, L^{RG2}, L^{RM}, and/or MOI) do not contain amine groups. In some embodiments, they do not contain primary amine groups (—NH₂). In some embodiments, they do not contain —CH₂NH₂. In some embodiments, they do not contain —CH₂CH₂NH₂. In some embodiments, they do not contain —CH₂CH₂CH₂NH₂. In some embodiments, they do not contain —CH₂CH₂CH₂CH₂NH₂. In some embodiments, amine groups, e.g., primary amine groups, are capped (e.g., by introduction of acyl groups (e.g., R—C(O)— (e.g., acetyl)) to form amide groups) to prevent or reduce undesired reactions.

In some embodiments, reactions are performed in buffer systems. In some embodiments, buffer systems of present disclosure maintain structures and/or functions of target agents, moiety of interest, etc. In some embodiments, a buffer is a phosphate buffer. In some embodiments, a buffer is a PBS buffer. In some embodiments, a buffer is a borate buffer. In some embodiments, buffers of the present disclosure provide and optionally maintain certain pH value or range. For example, in some embodiments, a useful pH is about 7-9, e.g., 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 9.0, etc. In some embodiments, a pH is 7.4. In some embodiments, a pH is 7.5. In some embodiments, a pH is 7.8. In some embodiments, a pH is 8.0. In some embodiments, a pH is 8.2. In some embodiments, a pH is 8.3.

Provided technologies can provide various advantages. Among other things, in some embodiments, connection of a moiety of interest in a provided reaction partner (e.g., a compound comprising a reactive group located between a first group and a moiety of interest (e.g., a compound of formula R—I or a salt thereof)) to a target agent and release of a target binding moiety in a provided reaction partner can be achieved in one reaction and/or in one pot. Thus, in many embodiments, no separate reactions/steps are performed to remove target binding moieties. As appreciated by those skilled in the art, by performing connection of moiety of interest and release of target binding moiety in a single reaction/operation, provided technologies can avoid separate steps for target binding moiety removal and can improve overall efficiency (e.g., by simplify operations, increasing overall yield, etc.), reduce manufacturing cost, improve product purity (e.g., by avoiding exposure to target binding moiety removal conditions, which typically involve one or more of reduction, oxidation, hydrolysis (e.g., of ester groups), etc., conditions and may damage target agent moieties (e.g., for protein agent moieties, protein amino acid residues, overall structures, and/or post-translational modifications (e.g., glycans of antibodies) thereof. Indeed, as demonstrated herein, provided technologies among other things can provided improved efficiency (e.g., in terms of reaction rates and/or conversion percentages), increased yield, increased purity/homogeneity, and/or enhanced selectivity, particularly compared to reference technologies wherein a reaction partner containing no target binding moieties is used, without introducing step(s) for target binding moiety removal (e.g., target binding moiety is removed in the same step as moiety of interest conjugation).

In some embodiments, the present disclosure provides products of provided processes, which, among other things, contain low levels of damage to target agent moieties compared to processes comprising steps which are performed for target binding moiety removal but not for substantial moiety of interest conjugation. In some embodiments, provided product compositions have high homogeneity compared to reference product compositions (e.g., those from technologies without using target binding moieties, or utilizing extra step(s) for target binding moiety removal (e.g., not utilizing reaction partners described herein which comprise a reactive group located between a target binding moiety and a moiety of interest).

In some embodiments, a product agent is an agent comprising:
a target agent moiety;
a moiety of interest, such as MMAD; and
optionally one or more linker moieties.

In some embodiments, a target agent moiety is a protein agent moiety. In some embodiments, a target agent moiety is an antibody agent moiety. In some embodiments, an antibody agent moiety comprises IgG Fc region. In some embodiments, a target agent moiety is connected to a moiety of interest through an amino group optionally through a linker. In some embodiments, it is through a lysine residue wherein the amino group of the side chain is connected to a moiety of interest optionally through a linker (e.g., forming —NH—C(O)— as part of an amide group, a carbamate group, etc.).

In some embodiments, selected locations of target agents are utilized for conjugation. For example, in some embodiments, K246 or K248 of an antibody agent (EU numbering, or corresponding residues) are conjugation locations. In some embodiments, a conjugation location is K246 of heavy chain (unless otherwise specified, locations herein include corresponding residues in, e.g., modified sequence (e.g., longer, shorter, rearranged, etc., sequences)). In some embodiments, a location is K248 of heavy chain. In some embodiments, a location is K288 or K290 of heavy chain. In some embodiments, a location is K288 of heavy chain. In some embodiments, a location is K290 of heavy chain. In some embodiments, a location is K317.

In some embodiments, when target agents are antibody agents, heavy chains are selectively labeled over light chains.

Among other things, the present disclosure can provide controlled moiety of interest/target agent ratios (e.g., for antibody-drug conjugates, drug/antibody ratio (DAR)). In some embodiments, a ratio is about 0.5-6, e.g., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, 5.0, 5.5, etc.). In some embodiments, a ratio is about 0.5-2.5. In some embodiments, a ratio is about 0.5-2. In some embodiments, a ratio is about 1-2. In some embodiments, a ratio is about 1.5-2. In some embodiments, a ratio is of moieties of interest conjugated to target agent moieties and target agent moieties conjugated to moieties of interest. In some embodiments, a ratio is of moieties of interest conjugated to target agent moieties and all target agent moieties in a composition.

In some embodiments, in provided agents (e.g., agents of formula P—I or P-II, or a salt thereof) substantially all conjugation sites of target agent moieties have the same modifications (e.g., all share the same moieties of interest optionally connected through the same linker moieties). In some embodiments, no conjugation sites bear different modifications (e.g., different moieties of interest and/or no moieties of interest and/or different linker moieties).

In some embodiments, in provided compositions comprising a plurality of provided agents (e.g., agents of formula P—I or P-II, or a salt thereof) substantially all conjugation sites of target agent moieties have the same modifications (e.g., all share the same moieties of interest optionally connected through the same linker moieties). In some embodiments, no conjugation sites bear different modifications (e.g., different moieties of interest and/or no moieties of interest and/or different linker moieties). In some embodiments, such compositions do not contain agents that share the same (or substantially the same) target agent moieties but different modifications (e.g., different moieties of interest and/or no moieties of interest and/or different linker moieties). In some embodiments, agents that share the same (or substantially the same) target agent moieties but different modifications (e.g., different moieties of interest and/or no moieties of interest and/or different linker moieties) are intermediates of multiple-step preparations (e.g., comprising steps for removal of target binding moieties in addition to steps for moiety of interest conjugation) of final product agents.

In some embodiments, the present disclosure provides a composition comprising a plurality of agents each of which independently comprising:
a target agent moiety,
a moiety of interest, and
optionally a linker moiety linking a target agent moiety and a moiety of interest; wherein agents of the plurality share the same or substantially the same target agent moiety, and a common modification independently at at least one common location; and
wherein about 1%-100% of all agents that comprise a target agent moiety and a moiety of interest are agents of the plurality.

In some embodiments, a target agent moiety is or comprises a protein moiety. In some embodiments, agents of the plurality share common modifications (e.g., conjugations of moieties of interest optionally through linker moieties) independently at at least one amino acid residues. In some embodiments, agents of the plurality are each independently of formula P—I or P-II, or a salt thereof.

In some embodiments, the present disclosure provides a composition comprising a plurality of agents each of which independently comprising:
a protein agent moiety,
a moiety of interest, and
optionally a linker moiety linking the protein agent moiety and a moiety of interest; wherein protein agent moieties of agents of the plurality comprise a common amino acid sequence, and agents of the plurality share a common modification independently at at least one common amino acid residue of protein agent moieties; and
wherein about 1%-100% of all agents that comprise a protein agent moiety that comprise the common amino acid sequence and a moiety of interest are agents of the plurality.

In some embodiments, agents of the plurality are each independently of formula P—I or P-II, or a salt thereof. In some embodiments, each protein agent moiety is independently an antibody agent moiety.

In some embodiments, the present disclosure provides a composition comprising a plurality of agents each of which independently comprising:
an antibody agent moiety,
a moiety of interest, and
optionally a linker moiety linking an antibody agent moiety and a moiety of interest; wherein antibody agent moieties of agents of the plurality comprise a common amino acid sequence or can bind to a common antigen, and agents of the plurality share a common modification independently at at least one common amino acid residue of protein agent moieties; and
wherein about 1%-100% of all agents that comprise an antibody agent moiety that comprise the common amino acid sequence or can bind to the common antigen and a moiety of interest are agents of the plurality.

In some embodiments, agents of the plurality are each independently of formula P—I or P-II, or a salt thereof. In some embodiments, antibody agent moieties of agents of the plurality comprise a common amino acid sequence. In some embodiments, antibody agent moieties of agents of the plurality comprise a common amino acid sequence in a Fc region. In some embodiments, antibody agent moieties of agents of the plurality comprise a common Fc region. In some embodiments, antibody agent moieties of agents of the plurality can bind a common antigen specifically. In some embodiments, antibody agent moieties are monoclonal antibody moieties. In some embodiments, antibody agent moieties are polyclonal antibody moieties. In some embodiments, antibody agent moieties bind to two or more different antigens. In some embodiments, antibody agent moieties bind to two or more different proteins. In some embodiments, antibody agent moieties are IVIG moieties.

As used in the present disclosure, in some embodiments, “at least one” or “one or more” is 1-1000, 1-500, 1-200, 1-100, 1-90, 1-80, 1-70, 1-60, 1-50, 1-40, 1-30, 1-20, 1-10, 1-5, or about 1, 2, 3, 4, 5, 6, 7, 8, 9, 10, 11, 12, 13, 14, 15, 16, 17, 18, 19, 20, or more. In some embodiments, it is one. In some embodiments, it is two or more. In some embodiments, it is about 3. In some embodiments, it is about 4. In some embodiments, it is about 5. In some embodiments, it is about 6. In some embodiments, it is about 7. In some embodiments, it is about 8. In some embodiments, it is about 9. In some embodiments, it is about 10. In some embodiments, it is about 10 or more.

In some embodiments, a common amino acid sequence comprises 1-1000, 1-500, 1-400, 1-300, 1-200, 1-100, 1-50, 10-1000, 10-500, 10-400, 10-300, 10-200, 10-100, 10-50, 20-1000, 20-500, 20-400, 20-300, 20-200, 20-100, 20-50, 50-1000, 50-500, 50-400, 50-300, 50-200, 50-100, 1, 2, 3, 4, 5, 6, 7, 8, 9, 10, 11, 12, 13, 14, 15, 16, 17, 18, 19, 20, 25, 30, 40, 50, 60, 70, 80, 90, 100, 110, 120, 130, 140, 150, 200, 250, 300, 400, 500, 600 or more amino acid residues. In some embodiments, a length is at least 5 amino acid residues. In some embodiments, a length is at least 10 amino acid residues. In some embodiments, a length is at least 50 amino acid residues. In some embodiments, a length is at least 100 amino acid residues. In some embodiments, a length is at least 150 amino acid residues. In some embodiments, a length is at least 200 amino acid residues. In some embodiments, a length is at least 300 amino acid residues. In some embodiments, a length is at least 400 amino acid residues. In some embodiments, a length is at least 500 amino acid residues. In some embodiments, a length is at least 600 amino acid residues.

In some embodiments, a common amino acid sequence is at least 10%-100%, 50%-100%, 50%, 55%, 60%, 65%, 70%, 75%, 80%, 85%, 90%, 91%, 92%, 93%, 94%, 95%, 96%, 97%, 98% or 99% of an amino acid sequence of a target agent moiety, a protein agent moiety, an antibody agent moiety, etc. In some embodiments, it is 100%.

In some embodiments, protein agent moieties share a high percentage of amino acid sequence homology. In some embodiments, it is 50%-100%. In some embodiments, it is 50%. In some embodiments, it is 60%. In some embodiments, it is 70%. In some embodiments, it is 80%. In some embodiments, it is 90%. In some embodiments, it is 91%. In some embodiments, it is 50%. In some embodiments, it is 92%. In some embodiments, it is 93%. In some embodiments, it is 94%. In some embodiments, it is 95%. In some embodiments, it is 96%. In some embodiments, it is 97%. In some embodiments, it is 98%. In some embodiments, it is 99%. In some embodiments, it is 100%. In some embodiments, it is at least 50%. In some embodiments, it is at least 60%. In some embodiments, it is at least 70%. In some embodiments, it is at least 80%. In some embodiments, it is at least 90%. In some embodiments, it is at least 91%. In some embodiments, it is at least 50%. In some embodiments, it is at least 92%. In some embodiments, it is at least 93%. In some embodiments, it is at least 94%. In some embodiments, it is at least 95%. In some embodiments, it is at least 96%. In some embodiments, it is at least 97%. In some embodiments, it is at least 98%. In some embodiments, it is at least 99%.

In some embodiments, a protein agent moiety or an antibody agent moiety is or comprises a protein complex. In some embodiments, at least one or each individual chain shares a common amino acid sequence and/or has a homology as described herein.

In some embodiments, agents of a plurality share a common moiety of interest. In some embodiments, each agent of a plurality is independently an agent of formula P—I or P-II, or a salt thereof. In some embodiments, each agent of a plurality is independently an agent of formula P—I or P-II, or a salt thereof, wherein MOI is the same for each agent of the plurality. In some embodiments, agents of a plurality are products of methods described herein. In some embodiments, compositions comprising agents of a plurality are products of methods described herein.

In some embodiments, a modification is or comprises a moiety of interest and optionally a linker. In some embodiments, a modification is or comprises -L^{P}M-MOI.

In some embodiments, agents of the plurality share a common modification independently at at least one location. In some embodiments, a modification is or comprises a moiety of interest and optionally a linker connecting the moiety of interest. As described herein, each location independently has its common modification. In some embodiments, common modifications at two or more or all locations comprise a common moiety of interest. In some embodiments, common modifications are the same. In some embodiments, agents of the plurality share a common modification at each location which has a modification that is or comprises a moiety of interest and optionally a linker. In some embodiments, agents of the plurality share a common modification at each location which has a modification that is or comprises -L^{PM}-MOI.

In some embodiments, protein agents (e.g., antibody agents) share a common modification at least one amino acid residue. In some embodiments, agents of the plurality share a common modification at each location which has a modification that is or comprises a moiety of interest and optionally a linker. In some embodiments, agents of the plurality share a common modification at each location which has a modification that is or comprises -L^{P}M-MOI.

In some embodiments, a location is selected from K246, K248, K288, K290, K317 of antibody agents and locations corresponding thereto. In some embodiments, a location is selected from K246 and K248, and locations corresponding thereto. In some embodiments, a location is selected from K288 and K290, and locations corresponding thereto. In some embodiments, a location is K246 or a location corresponding thereto. In some embodiments, a location is K248 or a location corresponding thereto. In some embodiments, a location is K288 or a location corresponding thereto. In some embodiments, a location is K290 or a location corresponding thereto. In some embodiments, a location is K317 or a location corresponding thereto. In some embodiments, a location is K185 of light chain or a location corresponding thereto. In some embodiments, a location is K187 of light chain or a location corresponding thereto. In some embodiments, a location is K133 of heavy chain or a location corresponding thereto. In some embodiments, a location is K246 or K248 of heavy chain or a location corresponding thereto. In some embodiments, a location is K414 of heavy chain or a location corresponding thereto.

In some embodiments, about 1%-100% of all agents that comprise a target agent moiety and a moiety of interest are agents of the plurality. In some embodiments, about 1%-100% of all agents that comprise a protein agent moiety that comprise the common amino acid sequence and a moiety of interest are agents of the plurality. In some embodiments, about 1%-100% of all agents that comprise an antibody agent moiety that comprise the common amino acid sequence or can bind to the common antigen and a moiety of interest are agents of the plurality. In some embodiments, about 1%-100% of all agents that comprise a target agent moiety are agents of the plurality. In some embodiments, about 1%-100% of all agents that comprise a protein agent moiety that comprise the common amino acid sequence are agents of the plurality. In some embodiments, about 1%-100% of all agents that comprise an antibody agent moiety that comprise the common amino acid sequence or can bind to the common antigen are agents of the plurality. In some embodiments, it is 50%-100%. In some embodiments, it is 50%. In some embodiments, it is 60%. In some embodiments, it is 70%. In some embodiments, it is 80%. In some embodiments, it is 90%. In some embodiments, it is 91%. In some embodiments, it is 50%. In some embodiments, it is 92%. In some embodiments, it is 93%. In some embodiments, it is 94%. In some embodiments, it is 95%. In some embodiments, it is 96%. In some embodiments, it is 97%. In some embodiments, it is 98%. In some embodiments, it is 99%. In some embodiments, it is 100%. In some embodiments, it is at least 50%. In some embodiments, it is at least 60%. In some embodiments, it is at least 70%. In some embodiments, it is at least 80%. In some embodiments, it is at least 90%. In some embodiments, it is at least 91%. In some embodiments, it is at least 50%. In some embodiments, it is at least 92%. In some embodiments, it is at least 93%. In some embodiments, it is at least 94%. In some embodiments, it is at least 95%. In some embodiments, it is at least 96%. In some embodiments, it is at least 97%. In some embodiments, it is at least 98%. In some embodiments, it is at least 99%.

In some embodiments, provided agents, compounds, etc., e.g., those of formula R—I, P—I, P-II, etc. and salts thereof have high purity. In some embodiments, it is 50%-100%. In some embodiments, it is 50%. In some embodiments, it is 60%. In some embodiments, it is 70%. In some embodiments, it is 80%. In some embodiments, it is 90%. In some embodiments, it is 91%. In some embodiments, it is 50%. In some embodiments, it is 92%. In some embodiments, it is 93%. In some embodiments, it is 94%. In some embodiments, it is 95%. In some embodiments, it is 96%. In some embodiments, it is 97%. In some embodiments, it is 98%. In some embodiments, it is 99%. In some embodiments, it is 100%. In some embodiments, it is at least 50%. In some embodiments, it is at least 60%. In some embodiments, it is at least 70%. In some embodiments, it is at least 80%. In some embodiments, it is at least 90%. In some embodiments, it is at least 91%. In some embodiments, it is at least 50%. In some embodiments, it is at least 92%. In some embodiments, it is at least 93%. In some embodiments, it is at least 94%. In some embodiments, it is at least 95%. In some embodiments, it is at least 96%. In some embodiments, it is at least 97%. In some embodiments, it is at least 98%. In some embodiments, it is at least 99%.

In some embodiments, the present disclosure provides product agent compositions comprising product agents (e.g., agents of formula P—I or P-II, or a salt thereof). In some embodiments, a product agent composition (e.g., a formed agent composition from certain methods) comprises a product agent comprising a target agent moiety and a moiety of interest and optionally a linker (e.g., an agent of formula P—I or P-II, or a salt thereof), a released target binding moiety (e.g., a compound comprising R^{LG}-(L^{LG1})₀₋₁-(L^{LG2})₀₋₁-(L^{LG3})₀₋₁-(L^{LG4})₀₋₁-) or a compound comprising a released target binding moiety (e.g., a compound having the structure of R^{LG}-(L^{LG1})₀₋₁-(L^{LG2})₀₋₁-(L^{LG3})₀₋₁-(L^{LG4})₀₋₁-H or a salt thereof), and a reaction partner (e.g., a compound of formula R—I or a salt thereof). In some embodiments, released target binding moieties may bind to target agent moieties in target agents and/or formed product agents. Various technologies are available to separate released target binding moieties from target agent moieties in accordance with the present disclosure, for example, in some embodiments, contacting a composition with a composition comprising glycine at certain pH.

### Certain Embodiments of Variables

As examples, exemplary embodiments of variables are described throughout the present disclosure. As appreciated by those skilled in the art, embodiments for different variables may be optionally combined.

In some embodiments, ABT is an antibody binding moiety as described herein. In some embodiments, an ABT is an ABT of a compound selected from MMAE-1, MMAE-2, MMAE-3, MMAE-4, MMAE-5, MMAE-6, and MMAE-7. In some embodiments, an ABT is a moiety selected from Table A-1.

In some embodiments, L is a linker moiety of a compound selected from those depicted in compounds MMAE-1, MMAE-2, MMAE-3, MMAE-4, MMAE-5, MMAE-6, and MMAE-7.

### General Methods, Reagents and Conditions

Various technologies may be utilized to provide compounds and agents herein in accordance with the present disclosure.

In some embodiments, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd}Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

In some embodiments, leaving groups include but are not limited to, halogens (e.g. fluoride, chloride, bromide, iodide), sulfonates (e.g. mesylate, tosylate, benzenesulfonate, brosylate, nosylate, triflate), diazonium, and the like.

In some embodiments, an oxygen protecting group includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

One of skill in the art will appreciate that compounds/agents may contain one or more stereocenters, and may be present as a racemic or diastereomeric mixture. One of skill in the art will also appreciate that there are many methods known in the art for the separation of isomers to obtain stereoenriched or stereopure isomers of those compounds, including but not limited to HPLC, chiral HPLC, fractional crystallization of diastereomeric salts, kinetic enzymatic resolution (e.g. by fungal-, bacterial-, or animal-derived lipases or esterases), and formation of covalent diastereomeric derivatives using an enantioenriched reagent.

One of skill in the art will appreciate that various functional groups present in compounds of the present disclosure such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5^{th} Ed., Ed.: Smith, M. B. and March, J., John Wiley & Sons, New York: 2001, the entirety of which is incorporated herein by reference. Such interconversions may require one or more of the aforementioned techniques, and certain methods for synthesizing compounds of the present disclosure are described below in the Exemplification.

### Uses, Formulation and Administration

Compounds, agents, compositions, etc. of the present disclosure may be provided as in various forms according to desired uses. In some embodiments, they are provided as pharmaceutical compositions. As appreciated by those skilled in the art, in many instances, pharmaceutical compositions comprise controlled amounts and are manufactured for administration to subjects such as human patients. In some embodiments, the present disclosure provides a composition comprising a compound, an agent, and/or a composition described herein or a pharmaceutically acceptable derivative thereof and a pharmaceutically acceptable carrier. In some embodiments, the present disclosure provides a pharmaceutical composition comprising a compound, agent or composition of the present disclosure and a pharmaceutically acceptable carrier. In some embodiments, the present disclosure provides a pharmaceutical composition comprising a therapeutically effective amount of a compound, an agent or a composition of the present disclosure and a pharmaceutically acceptable carrier. In some embodiments, a pharmaceutical composition is packaged for storage, transportation, administration, etc. In some embodiments, a pharmaceutical composition does not contain a significant amount of organic solvents (e.g., total amount of organic solvents no more than 50%, 40%, 30%, 25%, 20%, 15%, 10%, 5%, 4%, 3%, 2%, or 1% of weight and/or volume of a pharmaceutical composition).

In some embodiments, a pharmaceutically acceptable carrier is or comprises a non-toxic carrier, adjuvant, or vehicle that does not destroy the pharmacological activity of the compound with which it is formulated. Pharmaceutically acceptable carriers, adjuvants or vehicles that may be include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In some embodiments, a pharmaceutically acceptable derivative is a non-toxic salt, ester, salt of an ester or other derivative of a compound that, upon administration to a recipient, is capable of providing, either directly or indirectly, a compound or an active metabolite or residue thereof.

Compositions may be administered orally, parenterally, by inhalation spray, topically, rectally, nasally, buccally, vaginally or via an implanted reservoir. In some embodiments, parenteral administration includes subcutaneous, intravenous, intramuscular, intra-articular, intra-synovial, intrasternal, intrathecal, intrahepatic, intralesional and intracranial injection or infusion techniques. In some embodiments, compositions are administered orally, intraperitoneally or intravenously. Sterile injectable forms of compositions may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

In some embodiments, a bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

In some embodiments, pharmaceutically acceptable compositions may be administered in the form of suppositories for rectal administration. In some embodiments, these can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

In some embodiments, pharmaceutically acceptable compositions may be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, pharmaceutically acceptable compositions are formulated for oral administration. Such formulations may be administered with or without food. In some embodiments, pharmaceutically acceptable compositions are administered without food. In other embodiments, pharmaceutically acceptable compositions are administered with food.

Amounts of compounds that may be combined with the carrier materials to produce a composition in a single dosage form will vary depending upon the host treated, the particular mode of administration. In some embodiments, provided compositions are formulated so that a dosage of between 0.01-100 mg/kg body weight/day of the inhibitor can be administered to a patient receiving these compositions.

In some embodiments, the present invention is directed to compositions that include therapy enhancer agents containing moieties of interest conjugated to target agent moieties at specific locations.

In an embodiment, provided is a composition including:
a first compound having the structure of formula (P-II):

P—N-L^{PM}-MOI (P-II)
wherein:
P—N is a protein agent moiety including a lysine residue;
L^{PM} is a linker; and
MOI is a moiety of interest comprising monomethyl auristatin E (MMAE); and
a second compound having the structure:

LG-OH (LG-I)
wherein LG is a group including a target binding moiety that binds to a target agent.

In another embodiment, the composition further includes:
a third compound having the formula (R-I):

LG-RG-L^{RM}-MOI (R-I)
LG is a group including a target binding moiety that binds to a target agent, which is identical to LG in formula (LG-I);
RG is a reactive group;
L^{RM} is a linker, which is identical to in formula (P-II); and
MOI is a moiety of interest comprising monomethyl auristatin E (MMAE).
a fourth compound having the formula (R—III):

HO-RG-L^{RM}-MOI (R—III)
or a combination thereof.

In some embodiment, the compositions may include the first and second compounds in equimolar amount. In some embodiments, the amount of the second compound may be 50 mole percent (mole %) or less based on the total number of moles of the first and second compounds in the composition. In some embodiments, the amount of the second compound may be 50 mole % or less, 45 mole % or less, 40 mole % or less, 35 mole % or less, 30 mole % or less, 25 mole % or less, 20 mole % or less, 15 mole % or less, 10 mole % or less, or 5 mole % or less based on the total number of moles of the first and second compounds in the composition. In some embodiments, the amount of the second compound may be 5% or less, 4% or less, 3% or less, 2% or less, or 1% or less based on the total number of moles of the first and second compounds in the composition. In some embodiments, the amount of the second compound may be 1.0% or less, 0.9% or less, 0.8% or less, 0.7% or less, 0.6% or less, 0.5% or less, 0.4% or less, 0.3% or less, 0.2% or less, 0.1% or less based on the total number of moles of the first and second compounds in the composition. In some embodiments, the amount of the second compound may be 0.10% or less, 0.09% or less, 0.08% or less, 0.07% or less, 0.06% or less, 0.05% or less, 0.04% or less, 0.03% or less, 0.02% or less, 0.01% or less based on the total number of moles of the first and second compounds in the composition. In some embodiments, the amount of the second compound may be 0.010% or less, 0.009% or less, 0.008% or less, 0.007% or less, 0.006% or less, 0.005% or less, 0.004% or less, 0.003% or less, 0.002% or less, 0.001% or less based on the total number of moles of the first and second compounds in the composition. In some embodiments, the amount of the second compound may be 0.0010% or less, 0.0009% or less, 0.0008% or less, 0.0007% or less, 0.0006% or less, 0.0005% or less, 0.0004% or less, 0.0003% or less, 0.0002% or less, 0.0001% or less based on the total number of moles of the first and second compounds in the composition. In some embodiments, the amount of the second compound may be 0.00010% or less, 0.00009% or less, 0.00008% or less, 0.00007% or less, 0.00006% or less, 0.00005% or less, 0.00004% or less, 0.00003% or less, 0.00002% or less, 0.00001% or less based on the total number of moles of the first and second compounds in the composition. In some embodiments, the amount of the second compound may be 0.000010% or less, 0.000009% or less, 0.000008% or less, 0.000007% or less, 0.000006% or less, 0.000005% or less, 0.000004% or less, 0.000003% or less, 0.000002% or less, 0.000001% or less based on the total number of moles of the first and second compounds in the composition.

In some embodiment, the compositions may further include a third compound, a fourth compound, or a combination thereof. In some embodiments, the amount of the third compound, the fourth compound, or the combination thereof may be 5% or less, 4% or less, 3% or less, 2% or less, or 1% or less based on the number of moles of the first compound in the composition. In some embodiments, the amount of the third compound, the fourth compound, or the combination thereof may be 1.0% or less, 0.9% or less, 0.8% or less, 0.7% or less, 0.6% or less, 0.5% or less, 0.4% or less, 0.3% or less, 0.2% or less, 0.1% or less based on the number of moles of the first compound in the composition. In some embodiments, the amount of the third compound, the fourth compound, or the combination thereof may be 0.10% or less, 0.09% or less, 0.08% or less, 0.07% or less, 0.06% or less, 0.05% or less, 0.04% or less, 0.03% or less, 0.02% or less, 0.01% or less based on the number of moles of the first compound in the composition. In some embodiments, the amount of the third compound, the fourth compound, or the combination thereof may be 0.010% or less, 0.009% or less, 0.008% or less, 0.007% or less, 0.006% or less, 0.005% or less, 0.004% or less, 0.003% or less, 0.002% or less, 0.001% or less based on the number of moles of the first compound in the composition. In some embodiments, the amount of the third compound, the fourth compound, or the combination thereof may be 0.0010% or less, 0.0009% or less, 0.0008% or less, 0.0007% or less, 0.0006% or less, 0.0005% or less, 0.0004% or less, 0.0003% or less, 0.0002% or less, 0.0001% or less based on the number of moles of the first compound in the composition. In some embodiments, the amount of the third compound, the fourth compound, or the combination thereof may be 0.00010% or less, 0.00009% or less, 0.00008% or less, 0.00007% or less, 0.00006% or less, 0.00005% or less, 0.00004% or less, 0.00003% or less, 0.00002% or less, 0.00001% or less based on the number of moles of the first compound in the composition. In some embodiments, the amount of the third compound, the fourth compound, or the combination thereof may be 0.000010% or less, 0.000009% or less, 0.000008% or less, 0.000007% or less, 0.000006% or less, 0.000005% or less, 0.000004% or less, 0.000003% or less, 0.000002% or less, 0.000001% or less based on the number of moles of the first compound in the composition.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

Technologies (e.g., compounds, agents, compositions) of the present disclosure can be utilized for various purposes, e.g., detection, diagnosis, therapy, etc. In some embodiments, provided technologies are useful for treating conditions, disorders or diseases, e.g., various cancers. In some embodiments, provided technologies comprise target binding moieties, e.g., antibody agent moieties, that can bind antigens of cancer cells. In some embodiments, a target binding moiety is an antibody agent moiety. In some embodiments, an antibody agent is a therapeutic agent. Among other things, various antibody agents, including many developed and/or approved (e.g., by FDA, EMA, etc.) as therapeutics can be utilized in accordance with the present disclosure to provide therapeutics for various diseases.

Among other things, the present disclosure provides the following embodiments:
1. A compound having the structure of formula R-I:

LG-RG-L^{RM}-MOI (R-I)
or a salt thereof, wherein:
   LG is R^{LG}-L^{LG};
   R^{LG} is

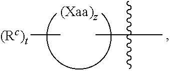

R^{c}-(Xaa)z-, a nucleic acid moiety, or a small molecule moiety;
each Xaa is independently a residue of an amino acid or an amino acid analog;
t is 0-50;
z is 1-50;
each R^{c} is independently -L^{a}-R′;
each of a and b is independently 1-200;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—; each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
L^{LG} is L^{LG1}-, -L^{LG1}-L^{LG2}-, -L^{LG1}-L^{LG2}-L^{LG3}- or -L^{LG1}-L^{LG2}-L^{LG3}-L^{LG4}-.
RG is -L^{RG1}-L^{RG2}-, -L^{LG4}-L^{RG1}-L^{RG2}-, -L^{LG3}-L^{LG4}-L^{RG1}-L^{RG2}-, -L^{LG2}-L^{LG3}-L^{LG4}-L^{RG1}-L^{RG2}-.
each of L^{LG1}, L^{LG2}, L^{LG3}, L^{LG4}, L^{RG1}, L^{RG2}, and L^{RM} is independently L;
each L is independently a covalent bond, or a bivalent optionally substituted, linear or branched C₁₋₁₀₀ group comprising one or more aliphatic moieties, aryl moieties, heteroaliphatic moieties each independently having 1-20 heteroatoms, heteroaromatic moieties each independently having 1-20 heteroatoms, or any combinations of any one or more of such moieties, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or —[(—O—C(R′)₂—C(R′)₂—)ₙ]—, wherein n is 1-20;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms; and
MOI is a moiety of interest that is or comprises vedotin (MMAD, MMAE, or MMAF), vedotin, ozogamicin, mafodotin, deruxtecan, emtansine, govitecan, tesirine, duocarmazine, soravtansine, ravtansine.
2. The compound of any one of the preceding embodiments, wherein LG is or comprises a target binding moiety that binds to a target agent, wherein a target agent is a protein agent.
3. The compound of any one of the preceding embodiments, wherein LG is or comprises a target binding moiety that binds to a target agent, wherein the target agent is an antibody agent.
4. The compound of any one of the preceding embodiments, wherein LG is or comprises a target binding moiety that binds to a Fc region or an antibody agent.
5. The compound or salt of any preceding claim wherein LG is or comprises a target binding moiety that binds to a target agent, wherein the target agent is an antibody agent that is or comprises enfortumab, brentuximab, belantamab, vorsetuzumab, inotuzumab, trastuzumab, gemtuzumab, polatuzumab, Sacituzumab, tisotumab, loncastuximab, datopotamab, depatuxizumab, mirvetuximab, tusamitamab, anetumab, camidanlumab, coltuximab, disitamab, labetuzumab, ladiratuzumab, lifastuzumab, naratuximab, cirmtuzumab, patritumab, pinatuzumab, polatuzumab, enapotamab, anetumab, or omburtamab.
6. The compound of any one of the preceding embodiments, wherein each L is independently a covalent bond, or a bivalent optionally substituted, linear or branched aliphatic group or heteroaliphatic group having 1-10 heteroatoms, wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or —[(—O—C(R′)₂—C(R′)₂—)ₙ]—, wherein n is 1-20.
7. The compound of any one of the preceding embodiments, wherein LG is R^{LG}-L^{LG}-, wherein R^{LG} is or comprises a target binding moiety, wherein L^{LG} is L^{LG1}, wherein L^{LG1} is L.
8. The compound of any one of the preceding embodiments, wherein RG is or comprises L^{LG2}-L^{LG3}-L^{LG4}-L^{RG1}-L^{RG2}- wherein each of L^{LG2}, L^{LG3} L^{LG4}, L^{RG1}, L^{RG2} is independently L.
9. The compound of any one of the preceding embodiments, wherein LG is R^{LG}-L^{LG}-, wherein R^{LG} is or comprises a target binding moiety, wherein L^{LG} is L^{LG1}-L^{LG2}-.
10. The compound of any one of the preceding embodiments, wherein RG is or comprises -L^{LG3}-L^{LG4}-L^{RG1}L^{RG2}-
11. The compound of any one of the preceding embodiments, wherein LG is R^{LG}-L^{LG}-, wherein R^{LG} is or comprises a target binding moiety, wherein L^{LG} is L^{LG1}-L^{LG2}-L^{LG3}-.
12. The compound of any one of the preceding embodiments, wherein RG is or comprises -L^{LG4}-L^{RG1}-L^{RG2}-
13. The compound of any one of the preceding embodiments, wherein LG is R^{LG}-L^{LG}-, wherein R^{LG} is or comprises a target binding moiety, wherein L^{LG} is L^{LG1}-L^{LG2}-L^{LG3}-L^{LG4}-.
14. The compound of any one of the preceding embodiments, wherein RG is or comprises -L^{RG1}-L^{RG2}-.
15. The compound of any one of the preceding embodiments, wherein R^{LG} is

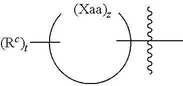

or R^{c}-(Xaa)z-.
16. The compound of any one of the preceding embodiments, wherein R^{LG} is or comprises WXL, wherein X is an amino acid residue.
17. The compound of any one of the preceding embodiments, wherein R^{LG} is or comprises AWXLGELVW (SEQ ID NO:16), wherein X is an amino acid residue.
18. The compound of any one of the preceding embodiments, wherein R^{LG} is or comprises DpLpAWXLGELVW (SEQ ID NO:20), wherein X is an amino acid residue.
19. The compound of any one of the preceding embodiments, wherein R^{LG} is or comprises DCAWXLGELVWCT (SEQ ID NO:18), wherein the two cysteine residues optionally form a disulfide bond, and X is an amino acid residue.
20. The compound of any one of the preceding embodiments, wherein R^{LG} is or comprises DpLpDCAWXLGELVWCT (SEQ ID NO:23), wherein the two cysteine residues optionally form a disulfide bond, and X is an amino acid residue.
21. The compound of any one of the preceding embodiments, wherein R^{LG} is or comprises CDCAWXLGELVWCTC (SEQ ID NO:26), wherein the first and the last cysteines, and the two cysteines in the middle of the sequence, are each independently and optionally form a disulfide bond, and X is an amino acid residue.
22. The compound of any one of embodiments 16-21, wherein R^{LG} is or comprises WXL, wherein X is an amino acid residue.
23. The compound of embodiment 15, wherein R^{LG} is selected from A-1 to A-50 in Table A-1.
24. The compound of embodiment 15, wherein R^{LG} is

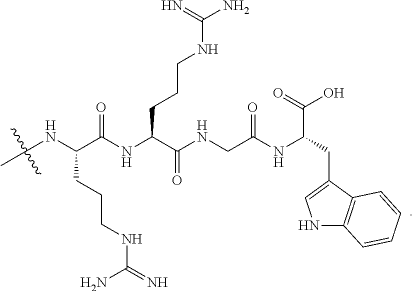 [SMILES: *N[C@@H](CCCNC(=N)N)C(=O)N[C@@H](CCCNC(=N)N)C(=O)NCC(=O)N[C@@H](Cc1c[nH]c2ccccc12)C(=O)O]

25. The compound of embodiment 15, wherein R^{LG} is

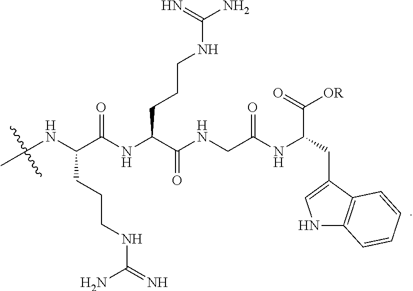

26. The compound of embodiment 15, wherein R^{LG} is

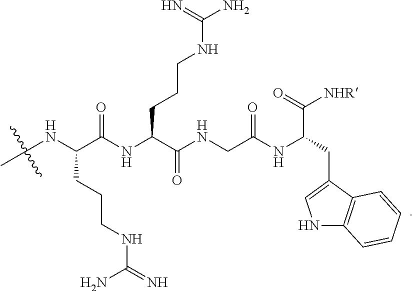

27. The compound of embodiment 15, wherein R^{LG} is

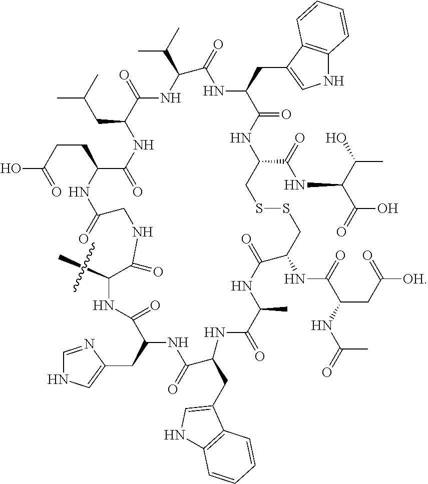

28. The compound of embodiment 15, wherein R^{LG} is

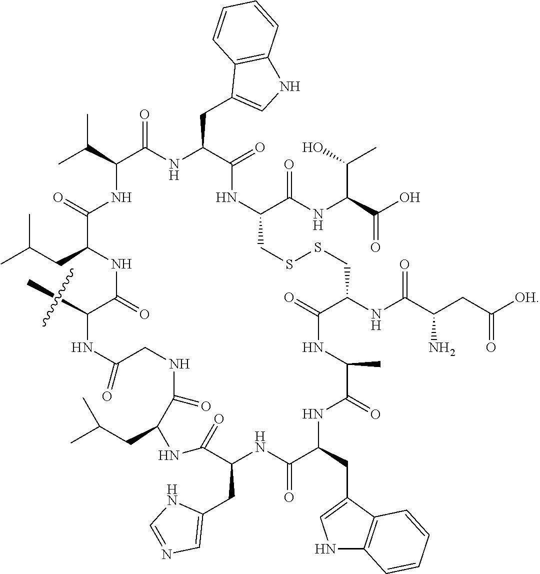

29. The compound of embodiment 15, wherein R^{LG} is

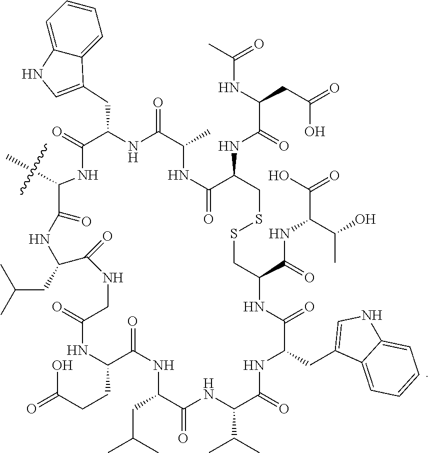

30. The compound of embodiment 15, wherein R^{LG}

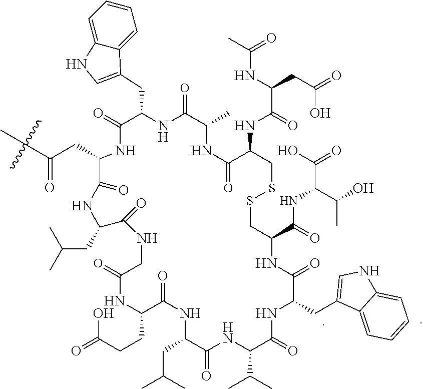 [SMILES: *C(=O)C[C@@H]1NC(=O)[C@H](Cc2c[nH]c3ccccc23)NC(=O)[C@H](C)NC(=O)[C@@H](NC(=O)[C@H](CC(=O)O)NC(C)=O)CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC1=O]

31. The compound of embodiment 15, wherein R^{LG} is

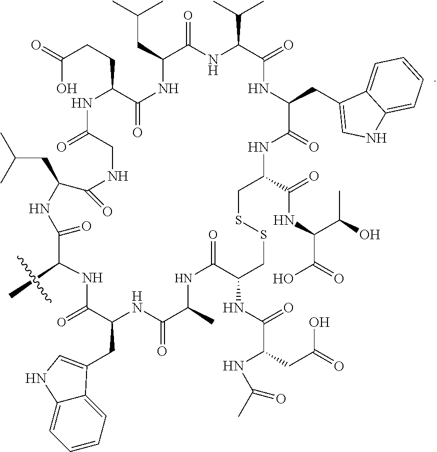

32. The compound of embodiment 15, wherein R^{LG} is

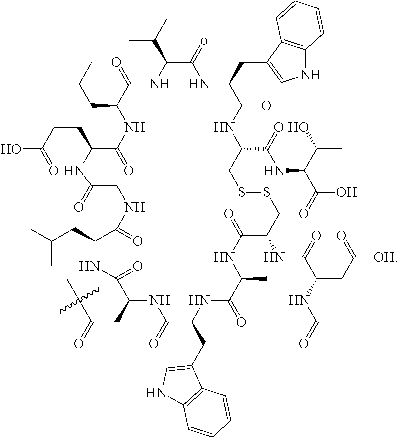 [SMILES: *C(=O)C[C@@H]1NC(=O)[C@H](Cc2c[nH]c3ccccc23)NC(=O)[C@H](C)NC(=O)[C@@H](NC(=O)[C@H](CC(=O)O)NC(C)=O)CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC1=O]

33. The compound of embodiment 15, wherein R^{LG} is

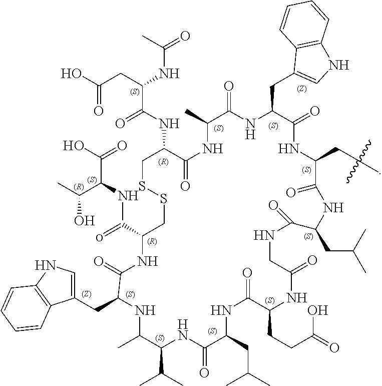

34. The compound of embodiment 15, wherein R^{LG} is

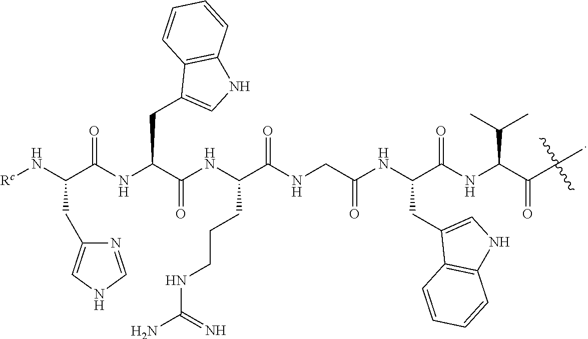

35. The compound of embodiment 15, wherein R^{LG} is NH

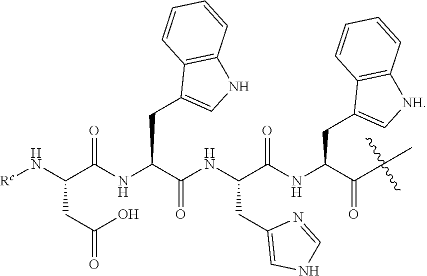

36. The compound of embodiment 15, wherein R^{LG} is

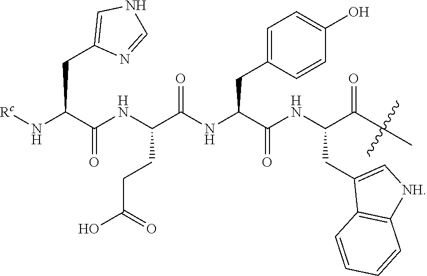

37. The compound of embodiment 15, wherein R^{LG} is

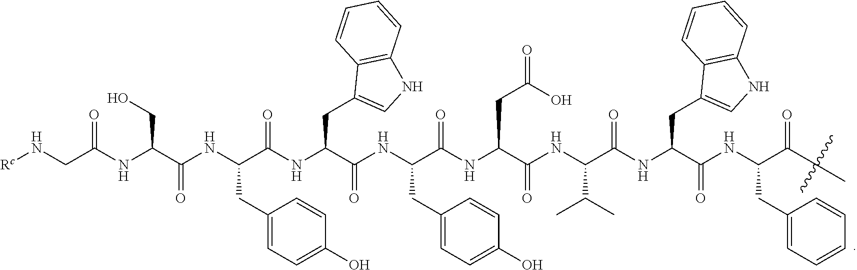

38. The compound of embodiment 15, wherein R^{LG} is

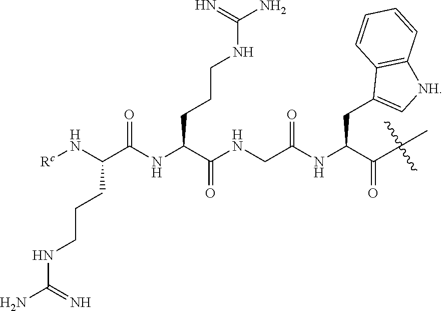

39. The compound of any one of embodiments 33-37, wherein R^{c} is R—C(O)—, wherein R is optionally substituted C₁₋₆ aliphatic.
40. The compound of any one of embodiments 33-37, wherein R^{c} is CH₃C(O)—.
41. The compound of any one of embodiments 1-14, wherein R^{LG} is a small molecule moiety.
42. The compound of any one of the preceding embodiments, wherein L^{LG1} is a covalent bond.
43. The compound of any one of embodiments 1-14, wherein L^{LG1} is or comprises —(CH₂CH₂O)n-.
44. The compound of any one of embodiments 1-14, wherein L^{LG1} is or comprises —(CH₂)n-O—(CH₂CH₂O)n-(CH₂)n-, wherein each n is independently 1-10, and each —CH₂— is independently optionally substituted.
45. The compound of any one of the preceding embodiments, wherein L^{LG2} is or comprises —NR′—.
46. The compound of any one of the preceding embodiments, wherein L^{LG2} is or comprises —C(O)—.
47. The compound of any one of the preceding embodiments, wherein L^{LG2} is or comprises —NR′C(O)—.
48. The compound of any one of the preceding embodiments, wherein L^{LGZ} is or comprises —(CH₂)n-OC(O)N(R′)—, wherein —(CH₂)n- is optionally substituted.
49. The compound of any one of embodiments 1-60, wherein L^{LG2} is a covalent bond.
50. The compound of any one of embodiments 1-60, wherein L^{LG2} is —CH₂N(CH₂CH₂CH₂S(O)₂OH)—C(O)—.
51. The compound of any one of embodiments 1-60, wherein L^{LG2} is —C(O)—NHCH₂—.
52. The compound of any one of embodiments 1-60, wherein L^{LG2} is —C(O)O—CH₂—.
53. The compound of any one of embodiments 1-60, wherein L^{LG2} is —NH—C(O)O—CH₂—.
54. The compound of any one of embodiments 62-63 and 66-69, wherein —C(O)— is bonded to L^{LG3}
55. The compound of any one of the preceding embodiments, wherein L^{LG3} is or comprises an optionally substituted aryl ring.
56. The compound of any one of the preceding embodiments, wherein L^{LG3} is or comprises an optionally substituted phenyl ring.
57. The compound of any one of embodiments 71-72, wherein the ring is substituted, and one or more substituents are independently an electron-withdrawing group.
58. The compound of embodiment 73, wherein a substituent is —F.
59. The compound of embodiment 73, wherein a substituent is —NO₂.
60. The compound of any one of embodiments 1-75, wherein L^{LG3} is

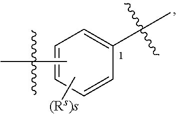

wherein s is 0-4, each R^{s} is independently halogen, —NO₂, -L-R′, —C(O)-L-R′, —S(O)-L-R′, —S(O)₂-L-R′, or —P(O)(-L-R′)₂.
61. The compound of any one of embodiments 1-75, wherein L^{LG3} is

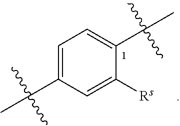

62. The compound of any one of embodiments 1-75, wherein L^{LG3} is

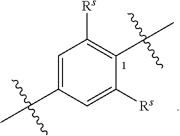

63. The compound of any one of embodiments 1-71, wherein L^{LG3} is F.

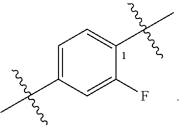 [SMILES: *c1ccc(*)c(F)c1]

64. The compound of any one of embodiments 1-71, wherein L^{LG3} is F.

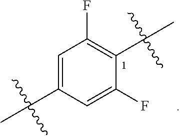 [SMILES: *c1cc(F)c(*)c(F)c1]

65. The compound of any one of embodiments 76-80, wherein C1 is bonded to L^{LG4}.
66. The compound of any one of embodiments 1-70, wherein L^{LG3} is a covalent bond.
67. The compound of any one of the preceding embodiments, wherein L^{LG4} is or comprises —O—.
68. The compound of any one of the preceding embodiments, wherein L^{LG4} is or comprises —NR′—.
69. The compound of any one of embodiments 1-83, wherein L^{LG4} is —O—.
70. The compound of any one of embodiments 1-83, wherein L^{LG4} is —NH—.
71. The compound of any one of embodiments 1-83, wherein L^{LG4} is a covalent bond.
72. The compound of any one of the preceding embodiments, wherein L^{RG1} is a covalent bond.
73. The compound of any one of embodiments 1-88, wherein L^{RG1} is or comprises —S(O)₂—.
74. The compound of any one of the preceding embodiments, wherein L^{RG2} is or comprises —C(O)—.
75. The compound of any one of the preceding embodiments, wherein L^{RG2} is or comprises -L^{RG3}-C(═CR^{RG1}R^{RG2})—CR^{RG3}R^{RG4}—, wherein each of R^{RG1}, R^{RG2}, R^{RG3} and R^{RG4} is independently -L-R′ and L^{RG3} is —C(O)—, —C(O)O—, —C(O)N(R′)—, —S(O)—, —S(O)₂—, —P(O)(OR′)—, —P(O)(SR′)—, or —P(O)(N(R′)₂)—.
76. The compound of any one of the preceding embodiments, wherein L^{RG2} is or comprises optionally substituted -L^{RG3}-C(═CHR^{RG2})—CHR^{RG4}—.
77. The compound of embodiment 91 or 92, wherein R^{RG2} and R^{RG4} are taken together with their intervening atoms to form an optionally substituted 3-10 membered monocyclic or bicyclic ring having 0-5 heteroatoms.
78. The compound of embodiment 91 or 92, wherein —C(═CHR^{RG2})—CHR^{RG4} or —C(═CR^{RG1}R^{RG2})—CR^{RG3}R^{RG4} is optionally substituted

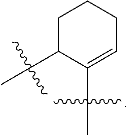 [SMILES: *C1=CCCCC1*]

79. The compound of any one of embodiments 1-89, wherein L^{RG2} is —C(O)—.
80. The compound of any one of embodiments 1-89, wherein L^{RG2} is

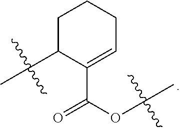 [SMILES: *OC(=O)C1=CCCCC1*]

81. The compound of any one of embodiments 1-89, wherein -L^{LG1}-L^{RG2}- is —C(O)—.
82. The compound of any one of embodiments 1-89, wherein -L^{LG1}-L^{RG2}- is

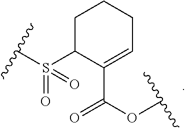 [SMILES: *OC(=O)C1=CCCCC1S(*)(=O)=O]

83. The compound of any one of the preceding embodiments, wherein L^{PM} is or comprises —(CH₂CH₂O)n-.
84. The compound of any one of the preceding embodiments, wherein L^{PM} is or comprises —(CH₂)n-O—(CH₂CH₂O)n-(CH₂)n-, wherein each n is independently 1-10, and each —CH₂— is independently optionally substituted.
85. The compound of any one of the preceding embodiments, wherein a moiety of interest is or comprises a cytotoxic agent, such as MMAD, MMAE, and MMAF.
86. The compound of any one of the preceding embodiments, wherein a moiety of interest is or comprises a peptide moiety that is or comprises MMAD, MMAE, or MMAF.
87. The compound of any one of the preceding embodiments, wherein a moiety of interest is or comprises a reactive moiety, additionally to MMAD, MMAE, or MMAF.
88. The compound of any one of the preceding embodiments, wherein a moiety of interest is or comprises a reactive moiety suitable for a bioorthogonal reaction.
89. The compound of any one of the preceding embodiments, wherein the compound comprises no cleavable groups whose cleavage can release LG except one or more optionally in RG.
90. The compound of any one of the preceding embodiments, wherein the compound comprises no —S—S—, acetal or imine groups except in RG or MOI.
91. The compound of any one of the preceding embodiments, wherein the compound comprises no —S—S—, acetal or imine groups except that the compound may have —S—S— formed by two amino acid residues.
92. The compound of any one of the preceding embodiments, wherein the compound comprises no —S—S—, acetal or imine groups except that the compound may have —S—S— formed by cysteine residues.
93. The compound of any one of the preceding embodiments, wherein the compound comprises no —S—S—, acetal or imine groups.
94. The compound of any one of the preceding embodiments, wherein the compound comprises one or more groups selected from:

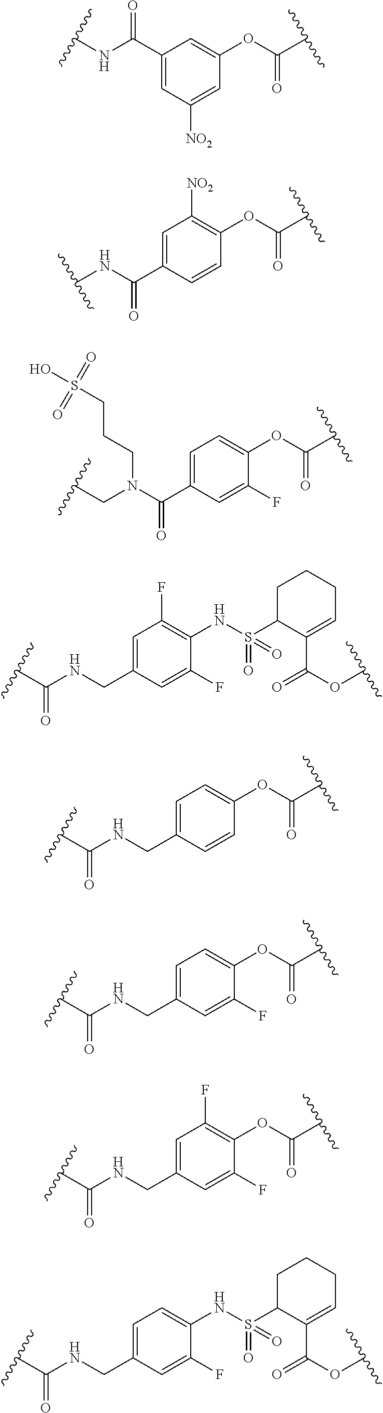

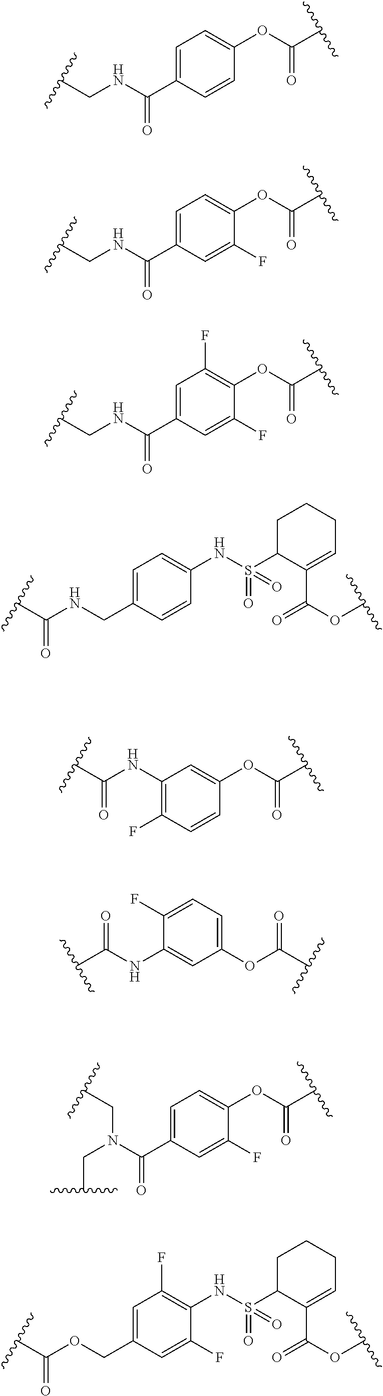

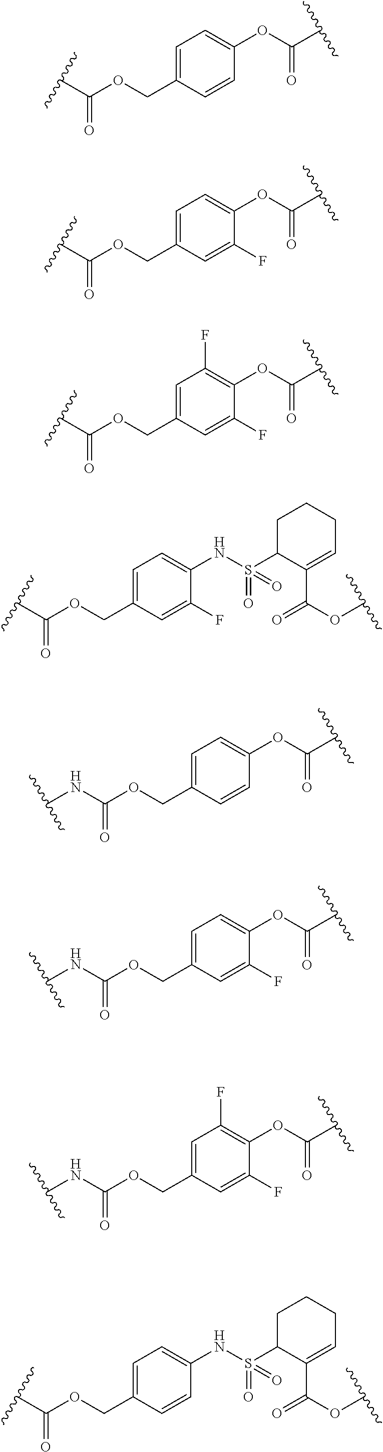

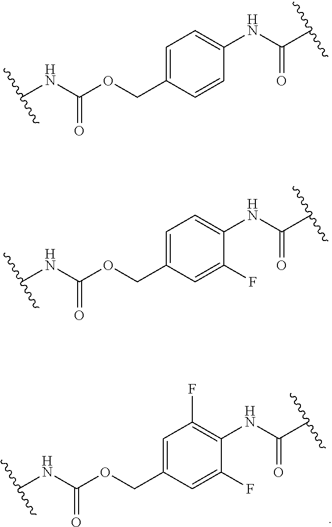 [SMILES: *NC(=O)OCc1ccc(NC(*)=O)cc1 ; *NC(=O)OCc1ccc(NC(*)=O)c(F)c1 ; *NC(=O)OCc1cc(F)c(NC(*)=O)c(F)c1]

95. The compound of any one of the preceding embodiments, wherein the compound comprises a structure selected from:

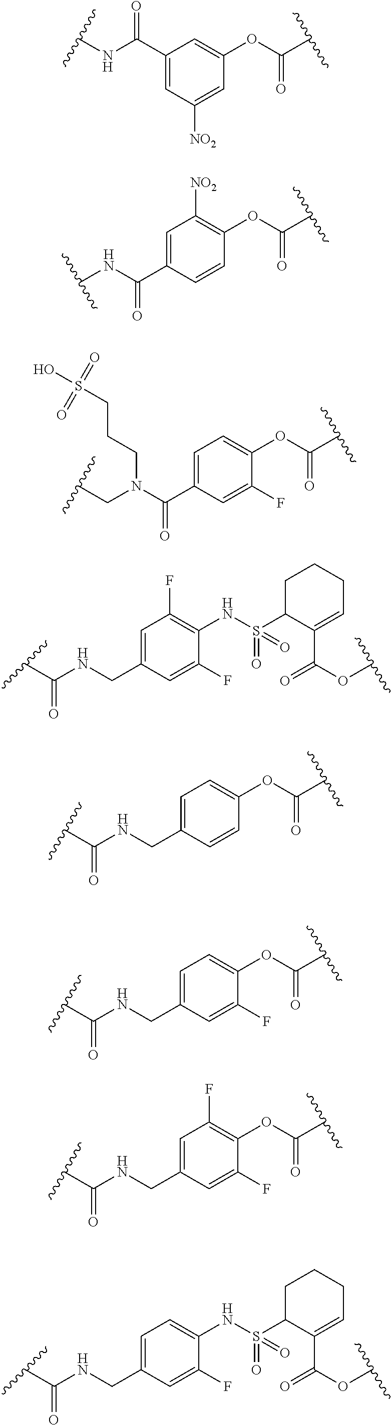

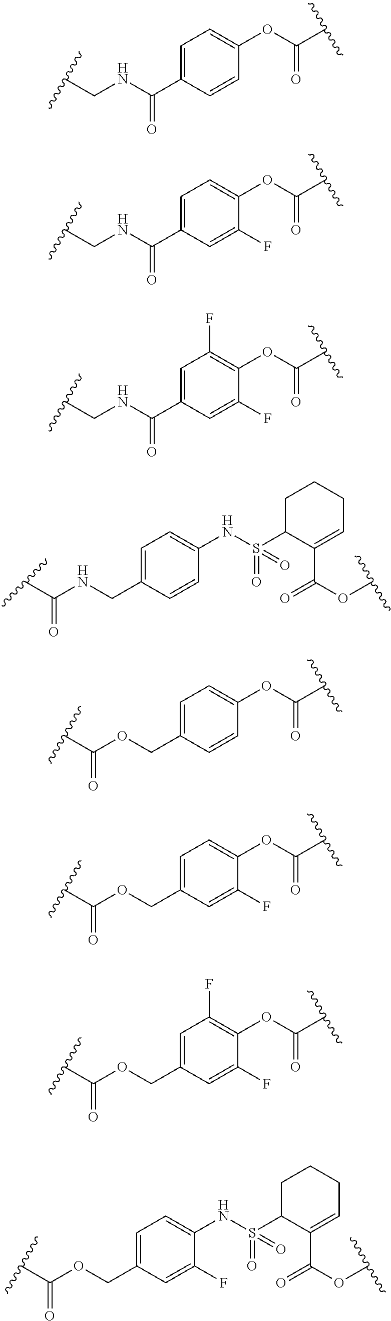

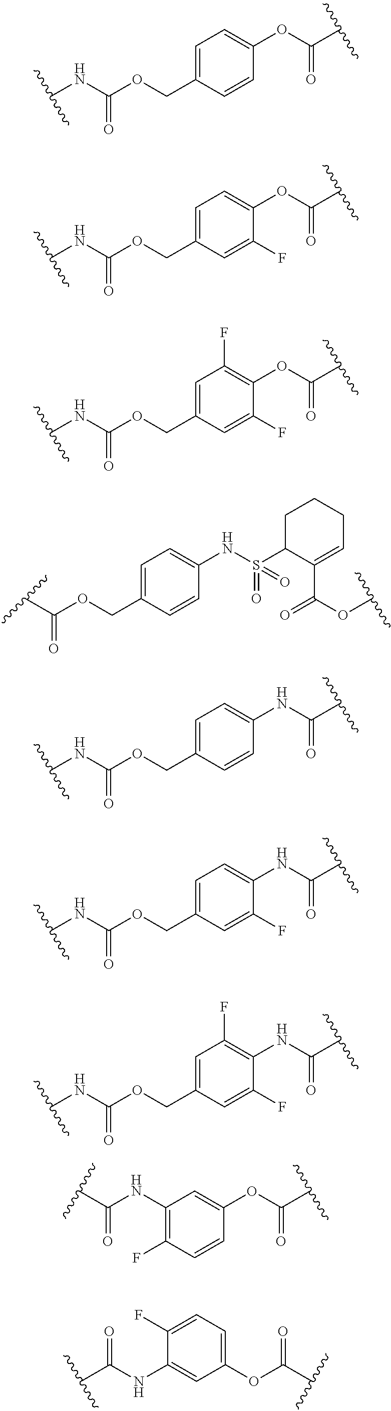

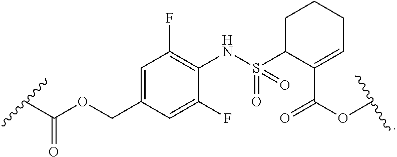 [SMILES: *OC(=O)C1=CCCCC1S(=O)(=O)Nc1c(F)cc(COC(*)=O)cc1F]

96. The compound of any one of embodiments 1-94, comprising a reactive group, wherein the reactive group comprises or is a group shown in embodiment 95.
97. The compound of any one of the preceding embodiments, wherein the compound comprises two or more target binding moieties.
98. A method, comprising steps of:
   1) contacting a target agent with a reaction partner comprising:
      a first group comprising a target binding moiety that binds to a target agent,
      a reactive group;
      a moiety of interest that is or comprises MMAD, MMAE, or MMAF; and
      optionally one or more linker moieties;
   2) forming an agent comprising:
      a target agent moiety;
      the moiety of interest; and
      optionally one or more linker moieties.
99. The method of embodiment 98, wherein a reactive group is located between a first group and a moiety of interest, and is connected to a first group and a moiety of interest independently and optionally through a linker moiety.
100. A method of preparing an agent having the structure of P-I:

P-L^{PM}-MOI (P—I)
or a salt thereof, wherein:
   P is a target agent moiety;
   L^{PM} is a linker; and
   MOI is a moiety of interest that is or comprises MMAD, MMAE, or MMAF.
comprising steps of:
   1) contacting a target agent with a reaction partner having the structure of formula R-I:

LG-RG-L^{RM}-MOI (P—I)
or a salt thereof, wherein:
   LG is a group comprising a target binding moiety that binds to a target agent,
   RG is the reactive group;
   L^{RM} is a linker; and
   MOI is the moiety of interest that is or comprises MMAD, MMAE, or MMAF; and
   2) forming an agent having the structure of formula P-I.
101. A method of preparing an agent having the structure of P-II:

P—N-L^{PM}-MOI (P-II)
wherein:
   P—N is a protein agent moiety comprising a lysine residue;
   L^{PM} is a linker; and
   MOI is the moiety of interest that is or comprises MMAD, MMAE, or MMAF;
the method comprising:
   contacting P—N with a reaction partner having a structure of formula R-I:

LG-RG-L^{RM}-MOI (R-I)
or a salt thereof, wherein:
   LG is a group comprising a protein-binding moiety that binds to P—N,
   RG is a reactive group;
   L^{RM} is a linker; and
   MOI is the moiety of interest that is or comprises MMAD, MMAE, or MMAF.
102. The method of any one of the proceeding embodiments, wherein a target agent is or comprises a protein agent.
103. The method of any one of the proceeding embodiments, wherein a target agent is or comprises an antibody agent, such as enfortumab, brentuximab, belantamab, vorsetuzumab, inotuzumab, trastuzumab, gemtuzumab, polatuzumab, Sacituzumab, tisotumab, loncastuximab, datopotamab, depatuxizumab, mirvetuximab, tusamitamab, anetumab, camidanlumab, coltuximab, disitamab, labetuzumab, ladiratuzumab, lifastuzumab, naratuximab, cirmtuzumab, patritumab, pinatuzumab, polatuzumab, enapotamab, anetumab, or omburtamab.
104. The method of embodiment 103, wherein a moiety of interest is selectively attached to the antibody agent at K246 or K248 or a corresponding location.
105. The method of embodiment 103, wherein a moiety of interest is selectively attached to the antibody agent at K288 or K290 or a corresponding location.
106. The method of embodiment 103, wherein a moiety of interest is selectively attached to the antibody agent at K251 or K253 of an IgG2 heavy chain or a corresponding location.
107. The method of embodiment 103, wherein a moiety of interest is selectively attached to the antibody agent at K239 or K241 of an IgG4 heavy chain or a corresponding location.
108. The method of embodiment 103, wherein a moiety of interest is selectively attached to the antibody agent at K317 or a corresponding location.
109. The method of embodiment 103, wherein a moiety of interest is selectively attached to the antibody agent at heavy chain residue(s) over light chain residue(s).
110. The method of any one of the proceeding embodiments, wherein a target agent is or comprise an IgG antibody agent.
111. The method of any one of the proceeding embodiments, wherein a target agent is or comprises an Fc region.
112. The method of any one of the preceding embodiments, wherein a reaction partner is a compound of any one embodiments 1-98.
113. The method of any one of the preceding embodiments, wherein the contacting and forming steps are performed in one pot.
114. The method of any one of the preceding embodiments, wherein the contacting and forming steps are performed in one chemical reaction.
115. The method of any one of the preceding embodiments, wherein the method comprises no reactions which are directed primarily to cleavage of a functional group in an agent comprising a target agent moiety.
116. The method of any one of the preceding embodiments, wherein the method comprises no reactions which are directed primarily to cleavage of a functional group in L^{RM} or L^{P}M.
117. The method of any one of the preceding embodiments, wherein the method comprises no reactions which are directed primarily to reduction of a functional group in an agent comprising target agent moiety.
118. The method of any one of the preceding embodiments, wherein the method comprises no reactions which are directed primarily to reduction of a functional group in L^{RM} or L^{P}M.
119. The method of any one of the preceding embodiments, wherein the method comprises no reactions which are directed primarily to oxidation of a functional group in an agent comprising a target agent moiety.
120. The method of any one of the preceding embodiments, wherein the method comprises no reactions which are directed primarily to oxidation of a functional group in L^{RM} or L^{P}M.
121. The method of any one of the preceding embodiments, wherein the method comprises no reactions which are directed primarily to hydrolysis of a functional group in an agent comprising a target agent moiety.
122. The method of any one of the preceding embodiments, wherein the method comprises no reactions which are directed primarily to hydrolysis of a functional group in L^{RM} or L^{P}M.
123. The method of any one of the preceding embodiments, wherein the method comprises no reactions which are directed primarily to hydrolysis of an ester group in L^{RM} or L^{P}M.
124. The method of any one of embodiments 164-172, wherein a target agent moiety is a protein agent moiety.
125. The method of any one of embodiments 164-172, wherein a target agent moiety is an antibody agent moiety.
126. The method of any one of the proceeding embodiments, wherein contacting is performed under conditions and for a time sufficient for a lysine residue of a target agent to react with a reactive group of a reaction partner.
127. The method of any one of the proceeding embodiments, wherein contacting is performed under conditions and for a time sufficient for a lysine residue of a target agent to react and form a bond with an atom of RG and release LG.
128. The method of any one of the preceding embodiments, wherein the agent and the reaction partner share the same moiety of interest.
129. The method of any one of the preceding embodiments, wherein moiety of interest is or comprises an antibody agent.
130. The method of any one of the preceding embodiments, wherein moiety of interest is or comprises a reactive moiety.
131. The method of any one of the preceding embodiments, comprising reacting a first agent comprising a first reactive moiety in a first moiety of interest with a second agent comprising a second reactive moiety.
132. The method of any one of the preceding embodiments, wherein a second agent comprises a second reactive moiety and a peptide moiety.
133. The method of any one of the preceding embodiments, wherein a second agent comprises a second reactive moiety and a protein moiety.
134. The method of any one of the preceding embodiments, wherein a second agent comprises a second reactive moiety and an antibody agent moiety.
135. The method of any one of the preceding embodiments, comprising reacting a first agent comprising a first reactive moiety in a first moiety of interest with a second agent comprising a second reactive moiety in a second moiety of interest.
136. The method of any one of embodiments 132-135, wherein the first agent is a product of a method of any one of embodiments 99-131.
137. The method of any one of embodiments 132-135, wherein the second agent is a product of a method of any one of embodiments 99-131.
138. The method of any one of embodiments 132-135, wherein each of the first and the second agent is independently a product of a method of any one of embodiments 99-131.
139. A method, comprising reacting a first agent comprising a first reactive moiety in a first moiety of interest with a second agent comprising a second reactive moiety in a second moiety of interest, wherein the first agent is prepared by a method of any one of embodiments 99-131.
140. A method, comprising reacting a first agent comprising a first reactive moiety in a first moiety of interest with a second agent comprising a second reactive moiety in a second moiety of interest, wherein the second agents is prepared by a method of any one of embodiments 99-131.
141. A method, comprising reacting a first agent comprising a first reactive moiety in a first moiety of interest with a second agent comprising a second reactive moiety in a second moiety of interest, wherein each of the first and the second agents is independently prepared by a method of any one of embodiments 99-131.
142. The method of any one of embodiments 132-141, wherein each of the first and the second agents independently has the structure of formula P—I or P-II, or a salt thereof.
143. The method of any one of embodiments 132-142, wherein the target agent moiety of the first agent is an antibody agent moiety.
144. The method of any one of embodiments 132-143, wherein the target agent moiety of the second agent is an antibody agent moiety.
145. The method of any one of embodiments 143-144, wherein the first and the second target moieties are independently antibody agent moieties toward different antigens.
146. The method of any one of embodiments 143-144, wherein the first and the second target moieties are independently antibody agent moieties toward different proteins.
147. The method of any one of embodiments 132-146, wherein the first agent comprises an anti CD30 antibody, such as brentuximab, or an anit-nectin-4-monoclonal antibody, such as enfortumab.
148. The method of any one of embodiments 132-147, wherein the second agent comprises an anti-CD3 agent moiety.
149. The method of any one of embodiments 132-147, wherein the second agent comprises scFv.
150. The method of any one of embodiments 184-202, wherein the second agent comprises cetuximab.
151. The product of embodiment 224, wherein the product is or comprise an agent of formula P—I or P-II, or a salt thereof.
152. The product of embodiment 224, wherein the product is a composition comprising an agent of formula P—I or P-II, or a salt thereof.
153. The product of any one of embodiments 151-152, wherein the agent does not contain —S-Cy-, wherein -Cy- is optionally substituted 5-membered monocyclic ring, does not contain —S—S— which is not formed by cysteine residues and does not contain —SH or salt form thereof that is not of a cysteine residue.
154. The product of any one of embodiments 151-153, wherein the product is a pharmaceutical composition.
155. A composition provides a plurality of agents each of which independently comprising: a target agent moiety, a moiety of interest that is or comprises MMAD, MMAE, or MMAF, and optionally a linker moiety linking a target agent moiety and a moiety of interest; wherein agents of the plurality share the same or substantially the same target agent moiety, and a common modification independently at at least one common location; and wherein about 1%-100% of all agents that comprise a target agent moiety and a moiety of interest are agents of the plurality.
156. A composition provides a plurality of agents each of which independently comprising: a protein agent moiety, a moiety of interest that is or comprises MMAD, MMAE, or MMAF, and optionally a linker moiety linking a protein agent moiety and a moiety of interest; wherein protein agent moieties of agents of the plurality comprise a common amino acid sequence, and agents of the plurality share a common modification independently at at least one common amino acid residue of protein agent moieties; and wherein about 1%-100% of all agents that comprise a protein agent moiety that comprise the common amino acid sequence and a moiety of interest are agents of the plurality.
157. A composition provides a plurality of agents each of which independently comprising:
an antibody agent moiety,
a moiety of interest, e.g. that is or comprises MMAD, MMAE, or MMAF, and
optionally a linker moiety linking an antibody agent moiety and a moiety of interest; wherein antibody agent moieties of agents of the plurality comprise a common amino acid sequence or can bind to a common antigen, and agents of the plurality share a common modification independently at at least one common amino acid residue of protein agent moieties; and
wherein about 1%-100% of all agents that comprise an antibody agent moiety that comprise the common amino acid sequence or can bind to the common antigen and a moiety of interest are agents of the plurality.
158. The composition of embodiment 157, wherein antibody agent moieties of agents of the plurality can bind to a common antigen.
159. The composition of embodiment 157, wherein antibody agent moieties of agents of the plurality can bind to two or more different antigens.
160. The composition of any one of embodiments 157-159, wherein antibody agent moieties of agents of the plurality comprise a common amino acid sequence.
161. The composition of any one of embodiments 157-159, wherein antibody agent moieties of agents of the plurality comprise a common amino acid sequence in a Fc region.
162. The composition of any one of embodiments 157-159, wherein antibody agent moieties of agents of the plurality comprise a common Fc region.
163. The composition of any one of the preceding embodiments, wherein a target, protein or antibody agent moiety is or comprises an anti-CD30 or anti-nectin-4 agent moiety.
164. The composition of any one of the preceding embodiments, wherein a target, protein or antibody agent moiety is or comprises bentuximab or enfortumab.
165. The composition of any one of embodiments 159-162, wherein antibody agent moieties of agents of the plurality are IVIG moieties.
166. The composition of any one of the preceding embodiments, wherein agents of the plurality comprises a common moiety of interest.
167. The composition of any one of embodiments 155-166, wherein each agent of the plurality is independently an agent of formula P—I or P-II, or a salt thereof.
168. The composition of any one of embodiments 155-167, wherein a moiety of interest is or comprises a detectable moiety.
169. The composition of any one of embodiments 155-167, wherein a moiety of interest is or comprises a reactive moiety.
170. The composition of any one of embodiments 155-167, wherein a moiety of interest is or comprises a reactive moiety which does not react with a target agent moiety, a protein agent moiety or an antibody moiety agent.
171. The composition of any one of embodiments 155-167, wherein a moiety of interest is or comprises a reactive moiety which does not react with an antibody moiety agent.
172. The composition of any one of embodiments 155-167, wherein a moiety of interest is or comprises a therapeutic agent moiety, such as a cytotoxic moiety, such as MMAD, MMAE, or MMAF.
173. The composition of any one of embodiments 155-172, wherein the linker is not a natural amino acid peptide linker.
174. The composition of any one of embodiments 155-173, wherein a linker comprises one or more —CH₂—CH₂—O—.
175. The composition or embodiment 174, wherein a linker is or comprises —(CH₂CH₂O)n- where n is independently selected at each occurrence from integers 2, 3, 4, 5, 6, 7, and 8.
176. The composition or embodiment 174, wherein a linker is or comprises —(CH₂CH₂O)n-(CH₂)n-NHC(O)—(CH₂)n-, —[(CH₂CH₂O)n-(CH₂)n-NHC(O)]m-(CH₂)n-, and —(CH₂CH₂O)n-(CH₂)n-N((CH₂CH₂O)n-(CH₂)n-)((CH₂CH₂O)n-(CH₂)n-) where m is independently selected at each occurrence from integers 1, 2, 3, and 4.
177. The composition of any one of embodiments 156-176 wherein the common amino acid sequence of the protein agent moiety, comprises one or more amino acid residues selected from K246 and K248 of an IgG1 heavy chain and amino acid residues corresponding thereto, K251 and K253 of an IgG2 heavy chain and amino acid residues corresponding thereto, and K239 and K241 of an IgG4 heavy chain and amino acid residues corresponding thereto.
178. The composition of any one of embodiments 156-177, wherein the common amino acid sequence is at least 10%-100% of that of the protein or antibody agent moiety.
179. The composition of any one of embodiments 156-177, wherein the common amino acid sequence is at least 50%-100% of that of the protein or antibody agent moiety.
180. The composition of any one of embodiments 156-177, wherein the protein agent moieties or the antibody agent moieties of agents of the plurality are of at least 50% amino acid sequence homology.
181. The composition of any one of embodiments 156-177, wherein the protein agent moieties or the antibody agent moieties of agents of the plurality are of at least 80% amino acid sequence homology.
182. The composition of any one of embodiments 156-177, wherein the protein agent moieties or the antibody agent moieties of agents of the plurality are of at least 90% amino acid sequence homology.
183. The composition of any one of the preceding embodiments, wherein a common modification is or comprises a moiety of interest and optionally a linker.
184. The composition of any one of the preceding embodiments, wherein all common modifications comprises a common moiety of interest and optionally a common linker.
185. The composition of any one of embodiments 156-184, wherein a common amino acid residue is K246 of an antibody heavy chain or an amino acid residue corresponding thereto.
186. The composition of any one of embodiments 156-185, wherein a common amino acid residue is K248 of an antibody heavy chain or an amino acid residue corresponding thereto.
187. The composition of any one of embodiments 156-186, wherein a common amino acid residue is K288 of an antibody heavy chain or an amino acid residue corresponding thereto.
188. The composition of any one of embodiments 156-187, wherein a common amino acid residue is K290 of an antibody heavy chain or an amino acid residue corresponding thereto.
189. The composition of any one of embodiments 156-188, wherein a common amino acid residue is K317 of an antibody heavy chain or an amino acid residue corresponding thereto.
190. The composition of any one of embodiments 156-189, wherein a common amino acid residue is K133 of an antibody heavy chain or an amino acid residue corresponding thereto.
191. The composition of any one of embodiments 156-190, wherein a common amino acid residue is K144 of an antibody heavy chain or an amino acid residue corresponding thereto.
192. The composition of any one of embodiments 156-191, wherein a common amino acid residue is K133 of an antibody heavy chain or an amino acid residue corresponding thereto.
193. The composition of any one of embodiments 156-192, wherein a common amino acid residue is K185 of an antibody light chain or an amino acid residue corresponding thereto.
194. The composition of any one of embodiments 156-193, wherein a common amino acid residue is K187 of an antibody light chain or an amino acid residue corresponding thereto.
195. The composition of any one of embodiments 156-194, wherein a common amino acid residue is K251 of an IgG2 antibody heavy chain or an amino acid residue corresponding thereto.
196. The composition of any one of embodiments 156-195, wherein a common amino acid residue is K253 of an IgG2 antibody heavy chain or an amino acid residue corresponding thereto.
197. The composition of any one of embodiments 156-196, wherein a common amino acid residue is K239 of an IgG4 antibody heavy chain or an amino acid residue corresponding thereto.
198. The composition of any one of embodiments 156-197, wherein a common amino acid residue is K241 of an IgG4 antibody heavy chain or an amino acid residue corresponding thereto.
199. The composition of any one of the preceding embodiments, wherein at least about 2% of all agents that comprise a target agent moiety and a moiety of interest are agents of the plurality, or at least about 2% of all agents that comprise a protein agent moiety that comprise the common amino acid sequence and a moiety of interest are agents of the plurality, or at least about 2% of all agents that comprise an antibody agent moiety that comprise the common amino acid sequence or can bind to the common antigen and a moiety of interest are agents of the plurality.
200. The composition of any one of the preceding embodiments, wherein about 1%-100% of all agents that comprise a target agent moiety are agents of the plurality, or at least about 1%-100% of all agents that comprise a protein agent moiety that comprise the common amino acid sequence are agents of the plurality, or about 1%-100% of all agents that comprise an antibody agent moiety that comprise the common amino acid sequence or can bind to the common antigen are agents of the plurality.
201. The composition of any one of embodiments 199-200, wherein the percentage is at least about 5%.
202. The composition of any one of embodiments 199-200, wherein the percentage is at least about 10%.
203. The composition of any one of embodiments 199-200, wherein the percentage is at least about 20%.
204. The composition of any one of embodiments 199-200, wherein the percentage is at least about 25%.
205. The composition of any one of embodiments 199-200, wherein the percentage is at least about 50%.
206. The composition of any one of embodiments 199-200, wherein the percentage is at least about 60%, 65%, 70%, 75%, 80%, 85%, 90% or 95%.
207. The composition of any one of the preceding embodiments, wherein each agent of the plurality does not contain —S-Cy-, wherein -Cy- is optionally substituted 5-membered monocyclic ring, does not contain —S—S— which is not formed by cysteine residues and does not contain —SH or salt form thereof that is not of a cysteine residue.
208. The composition of any one of the preceding embodiments, wherein each agent of the plurality does not contain —S—CH₂—CH₂—.
209. The composition of any one the preceding embodiments, wherein each agent of the plurality does not contain a moiety that can specifically bind to an antibody agent.
210. The composition of any one the preceding embodiments, wherein each agent of the plurality independently comprises an antibody agent moiety, and each agent can independently bind to an Fc receptor.
211. The composition of any one of the preceding embodiments, wherein the composition is a product of a method of any one of the preceding embodiments.
212. The composition of any one of the preceding embodiments, wherein the composition is a pharmaceutical composition.
213. An agent, wherein the agent is an agent of the plurality of any one of embodiments 155-211.
214. A pharmaceutical composition, comprising an agent of embodiment 213 and a pharmaceutically acceptable carrier.
215. The composition of embodiment 212 or 214, wherein the composition is in a solid form.
216. The composition of embodiment 212 or 214, wherein the composition is in a liquid form, and contains no more than 5%, 10%, 15%, 20%, 25%, 30%, 35%, 40%, 45% or 50% (v/v) organic solvents.
217. The method, product, composition or agent of any one of the preceding embodiments, wherein the ratio of moieties of interest conjugated to target agent moieties and target agent moieties, or the ratio of moieties of interest conjugated to protein agent moieties and protein agent moieties, or the ratio of moieties of interest conjugated to antibody agent moieties and antibody agent moieties, is about 0.5-6.
218. The method, product, composition or agent of any embodiment 217, wherein the ratio is about 0.5-2.5.
219. The method, product, composition or agent of any embodiment 217, wherein the ratio is about 0.5, 0.6, 0.7, 0.8, 0.9, 1.0, 1.1, 1.2, 1.3, 1.4, 1.5, 1.6, 1.7, 1.8, 1.9, 2.0, 2.5 or 3.
220. The compound, method, product, composition or agent of any one of the preceding embodiments, wherein each heteroatom is independently selected from oxygen, nitrogen, sulfur, phosphorus and silicon.
221. An agent comprising an amino acid residue of a of any of the following compounds, or an amino acid residue of an ester of any of the following compounds:

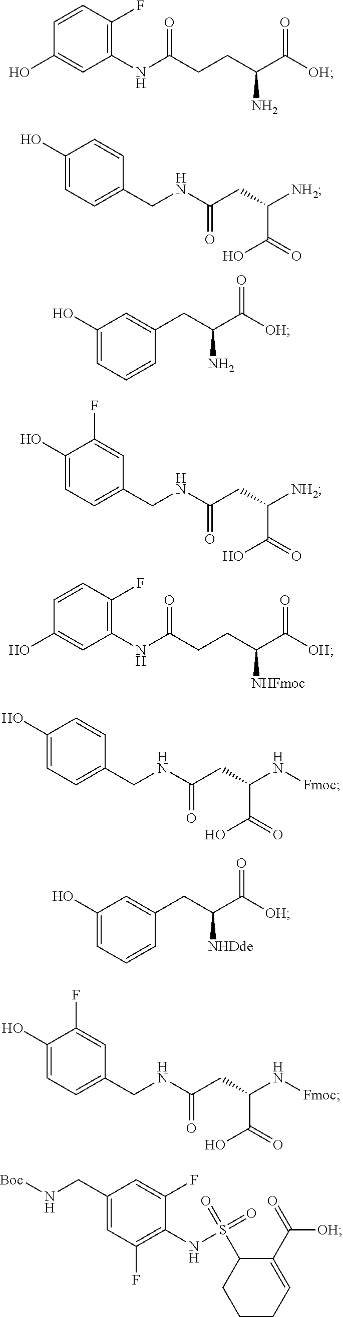

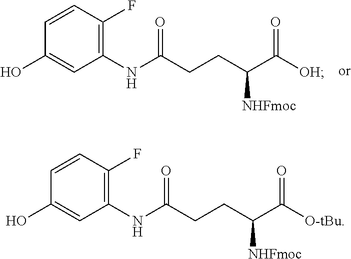

222. The agent of embodiment 221, wherein the agent has the structure of formula R—I or a salt thereof.
223. A polypeptide agent comprising an amino acid residue of a compound of any one of embodiments 221.
224. A method for preparing a compound, comprising providing a compound of any one of embodiments 221.
225. A compound having the structure of formula R-I:

LG-RG-L^{RM}-MOI (R-I)
or a salt thereof, wherein:
   LG is a group comprising a target binding moiety that binds to a target agent,
   RG is a reactive group;
   L^{RM} is a linker; and
   MOI is a moiety of interest comprising monomethyl auristatin E (MMAE).
226. A composition including:
   a first compound having the structure of formula (P-II):

P—N-L^{PM}-MOI (P-II)
wherein:
P—N is a protein agent moiety including a lysine residue;
L^{PM} is a linker; and
MOI is a moiety of interest comprising monomethyl auristatin E (MMAE); and
a second compound having the structure:

LG-OH (LG-I)
wherein LG is a group including a target binding moiety that binds to a target agent.
227. The composition of embodiment 226, further including:
   a third compound having the formula (R-I):

LG-RG-L^{RM}-MOI (R-I)
LG is a group including a target binding moiety that binds to a target agent, which is identical to LG in formula (LG-I);
RG is a reactive group;
L^{RM} is a linker, which is identical to in formula (P-II); and
MOI is a moiety of interest comprising monomethyl auristatin E (MMAE).
a fourth compound having the formula (R—III):

HO-RG-L^{RM}-MOI (R—III)
or a combination thereof.

### EXAMPLES

As depicted in the Examples below, in certain exemplary embodiments, compounds, agents, compositions, etc. are prepared and/or assessed according to the following procedures as examples. It will be appreciated that, although the general methods depict the synthesis of certain compounds, agents, compositions of the present disclosure, the following general methods, and other methods known to one of ordinary skill in the art, can be applied to in accordance with the present disclosure to provide technologies of the present disclosure.

### Abbreviations

The following abbreviations are used in the examples that follow.
DCM Dichloromethane
DIC N,N′-Diisopropylcarbodiimide
DIEA N,N-Diisopropylethylamine
DMAP 4-Dimethylaminopyridine
DMF Dimethylfuran
EDCI 1-Ethyl-3-(3-dimethylaminopropyl)carbodiimide
Fmoc Fluorenylmethyloxycarbonyl chloride
HATU Hexaflurophosphate Azabenzotriazole Tetramethyl Uronium
HBTU (1H-Benzotriazol-1-yloxy)(dimethylamino)-N,N-dimethylmethaniminium
hexafluorophosphate
HOBt Hydroxybenzotriazole
NMM N-Methylmorpholine
SPPS Solid Phase Peptide Synthesis
TEA Triethylamine
THF Tetrahydrofuran
TFA Trifluoro acetic acid

### Example 1. Synthesis of 3-Fluoro-4-hydroxybenzylamine-containing Reactive Group (Cmp. 4)

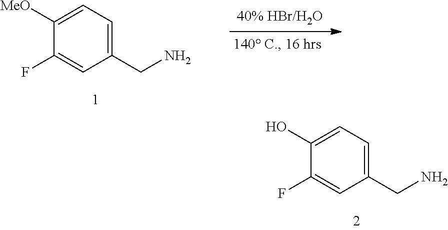 [SMILES: COc1ccc(CN)cc1F ; NCc1ccc(O)c(F)c1]

A mixture of intermediate 1 (10 g, 64.45 mmol) in HBr/H₂O (40% HBr, 300 mL in total) was stirred at 140° C. for 16 hrs. The solvent was removed at 70° C. under reduced pressure, the residue was triturated in MeCN (50 mL) for 10 mins. After filtered, the solid was dried under lyophilization to provide intermediate 2 (13.0 g, 58.5 mmol, 90.8% yield, HBr salt) as a brown solid. 1H NMR: (400 MHz DMSO-d₆) δ ppm 10.04 (s, 1H) 8.18 (s, 3H) 7.32 (dd, J=12.17, 1.88 Hz, 1H) 7.11 (dd, J=8.28, 1.51 Hz, 1H) 6.96-7.03 (m, 1H) 3.93 (q, J=5.52 Hz, 2H).

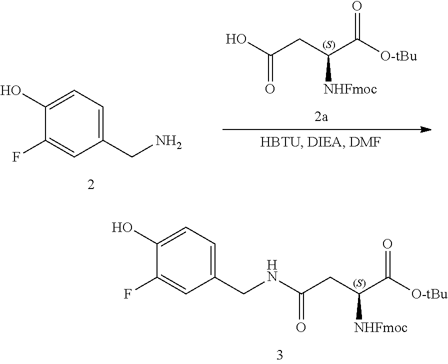

To a mixture of intermediate 2 (13.0 g, 58.5 mmol, 1 eq, HBr), intermediate 2a (24.1 g, 58.5 mmol, 1 eq), DIEA (3.78 g, 29.2 mmol, 5.10 mL, 0.5 eq) and HOBt (11.87 g, 87.8 mmol, 1.5 eq) in DMF (200 mL) was added EDCI (12.35 g, 64.4 mmol, 1.1 eq) at 15° C., the mixture was stirred at 15° C. for 3 hr. The mixture was dropwise added to 0.5 M HCl (cold, 1 L) and white solid was precipitated. After filtration, the solid was dried under lyophilization to afford intermediate 3 (31 g, crude) as a white solid.

Alternatively, the reaction can be conducted with 60.0 g of compound 2 starting material at 20° C. After precipitation with HCl and filtration the solid can be dissolved in DCM (2 L), washed with 0.5 M HCl (800 mL), H₂O (800 mL), brine (800 mL), dried over anhydrous Na₂SO₄ and concentrated under reduced pressure. The residue was purified by silica gel column (DCM/MeOH=from I/O to 20/1) to afford Intermediate compound 3 (120.0 g, 90% purity, containing a small amount of DMF, 83.3% yield) as a white solid. ¹H NMR (400 MHz, DMSO-d₆) δ ppm 9.70 (s, 1H) 8.34 (t, J=5.77 Hz, 1H) 7.90 (d, J=7.53 Hz, 2H) 7.71 (d, J=7.53 Hz, 2H) 7.61 (d, J=8.28 Hz, 1H) 7.39-7.47 (m, 2H) 7.29-7.36 (m, 2H) 7.02 (d, J=12.30 Hz, 1H) 6.85-6.92 (m, 2H) 4.20-4.39 (m, 4H) 4.11-4.19 (m, 2H) 1.36 (s, 9H).

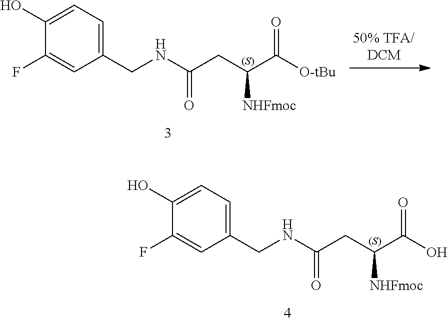

A mixture of compound 3 (30 g, 56.12 mmol, 1.0 eq) in TFA (300 mL) and DCM (300 mL) was stirred at 15° C. for 0.5 hr. The solvent was removed under reduced pressure. The residue was purified by flash C18 (ISCO®; 120 g SepaFlash® C18 Flash Column, Eluent of 0˜90% MeCN/H₂O gradient @75 mL/min) directly to get compound 4 (18 g, 37.6 mmol, 67.0% yield) as a white solid. ¹H NMR (400 MHz, DMSO-d₆) δ ppm 9.69 (s, 1H) 8.34 (t, J=5.90 Hz, 1H) 7.90 (d, J=7.28 Hz, 2H) 7.71 (d, J=7.53 Hz, 2H) 7.54 (d, J=6.53 Hz, 1H) 7.42 (t, J=7.40 Hz, 2H) 7.27-7.37 (m, 1H) 7.27-7.37 (m, 1H) 7.02 (d, J=12.05 Hz, 1H) 6.82-6.93 (m, 2H) 4.35-4.43 (m, 1H) 4.20-4.31 (m, 3H) 4.13-4.19 (m, 2H).

### Example 2. Procedure for Preparation of Antibody Binding Moiety Coupled to Reactive Group (Intermediate Cmp. 5a)

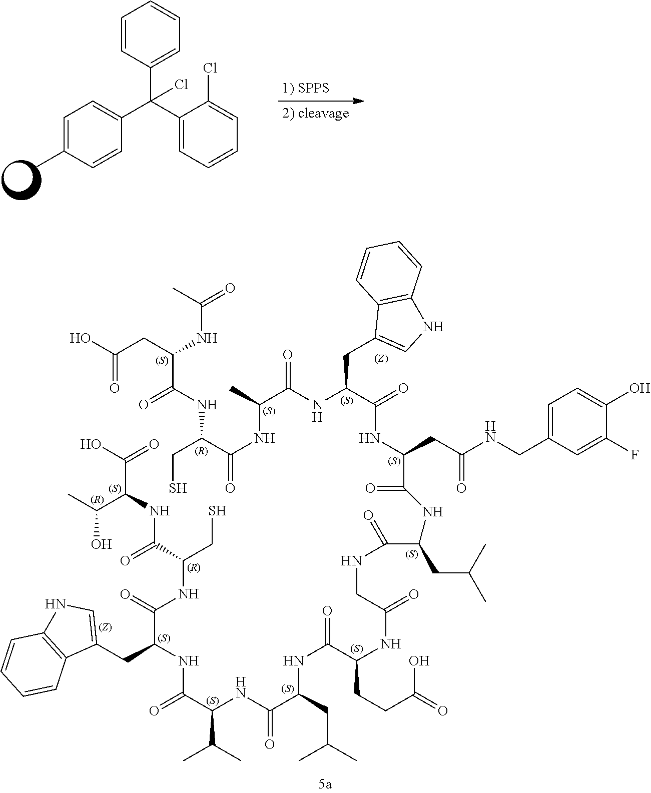

Peptide was synthesized using standard Fmoc chemistry.

1) Resin preparation: To the vessel containing CTC Resin (3.0 mmol, 3.0 g, 1.00 mmol/g) and Fmoc-Thr(tBu)-OH (1.19 g, 3.0 mmol, 1.00 eq) in DCM (30 mL) was added DIEA (4.00 eq) dropwise and mixed for 2 hrs with N₂ bubbling at 15° C. Then MeOH (3.0 mL) was added and bubbled with N₂ for another 30 mins. The resin was washed with DMF (60 mL), followed by adding 20% piperidine in DMF (60 mL) and bubbled with N₂ for 30 mins at 15° C. for Fmoc deprotection. The mixture was filtered and the resin was washed with DMF (60 mL) before proceeding to next step. Alternatively this reaction can be conducted at 20° C.

2) Coupling: A solution of Fmo-C≡Cys(Trt)-OH (5.25 g, 3.00 eq), HBTU (3.24 g, 2.85 eq) in DMF (30 mL) was added to the resin with N₂ bubbling. Then DIEA (6.00 eq) was added to the mixture dropwise and bubbled with N₂ for 30 mins at 15° C. (or 20° C.). The coupling reaction was monitored by ninhydrin test, if it showed colorless, the coupling was completed. The resin was then washed with DMF (60 mL).

3) Deprotection: 20% piperidine in DMF (60 mL) was added to the resin and the mixture was bubbled with N₂ for 30 mins at 15° C. The deprotection reaction was monitored by ninhydrin test, if it showed blue or other brownish red, the reaction was completed. The resin was then washed with DMF (60 mL).

4) Step 2 and 3 were repeated for amino acids: (3-13 in table below).

5) Coupling for compound: A solution of compound 4 (2.87 g, 2.00 eq), DIC (0.76 g, 2.00 eq) and HOBt (0.82 g, 2.00 eq) in DMF (30 mL) was added to the resin with N₂ bubbling for 60 mins at 15° C. The coupling reaction was monitored by ninhydrin test, if it showed colorless, the coupling was completed. The resin was then washed with DMF (60 mL).

6) Repeat step 3 for Fmoc deprotection.

7) Step 5 and 6 were repeated for amino acids (10-13 in table below).

8) Acetylation: A solution of 10% Ac₂O/5% NMM/85% DMF (60 mL) was added to resin and the mixture was bubbled with N₂ for 20 mins. The acetylation reaction was monitored by ninhydrin test, if it showed colorless, the coupling was completed. The resin was then washed with DMF (60 mL) to afford intermediate 5a

9) 3% hydrazine in DMF (60 mL) was added to resin with N₂ bubbling for 15 min to liberate hydroxyl group on compound 4, and resin was then washed with DMF (60 mL).

TABLE 1
List of amino acids and the corresponding reagents used on SPPS

| # | Materials | Coupling reagents |
|---|---|---|
| 1 | Fmoc-Thr(tBu)-OH (1.00 eq) | DIEA (4.00 eq) |
| 2 | Fmoc-Cys(Trt)-OH (3.00 eq) | HBTU (2.85 eq) and DIEA (6.00 eq) |
| 3 | Fmoc-Trp-OH (3.00 eq) | HBTU (2.85 eq) and DIEA (6.00 eq) |
| 4 | Fmoc-Val-OH (3.00 eq) | HBTU (2.85 eq) and DIEA (6.00 eq) |
| 5 | Fmoc-Leu-OH (3.00 eq) | HBTU (2.85 eq) and DIEA (6.00 eq) |
| 6 | Fmoc-Glu(OtBu)-OH (3.00 eq) | HBTU (2.85 eq) and DIEA (6.00 eq) |
| 7 | Fmoc-Gly-OH (3.00 eq) | HBTU (2.85 eq) and DIEA (6.00 eq) |
| 8 | Fmoc-Leu-OH (3.00 eq) | HBTU (2.85 eq) and DIEA (6.00 eq) |
| 9 | Intermediate compound 4 (2.00 eq) | DIC (2.00 eq) and HOBt (2.00 eq) |
| 10 | Fmoc-Trp-OH (3.00 eq) | DIC (3.00 eq) and HOBt (3.00 eq) |
| 11 | Fmoc-Ala-OH (3.00 eq) | DIC (3.00 eq) and HOBt (3.00 eq) |
| 12 | Fmoc-Cys(Trt)-OH (3.00 eq) | DIC (3.00 eq) and HOBt (3.00 eq) |
| 13 | Fmoc-Asp(OtBu)-OH (3.00 eq) | DIC (3.00 eq) and HOBt (3.00 eq) |
| 14 | Acetylation | AC₂O/NMM/DMF (10/5/85, 60 mL) |
| 15 | 3-(2-(prop-2-yn-1- | DIC (3.00 eq), HOBt (3.00 eq) and |
| | yloxy)ethoxy)propanoic acid (3.00 eq) | DMAP (3.00 eq) |

### Peptide Cleavage and Purification:

1) Cleavage buffer (95% TFA/2.5% TIS/2.5% H₂O, 60.0 mL) was added to the flask containing the side chain protected peptide resin at room temperature and stirred for 1 hr.

2) The filtrate was collected.

3) The peptide was precipitated with cold isopropyl ether (300 mL) and centrifuged (3 mins at 3000 rpm).

4) Isopropyl ether washed two additional times, and the crude peptide was dried under vacuum for 2 hrs.

5) Compound 5a (4.2 g, crude) was obtained as a white solid.

### Example 3. Coupling Cmp. 5a to an Ethoxy Containing Linker

Either following step 9, above, while the peptide is still coupled to its solid phase support or after peptide cleavage and purification a 3-(2-(prop-2-yn-1-yloxy)ethoxy)ethyl linker can be coupled to 5. A solution of 3-(2-(prop-2-yn-1-yloxy)ethoxy)propanoic acid (1.54 g, 9.0 mmol, 3.00 eq), DIC (1.13 g, 3.00 eq), HOBt (1.23 g, 3.00 eq) and DMAP (1.10 g, 3.00 eq) was added to resin and the mixture was bubbled with N₂ for 36 hrs. The coupling reaction was monitored by LCMS after a mini-cleavage, almost 50% was desired MS. The resin was then washed with DMF (60 mL), MeOH (60 mL), and then dried under vacuum.

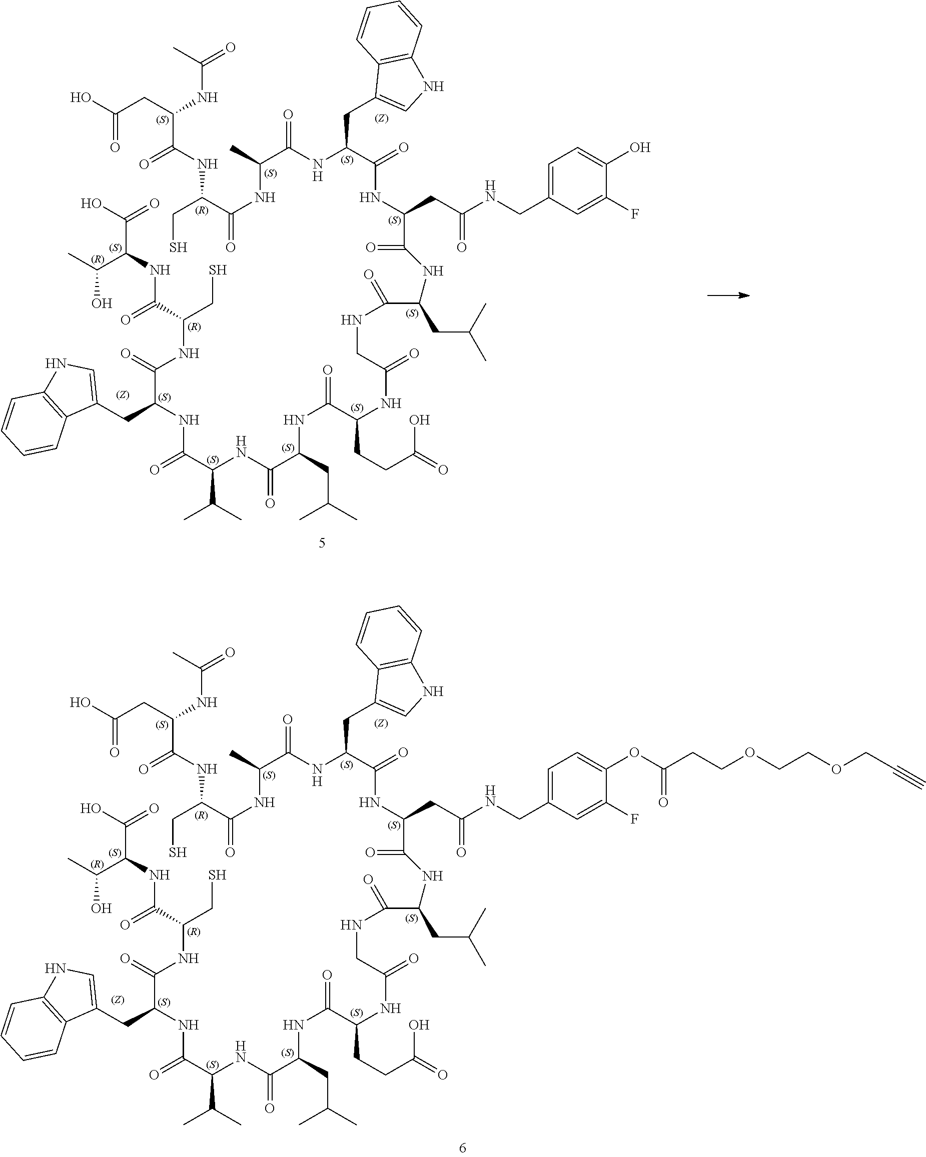

### Example 4. Procedure for Preparation of MMAE1/Compound 1100

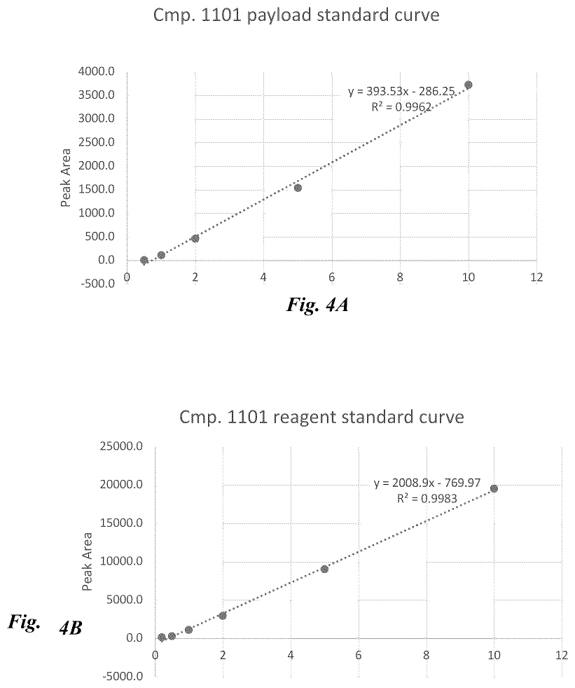

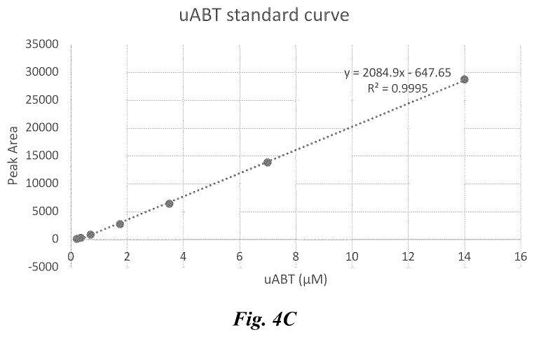

The complete reaction scheme for the preparation of compound 1100 is shown in FIG. 4

### A. Preparation of Intermediate 5

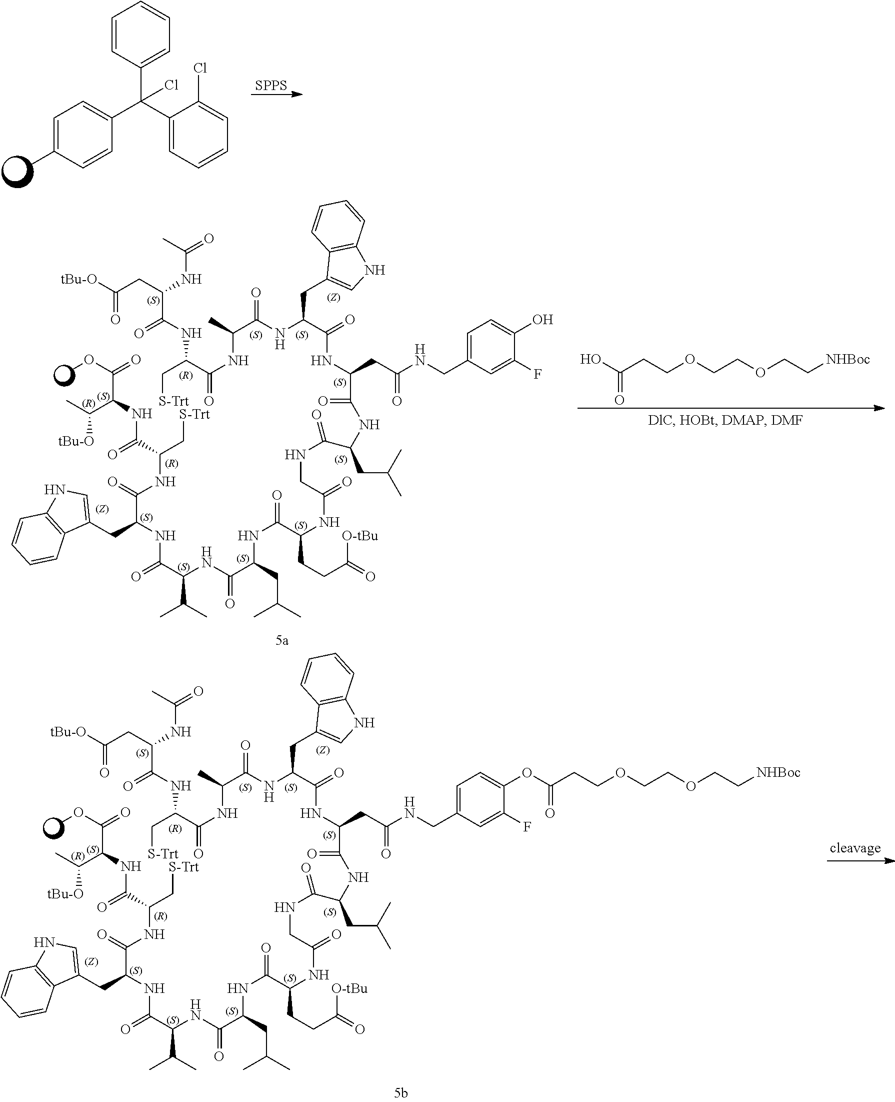

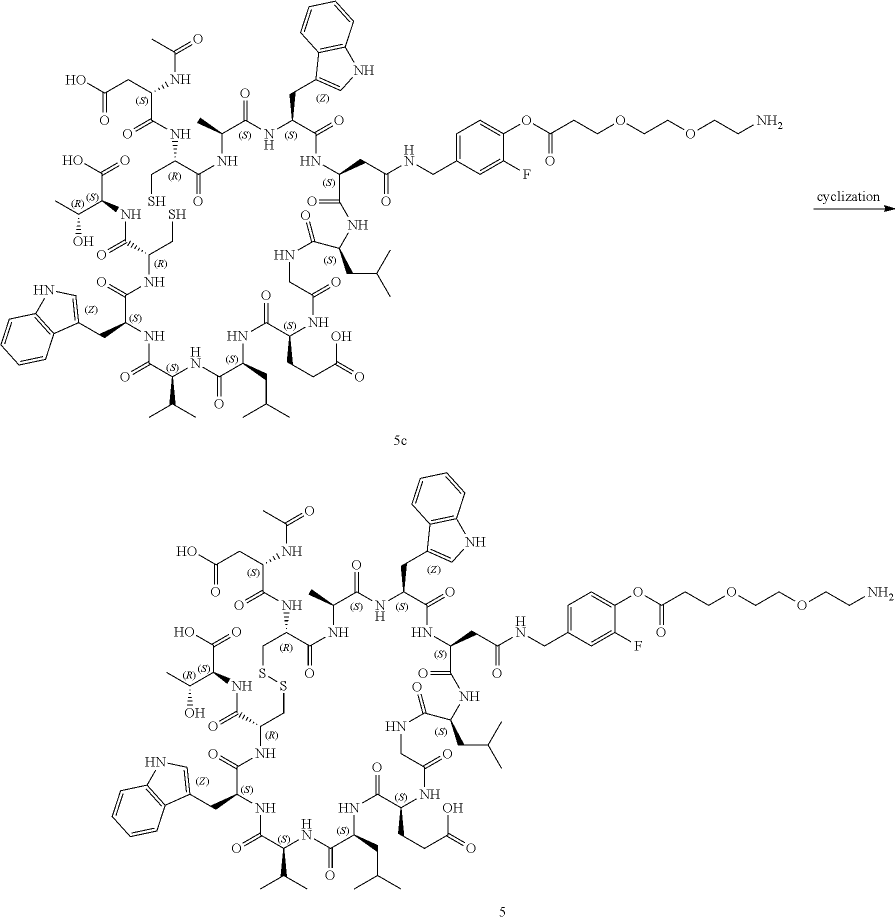

Intermediate compound 5a is prepared using the Resin preparation (1), Coupling (2), and Deprotection steps (3) from Example 1.

Steps 2 and 3 were repeated for the following amino acids elongation: Number #3-13, Table 1.

4) Acetylation: A solution of Ac₂O/NMM/DMF (10/5/85, v/v/v, 2 L) was added to resin and the mixture was bubbled with N₂ for 20 min. The coupling reaction was monitored by ninhydrin test, it showed colorless, indicating that the coupling was completed. The resin was then washed with DMF (2 L) to afford Intermediate 5a.

5) 3% hydrazine in DMF (2 L) was added to resin with N₂ bubbling for 15 min to liberate hydroxyl group on Intermediate 5a, the resin was then washed with DMF (2 L).

6) Coupling with phenol: A solution of Boc-NH-PEG₂-CH₂CH₂COOH (55.4 g, 200.0 mmol, 2.00 equiv.), DIC (25.2 g, 200.0 mmol, 2.00 equiv.), HOBt (27.0 g, 200.0 mmol, 2.00 equiv.) and DMAP (12.2 g, 200.0 mmol, 2.00 equiv.) was added to resin and the mixture was bubbled with N₂ for 36 h. The coupling reaction was monitored by LCMS after a mini-cleavage, almost 70% was desired MS. The resin was then washed with DMF (2 L), MeOH (2 L), and dried under reduced pressure to afford Intermediate 5b (CTC resin, 100.0 mmol).

### Peptide Cleavage and Cyclization:

1) Cleavage: Add cleavage solution (TFA/Tis/H₂O, 95/2.5/2.5, v/v/v, 2 L) to the flask containing the side chain protected peptide at room temperature and stirred for 1 h. After filtration, the filtrate was precipitated with isopropyl ether (cold, 10 L). After filtration, the solid was washed with isopropyl ether (cold, 1 L) for two additional times, and dried under reduced pressure for 2 h to afford Intermediate 5c (140.5 g, crude) as a white solid.

2) Cyclization: To a mixture of the crude peptide (Intermediate 5c) in HOAc/MeCN/H₂O (4/3/3, v/v/v, 80 L) was added 0.1 M I₂/AcOH dropwise until a yellow color persisted, then the mixture was stirred at 20° C. for 5 min. The mixture was quenched by addition of 0.1 M aq. Na₂S₂O₃ dropwise until the yellow color disappeared. After filtration, the filtrate was purified by prep-HPLC (A: 0.075% TFA/H₂O, B:MeCN), followed by lyophilization to afford intermediate 5 (22.0 g, 93.3% purity, 12.0% yield) as a white solid. LCMS: RT=0.907 min, MS calcd.: Mₐᵥ=1833.02, mass observed: [M+H]⁺=1833.81, [M+2H]²⁺=917.00.

B. Preparation of intermediate 7

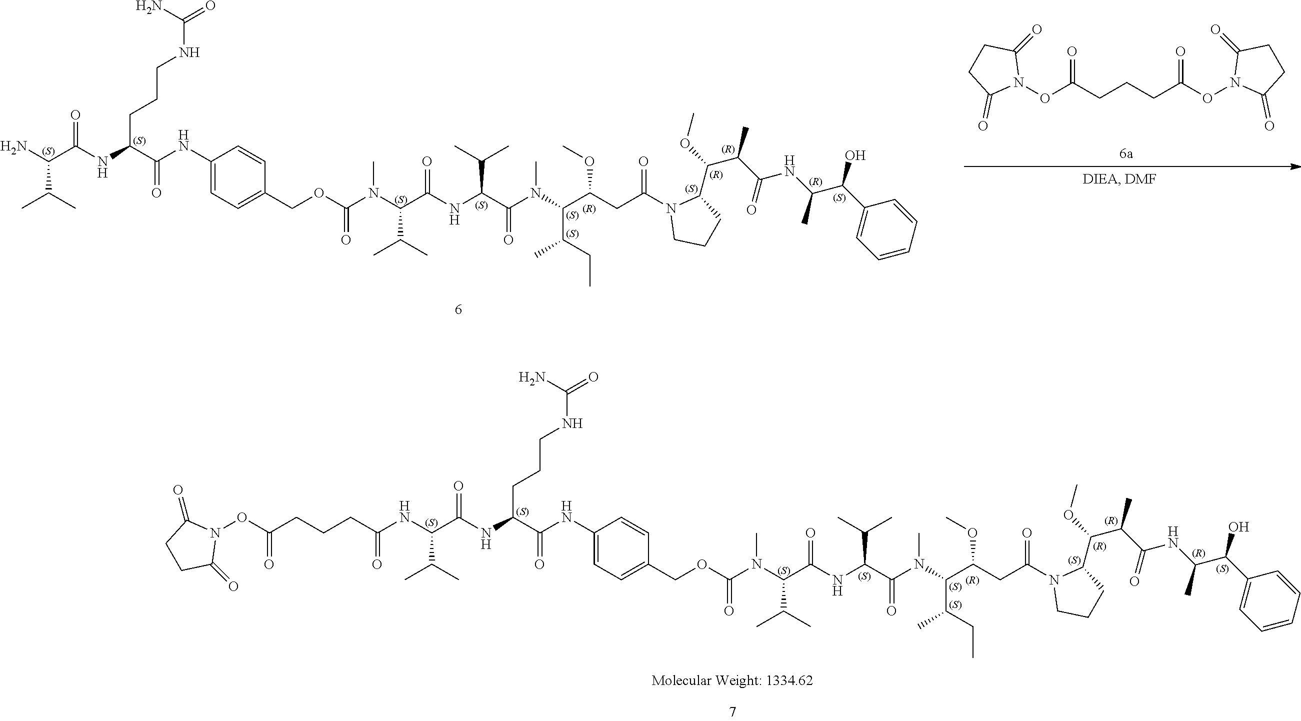

A mixture of Intermediate 6 (500.0 mg, 44.5 μmol, 1.00 equiv.), DIEA (11.5 mg, 89.0 μmol, 2.00 equiv.) in DMF (10.0 mL) was added dropwise to a solution of Compound 6a (700.0 mg, 2.15 mmol, 4.80 equiv.) in DMF (10.0 mL) at 0° C. dropwise. The mixture was stirred at 20° C. for 10 min. LCMS showed the starting material was consumed completely. The mixture was purified by prep-HPLC (TFA condition) directly to afford Compound 7 (490.0 mg, 36.8 μmol, 82.6% yield) as a white solid. LCMS: RT=0.942 min, MS cal.: Mₐᵥ=1334.6, [M+2H]²⁺=668.3.

### C. Preparation of Compound 1100

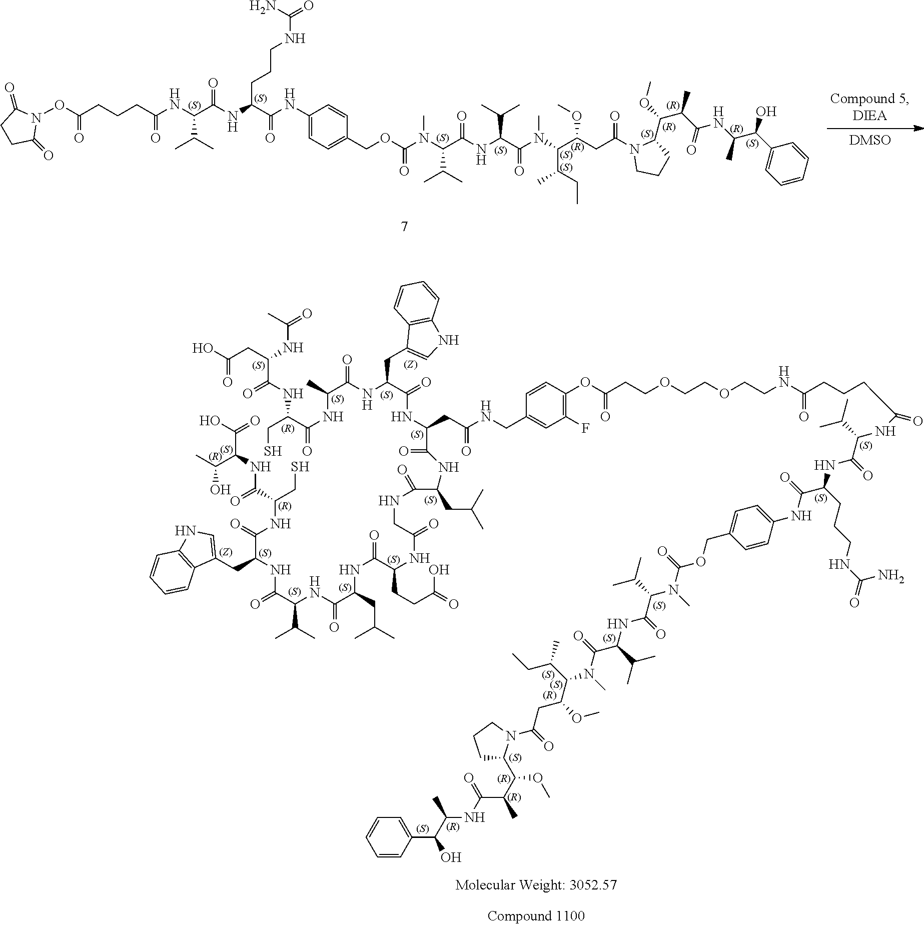

To a solution of Intermediate 5 (14.42 mg, 7.87 umol, 1.05 equiv.), Intermediate 7 (10 mg, 7.49 umol, 1.00 equiv.) in DMSO (500 μL) was added DIEA (4.84 mg, 37.46 μmol, 6.53 uL, 5.00 equiv.). Then the mixture was stirred at 20° C. for 1 h. LCMS showed Intermediate 7 was consumed completely and one main peak was desired m/z. The reaction was filtered, and the filtrate was purified by prep-HPLC (TFA condition) directly, followed by lyophilization to afford compound 1100 (9.7 mg, 3.18 μmol, 42.4% yield) as a white solid. LCMS: RT=1.16 min, MS cal.: Mₐᵥ=3052.53, [M+2H]²⁺=1526.5.

### Example 5. Procedure for Preparation of Compound 1101

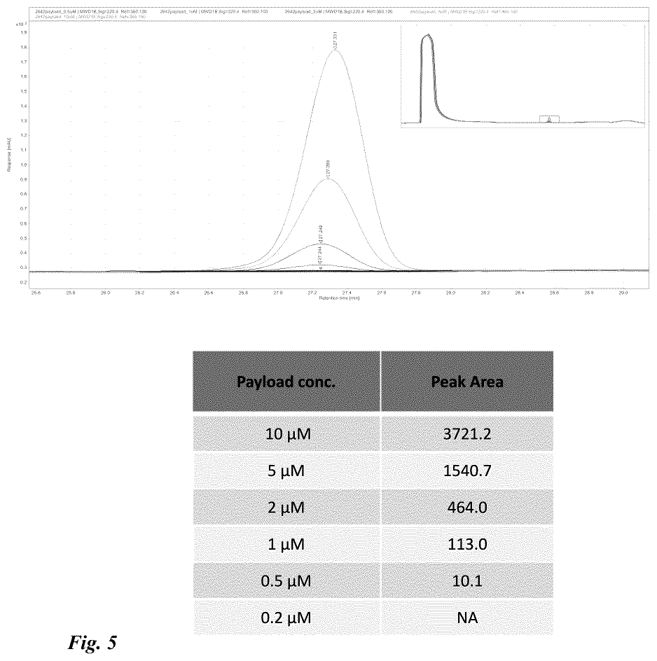

The complete reaction scheme for the preparation of compound 1101 is shown in FIG. 5.

### A. Preparation of Intermediate 9

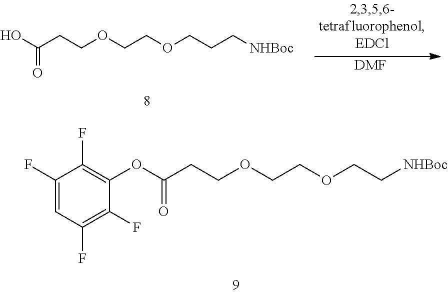

To a solution of Intermediate 8 (1.56 g, 5.64 mmol, 1.00 equiv.) in DMF (5 mL) was added 2,3,4,6-tetrafluorophenol (2.81 g, 16.93 mmol, 3.00 equiv.), EDCI (1.62 g, 8.47 mmol, 1.50 equiv.). The reaction was stirred at 20° C. for 16 h. LCMS indicated Intermediate 8 was consumed completely and one main peak was desired MS. The mixture was purified by prep-Flash (C18, TFA condition) to afford Intermediate 9 (2.00 g, 4.70 mmol, 95.9% purity, 83.3% yield) as yellow oil. LCMS: RT=1.30 min, MS cal.: Mₐᵥ=425.37, [M+Na]⁺=447.99.

### B. Preparation of Intermediate 10

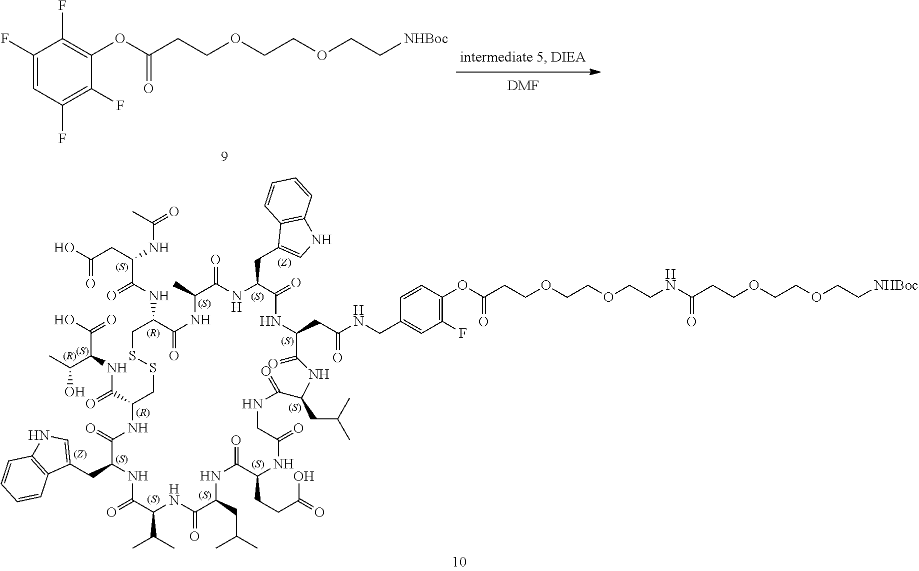

Intermediate 5 (300.0 mg, 163.66 μmol, 1.00 equiv.), Intermediate 9 (90.5 mg, 212.76 μmol, 1.30 equiv.), DIEA (105.7 mg, 818.32 μmol, 142.54 μL, 5.00 equiv.) in DMSO (9 mL) was stirred at 15° C. for 1 h. LCMS indicated the main peak was desired MS. The mixture was purified by flash C18 (ISCO®; 120 g SepaFlash® C18 Flash Column, Eluent of 0-90% MeCN/H₂O ether gradient @75 mL/min) directly to afford Intermediate 10 (200.0 mg, 95.59 μmol, 58.40% yield) as a white solid. LCMS: RT=1.05 min, MS cal.: Mₐᵥ=2092.32, [M+2H]²⁺=1046.60.

### C. Preparation of Intermediate 11

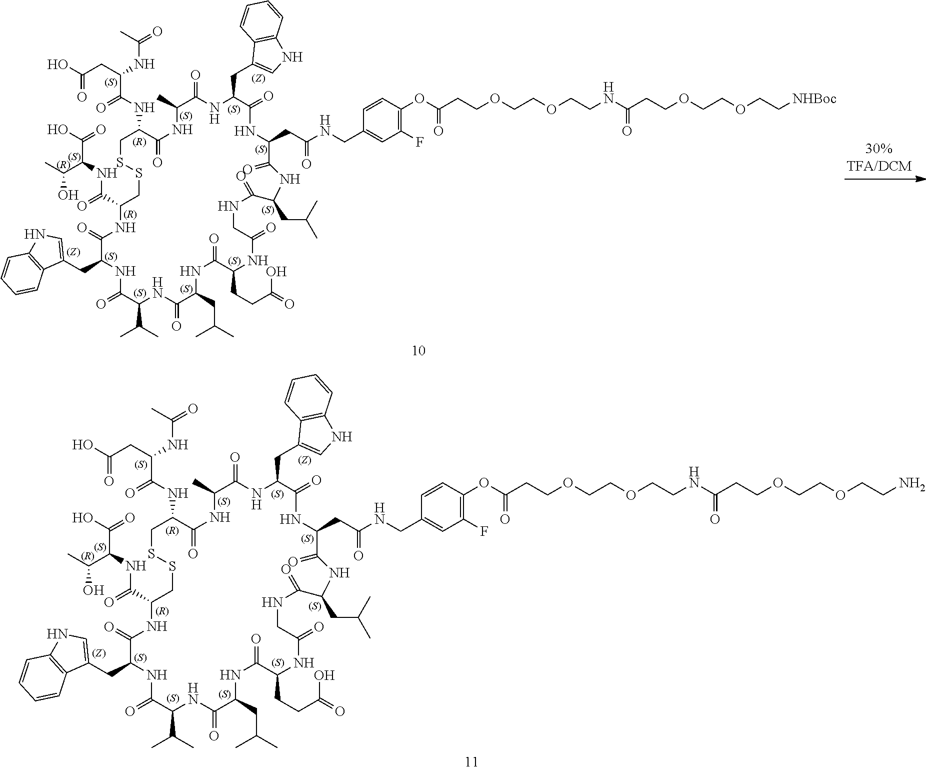

A mixture of Intermediate 10 (200.0 mg, 95.59 umol, 1.00 equiv.) in TFA/DCM (3/7, 4 mL) was stirred at 0° C. for 0.5 h. LCMS indicated Intermediate 10 was consumed completely and the main peak was desired MS. The mixture was purified by prep-HPLC (TFA condition) directly, followed by lyophilized to afford Intermediate 11 (120.0 mg, 60.23 μmol, 63.0% yield) as a white solid. LCMS: RT=0.95 min, MS cal.: Mₐᵥ=1992.20, [M+2H]²⁺=996.60.

### D. Preparation of Compound 1101

The Compound 1101 structure is split in two parts. The dashed lines indicate a covalent bond that is shared between the upper and lower part of the 1101 structure.

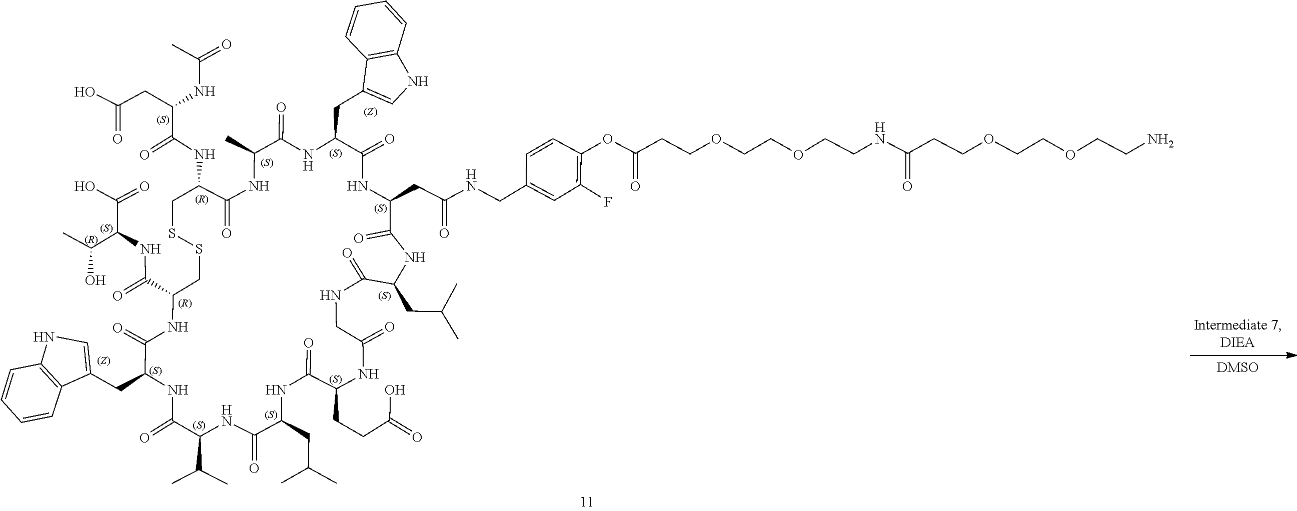

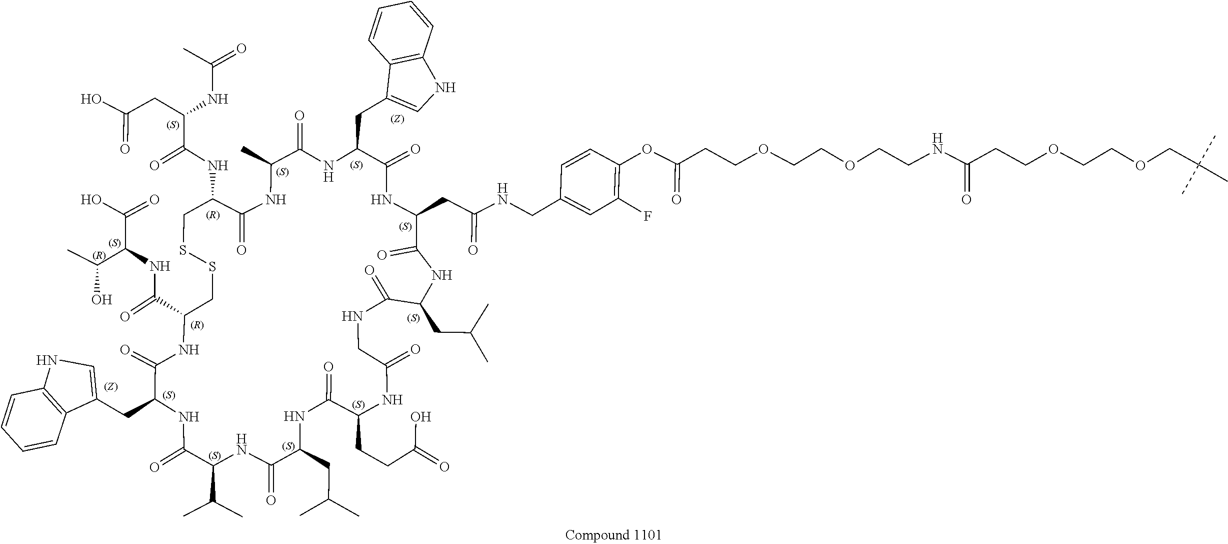

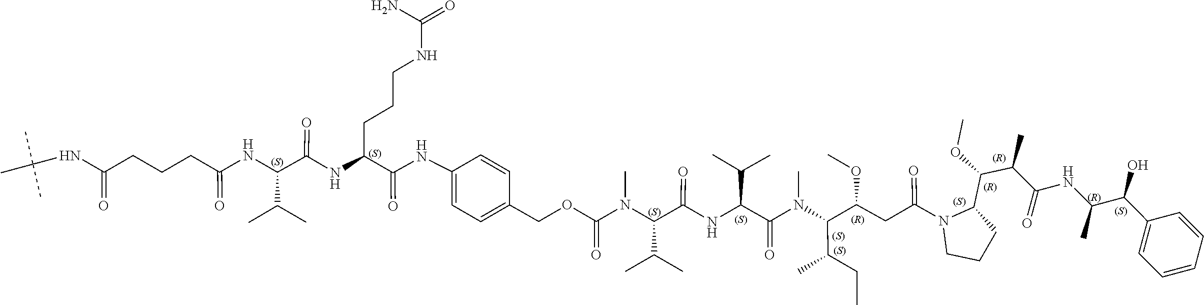

To a mixture of Intermediate 11 (29.85 mg, 14.99 μmol, 1.00 equiv.), DIEA (9.68 mg, 74.93 μmol, 13.05 μL, 5.00 equiv.) in DMSO (1.0 mL) was added Intermediate 7 (20.0 mg, 14.99 μmol, 1.00 equiv.) at 20° C. Then the mixture was stirred at 20° C. for 1 h. LCMS indicated Intermediate 7 was consumed completely and one main peak was desired MS. The mixture was purified by prep-HPLC (TFA condition) to afford compound 1101 (22.8 mg, 98.6% purity, 47.3% yield) as a white solid. LCMS: RT=1.10 min, MS cal.: Mₐᵥ=3211.71, [M+2H]²⁺=1606.50.

### Example 6. Procedure for Preparation of Compound 1102

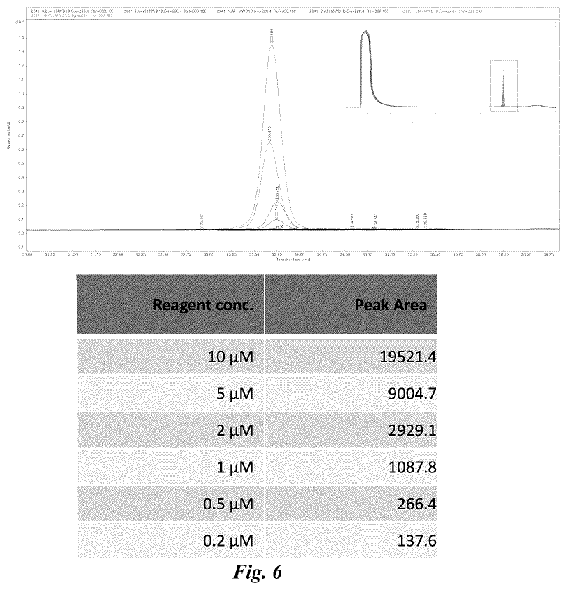

The complete reaction scheme for the preparation of compound 1102 is shown in FIG. 6.

### Preparation of Intermediate 13

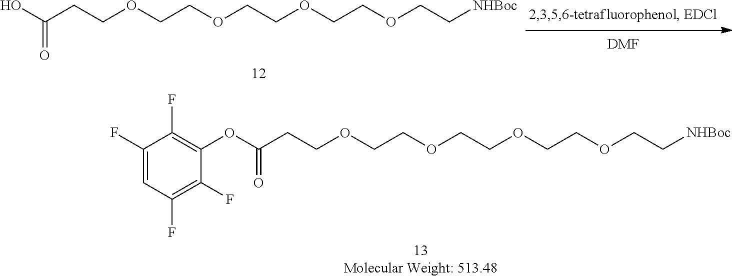

To a solution of Intermediate 12 (0.50 g, 1.37 mmol, 1.00 equiv.) in DMF (3 mL) was added 2,3,4,6-tetrafluorophenol (568.09 mg, 3.42 mmol, 2.50 equiv.), EDCI (393.46 mg, 2.05 mmol, 1.50 equiv.). The reaction was stirred at 20° C. for 16 h. LCMS indicated Intermediate 12 was consumed completely and one main peak was desired MS. The mixture was purified by prep-Flash (C18, TFA condition) to afford Intermediate 13 (0.625 g, 1.22 mmol, 88.96% yield) as yellow oil. LCMS: RT=1.16 min, MS cal.: Mₐᵥ=513.48, [M+Na]⁺=536.1, [M+H]⁺=514.1.

### Preparation of Intermediate 14:

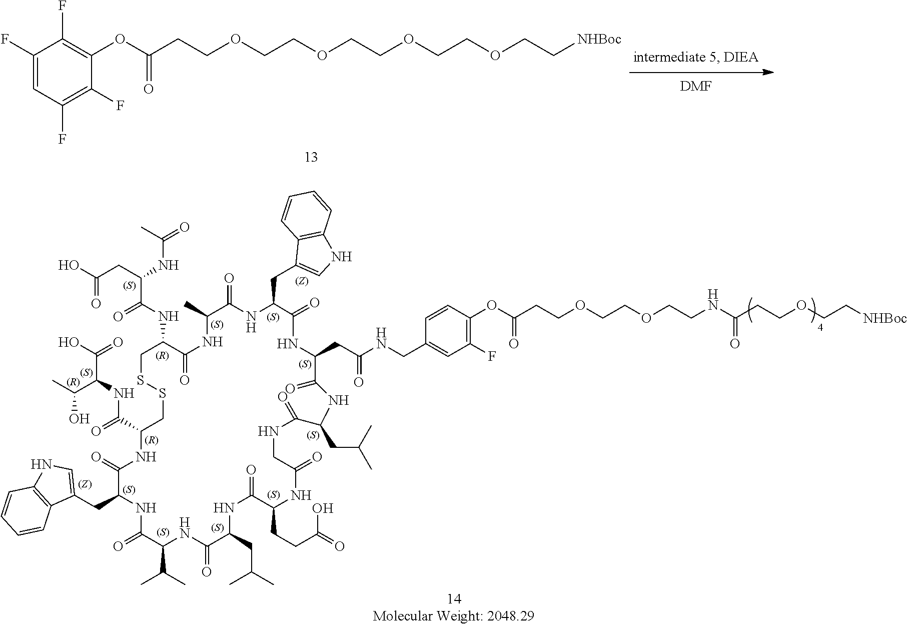

Intermediate 5 (300.0 mg, 163.66 μmol, 1.00 equiv.), Intermediate 13 (9.25 mg, 212.76 μmol, 1.30 equiv.), DIEA (105.76 mg, 818.32 μmol, 142.54 μL, 5.00 equiv.) in DMSO (9 mL) was stirred at 20° C. for 1 h. LCMS indicated the main peak was desired MS. The mixture was purified by flash C18 (ISCO®; 120 g SepaFlash® C18 Flash Column, Eluent of 0-90% MeCN/H₂O ether gradient @75 mL/min) directly to afford Intermediate 14 (200.0 mg, 86.22 μmol, 52.6% yield, 94.0% purity) as a white solid. LCMS: RT=1.013 min, MS cal.: Mₐᵥ=2180.42, [M−Boc+2H]²⁺=1090.60, [M−Boc+2H]²⁺=1040.50.

### Preparation of Intermediate 15:

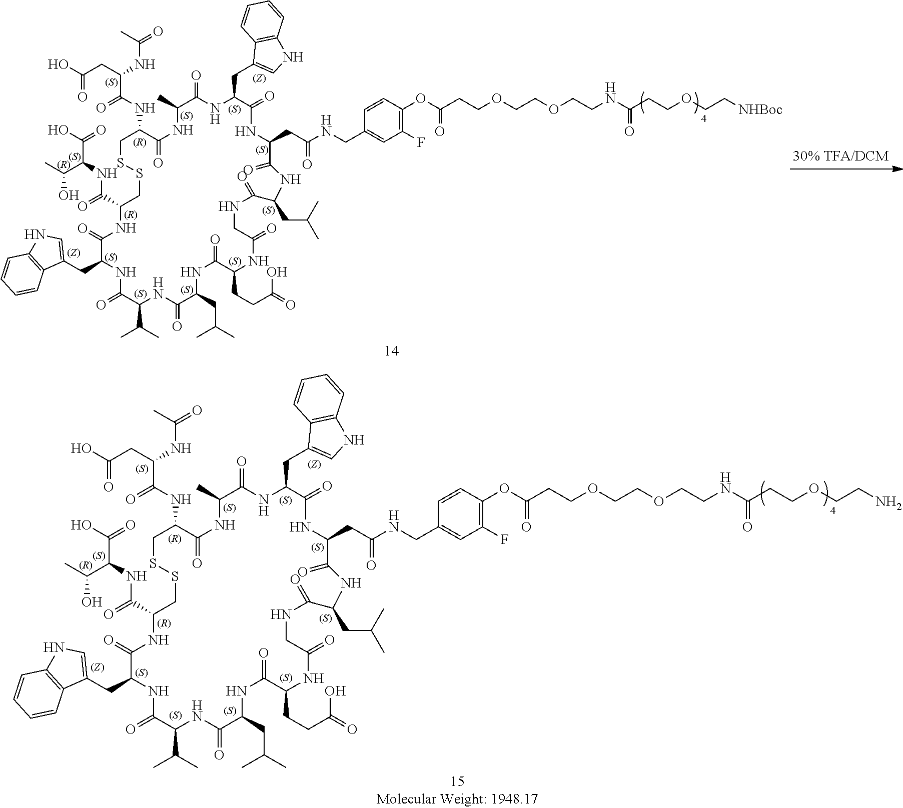

A mixture of Intermediate 14 (200.0 mg, 95.59 μmol, 1.00 equiv.) in TFA/DCM (3/7, 4 mL) was stirred at 0° C. for 0.5 h. LCMS indicated Intermediate 14 was consumed completely and the main peak was desired MS. The mixture was purified by prep-HPLC (TFA condition) directly, followed by lyophilized to afford Intermediate 15 (150.0 mg, 68.36 μmol, 74.5% yield, TFA salt) as a white solid. LCMS: RT=0.95 min, MS cal.: Mₐᵥ=2080.31, [M+2H]²⁺=1040.59.

### Preparation of Compound 1102:

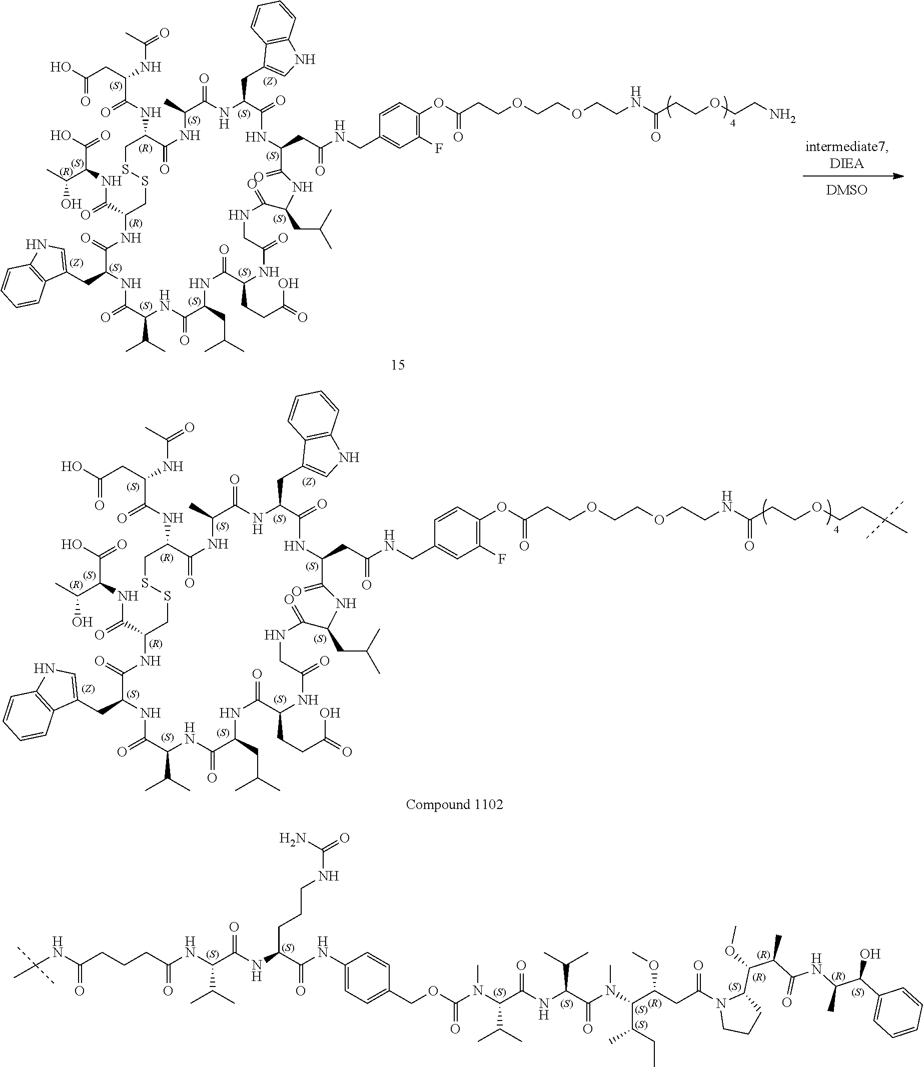

To a mixture of Intermediate 15 (29.85 mg, 14.99 μmol, 1.00 equiv.), DIEA (9.68 mg, 74.93 μmol, 13.05 μL, 5.00 equiv.) in DMSO (1.0 mL) was added Intermediate 7 (20.0 mg, 14.99 μmol, 1.00 equiv.) at 20° C. Then the mixture was stirred at 20° C. for 1 h. LCMS indicated Intermediate 7 was consumed completely and one main peak was desired MS. The mixture was purified by prep-HPLC (TFA condition) to afford Compound 1102 (30.5 mg, 98.6% purity, 63.2% yield) as a white solid. LCMS: RT=1.10 min, MS cal.: Mₐᵥ=3299.82, [M+2H]²⁺=1650.41, [M+3H]³⁺=1100.50. The dashed lines in Compound 1102's structure indicate a single covalent between the top and bottom structures.

### Example 7. Procedure for Preparation of Compound 1103

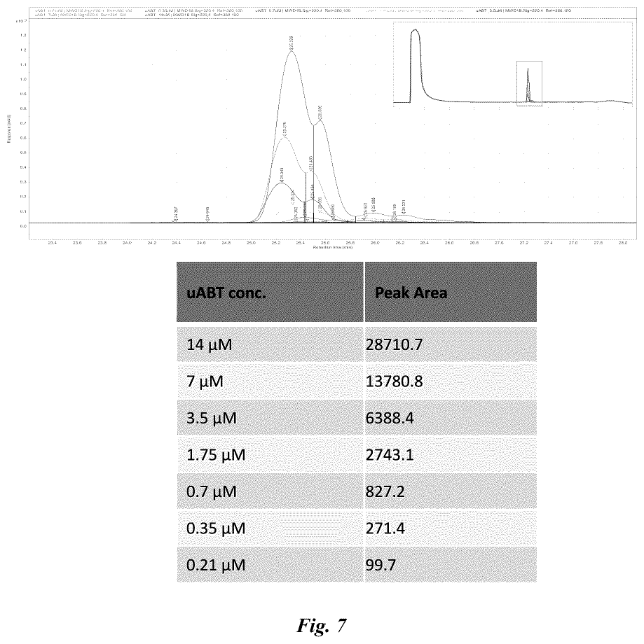

The complete reaction scheme for the preparation of compound 1103 is shown in FIG. 7.

### Preparation of Intermediate 17:

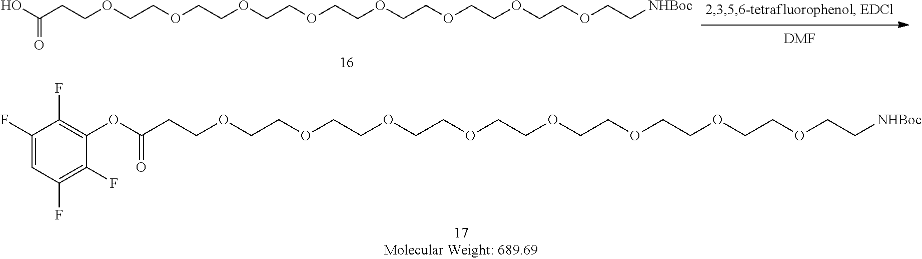

To a solution of Intermediate 16 (1.00 g, 1.85 mmol, 1.00 equiv.) in DMF (3 mL) was added 2,3,4,6-tetrafluorophenol (919.86 mg, 5.54 mmol, 3.00 equiv.), EDCI (530.91 mg, 2.77 mmol, 1.50 equiv.). The reaction was stirred at 20° C. for 16 h. LCMS indicated Intermediate 16 was consumed completely and one main peak was desired MS. The mixture was purified by prep-Flash (C18, TFA condition) to afford Intermediate 17 (1.10 g, 1.59 mmol, 86.4% yield) as colorless oil. LCMS: RT=1.152 min, MS cal.: Mₐᵥ=689.69, [M+H]⁺=690.1, [M+Na]⁺=707.2. Preparation of intermediate 18:

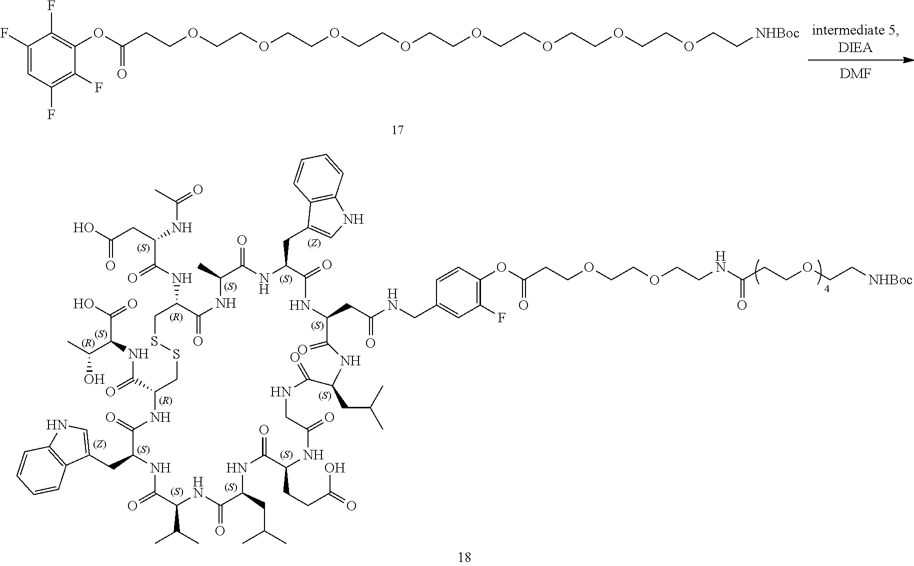

Intermediate 5 (300.0 mg, 163.66 μmol, 1.00 equiv.), Intermediate 17 (112.8 mg, 163.66 μmol, 1.10 equiv.), DIEA (105.76 mg, 818.32 μmol, 142.54 μL, 5.00 equiv.) in DMSO (9 mL) was stirred at 20° C. for 1 h. LCMS indicated the main peak was desired MS. The mixture was purified by flash C18 (ISCO®; 120 g SepaFlash® C18 Flash Column, Eluent of 0-90% MeCN/H₂O ether gradient @75 mL/min) directly to afford Intermediate 18 (200.0 mg, 84.87 μmol, 51.8% yield) as a white solid. LCMS: RT=1.05 min, MS cal.: Mₐᵥ=2356.63, [M+2H]²⁺=1179.10, [M−Boc+2H]²⁺=1129.20.

### Preparation of Intermediate 19:

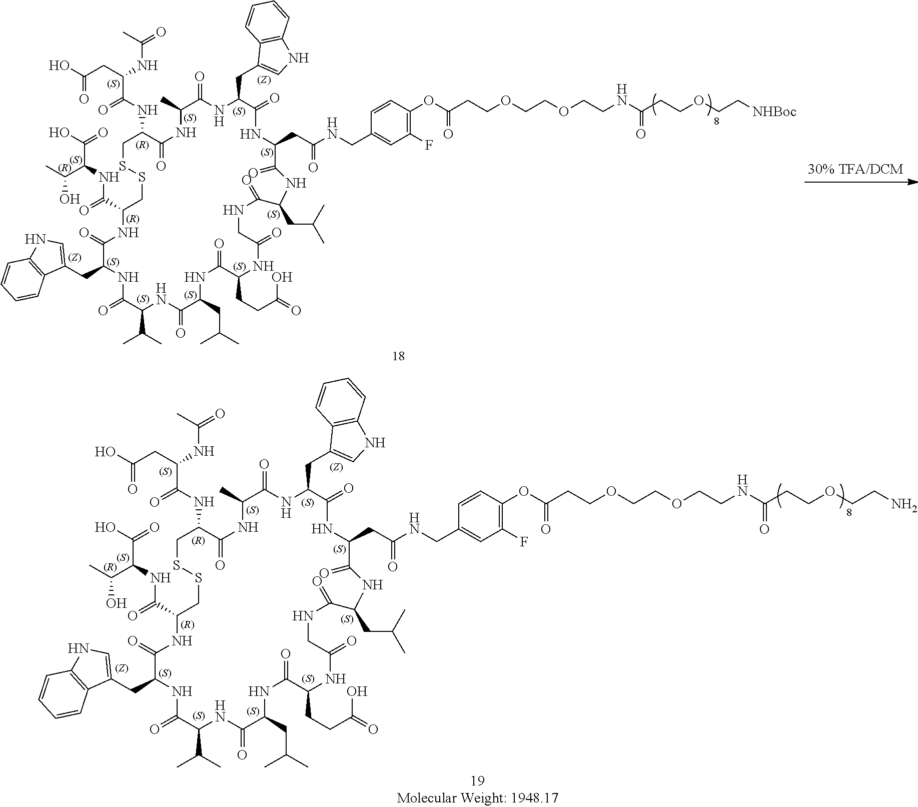

A mixture of Intermediate 18 (200.0 mg, 84.87 μmol, 1.00 equiv.) in TFA/DCM (3/7, 4 mL) was stirred at 0° C. for 0.5 h. LCMS indicated Intermediate 18 was consumed completely and the main peak was desired MS. The mixture was purified by prep-HPLC (TFA condition) directly, followed by lyophilized to afford Intermediate 19 (160.0 mg, 67.50 μmol, 79.5% yield, TFA salt) as a white solid. LCMS: RT=0.95 min, MS cal.: Mₐᵥ=2256.52, [M+2H]²⁺=1128.79, [M+3H]³⁺=752.89.

### Preparation of Compound 1103:

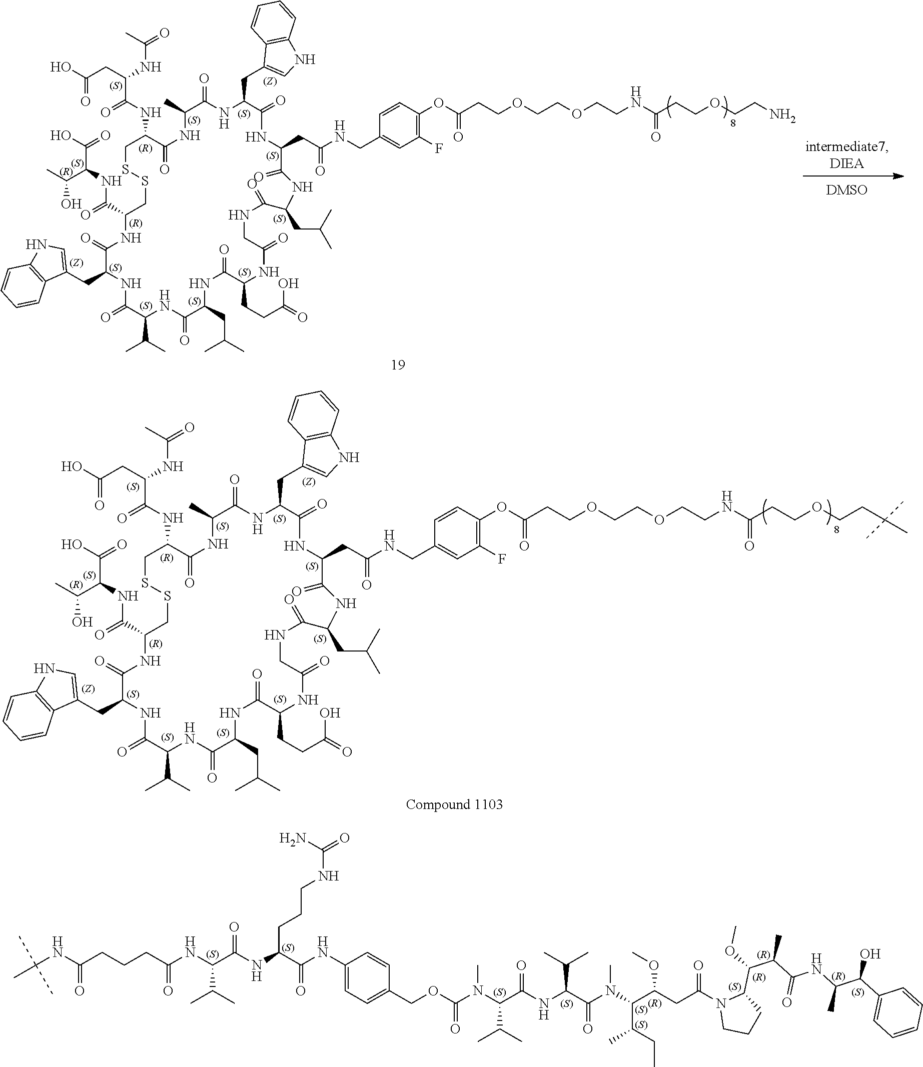

To a mixture of Intermediate 19 (37.20 mg, 16.48 μmol, 1.10 equiv.), DIEA (9.68 mg, 74.93 μmol, 13.05 μL, 5.00 equiv.) in DMSO (1.0 mL) was added Intermediate 7 (20.0 mg, 14.99 μmol, 1.00 equiv.) at 20° C. Then the mixture was stirred at 20° C. for 1 h. LCMS indicated Intermediate 7 was consumed completely and one main peak was desired MS. The mixture was purified by prep-HPLC (TFA condition) to afford Compound 1103 (31.4 mg, 9.03 μmol, 60.2% yield) as a white solid. LCMS: RT=1.09 min, MS cal.: Mₐᵥ=3476.03, [M+2H]²⁺=1738.51, [M+3H]³⁺=1159.23. The dashed lines in Compound 1103's structure indicate a single covalent between the top and bottom structures.

### Example 8. Procedure for Preparation of Compound 1104

The complete reaction scheme for the preparation of compound 1104 is shown in FIG. 8.

### Preparation of Intermediate 21

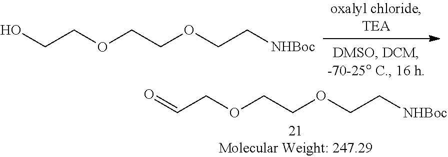 [SMILES: CC(C)(C)OC(=O)NCCOCCOCCO ; CC(C)(C)OC(=O)NCCOCCOCC=O]

To a solution of DMSO (7.84 g, 100 mmol, 2.50 equiv.) in DCM (150 mL) was added to a solution of oxalyl chloride (10.20 g, 80.2 mmol, 2.00 equiv.) in DCM (50 mL), then the reaction mixture was stirred at −70° C. for 10 min, then a solution of Intermediate 20 (10.0 g, 40.1 mmol, 1.00 equiv.) in DCM (50 mL) was added dropwise. After stirred at −70° C. for 50 min, TEA (32.5 g, 320 mmol, 8.00 equiv.) was added and the reaction mixture was allowed to warm to 20° C. and stirred for 15 h. TLC (Dichloromethane:Methanol=10:1, R_{f}=0.48) showed the reactant was consumed completely and one new spot formed. The reaction mixture was quenched with H₂O (50 mL), the aqueous phase was extracted with DCM (300 mL). The combined organic layers were washed with brine (300 mL), dried over Na₂SO₄, filtered and concentrated under reduced pressure to give a residue. The residue was purified by silica gel column (DCM/EtOAc from 95/5 to 50/50) to afford Intermediate 21 (5.10 g, 20.6 mmol, 51.4% yield) as yellow oil. 1H NMR (400 MHz, CHLOROFORM-d) δ ppm 9.71-9.77 (m, 1H) 3.39-3.90 (m, 8H) 3.25-3.36 (m, 2H) 1.40-1.52 (m, 9H).

### Preparation of Intermediate 22:

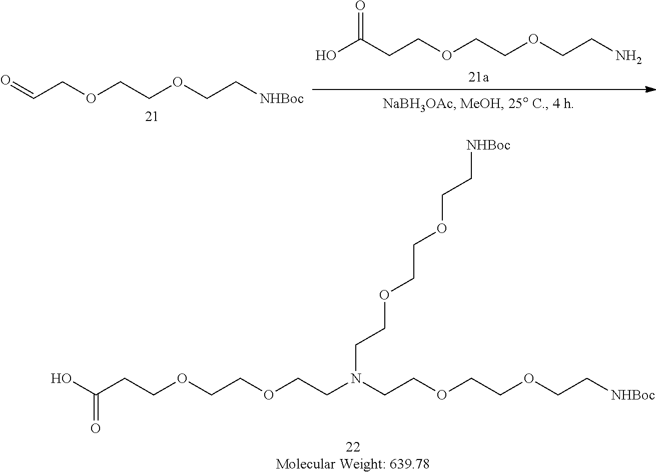 [SMILES: NCCOCCOCCC(=O)O ; CC(C)(C)OC(=O)NCCOCCOCC=O ; CC(C)(C)OC(=O)NCCOCCOCCN(CCOCCOCCNC(=O)OC(C)(C)C)CCOCCOCCC(=O)O]

To a solution of Intermediate 21 (5.02 g, 20.3 mmol, 1.00 equiv.), Intermediate 21a (1.20 g, 6.77 mmol, 0.40 equiv.) in MeOH (40 mL) was added NaBH_{3OAc (}2.15 g, 10.2 mmol, 0.60 equiv.) slowly at 20° C. After addition, the reaction mixture was stirred at 20° C. for 4 h. LCMS found desired MS. The reaction mixture was quenched with H₂O (10 mL) dropwise. After filtration, the filtrate was concentrated. The residue was purified by prep-HPLC (column: Welch Ultimate XB—C18 250*50 mm, 10 μm, 120 A+Welch Ultimate 250*50 mm, 10 μm, 120 A; mobile phase: [water (0.1% TFA)-ACN]; B %: 24%-54%, 24 min) to afford Intermediate 22 (0.80 g, 1.25 μmol, 18.5% yield) as yellow oil. LCMS: RT=0.833 min, MS cal.: Mₐᵥ=639.78, [M+H]⁺=640.5. ¹H NMR (400 MHz, DEUTERIUM OXIDE) 6 ppm 7.87 (s, 1H) 7.37 (br d, J=7.53 Hz, 1H) 3.79-3.91 (m, 4H) 3.70-3.79 (m, 4H) 3.60-3.70 (m, 12H) 3.45-3.58 (m, 8H) 2.99-3.40 (m, 6H) 2.59 (t, J=5.52 Hz, 2H) 1.36 (s, 18H).

### Preparation of Intermediate 23 (Sidechain Protected Resin-Bound Peptide):

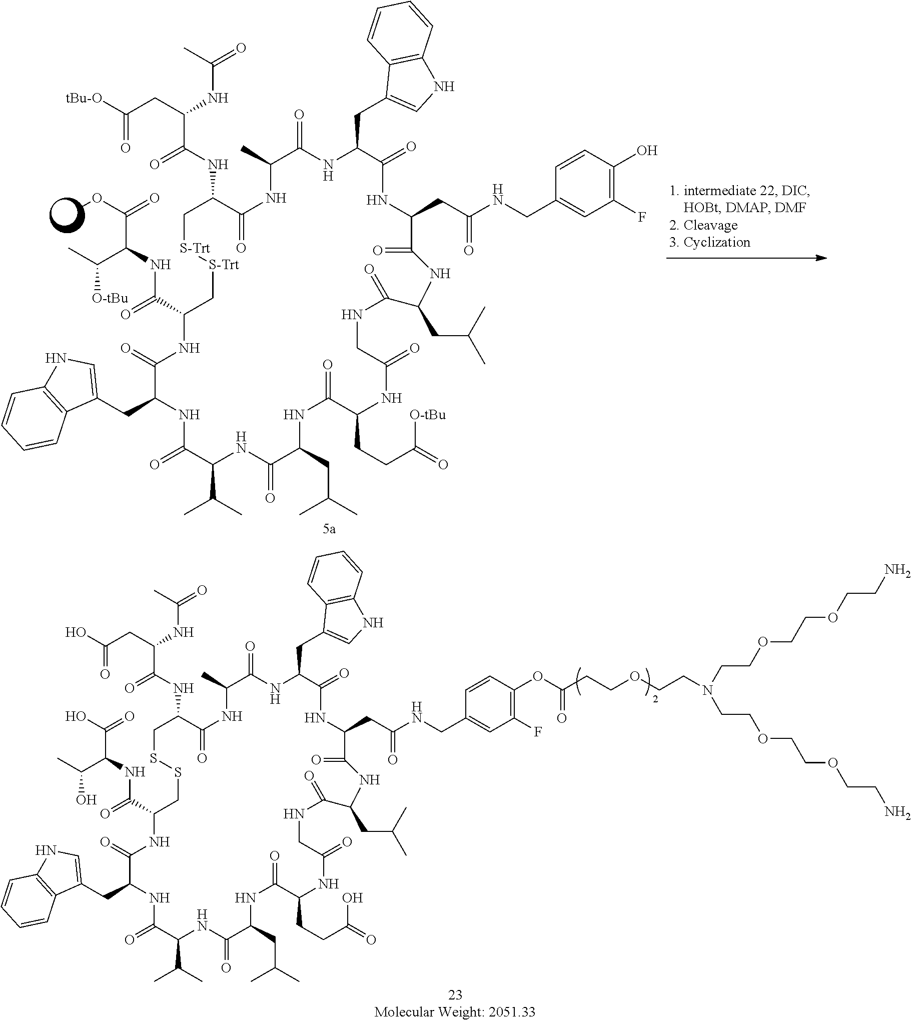

Intermediate 23 was synthesized by following the procedure mentioned in section [0008]-[0009], (Page 3-5), by the treatment of Intermediate 22. 0.50 mmol CTC resin afforded Intermediate 23 (20.0 mg, 91.4% purity, 2.0% yield) as a white solid. LCMS: RT=1.61 min, MS cal.: Mₐᵥ=2095.37, [M+H]⁺=2095.0, [M+2H]²⁺=1048.47.

### Preparation of KP-0002645/Compound 1104:

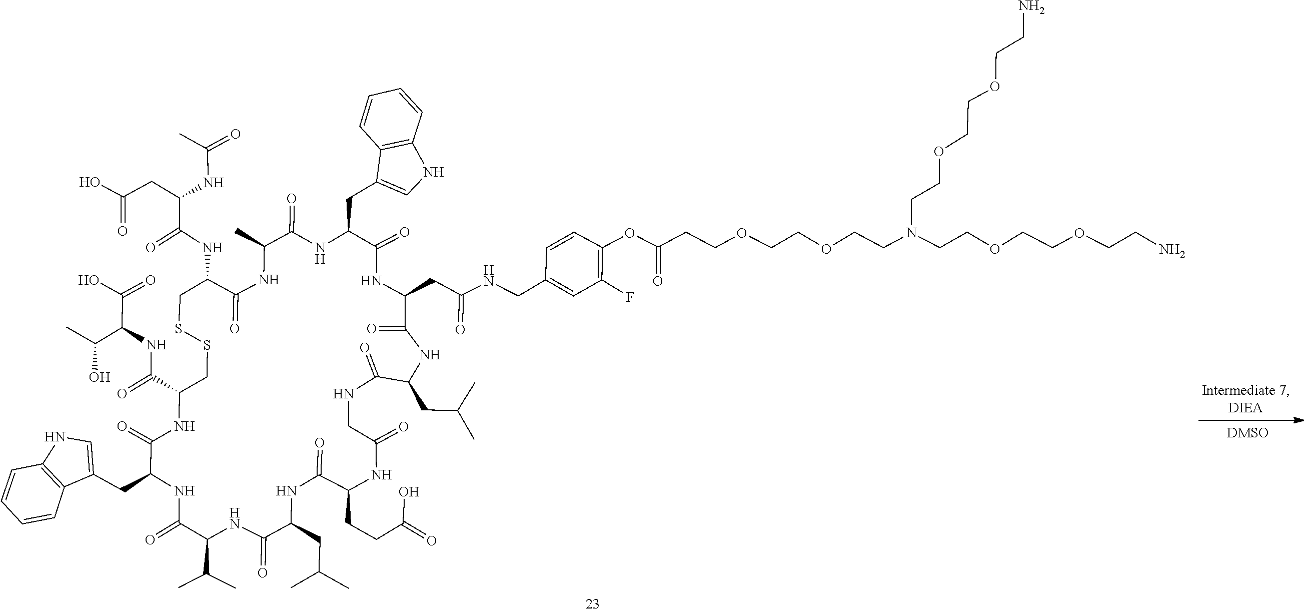 [SMILES: CC(=O)N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](CC(=O)NCc2ccc(OC(=O)CCOCCOCCN(CCOCCOCCN)CCOCCOCCN)c(F)c2)NC(=O)[C@H](Cc2c[nH]c3ccccc23)NC(=O)[C@H](C)NC1=O]

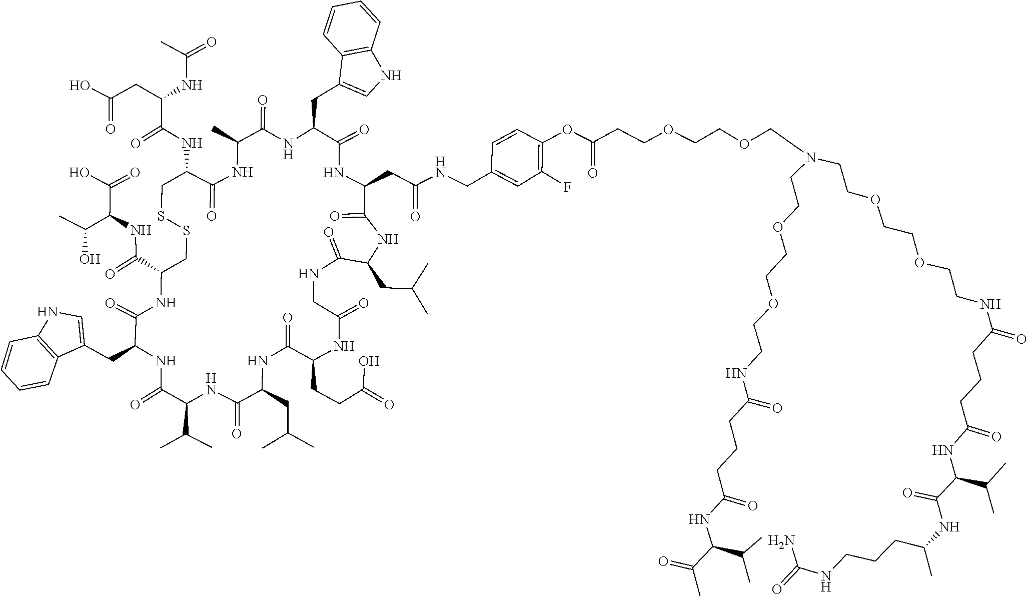 [SMILES: CC(=O)N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](CC(=O)NCc2ccc(OC(=O)CCOCCOCN(CCOCCOCCNC(=O)CCCC(=O)N[C@H](C(C)=O)C(C)C)CCOCCOCCNC(=O)CCCC(=O)N[C@H](C(=O)N[C@H](C)CCCNC(N)=O)C(C)C)c(F)c2)NC(=O)[C@H](Cc2c[nH]c3ccccc23)NC(=O)[C@H](C)NC1=O]

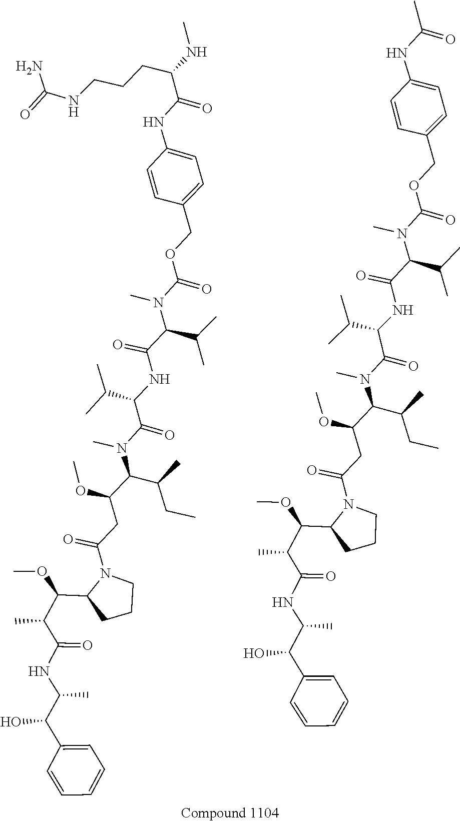 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(C)=O)cc1)C(C)C]

To a solution of Intermediate 23 (17.0 mg, 8.11 μmol, 1.00 equiv.), DIEA (40.49 mg, 81.13 μmol, 14.3 μL, 10.00 equiv.) in DMSO (500 μL) was added Intermediate 7 (21.66 mg, 16.23 μmol, 2.00 equiv.) at 20° C. Then the mixture was stirred at 20° C. for 1 h. LCMS indicated reactant was consumed completely and one main peak was desired MS. The mixture was purified by prep-HPLC (TFA condition) to afford Compound 1104 (9.1 mg, 1.95 μmol, 97.2% purity, 24.0% yield) as a white solid. LCMS: RT=1.092 min, MS cal.: Mₐᵥ=4534.39, [M+3H]³⁺-1511.90, [M+4H]⁴⁺=1134.10.

### Example 9. Procedure for Preparation of Compound 1105

The complete reaction scheme for the preparation of compound 1105 is shown in FIG. 9.

### Preparation of Intermediate 25:

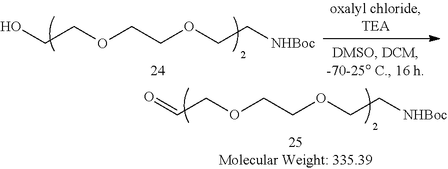

To a solution of DMSO (4.92 g, 62.98 mmol, 4.92 mL, 2.50 equiv.) in DCM (50 mL) was added to a solution of oxalyl chloride (6.40 g, 50.38 mmol, 4.41 mL, 2.00 equiv.) in DCM (50 mL), then the reaction mixture was stirred at −70° C. for 10 min, then a solution of Intermediate 24 (8.50 g, 25.19 mmol, 1.00 equiv.) in DCM (50 mL) was added dropwise. After stirred at −70° C. for 50 min, TEA (20.39 g, 201.54 mmol, 28.05 mL, 8.00 equiv.) was added and the reaction mixture was allowed to warm to 20° C. and stirred for 15 h. TLC (Dichloromethane:Methanol=10:1, R_{f}=0.48) showed the reactant was consumed completely and one new spot formed. The reaction mixture was quenched with H₂O (50 mL), the aqueous phase was extracted with DCM (300 mL*3). The combined organic layers were washed with brine (300 mL), dried over Na₂SO₄, filtered and concentrated under reduced pressure to give a residue. The residue was purified by silica gel column (DCM/EtOAc from 95/5 to 50/50) to afford Intermediate 25 (3.91 g, 11.66 mmol, 46.3% yield) as yellow oil. 1H NMR (400 MHz, CHLOROFORM-d) δ ppm 9.71-9.77 (m, 1H) 3.39-3.90 (m, 8H) 3.25-3.36 (m, 2H) 1.40-1.52 (m, 9H).

### Preparation of Intermediate 26:

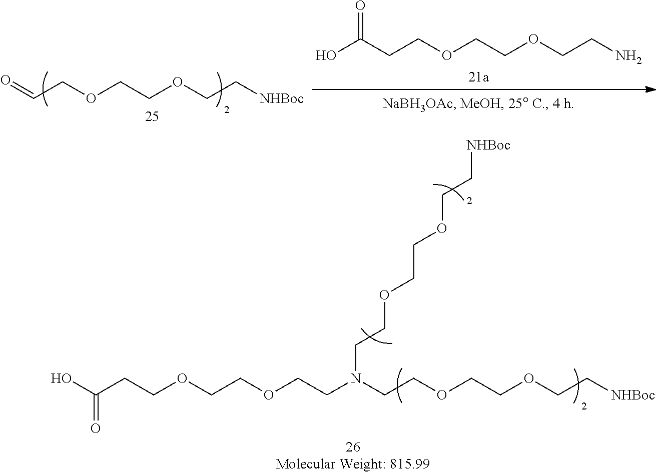

To a solution of Intermediate 25 (3.79 g, 11.29 mmol, 2.50 equiv.), Intermediate 21a (0.8 g, 4.51 mmol, 1.00 equiv.) in MeOH (40 mL) was added NaBH₃OAc (1.44 g, 6.77 mmol, 1.50 equiv.) slowly at 20° C. After addition, the reaction mixture was stirred at 20° C. for 4 h. LCMS found desired MS. The reaction mixture was quenched with H₂O (10 mL) dropwise. After filtration, the filtrate was concentrated. The residue was purified by prep-HPLC (column: Welch Ultimate XB—C18 250*50 mm, 10 μm, 120 A+Welch Ultimate 250*50 mm, 10 μm, 120 A; mobile phase: [water (0.1% TFA)-ACN]; B %: 24%-54%, 24 min) to afford Intermediate 26 (2.41 g, 2.95 mmol, 65.4% yield) as yellow oil. LCMS: RT=0.833 min, MS cal.: Mₐᵥ=815.99, [M+H]⁺=816.5. ¹H NMR (400 MHz, DEUTERIUM OXIDE) 6 ppm 7.87 (s, 1H) 7.37 (br d, J=7.53 Hz, 1H) 3.79-3.91 (m, 4H) 3.70-3.79 (m, 4H) 3.60-3.70 (m, 12H) 3.45-3.58 (m, 8H) 2.99-3.40 (m, 6H) 2.59 (t, J=5.52 Hz, 2H) 1.36 (s, 18H).

### Preparation of Intermediate 27:

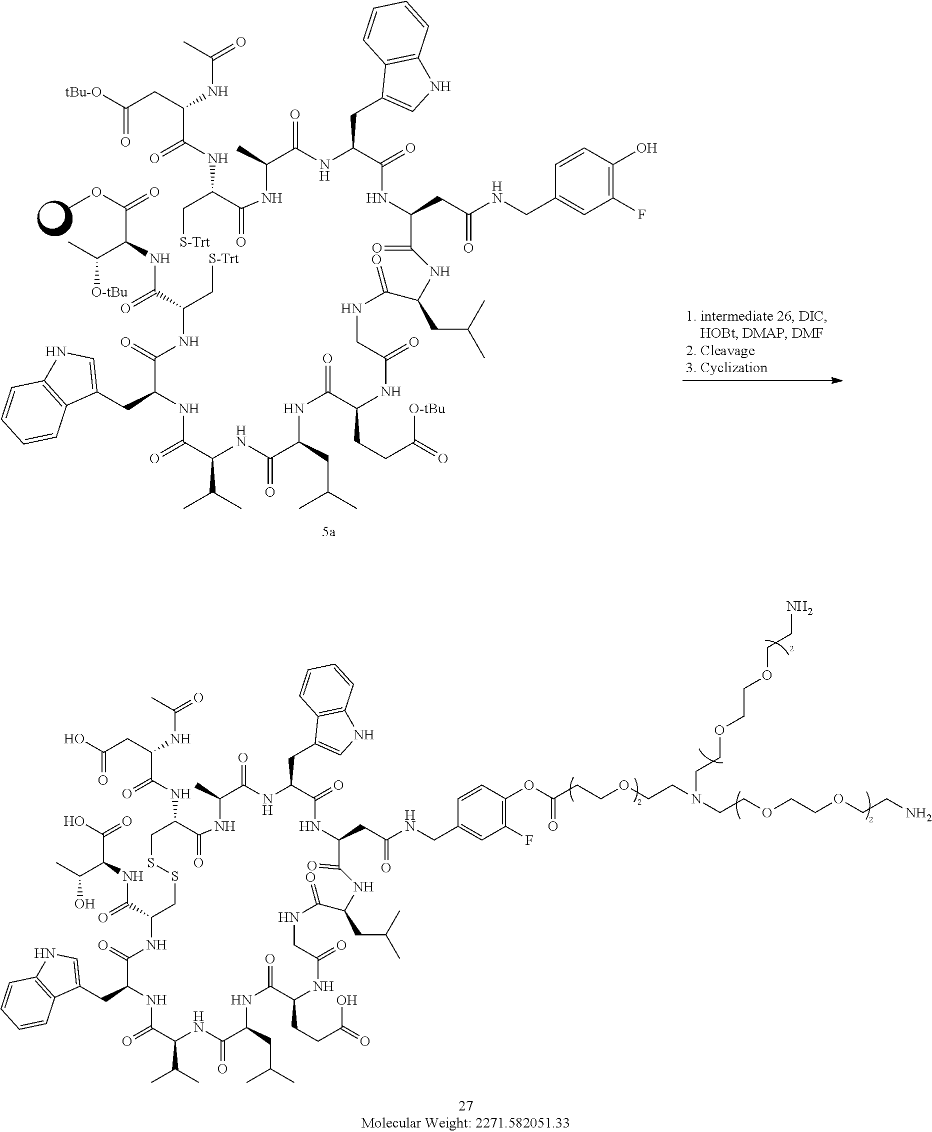

Intermediate 27 was synthesized by following the procedure mentioned in section [0008]-[0009], (Page 3-5), with the treatment of Intermediate 26. 0.50 mmol CTC resin afforded Intermediate 27 (50.0 mg, 2.0% yield) as a white solid. LCMS: RT=1.61 min, MS cal.: Mₐᵥ=2271.58, [M+2H]²⁺=1137.64, [M+3H]³⁺=758.50.

### Preparation of Compound 1105:

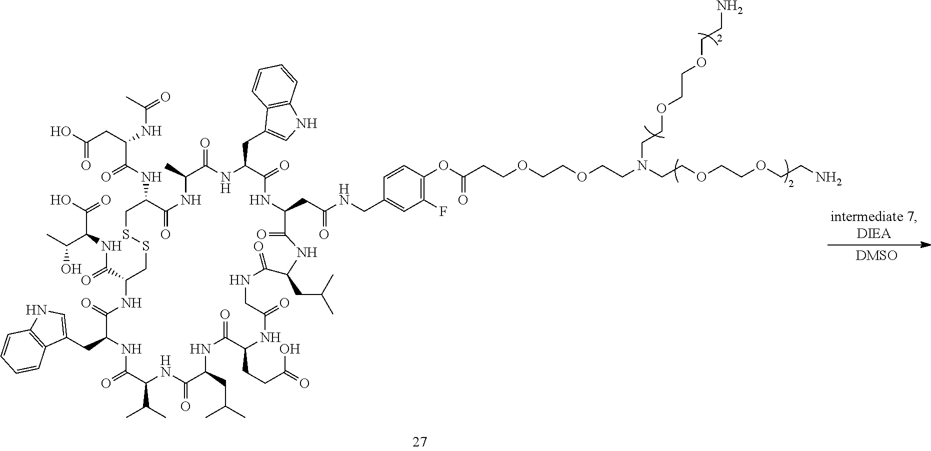

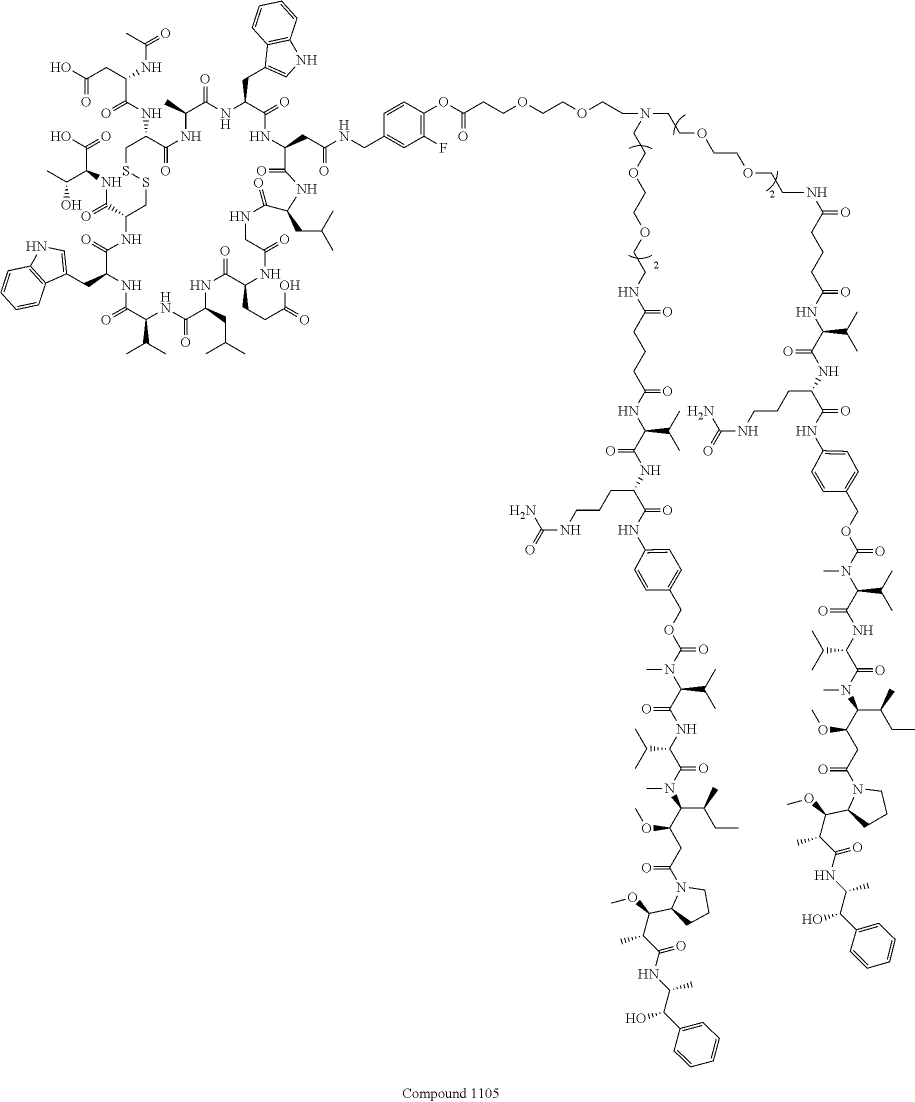

To a solution of Intermediate 27 (17.3 mg, 7.62 μmol, 1.00 equiv.), DIEA (9.84 mg, 76.16 μmol, 13.27 μL, 10.00 equiv.) in DMSO (600 μL) was added Intermediate 7 (20.84 mg, 15.61 μmol, 2.05 equiv.) at 20° C. Then the mixture was stirred at 20° C. for 1 h. LCMS indicated reactant was consumed completely and one main peak was desired MS. The mixture was purified by prep-HPLC (TFA condition) to afford Compound 1105 (17.1 mg, 47.7% yield, 97.2% purity) as a white solid. LCMS: RT=1.092 min, MS cal.: Mₐᵥ=4710.60, [M+3H]³⁺=1570.71, [M+4H]⁴⁺=1178.40, [M+5H]⁵⁺=942.69.

### Example 7. Procedure for Preparation of Compound 1106

### Preparation of Intermediate 28:

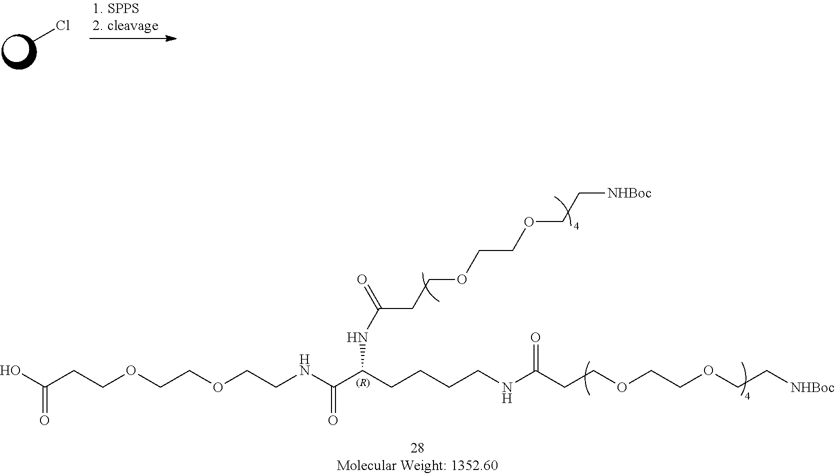

Peptide was synthesized using standard Fmoc chemistry (CTC resin).

1) Resin preparation: To the vessel containing CTC Resin (2.00 mmol, 2.00 g, 1.00 mmol/g) and Fmoc-HN-PEG₂-CH₂CH₂COOH (798.0 mg, 2.00 mmol, 1.00 equiv.) in DCM (20 mL) was added DIEA (4.00 equiv.) dropwise and mix for 2 h with N₂ bubbling at 20° C. Then added MeOH (2 mL) and bubbled with N₂ for another 30 min. The resin was washed with DMF (40 mL). Then 20% piperidine in DMF (40 mL) was added and the mixture was bubbled with N₂ for 30 min at 20° C. Then the mixture was filtered to obtain the resin. The resin was washed with DMF (40 mL) before proceeding to next step.

2) Coupling: A solution of Fmoc-D-Lys(Fmoc)-OH (2.21 g, 6.00 mmol, 3.00 equiv.), HBTU (2.19 g, 2.85 equiv.) in DMF (20 mL) was added to the resin with N₂ bubbling. Then DIEA (6.00 equiv.) was added to the mixture dropwise and bubbled with N₂ for 30 min at 20° C. The coupling reaction was monitored by ninhydrin test, if it showed colorless, the coupling was completed. The resin was then washed with DMF (40 mL).

3) Deprotection: 20% piperidine in DMF (40 mL) was added to the resin and the mixture was bubbled with N₂ for 30 mins at 20° C. The resin was then washed with DMF (40 L). The Deprotection reaction was monitored by ninhydrin test, if it showed blue or brownish red, the reaction was completed.

4) Coupling: A solution of Fmoc-HN-PEG₈-CH₂CH₂COOH (5.30 g, 8.00 mmol, 4.00 equiv.), HATU (2.93 g, 3.80 equiv.) in DMF (20 mL) was added to the resin with N₂ bubbling. Then DIEA (8.00 equiv.) was added to the mixture dropwise and bubbled with N₂ for 30 min at 20° C. The coupling reaction was monitored by ninhydrin test, if it showed colorless, the coupling was completed. The resin was then washed with DMF (20 mL).

5) Step 3) was repeated once.

6) Boc protection: A solution of Boc₂O (2.59 g, 12.00 mmol, 6.00 equiv.) and DIEA (12.00 equiv.) was added to resin and the mixture was bubbled with N₂ for 20 min. The coupling reaction was monitored by ninhydrin test, if it showed colorless, the coupling was completed. The resin was then washed with DMF (40 mL), MeOH (40 mL), and dried under reduced pressure.

TABLE 2
The list of amino acids and the corresponding reagents used on SPPS.

| # | Materials | Coupling reagents |
|---|---|---|
| 1 | Fmoc-HN-PEG₂-CH₂CH₂COOH | DIEA (4.00 equiv.) |
| | (4.00 equiv.) | |
| 2 | Fmoc-D-Lys(Fmoc)-OH (3.00 | HBTU (2.85 equiv.) and DIEA |
| | equiv.) | (6.00 equiv.) |
| 3 | Fmoc-HN-PEG₈-CH₂CH₂COOH | HATU (3.80 equiv.) and DIEA |
| | (4.00 equiv.) | (8.00 equiv.) |
| 4 | Boc₂O (6.00 equiv.) | DIEA (12.00 equiv.) |

### Peptide Cleavage:

1) Cleavage: Cleavage solution (20% HFIP/H₂O, v/v, 50 mL) was added to the flask containing the side chain protected peptide at room temperature and stirred for 1 h. After filtration, the filtrate was collected.

2) The combined filtrate was concentrated under reduced pressure, followed by lyophilization to afford Intermediate 28 (1.7 g, crude) as colorless oil. LCMS: RT=0.95 min, MS cal.: Mₐᵥ=1352.60, [M+H]⁺=1352.86.

### Preparation of Intermediate 29 (Sidechain Protected Resin-Bound Peptide):

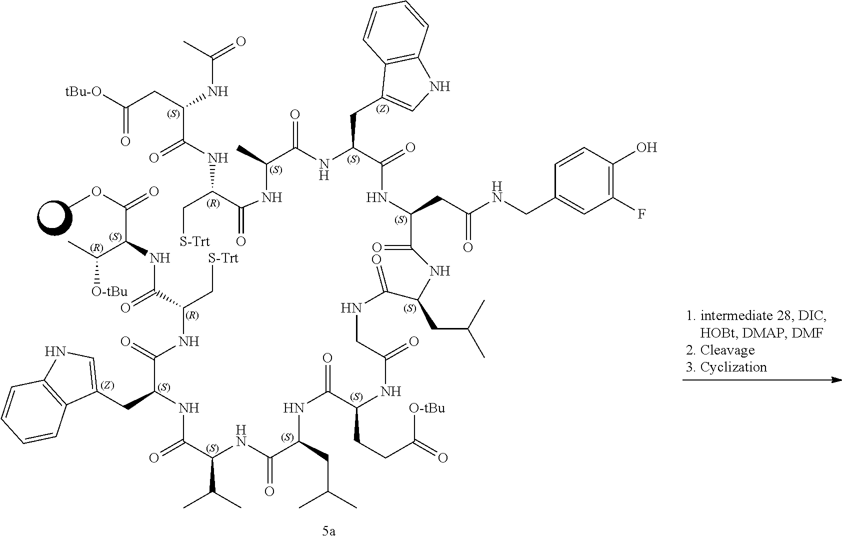

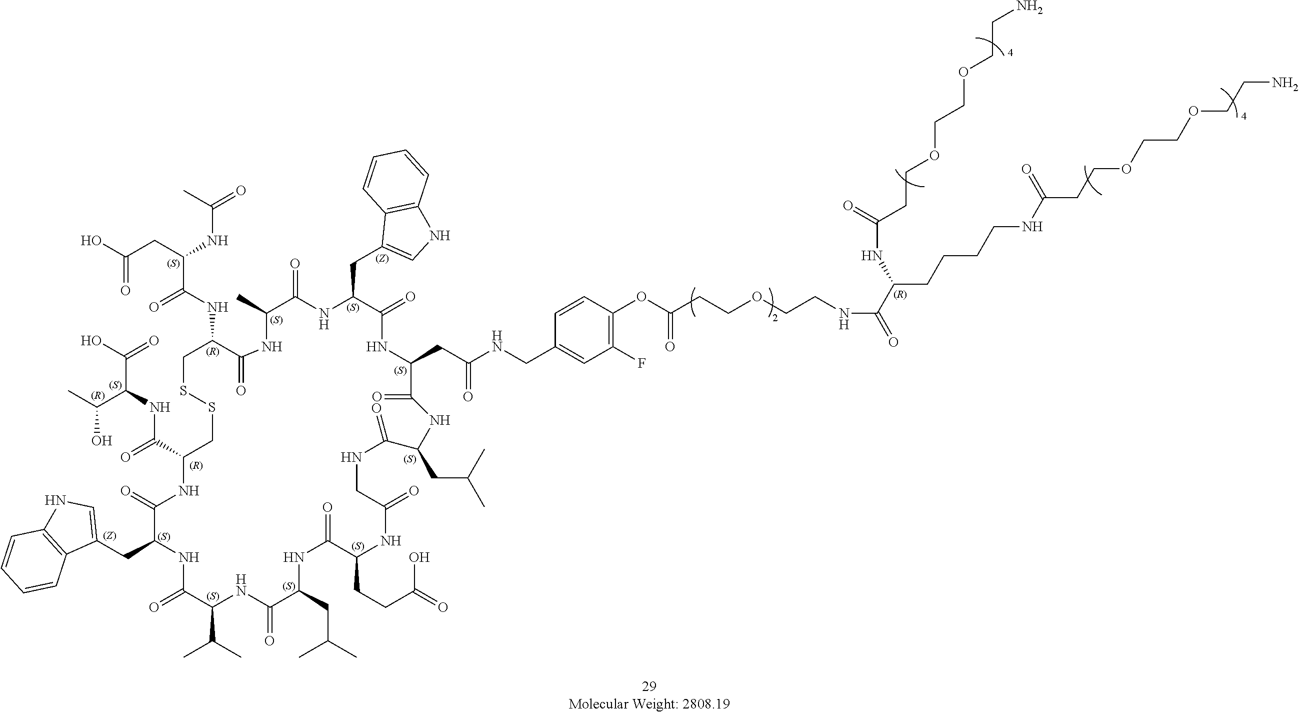

Intermediate 29 was synthesized by following the procedure mentioned in section [0008]-[0009], (Page 3-5), with the treatment of Intermediate 28. 0.50 mmol CTC resin afforded Intermediate 29 (300.0 mg, 91.4% purity, 9.7% yield) as a white solid. LCMS: RT=0.872 min, MS cal.: Mₐᵥ=2808.19, [M+2H]²⁺=1404.79, [M+3H]³⁺=936.52, [M+4H]⁴⁺=702.73.

### Preparation of Compound 1106:

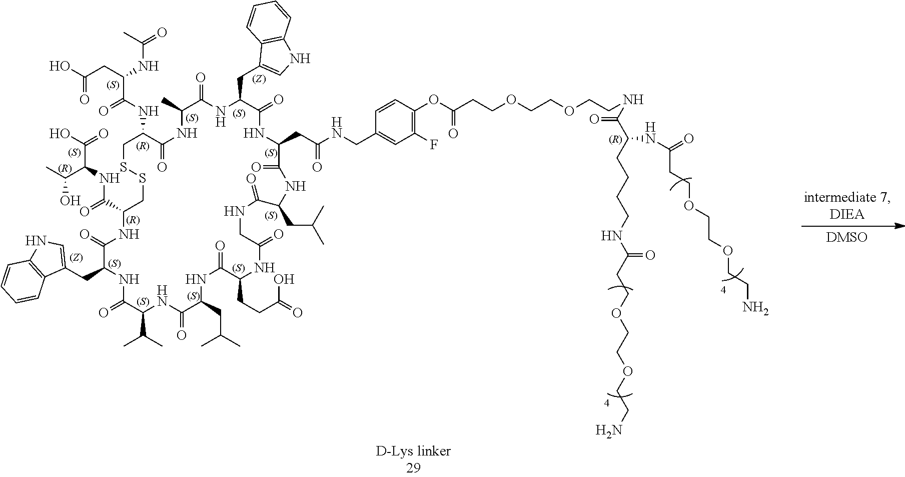

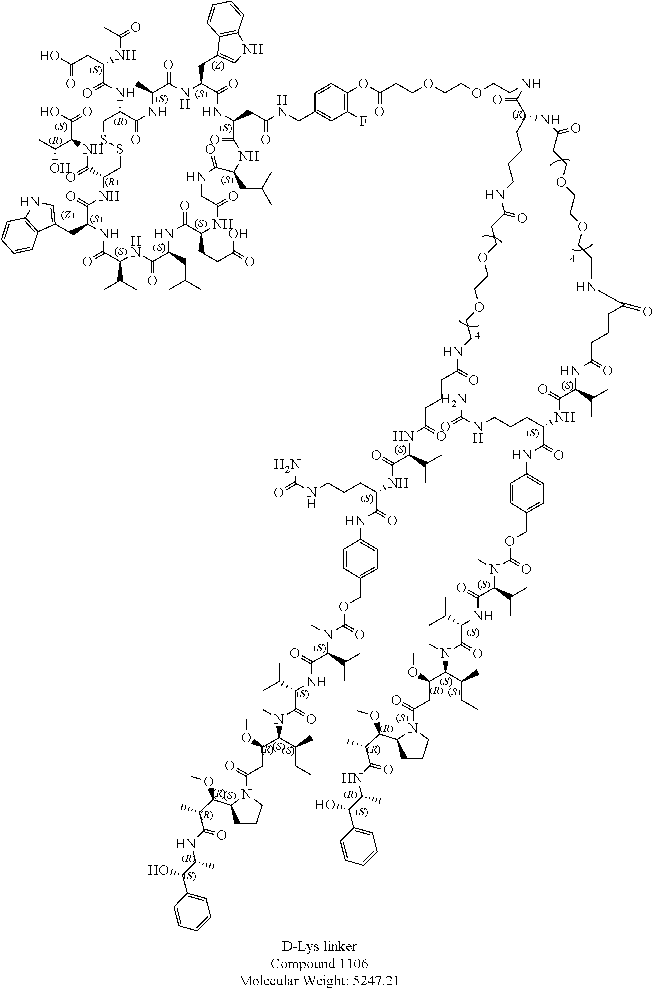

To a mixture of Intermediate 29 (20.0 mg, 7.12 umol, 1.00 equiv.), DIEA (9.20 mg, 71.22 μmol, 12.41 μL, 10.00 equiv.) in DMSO (400 μL) was added Intermediate 7 (19.9 mg, 14.96 μmol, 2.10 equiv.) at 20° C. Then the mixture was stirred at 20° C. for 1 h. LCMS indicated reactant was consumed completely and one main peak was desired MS. The mixture was purified by prep-HPLC (TFA condition) to afford Compound 1106 (16.7 mg, 3.02 μmol, 95.0% purity, 42.4% yield) as a white solid. LCMS: RT=1.61 min, MS cal.: Mₐᵥ=5247.21, [M+3H]³⁺-1749.87, [M+4H]⁴⁺=1312.53, [M+5H]⁵⁺=1050.28, [M+6H]⁶⁺=875.27.

### Example 10. Procedure for Preparation of Compound 1199

### Preparation of Intermediate 32:

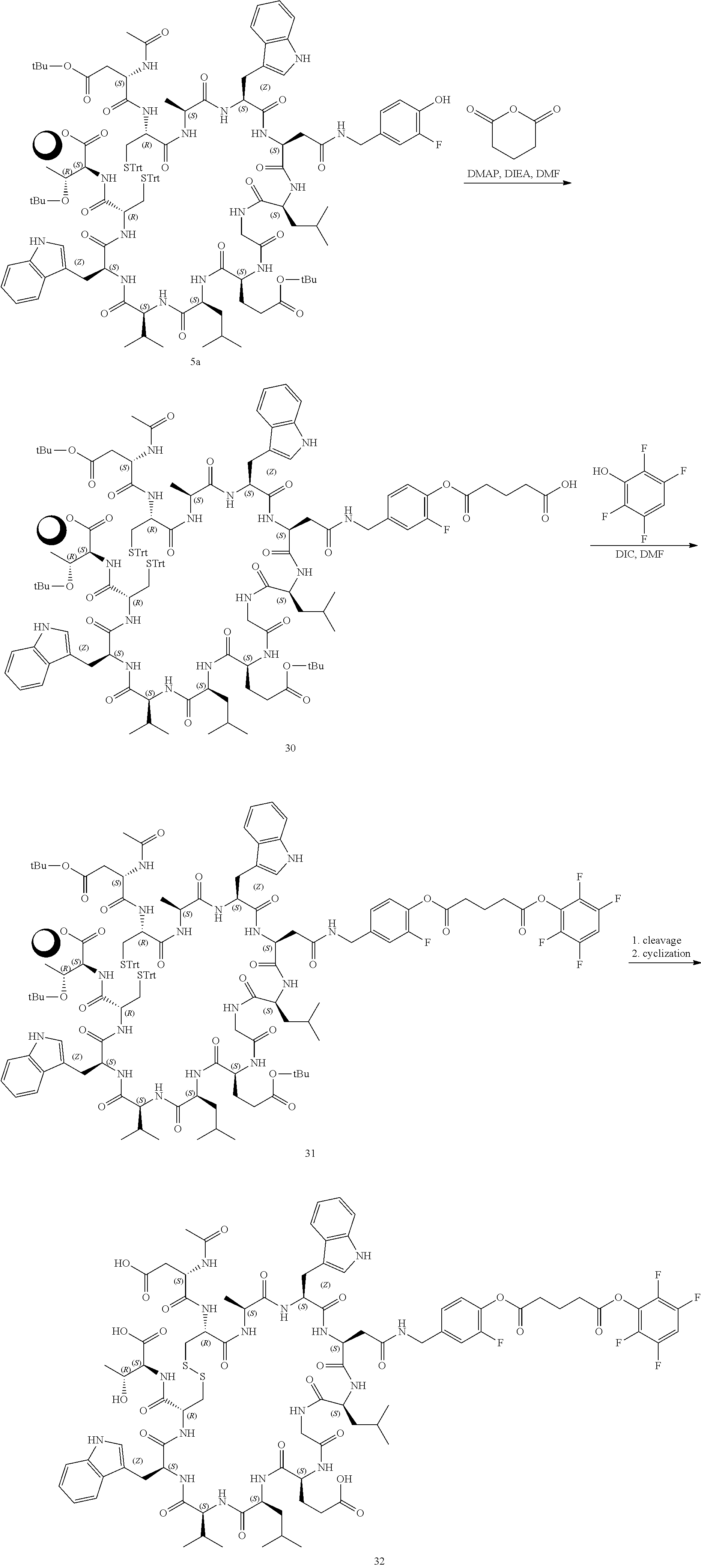

Peptide was synthesized using standard Fmoc chemistry (CTC resin).

1) Intermediate 5a (0.50 mmol, peptide resin) was synthesized by following the procedure mentioned in Example 4.

2) Coupling: To a mixture of Compound 5a (CTC resin, 0.50 mmol), DIEA (387.7 mg, 3.00 mmol, 522.53 uL, 6.00 equiv.), DMAP (183.26 mg, 1.50 mmol, 3.00 equiv.) in anhydrous DMF (10 mL) was added dihydro-2H-pyran-2,6(3H)-dione (342.3 mg, 3.00 mmol, 6.00 equiv.) at 20° C. with N₂ bubbling. Then the mixture was bubbled by N₂ for 2 h. After a mini-cleavage test, LCMS showed the reaction was completed. The peptide resin (Intermediate 30) was washed with DMF (20 mL), used for next step directly. LCMS: RT=1.21 min, MS cal.: Mₐᵥ=1789.95, [M+2H]²⁺=896.48.

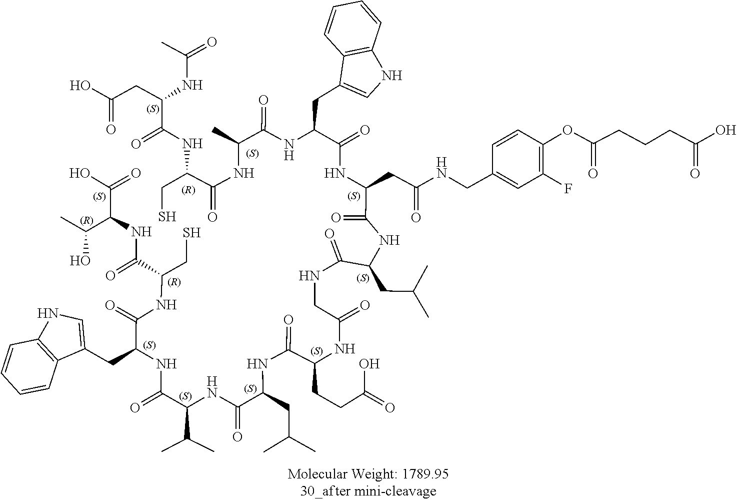

3) TFP ester formation: A solution of TFP (830.37 mg, 5.00 mmol, 10.00 equiv.) and DIC (631.00 mg, 5.00 mmol, 774.23 μL, 10.00 equiv.) in anhydrous DMF (5 mL) was added to the resin-bound peptide (Intermediate 30) at 20° C. with N₂ bubbling. Then the mixture was bubbled by N₂ for 2 h. After a mini-cleavage test, LCMS showed the reaction was completed. The resin was washed with DMF (20 mL, 2-isopropoxypropane (20 mLs, dried by N₂ bubbling to afford Intermediate 31 (CTC resin, 0.5 mmol) as a bright yellow solid.

4) Cleavage: Cleavage solution (TFA/Tis/H₂O, 95/2.5/2.5, v/v/v 20 mL) was added to the flask containing the side chain protected peptide at room temperature and stirred for 1 h. After filtration, the filtrate was precipitated with isopropyl ether (cold, 100 L). After filtration, the solid was washed with isopropyl ether (cold, 50 mL) for two additional times, and dried under reduced pressure for 2 h.

5) Cyclization: The crude peptide was dissolved in HOAc/MeCN/H₂O (4/3/3, v/v/v, 500 mL). Then the mixture was added 0.1 M I₂/AcOH dropwise until a yellow color persisted, then the mixture was stirred at 20° C. for 5 min. The mixture was quenched by addition of 0.1 M aq. Na₂S₂O₃ dropwise until the yellow color disappeared. After filtration, the filtrate was purified by prep-HPLC (A: 0.075% TFA/H₂O, B: MeCN), followed by lyophilization to afford Intermediate 32 (73.0 mg, 89.2% purity, 6.7% yield) as a white solid. LCMS: RT=1.31 min, MS cal.: Mₐᵥ=1935.99, [M+2H]²⁺=968.03.

### Preparation of Compound 1199:

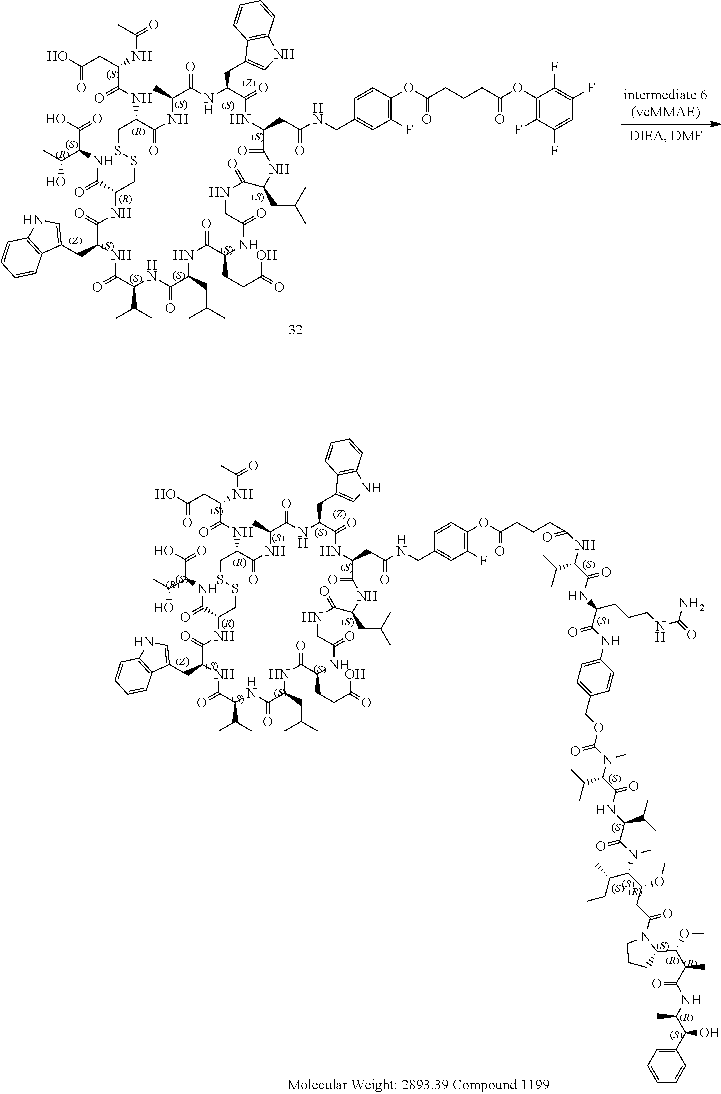

To a solution of Intermediate 6 (vcMMAE, 11.9 mg, 10.6 μmol, 1.00 equiv.) in DMSO (400 μL) was added Intermediate 32 (20.0 mg, 10.6 μmol, 1.00 equiv.) and DIEA (6.82 mg, 53.0 μmol, 5.00 equiv.) at 20° C. The mixture was stirred at 20° C. for 2 h. LC-MS showed the starting material was consumed completely. The solvent was removed under reduced pressure. The residue was purified by prep-HPLC (TFA condition) to obtained Compound 1199 (16.8 mg, 5.89 μmol, 55.8% yield, 96.8% purity) as a white solid. LCMS: RT=1.118 min, MS cal.: Mₐᵥ=2893.35, [M+2H]²⁺=1447.20, [M+3H]³⁺=965.15.

### Example 11. Procedure for Preparation of Compound 1434

### Preparation of Intermediate 36:

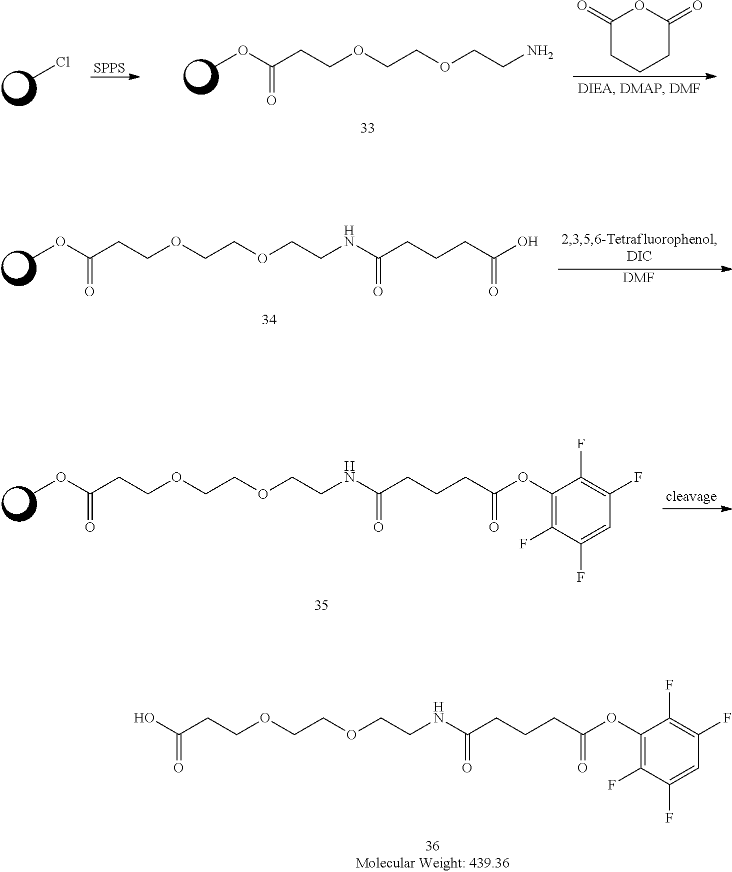

Peptide was synthesized using standard Fmoc chemistry (CTC resin).

1) Resin preparation: To the vessel containing CTC Resin (1.00 mmol, 1.00 g, 1.00 mmol/g) and Fmoc-HN-PEG₂-CH₂CH₂COOH (399.0 mg, 1.00 mmol, 1.00 equiv.) in DCM (10 mL) was added DIEA (4.00 equiv.) dropwise and mixed for 2 h with N₂ bubbling at 20° C. Then added MeOH (1 mL) and bubbled with N₂ for another 30 min. The resin was washed with DMF (20 mL). Then 20% piperidine in DMF (20 mL) was added and the mixture was bubbled with N₂ for 30 min at 20° C. Then the mixture was filtered to obtain the resin. The resin was washed with DMF (20 mL) before proceeding to next step.

2) Deprotection: 20% piperidine in DMF (20 mL) was added to the resin and the mixture was bubbled with N₂ for 30 mins at 20° C. The resin was then washed with DMF (20 mL). The deprotection reaction was monitored by ninhydrin test, if it showed blue or brownish red, the reaction was completed to afford Intermediate 33.

3) Coupling: A solution of dihydro-2H-pyran-2,6(3H)-dione (684.0 mg, 6.00 mmol, 6.00 equiv.) and DIEA (12.00 equiv.) was added to resin and the mixture was bubbled with N₂ for 20 min. The coupling reaction was monitored by ninhydrin test, if it showed colorless, the coupling was completed. The resin was then washed with DMF (20 mL) to afford Intermediate 34.

4) TFP ester formation: A solution of 2,3,5,6-tetrafluorophenol (1.65 g, 10.00 mmol, 10.00 equiv.) and DIC (1.26 g, 10.00 mmol, 774.23 μL, 10.00 equiv.) in anhydrous DMF (10 mL) was added to the peptide resin (Intermediate 34) at 20° C. with N₂ bubbling. Then the mixture was bubbled by N₂ for 2 h. After a mini-cleavage test, LCMS showed the reaction was completed. The resin was washed with DMF (20 mL), 2-isopropoxypropane (20 mL), isopropyl ether (20 mL), dried by N₂ bubbling to afford Intermediate 35 (CTC resin, 1.0 mmol).

TABLE 3
The list of amino acids and the corresponding reagents used on SPPS.

| | Materials | Coupling reagents |
|---|---|---|
| 1 | Fmoc-HN-PEG₂-CH₂CH₂COOH (1.00 equiv.) | DIEA (4.00 equiv.) |
| 2 | dihydro-2H-pyran-2,6(3H)-dione (3.00 | DIEA (6.00 equiv.) |
| | equiv.) | |
| 3 | 2,3,5,6-tetrafluorophenol (10.00 equiv.) | DIEA (10.00 equiv.) |

### Peptide Cleavage and Purification:

1) Cleavage: A solution of 1% TFA/DCM (v/v, 40 mL) was added to the resin-bound peptide (Intermediate 35) and stirred at 20° C. for 5 min. after filtration, the filtrate was concentrated under reduced pressure.

2) Purification: the residue was purified by prep-HPLC (A: 0.075% TFA/H₂O; B: MeCN) directly to afford Intermediate 36 (250.0 mg, 41.8% yield) as colorless oil. LCMS: RT=0.947 min, MS calcd.: Mav=439.36, mass observed: [M+H]⁺=440.23, [M+Na]⁺=462.11.

### Preparation of Compound 1434:

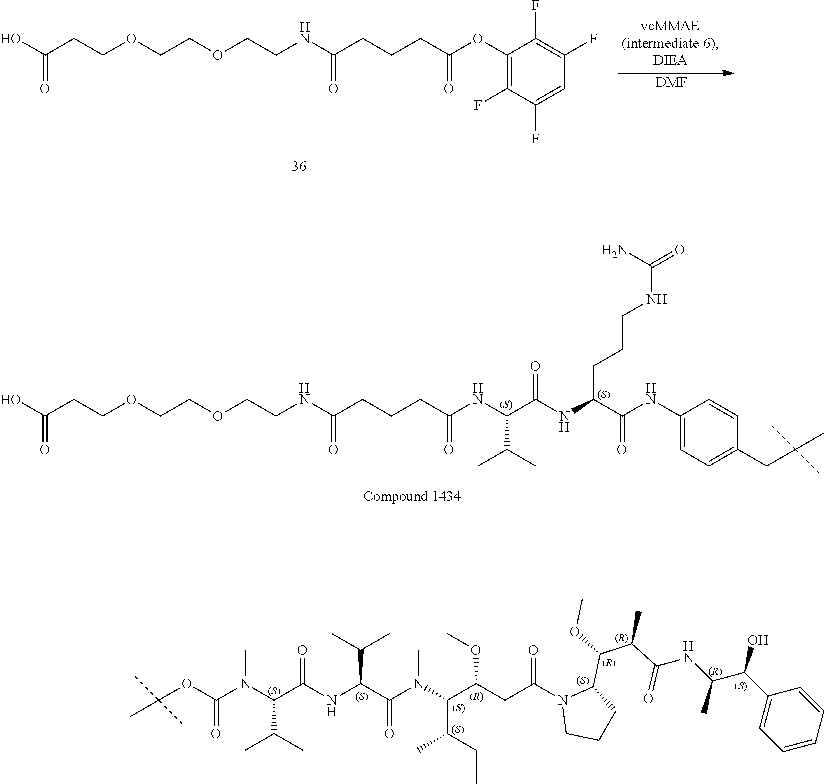

To a mixture of Intermediate 36 (39.1 mg, 89.02 μmol, 2.00 equiv.) and Intermediate 6 (vcMMAE, 50.0 mg, 44.51 μmol, 1.00 equiv.) in DMF (1 mL) was added DIEA (23.01 mg, 178.04 μmol, 31.01 μL, 4.00 equiv.) in one portion at 20° C. The mixture was stirred at 20° C. for 30 min. the mixture was purified by prep-HPLC (A: 0.075% TFA/H₂O; B: MeCN) directly, followed by lyophilization to afford Compound 1434 (36.9 mg, 26.34 μmol, 59.2% yield, 99.7% purity) as a white solid. LCMS: RT=1.955 min, MS calcd.: Mₐᵥ=1396.71, mass observed: [M+H]⁺=1398.1, [M+2H]²⁺=699.6. The dashed lines indicate a single covalent bond between the upper and lower portions of compound 1434.

Compound 1435, Compound 1436, Compound 1437 were synthesized by following the procedure for compound 1434. The dashed lines indicate a single covalent bond between the upper and lower portions of each compound's structure.

Compound 1435 was obtained (39.9 mg, 25.6 μmol, 99.9% purity, 50.8% yield) as a white solid. LCMS: RT=1.941 min, MS calcd.: Mₐᵥ=1555.89, [M+H]⁺=1557.0, [M+2H]²⁺=778.6.

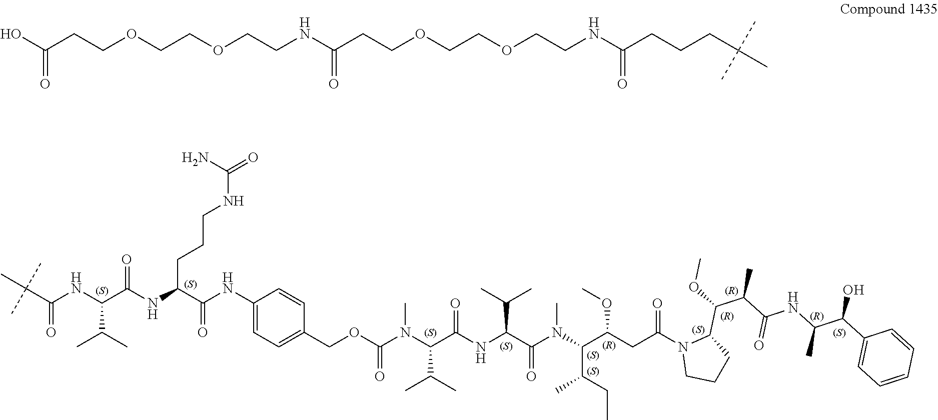

Compound 1436 was obtained (41.8 mg, 25.4 μmol, 99.6% purity, 57.9% yield) as a white solid. LCMS: RT=1.934 min, MS calcd.: Mₐᵥ=1644.00, [M+H]⁺=1645.3, [M+2H]²⁺=822.8.

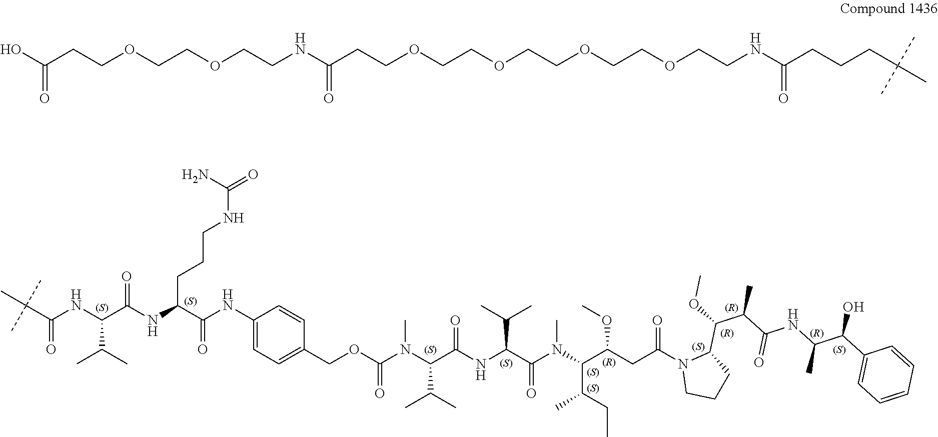

Compound 1437 was obtained (49.4 mg, 27.1 μmol, 99.8% purity, 49.9% yield) as a white solid. LCMS: RT=1.937 min, MS calcd.: Mₐᵥ=1820.21, [M+H]⁺=1821.4, [M+2H]²⁺=910.8, [M+3H]³⁺=607.7.

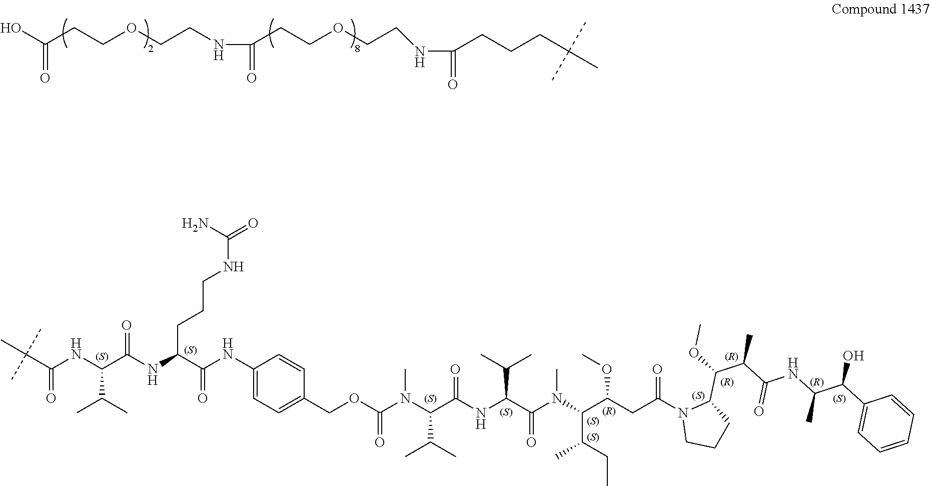

### Example 12. Procedure for Preparation of Compound 1438

### Preparation of Intermediate 37:

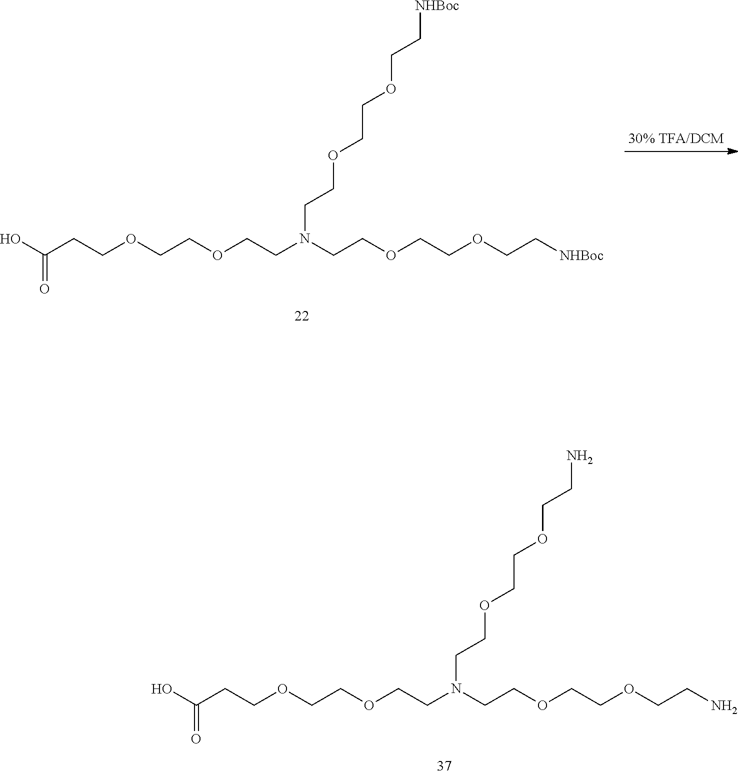 [SMILES: CC(C)(C)OC(=O)NCCOCCOCCN(CCOCCOCCNC(=O)OC(C)(C)C)CCOCCOCCC(=O)O ; NCCOCCOCCN(CCOCCOCCN)CCOCCOCCC(=O)O]

A mixture of Intermediate 22 (0.10 g) in TFA/DCM (3/7, v/v, 2 mL) was stirred at 20° C. for 0.5 h. The solvent was removed under reduced pressure. The residue was lyophilized to afford Intermediate 37 (0.10 g, crude, TFA salt) as colorless oil.

### Preparation of Compound 1438:

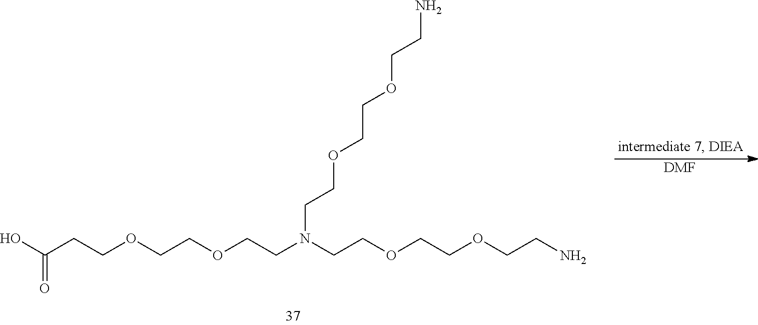

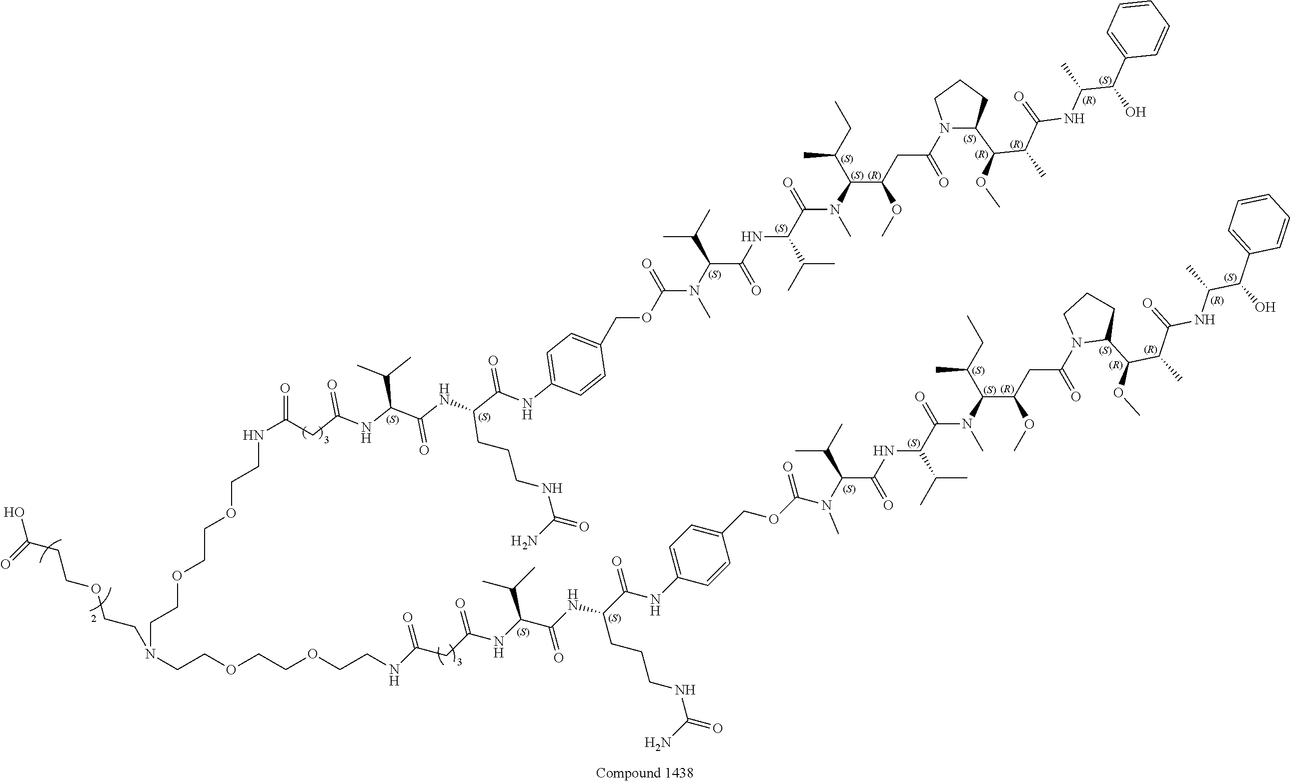

To a solution of Intermediate 37 (3.00 mg, 6.83 μmol, 1.00 equiv.), DIEA (5.29 mg, 40.95 μmol, 7.13 μL, 6.00 equiv.) in DMF (0.5 mL) was added Intermediate 7 (27.33 mg, 20.48 μmol, 3.00 equiv.) at 20° C. Then the reaction mixture was stirred at 20° C. for 1 h. The mixture was purified by prep-HPLC (A: 0.075% TFA/H₂O; B: MeCN) directly, followed by lyophilization to afford Compound 1438 (6.3 mg, 2.19 μmol, 96.5% purity, 32.0% yield) as a white solid. LCMS: RT=1.709 min, MS calcd.: Mₐᵥ=2878.57, [M+2H]²⁺=1440.3, [M+3H]³⁺-960.5.

### Preparation of Intermediate 37:

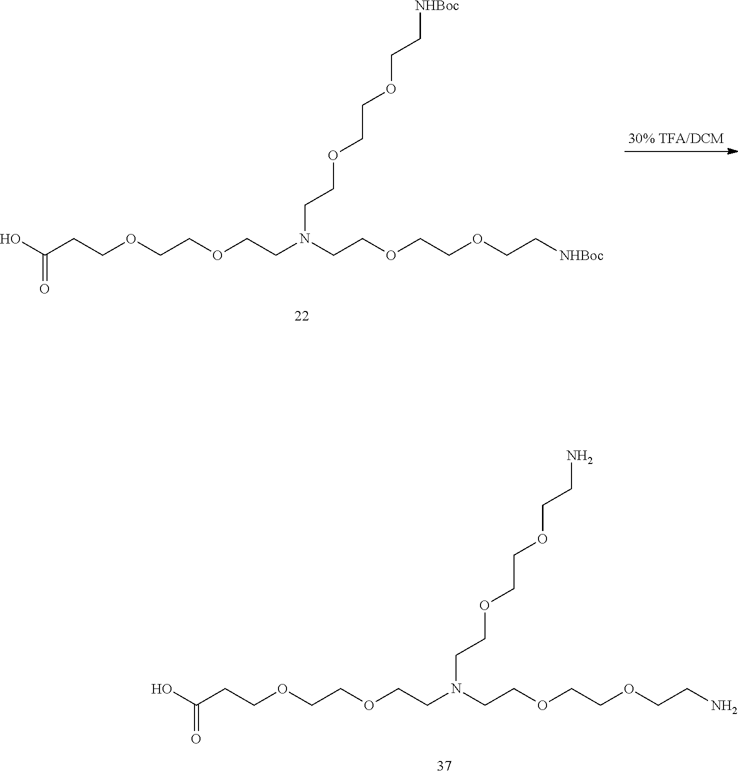 [SMILES: CC(C)(C)OC(=O)NCCOCCOCCN(CCOCCOCCNC(=O)OC(C)(C)C)CCOCCOCCC(=O)O ; NCCOCCOCCN(CCOCCOCCN)CCOCCOCCC(=O)O]

A mixture of Intermediate 22 (0.10 g) in TFA/DCM (3/7, v/v, 2 mL) was stirred at 20° C. for 0.5 h. The solvent was removed under reduced pressure. The residue was lyophilized to afford Intermediate 37 (0.10 g, crude, TFA salt) as colorless oil.

### Preparation of Compound 1438:

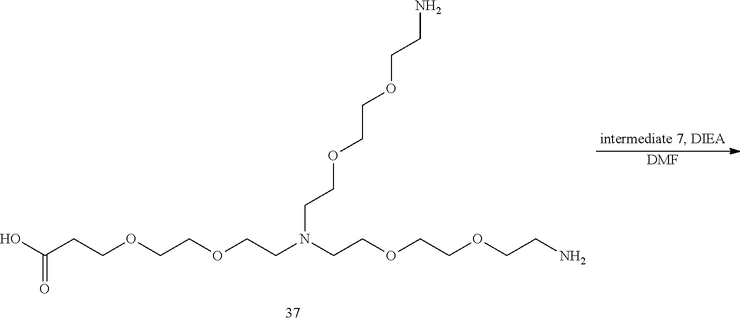

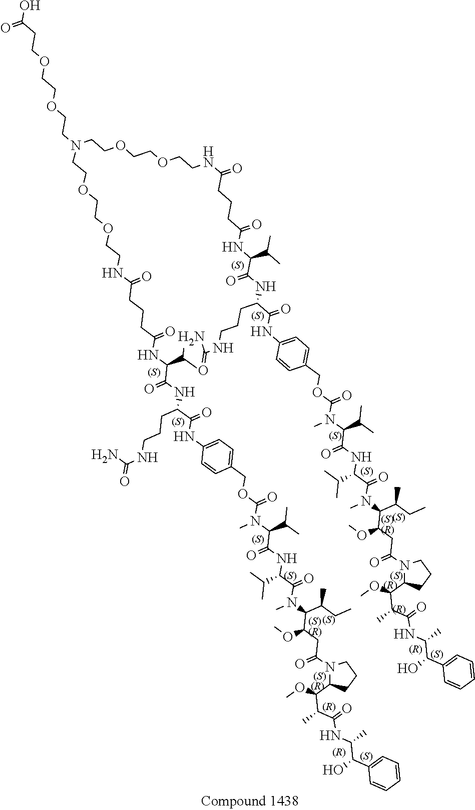

To a solution of Intermediate 37 (3.00 mg, 6.83 μmol, 1.00 equiv.), DIEA (5.29 mg, 40.95 μmol, 7.13 μL, 6.00 equiv.) in DMF (0.5 mL) was added Intermediate 7 (27.33 mg, 20.48 μmol, 3.00 equiv.) at 20° C. Then the reaction mixture was stirred at 20° C. for 1 h. The mixture was purified by prep-HPLC (A: 0.075% TFA/H₂O; B: MeCN) directly, followed by lyophilization to afford Compound 1438 (6.3 mg, 2.19 μmol, 96.5% purity, 32.0% yield) as a white solid. LCMS: RT=1.709 min, MS calcd.: Mₐᵥ=2878.57, [M+2H]²⁺=1440.3, [M+3H]³⁺-960.5.

### Example 13. Procedure for Preparation of Compound 1439

### Preparation of Intermediate 38:

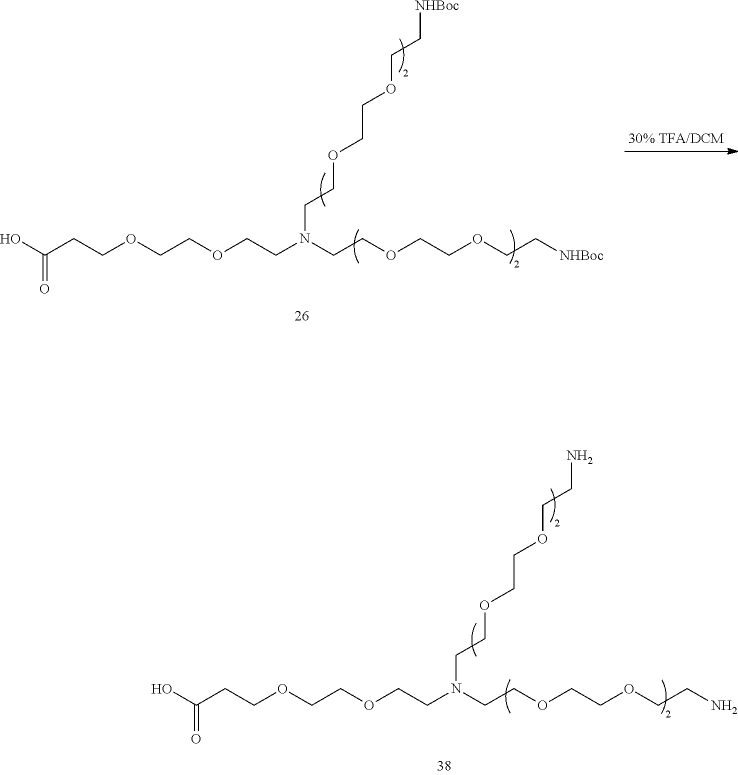

A mixture of Intermediate 26 (0.10 g) in TFA/DCM (3/7, v/v, 2 mL) was stirred at 20° C. for 0.5 h. The solvent was removed under reduced pressure. The residue was lyophilized to afford Intermediate 38 (0.10 g, crude, TFA salt) as colorless oil.

### Preparation of Compound 1439:

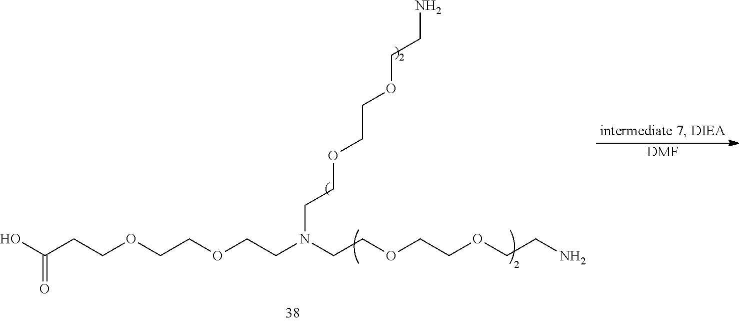

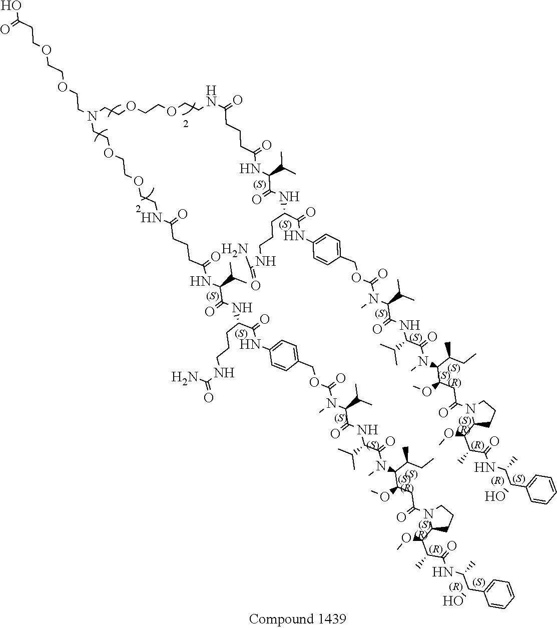

To a solution of Intermediate 38 (20.0 mg, 32.48 μmol, 1.00 equiv.), DIEA (25.19 mg, 194.88 μmol, 33.95 μL, 6.00 equiv.) in DMF (2 mL) was added Intermediate 7 (130.0 mg, 97.44 μmol, 3.00 equiv.) at 20° C. Then the reaction mixture was stirred at 20° C. for 1 h. The mixture was purified by prep-HPLC (A: 0.075% TFA/H₂O; B: MeCN) directly, followed by lyophilization to afford Compound 1439 (10.8 mg, 3.54 μmol, 97.1% purity, 10.8% yield) as a white solid. LCMS: RT=2.221 min, MS calcd.: Mₐᵥ=3054.78, [M+2H]²⁺=1528.4, [M+3H]³⁺=1019.1, [M+4H]⁴⁺=764.7.

### Example 14. Procedure for Preparation of Compound 1440

### Preparation of Intermediate 39:

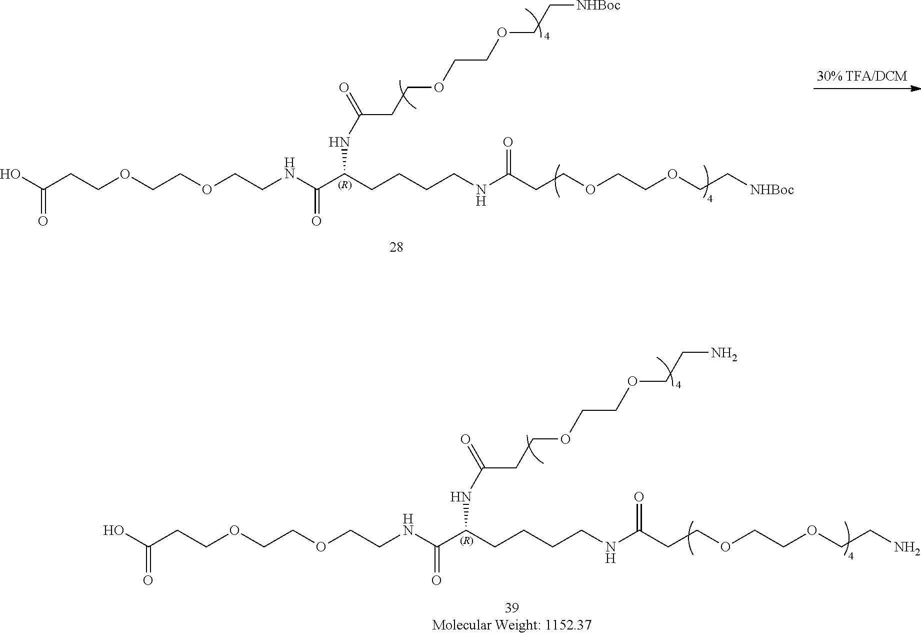

A mixture of Intermediate 28 (0.30 g) in TFA/DCM (3/7, v/v, 6 mL) was stirred at 20° C. for 0.5 h. The solvent was removed under reduced pressure. The residue was lyophilized to afford Intermediate 39 (0.20 g, crude, TFA salt) as colorless oil. LCMS: RT=0.637 min, MS calcd.: M, =1152.37, [M+H]⁺=1152.7, [M+2H]²⁺=576.99.

### Compound 1440:

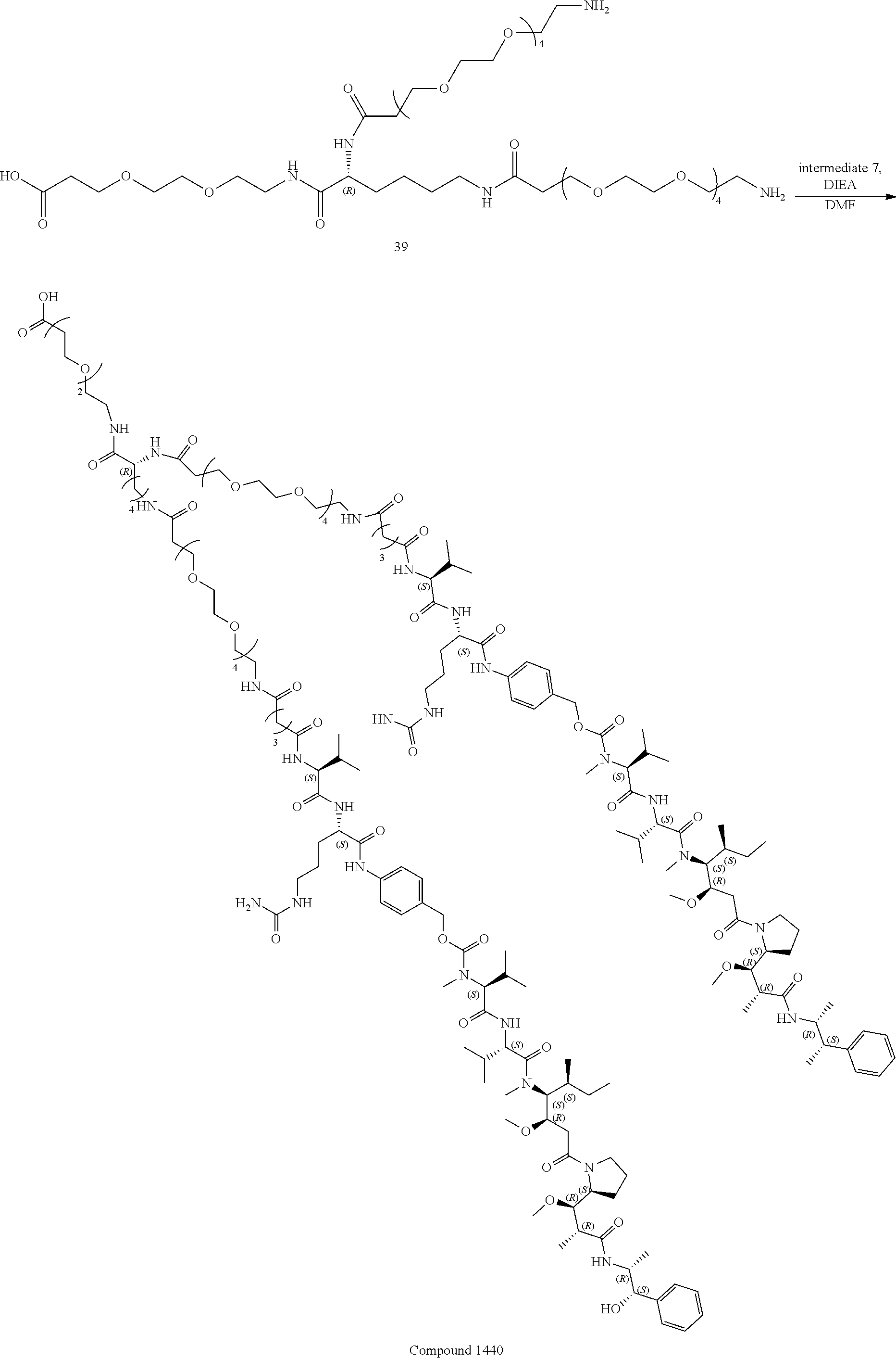

To a solution of Intermediate 39 (17.27 mg, 14.99 μmol, 1.00 equiv.), DIEA (9.68 mg, 74.93 μmol, 13.05 μL, 6.00 equiv.) in DMF (0.5 mL) was added Intermediate 7 (50.0 mg, 37.46 μmol, 2.50 equiv.) at 20° C. Then the reaction mixture was stirred at 20° C. for 1 h. The mixture was purified by prep-HPLC (A: 0.075% TFA/H₂O; B: MeCN) directly, followed by lyophilization to afford Compound 1440 (31.7 mg, 8.68 μmol, 57.9% yield, 98.3% purity) as a white solid. LCMS: RT=1.689 min, MS calcd.: Mₐᵥ=3591.39, [M+2H]²⁺=1797.0, [M+3H]³⁺=1197.9, [M+4H]⁴⁺=898.8, [M+5H]⁵⁺=719.8.

### Example 15. Procedure for Preparation of Compound 1441

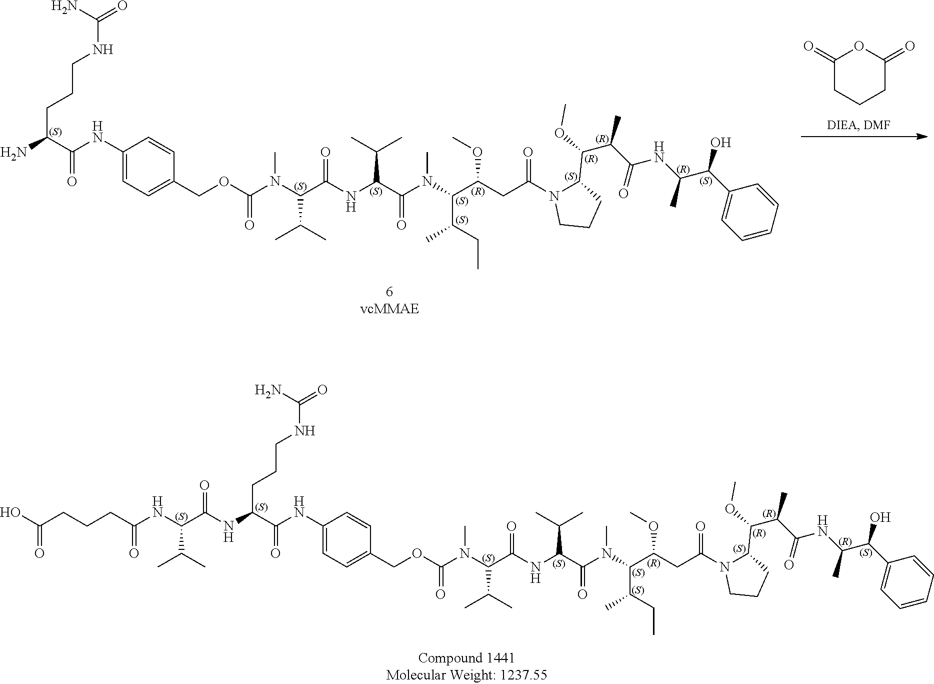

A mixture of dihydro-2H-pyran-2,6(3H)-dione (6.09 mg, 53.41 μmol, 1.50 equiv.), Intermediate 6 (vcMMAE, 40.00 mg, 35.61 μmol, 1.00 equiv.), DIEA (13.81 mg, 106.82 μmol, 18.61 μL, 3.00 equiv.) in DMF (0.4 mL) was stirred at 20° C. for 0.5 h. The mixture was purified by prep-HPLC (A: 0.075% TFA/H₂O; B: MeCN) directly, followed by lyophilization to afford Compound 1441 (34.5 mg, 27.26 μmol, 76.5% yield, 97.8% purity) as a white solid. LCMS: RT=1.983 min, MS calcd.: Mₐᵥ=1237.83, [M+H]⁺=1238.8, [M+2H]²⁺=619.6.

### Example 16. Procedure for Preparation of Compound 1574

### Preparation of Intermediate 40:

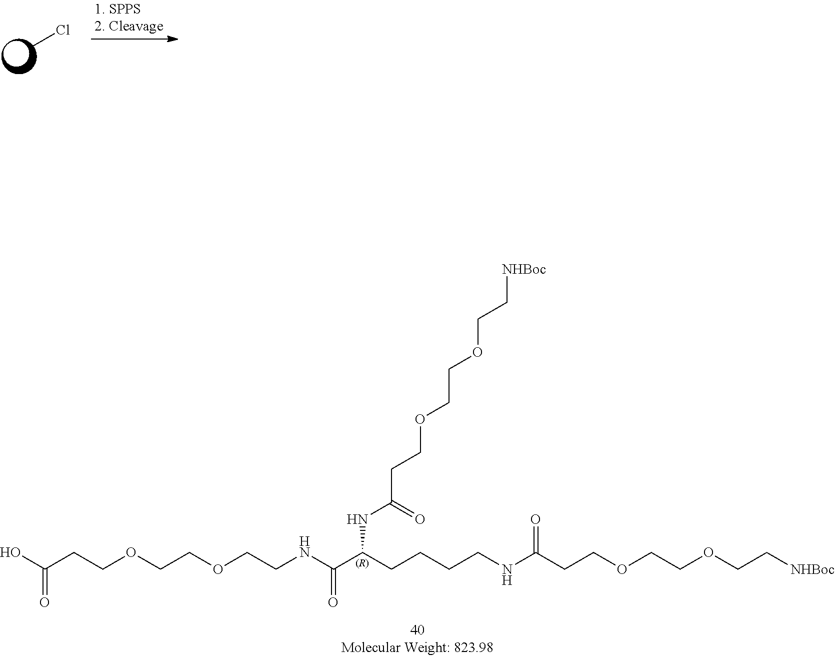

Intermediate 40 was synthesized by following the procedure for the synthesis of compound 1106.

TABLE 4
The list of amino acids and the corresponding reagents used on SPPS

| # | Materials | Coupling reagents |
|---|---|---|
| 1 | Fmoc-HN-PEG₂-CH₂CH₂COOH | DIEA (4.00 equiv.) |
| | (1.00 equiv.) | |
| 2 | Fmoc-D-Lys(Fmoc)-OH (3.00 | HBTU (2.85 equiv.) and DIEA |
| | equiv.) | (6.00 equiv.) |
| 3 | Fmoc-HN-PEG₂-CH₂CH₂COOH | HATU (3.80 equiv.) and DIEA |
| | (4.00 equiv.) | (8.00 equiv.) |
| 4 | Boc₂O (6.00 equiv.) | DIEA (12.00 equiv.) |

3.0 mmol resin afforded Intermediate 40 (2.46 g, crude) as a white solid. LCMS: RT=0.484 min, MS cal.: Mₐᵥ=823.97, [M+H]⁺=825.49, [M−Boc+H]⁺=724.37.

### Preparation of Intermediate 41:

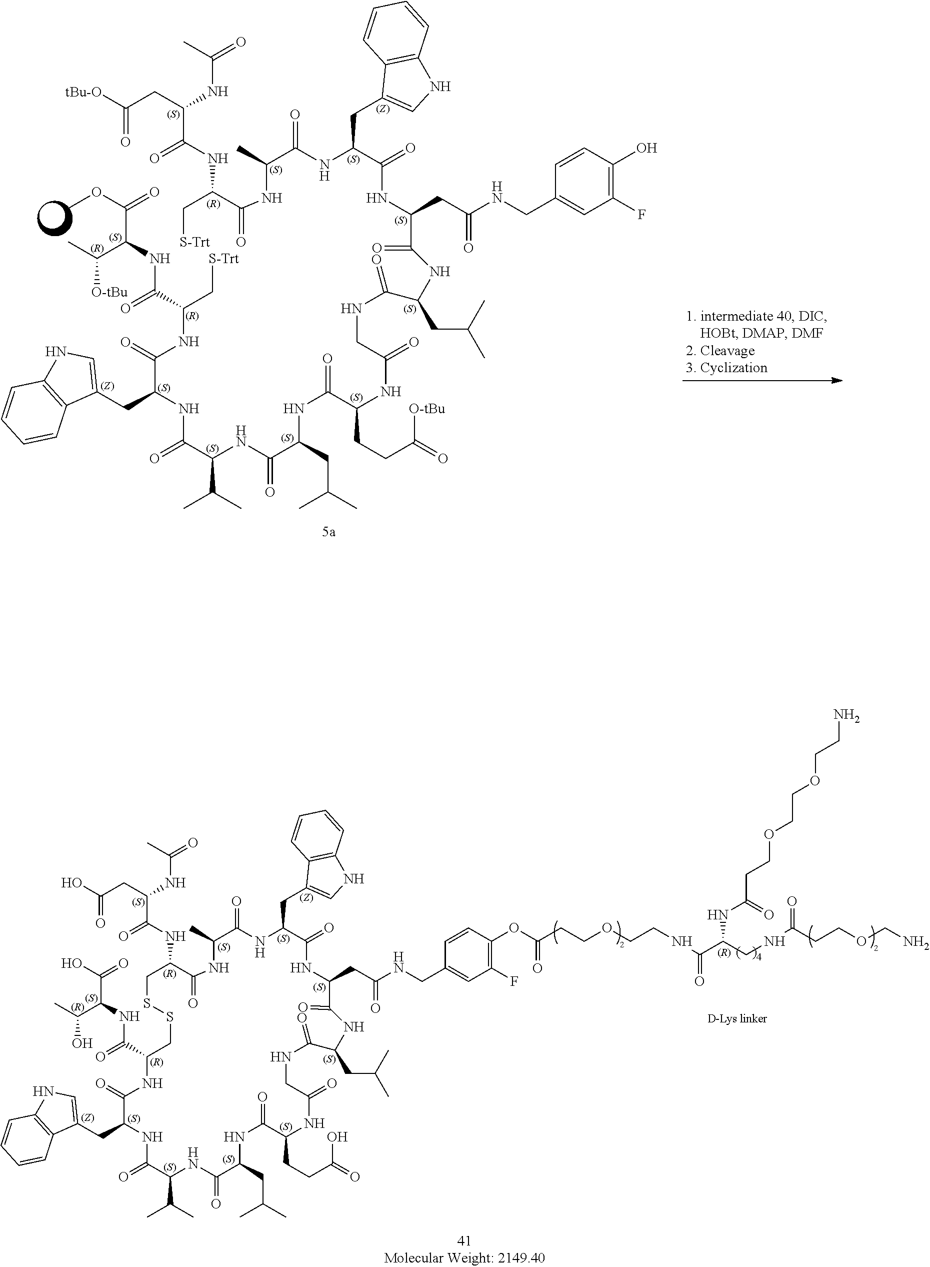

Intermediate 41 was synthesized by following the procedure mentioned in the synthesis of intermediate 5, with the treatment of Intermediate 40. 0.50 mmol CTC resin afforded Intermediate 41 (148.1 mg, 90.0% purity, 12.9% yield) as a white solid.

### Preparation of Compound 1574:

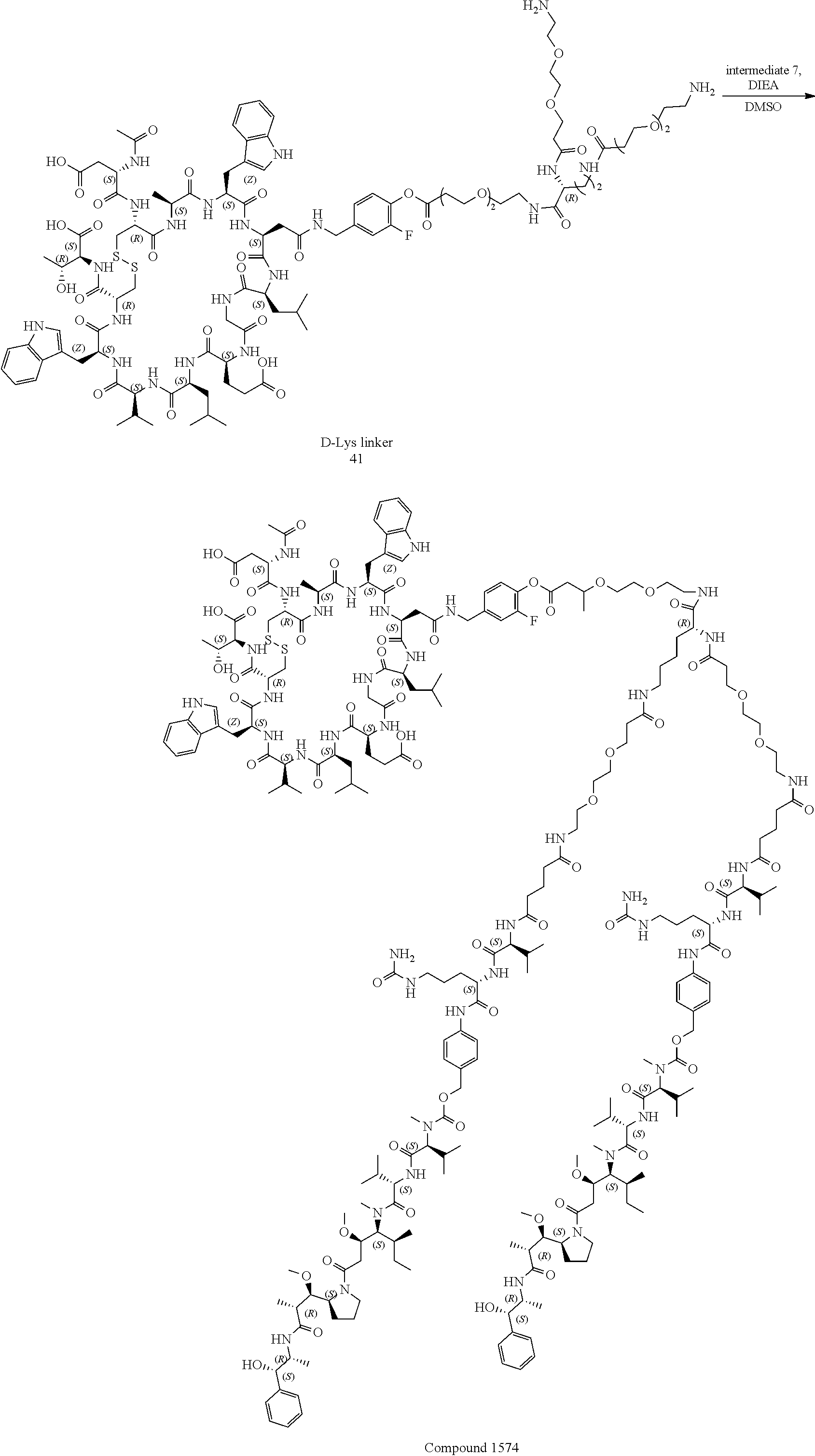

To a mixture of Intermediate 41 (50.00 mg, 23.58 μmol, 1.00 equiv.), DIEA (18.29 mg, 141.48 μmol, 24.64 μL, 6.00 equiv.) in DMSO (600 μL) was added Intermediate 7 (77.49 mg, 51.88 μmol, 2.20 equiv.) at 20° C. Then the mixture was stirred at 20° C. for 1 h. LCMS indicated reactant was consumed completely and one main peak was desired MS. The mixture was purified by prep-HPLC (TFA condition) to afford Compound 1574 (85.0 mg, 17.49 μmol, 74.1% yield, 97.1% purity) as a white solid. LCMS: RT=1.856 min, MS cal.: Mₐᵥ=4718.58, [M+3H]³⁺-1573.8, [M+4H]⁴⁺=1180.6, [M+5H]⁵⁺=944.7.

### Example 17. Procedure for Preparation of Compound 1575

### Preparation of Intermediate 42:

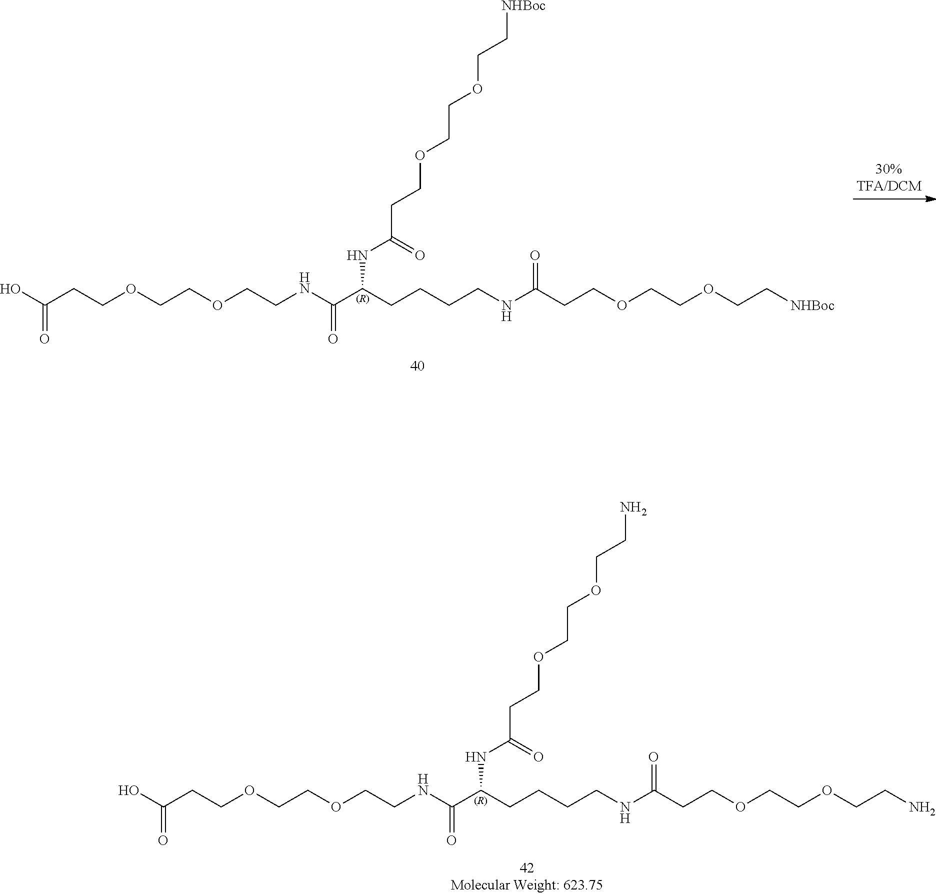

A mixture of Intermediate 40 (0.20 g) in TFA/DCM (3/7, v/v, 4 mL) was stirred at 20° C. for 0.5 h. The solvent was removed under reduced pressure. The residue was lyophilized to afford Intermediate 42 (0.20 g, crude, TFA salt) as colorless oil. LCMS: RT=0.637 min, MS calcd.: Mₐᵥ=623.74, [M+H]⁺=624.5.

### Preparation of Compound 1575:

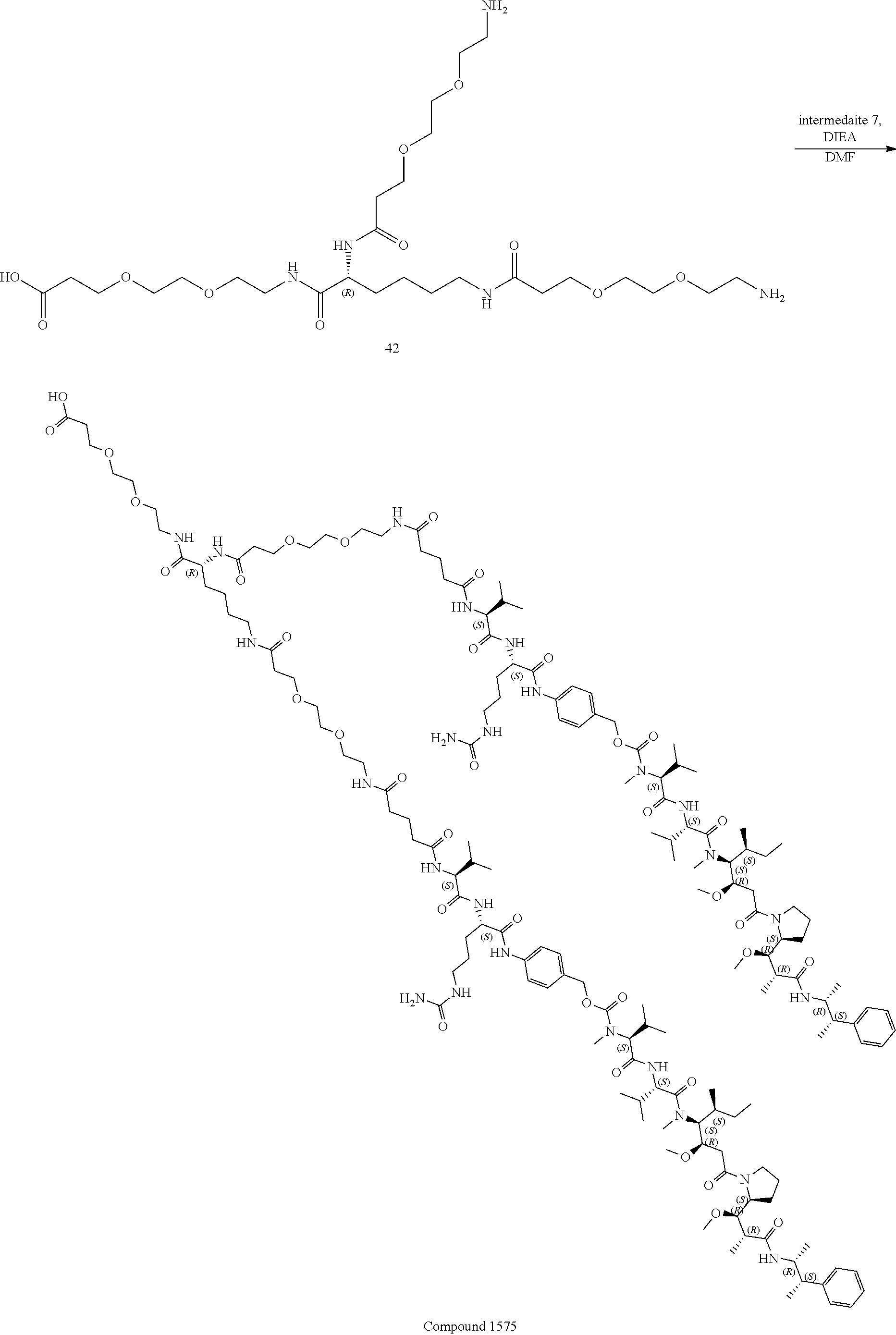

To a mixture of Intermediate 42 (10.00 mg, 16.03 μmol, 1.00 equiv.), DIEA (12.43 mg, 96.19 μmol, 16.76 μL, 6.00 equiv.) in DMSO (600 uL) was added Intermediate 7 (47.07 mg, 35.27 μmol, 2.20 equiv.) at 20° C. Then the mixture was stirred at 20° C. for 1 h. LCMS indicated reactant was consumed completely and one main peak was desired MS. The mixture was purified by prep-HPLC (TFA condition) to afford Compound 1575 (28.90 mg, 9.22 μmol, 57.5% yield, 97.7% purity) as a white solid. LCMS: RT=1.728 min, MS cal.: Mₐᵥ=3062.76, [M+2H]²⁺=1532.8, [M+3H]³⁺=1021.8, [M+4H]⁴⁺=766.7.

### Example 18. Synthesis of Linkers

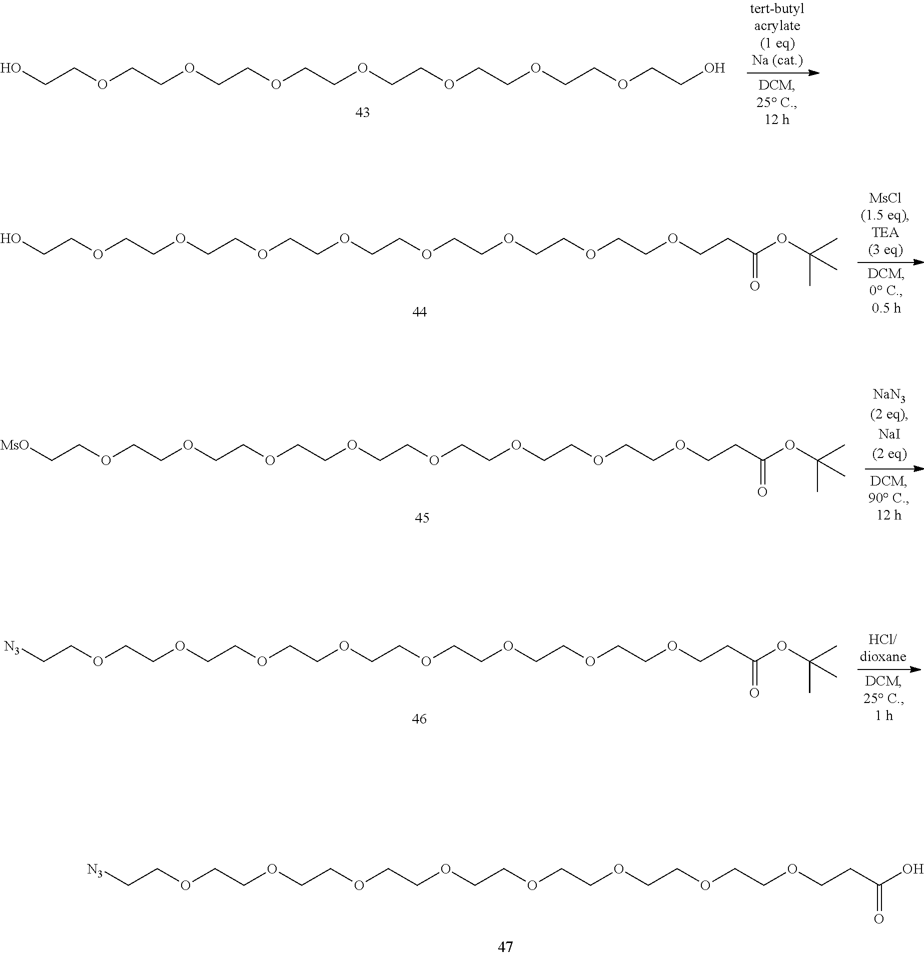 [SMILES: OCCOCCOCCOCCOCCOCCOCCOCCO ; CC(C)(C)OC(=O)CCOCCOCCOCCOCCOCCOCCOCCOCCO ; CC(C)(C)OC(=O)CCOCCOCCOCCOCCOCCOCCOCCOCCOS(C)(=O)=O ; CC(C)(C)OC(=O)CCOCCOCCOCCOCCOCCOCCOCCOCCN=[N+]=[N-] ; [N-]=[N+]=NCCOCCOCCOCCOCCOCCOCCOCCOCCC(=O)O]

### General Procedure for Preparation of Compound 44:

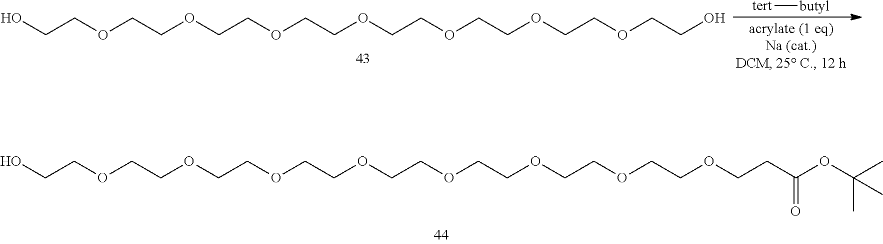

To a solution of compound 43 (16 g, 43.19 mmol, 1 eq) in DCM (70 mL) was added Na (29.79 mg, 1.30 mmol, 30.71 uL, 0.03 eq) and tert-butyl acrylate (5.54 g, 43.19 mmol, 6.27 mL, 1 eq). The mixture was stirred at 25° C. for 12 hr. TLC (Dichloromethane:Methanol=10:1 R_{f}=0.43) showed the reaction was complete. The reaction mixture was concentrated under reduced pressure to remove DCM. The residue was diluted with H₂O 100 mL and extracted with EtOAc (200 mL*3). The combined organic layers were washed with brine (300 mL*1), dried over Na₂SO₄, filtered and concentrated under reduced pressure to give a residue. Compound 44 (16 g, 35.20 mmol, 81.49% yield) was obtained as a yellow oil.

### General Procedure for Preparation of Compound 15:

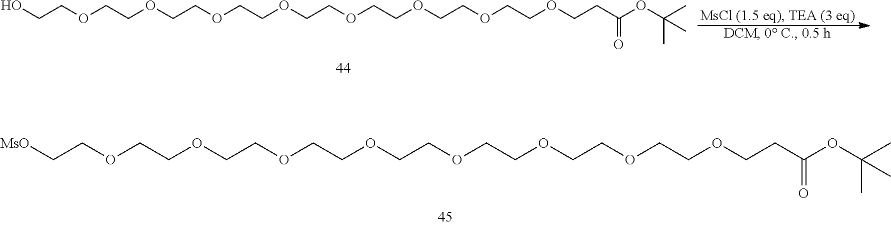 [SMILES: CC(C)(C)OC(=O)CCOCCOCCOCCOCCOCCOCCOCCOCCO ; CC(C)(C)OC(=O)CCOCCOCCOCCOCCOCCOCCOCCOCCOS(C)(=O)=O]

To a solution of compound 44 (17 g, 34.10 mmol, 1 eq) in DCM (130 mL) was added MsCl (5.86 g, 51.14 mmol, 3.96 mL, 1.5 eq) and TEA (10.35 g, 102.29 mmol, 14.24 mL, 3 eq). The mixture was stirred at 0° C. for 0.5 hr. TLC (Dichloromethane:Methanol=10:1 R_{f}=0.6) showed the reaction was complete. The residue was diluted with H₂O 300 mL and extracted with DCM (200 mL*2). The combined organic layers were washed with 0.5 M HCl (200 mL*2), dried over Na₂SO₄, filtered and concentrated under reduced pressure to give a residue. Compound 45 (19 g, 32.95 mmol, 96.63% yield) was obtained as a yellow oil.

### General Procedure for Preparation of Compound 46:

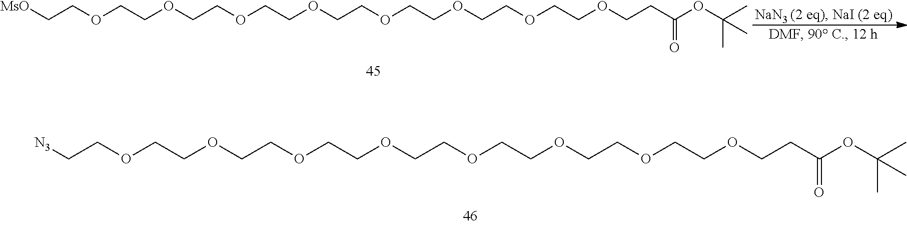 [SMILES: CC(C)(C)OC(=O)CCOCCOCCOCCOCCOCCOCCOCCOCCOS(C)(=O)=O ; CC(C)(C)OC(=O)CCOCCOCCOCCOCCOCCOCCOCCOCCN=[N+]=[N-]]

To a solution of compound 45 (19 g, 32.95 mmol, 1 eq) in DMF (190 mL) was added NaN₃ (4.28 g, 65.89 mmol, 2 eq) and NaI (9.88 g, 65.89 mmol, 2 eq). The mixture was stirred at 90° C. for 12 hr. TLC (Dichloromethane:Methanol=10:1 R_{f}=0.6) showed the reaction was complete. The residue was diluted with H₂O 500 mL and extracted with EtOAc (500 mL*3). The combined organic layers were washed with brine (500 mL*1), dried over Na₂SO₄, filtered and concentrated under reduced pressure to give a residue. Compound 46 (17 g, 32.47 mmol, 98.54% yield) was obtained as a yellow oil.

### General Procedure for Preparation of Compound 47:

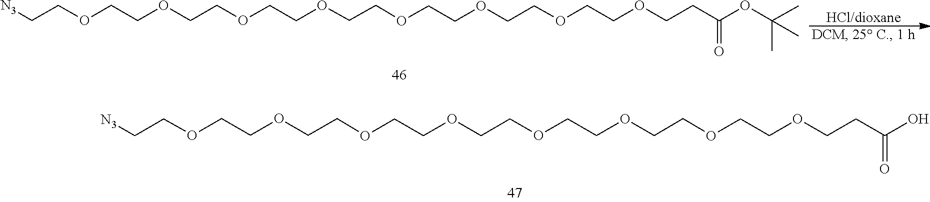 [SMILES: CC(C)(C)OC(=O)CCOCCOCCOCCOCCOCCOCCOCCOCCN=[N+]=[N-] ; [N-]=[N+]=NCCOCCOCCOCCOCCOCCOCCOCCOCCC(=O)O]

Three reactions were run in parallel. To a solution of compound 46 (6 g, 11.46 mmol, 1 eq) in DCM (120 mL) was added HCl/dioxane (4 M, 48.00 mL, 16.76 eq). The mixture was stirred at 25° C. for 0.5 hr. TLC (Dichloromethane:Methanol=10:1 R_{f}=0.1) showed the reaction was complete. The three reactions were worked up together. The reaction mixture was concentrated under reduced pressure to give a residue. The residue was purified by column chromatography (SiO₂, DCM:MeOH=200/1 to 1/1). Compound 7 (10 g, 21.39 mmol, 62.22% yield) was obtained as a yellow oil.

### Example 19. Synthesis of Additional Linkers

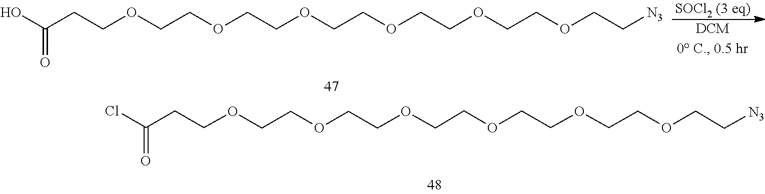 [SMILES: [N-]=[N+]=NCCOCCOCCOCCOCCOCCOCCC(=O)O ; [N-]=[N+]=NCCOCCOCCOCCOCCOCCOCCC(=O)Cl]

### Preparation of Compound 48:

A mixture of compound 47 (140 mg, 369.00 umol, 1 eq), SOCl₂ (131.70 mg, 1.11 mmol, 80.30 uL, 3 eq) in DCM (2 mL) at 0° C. was degassed and purged with N₂ 3 times, and then the mixture was stirred at 0-20° C. for 0.5 hr under N₂ atmosphere. TLC (Dichloromethane:Methanol=10:1 R_{f}=0.46) indicated compound 47 was consumed completely. The reaction mixture was concentrated to give the crude product. Compound 48 (146.81 mg, crude) was obtained as a yellow oil.

### Example 6. Preparation of Di-Fluorophenyl Reactive Group with Linker

### Preparation of Compound 50:

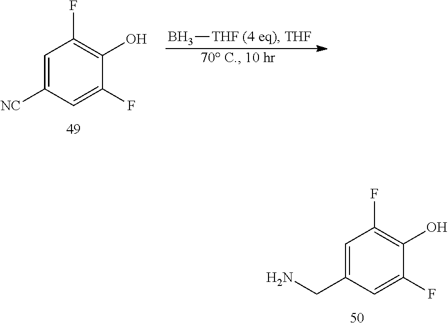

BH₃-THF (1 M, 25.79 mL, 4 eq) was added carefully to a solution of compound 49 (1 g, 6.45 mmol, 1 eq) in anhydrous THF (70 mL). The resultant solution was stirred and heated to reflux for 10 hr (70° C.). TLC (Petroleum ether:Ethyl acetate=1:1, R_{f}=0.01) indicated compound 49 was consumed completely and one new spot formed. After the mixture was cooled, 6 N HCl (2 mL) was carefully added to the solution, and heating was continued at reflux for 30 min. The mixture was concentrated under reduced pressure to give a residue. Compound 50 (2.5 g, crude, HCl) was obtained as a white solid.

### Procedure for Preparation of Compound 51:

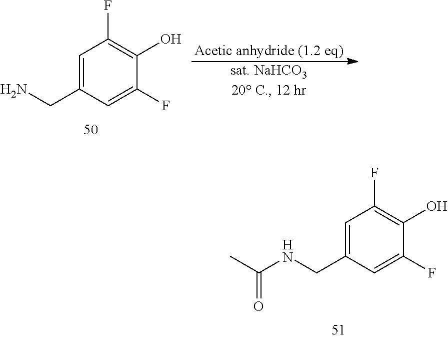

A mixture of compound 50 (270 mg, 690.20 umol, 1 eq, HCl), acetic anhydride (84.55 mg, 828.25 umol, 77.57 uL, 1.2 eq) in NaHCO₃ (5 mL) was degassed and purged with N₂ 3 times, and then the mixture was stirred at 20° C. for 24 hr under N₂ atmosphere. LCMS showed the starting material was consumed completely. TLC indicated compound 50 was consumed completely. The reaction mixture was acidified to pH 4-5 with 1 M HCl. The reaction mixture was extracted with EtOAc (30 mL*2). The combined organic layers were washed with brine (30 mL), dried over Na₂SO₄, filtered and concentrated under reduced pressure to give a residue. The residue was purified by column chromatography (SiO₂, Petroleum ether:Ethyl acetate=10:1 to 1:3). Compound 51 (67 mg, 333.05 umol, 48.25% yield) was obtained as a white solid. LCMS: RT=0.609 min, MS cal.: 201.0, [M+H]⁺=202.2. ¹H NMR (400 MHz, DMSO-d₆) δ ppm 9.99 (s, 1H) 8.28 (br s, 1H) 6.83-6.93 (m, 2H) 4.12 (d, J=5.95 Hz, 2H) 1.84 (s, 3H).

### Preparation of Compound 52:

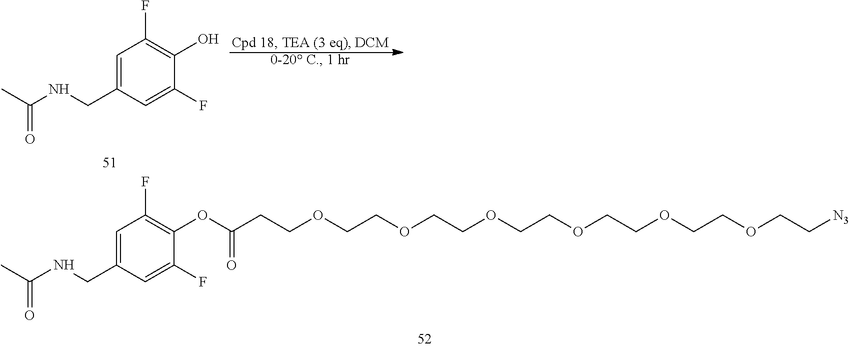 [SMILES: CC(=O)NCc1cc(F)c(O)c(F)c1 ; CC(=O)NCc1cc(F)c(OC(=O)CCOCCOCCOCCOCCOCCOCCN=[N+]=[N-])c(F)c1]

To a solution of compound 51 (67 mg, 333.05 umol, 1 eq) in DCM (1 mL) was added TEA (101.10 mg, 999.16 umol, 139.07 uL, 3 eq), and then compound 48 (145.76 mg, 366.36 umol, 1.1 eq) in DCM (1 mL) was added at 0° C. The resulting mixture was stirred at 20° C. for 2 hr. LCMS showed the starting material was consumed completely. The reaction mixture was filtered and the filtrate was concentrated. The crude product was purified by reversed-phase HPLC (column: Welch Ultimate AQ-C18 150*30 mm*5 um; mobile phase: [water (0.1% TFA)-ACN]; B %: 35%-65%, 12 min). Compound 52 (100 mg, 177.76 umol, 53.37% yield) was obtained as a yellow oil. LCMS: RT=2.129 min, MS cal.: 562.2, [M+H]⁺=563.3. ¹H NMR (400 MHz, CHLOROFORM-d) δ ppm 6.84 (d, J=7.95 Hz, 2H) 5.86 (br s, 1H) 4.33 (d, J=6.11 Hz, 2H) 3.81 (t, J=6.42 Hz, 2H) 3.53-3.64 (m, 23H) 3.32 (br t, J=5.01 Hz, 3H) 2.85 (t, J=6.36 Hz, 2H) 1.99 (s, 3H) 1.50 (s, 2H).

### Preparation of Compound 53:

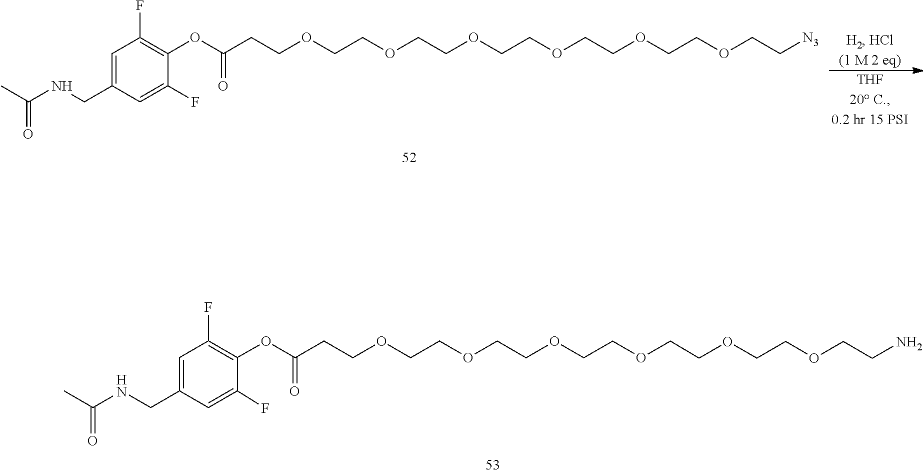 [SMILES: CC(=O)NCc1cc(F)c(OC(=O)CCOCCOCCOCCOCCOCCOCCN=[N+]=[N-])c(F)c1 ; CC(=O)NCc1cc(F)c(OC(=O)CCOCCOCCOCCOCCOCCOCCN)c(F)c1]

To a solution of compound 52 (100 mg, 177.76 umol, 1 eq) in THF (3 mL) was added HCl (0.5 M, 711.04 uL, 2 eq) and Pd/C (100 mg, 177.76 umol, 10% purity, 1.00 eq) under N₂. The suspension was degassed under vacuum and purged with H₂ several times. The mixture was stirred under H₂ (15 psi) at 20° C. for 0.2 hours. LCMS showed the starting material was consumed completely. The reaction mixture was filtered and the filtrate was concentrated. The crude product was purified by reversed-phase HPLC (column: Welch Ultimate AQ-C18 150*30 mm*5 um; mobile phase: [water (0.1% TFA)-ACN]; B %: 15%-45%, 12 min). Compound 53 (60 mg, 111.82 umol, 62.91% yield) was obtained as a white oil. LCMS: RT=1.272 min, MS cal.: 536.2, [M+H]⁺=537.3. ¹H NMR (400 MHz, CHLOROFORM-d) δ ppm 7.79 (br s, 3H) 6.98 (br d, J=8.19 Hz, 2H) 6.72 (br s, 1H) 4.42 (d, J=5.99 Hz, 2H) 3.88 (t, J=5.93 Hz, 2H) 3.80-3.85 (m, 2H) 3.72-3.76 (m, 2H) 3.65-3.71 (m, 12H) 3.13 (br s, 2H) 2.92 (t, J=5.87 Hz, 2H) 2.68 (br s, 4H) 2.08 (s, 3H).

### Example 20. Preparation of Fluoro-Phenyl Reactive Group with Linker

### Preparation of Compound 24:

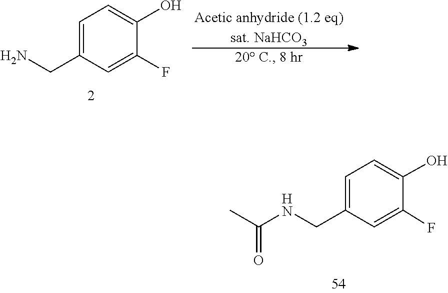

A mixture of compound 2 (1 g, 2.82 mmol, 1 eq, HCl), acetyl acetate (316.15 mg, 3.10 mmol, 290.04 uL, 1.1 eq) in NaHCO₃ (10 mL) was degassed and purged with N₂ 3 times, and the mixture was then stirred at 20° C. for 8 hr under N₂ atmosphere. LCMS showed the starting material was consumed completely. TLC indicated compound 2 was consumed completely. The reaction mixture was acidified to pH 4-5 with 1 M HCl. The reaction mixture was extracted with EtOAc (30 mL*2). The combined organic layers were washed with brine (30 mL), dried over Na₂SO₄, filtered and concentrated under reduced pressure to give a residue. The residue was purified by column chromatography (SiO₂, Petroleum ether:Ethyl acetate=20:1 to 1:2). Compound 54 (250 mg, 1.36 mmol, 48.48% yield) was obtained as a white solid. LCMS: RT=0.413 min, MS cal.: 183.0, [M+H]⁺=184.0. ¹H NMR (400 MHz, DMSO-d₆) δ ppm 9.76 (s, 1H) 8.32 (br s, 1H) 7.03-7.09 (m, 1H) 6.90-6.95 (m, 2H) 4.19 (d, J=5.87 Hz, 2H) 1.91 (s, 3H).

### Preparation of Compound 55:

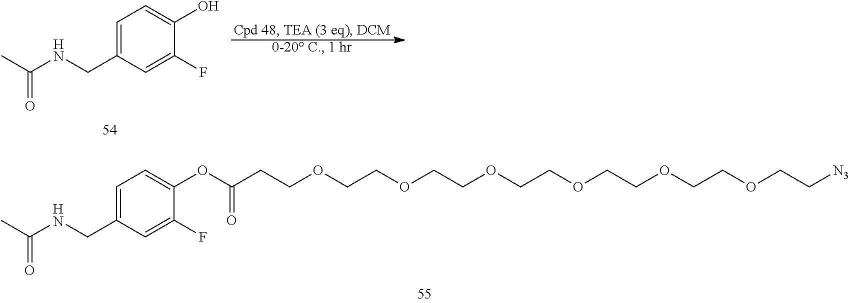 [SMILES: CC(=O)NCc1ccc(O)c(F)c1 ; CC(=O)NCc1ccc(OC(=O)CCOCCOCCOCCOCCOCCOCCN=[N+]=[N-])c(F)c1]

To a solution of compound (18 (Acid chloride PEG linker), 104 mg, 261.40 umol, 1 eq) in DCM (1 mL) was added TEA (79.35 mg, 784.20 umol, 109.15 uL, 3 eq) at 0° C. and then compound 54 (47.88 mg, 261.40 umol, 1 eq) in DCM (1 mL) was added at 0° C. The resulting mixture was stirred at 20° C. for 2 hr. LCMS showed formation of desired product. The reaction mixture was filtered and the filtrate was concentrated. The crude product was purified by reversed-phase HPLC (column: Welch Ultimate AQ-C18 150*30 mm*5 um; mobile phase: [water (0.1% TFA)-ACN]; B %: 25%-55%, 12 min). Compound 55 (82 mg, 150.58 umol, 57.60% yield) was obtained as a white oil. LCMS: RT=1.789 min, MS cal.: 544.2, [M+H]⁺=545.5. ¹H NMR (400 MHz, CHLOROFORM-d) δ ppm 7.04-7.17 (m, 3H) 5.92 (br s, 1H) 4.44 (d, J=5.87 Hz, 2H) 3.89 (t, J=6.36 Hz, 2H) 3.60-3.73 (m, 24H) 3.40 (br t, J=5.07 Hz, 3H) 2.90 (t, J=6.36 Hz, 2H) 2.44 (br s, 2H) 2.07 (s, 3H).

### Preparation of Compound 56:

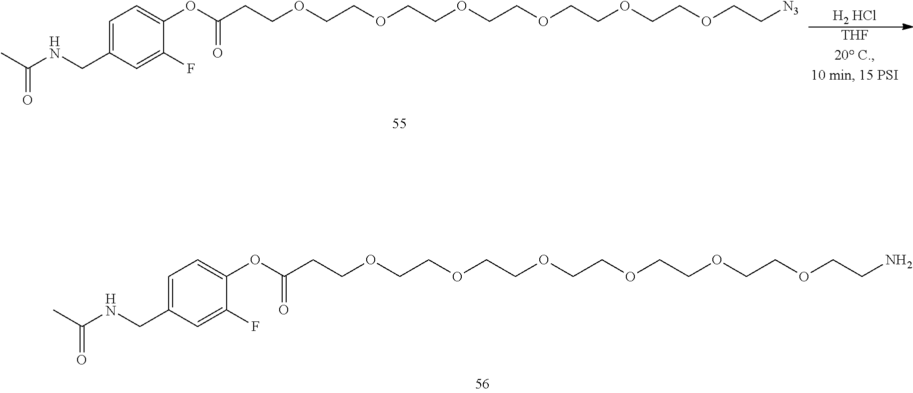 [SMILES: CC(=O)NCc1ccc(OC(=O)CCOCCOCCOCCOCCOCCOCCN=[N+]=[N-])c(F)c1 ; CC(=O)NCc1ccc(OC(=O)CCOCCOCCOCCOCCOCCOCCN)c(F)c1]

To a solution of compound 55 (82 mg, 150.58 umol, 1 eq) in THF (5 mL) was added HCl (1 M, 301.16 uL, 2 eq) and Pd/C (150.58 umol, 10% purity, 1 eq) under N₂. The suspension was degassed under vacuum and purged with H₂ several times. The mixture was stirred under H₂ (15 psi) at 20° C. for 10 min. LCMS showed the starting material was consumed completely. The reaction mixture was dried under nitrogen gas. The crude product was purified by reversed-phase HPLC (column: Welch Ultimate AQ-C18 150*30 mm*5 um; mobile phase: [water (0.1% TFA)-ACN]; B %: 12%-42%, 12 min). Compound 26 (4 mg, 7.71 umol, 5.12% yield) was obtained as a white oil. LCMS: RT=1.356 min, MS cal.: 518.2, [M+H]⁺=519.2. ¹H NMR (400 MHz, CHLOROFORM-d) δ ppm 7.94 (br s, 1H) 7.05-7.18 (m, 1H) 6.31 (br s, 1H) 4.43 (d, J=5.75 Hz, 1H) 3.78-3.91 (m, 2H) 3.56-3.77 (m, 10H) 3.11 (br s, 1H) 2.88 (t, J=5.93 Hz, 1H) 2.06 (s, 1H).

### Example 21. Antibody Drug Conjugates for Delivering MMAE

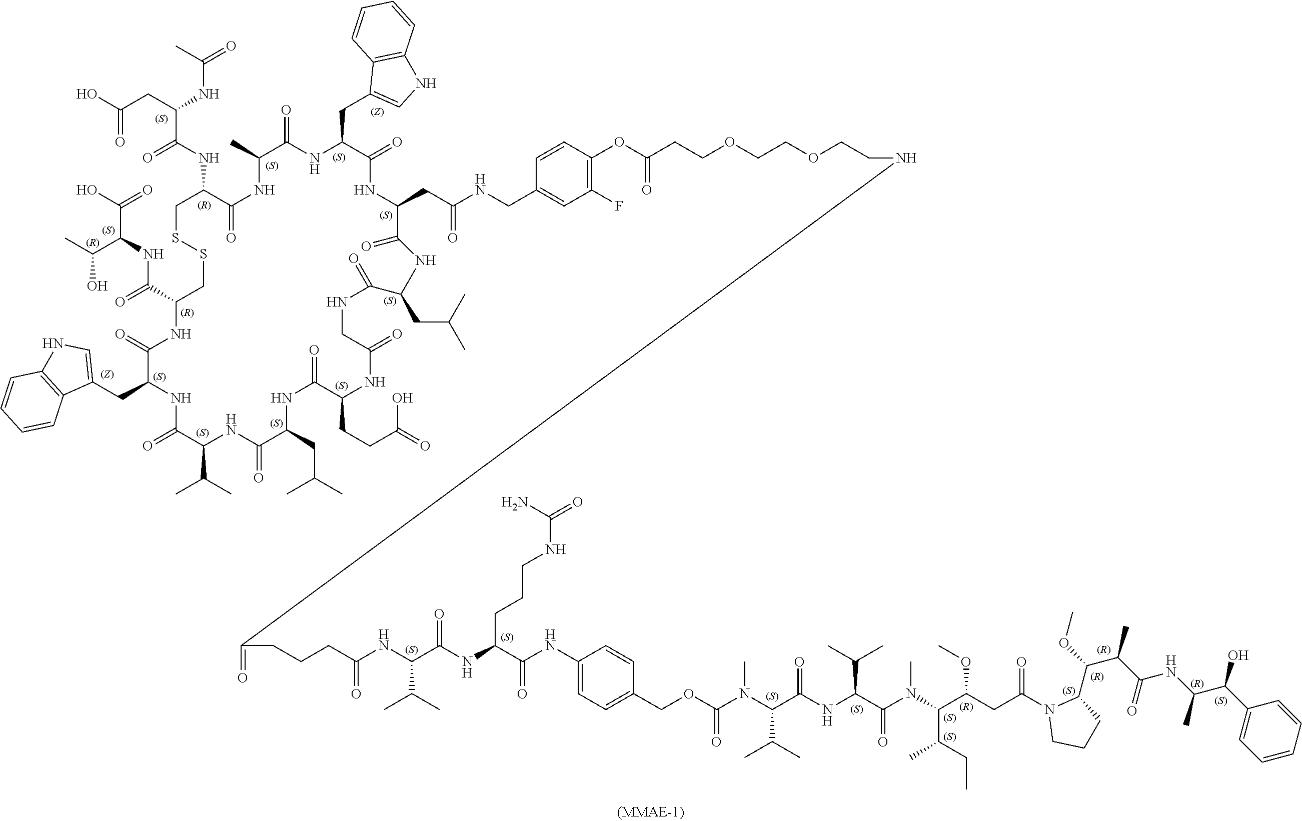

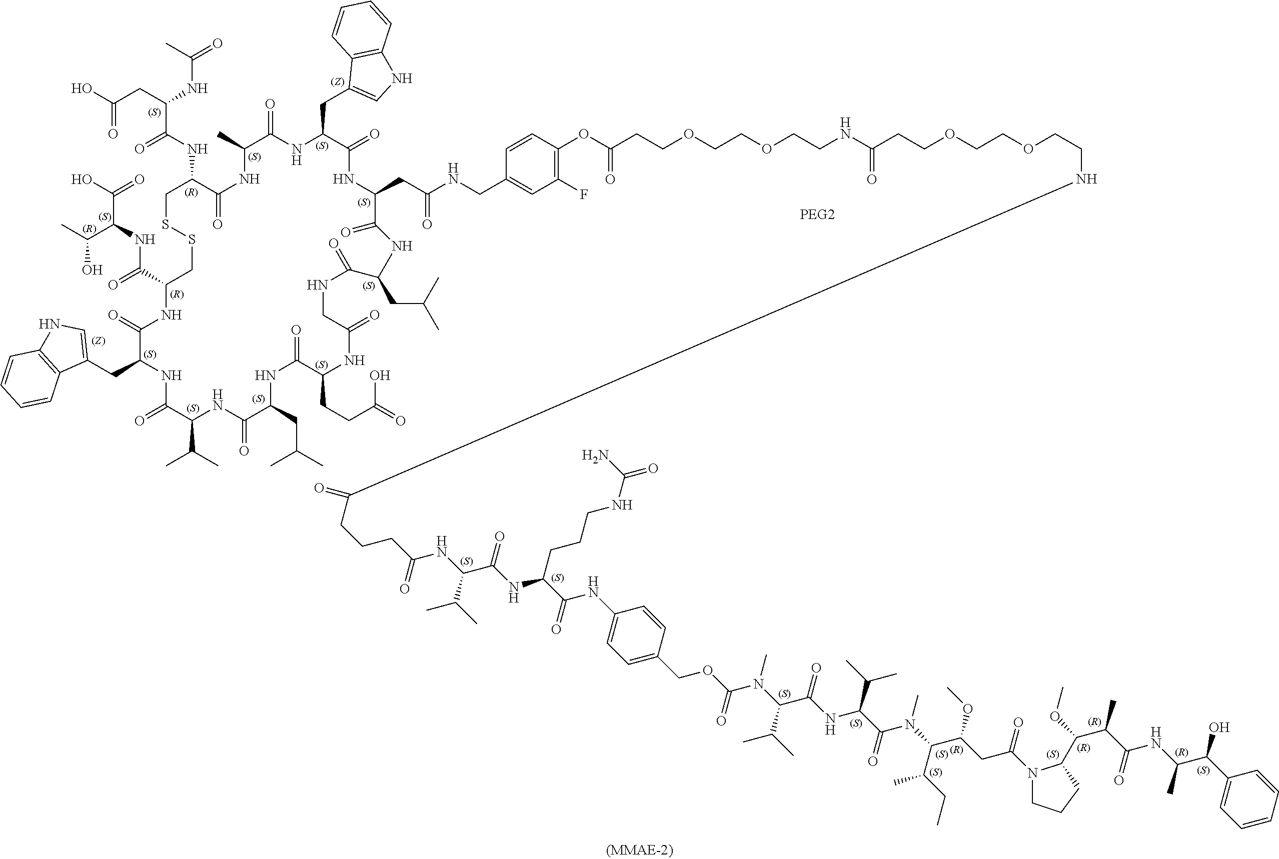

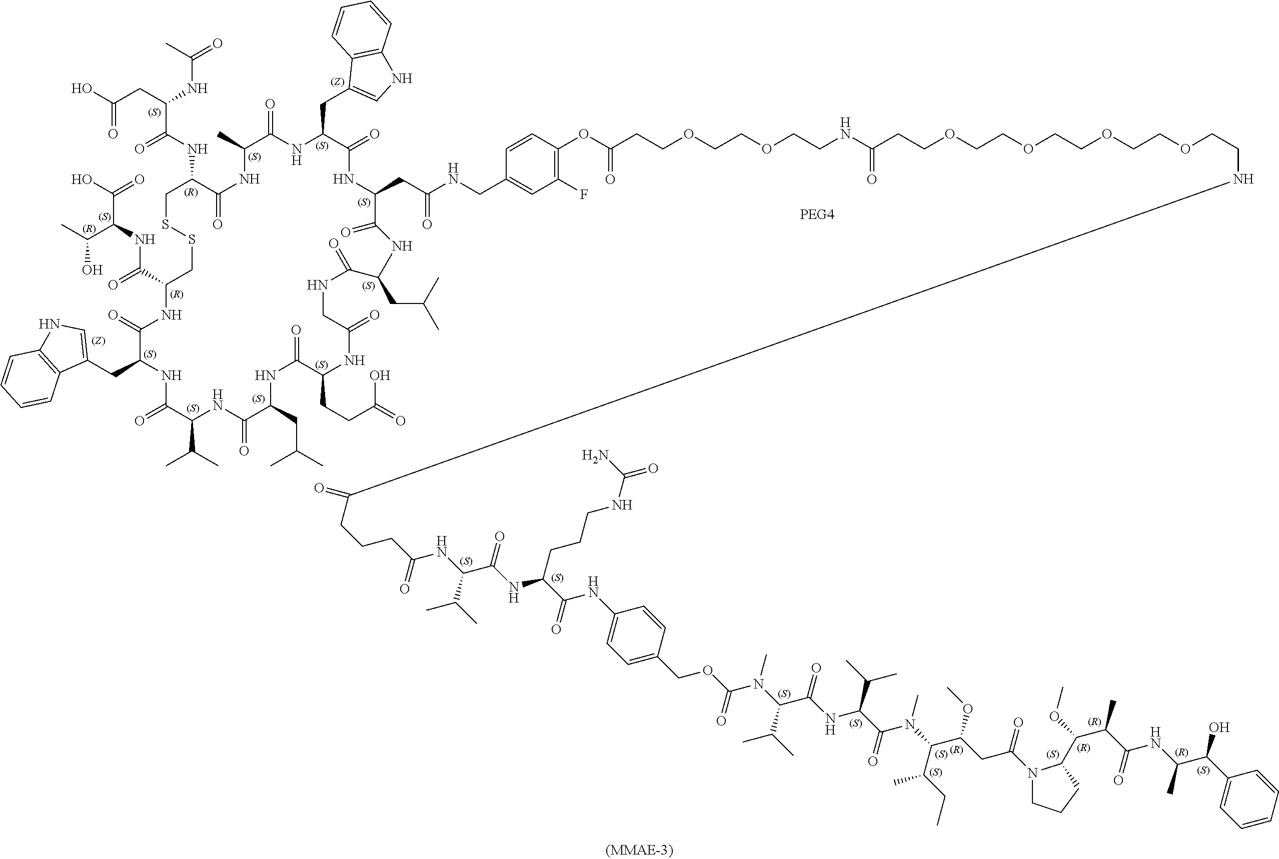

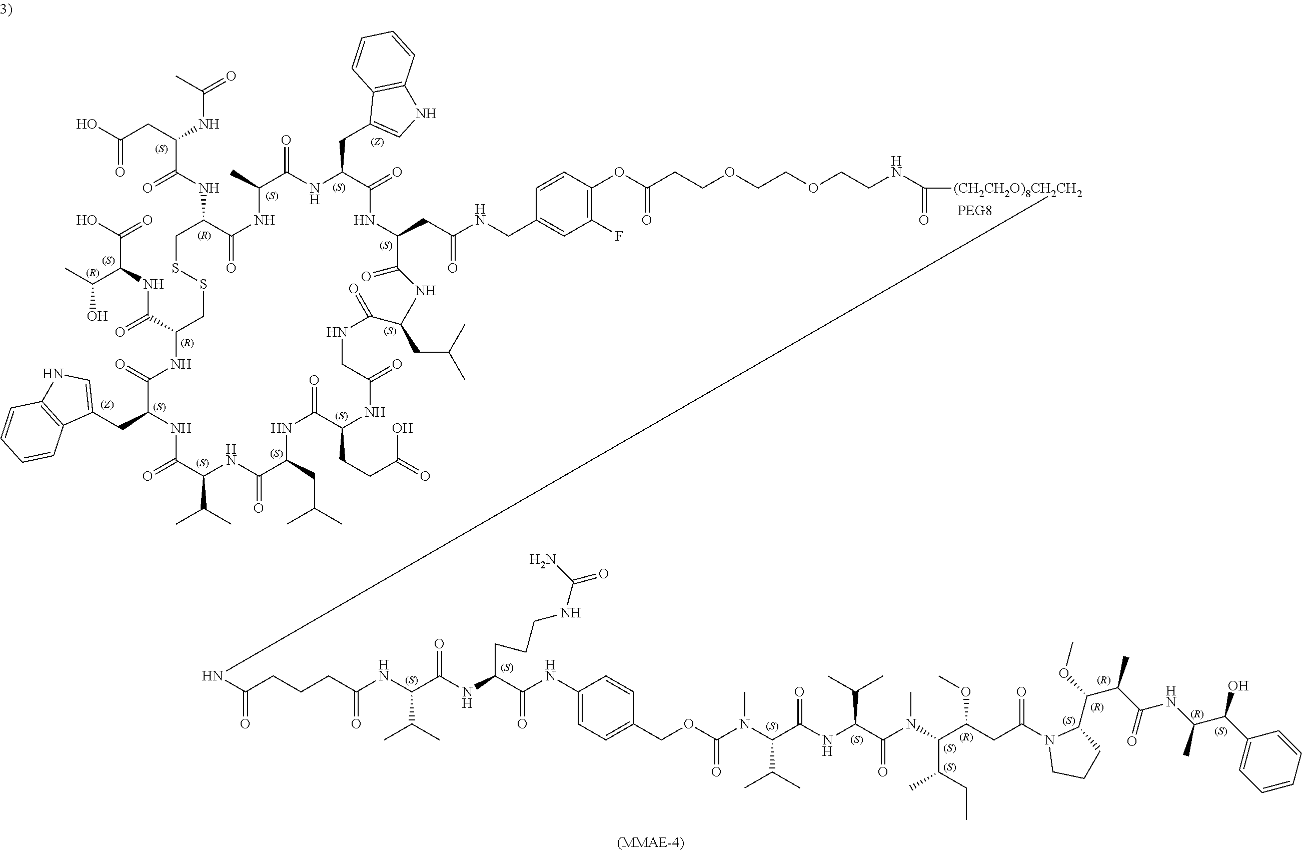

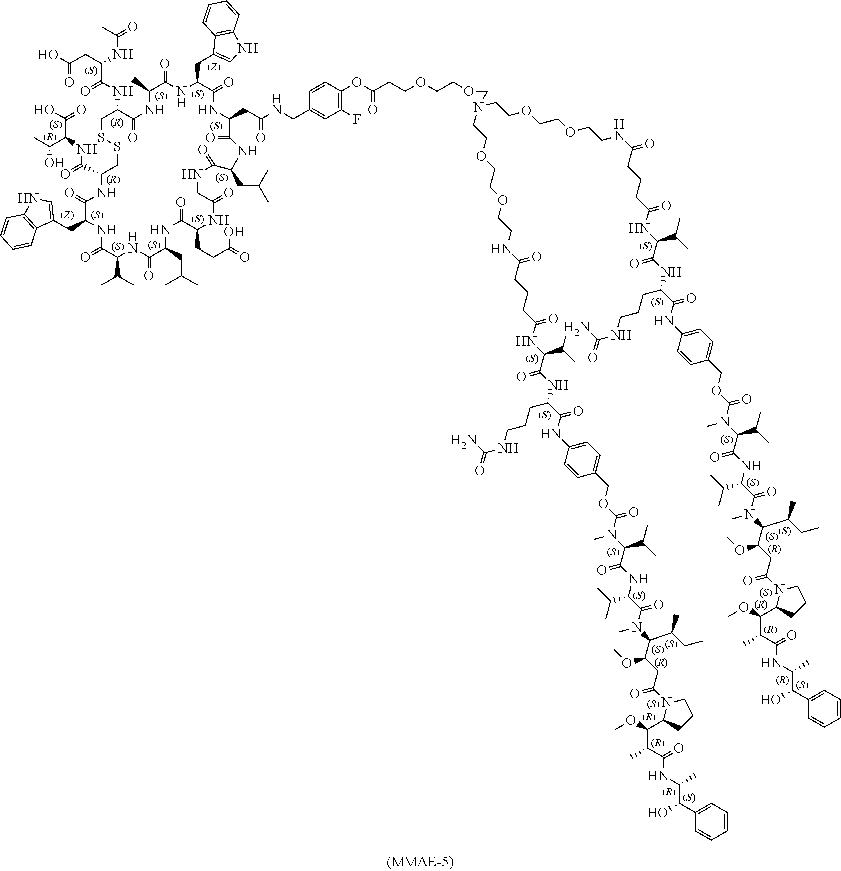

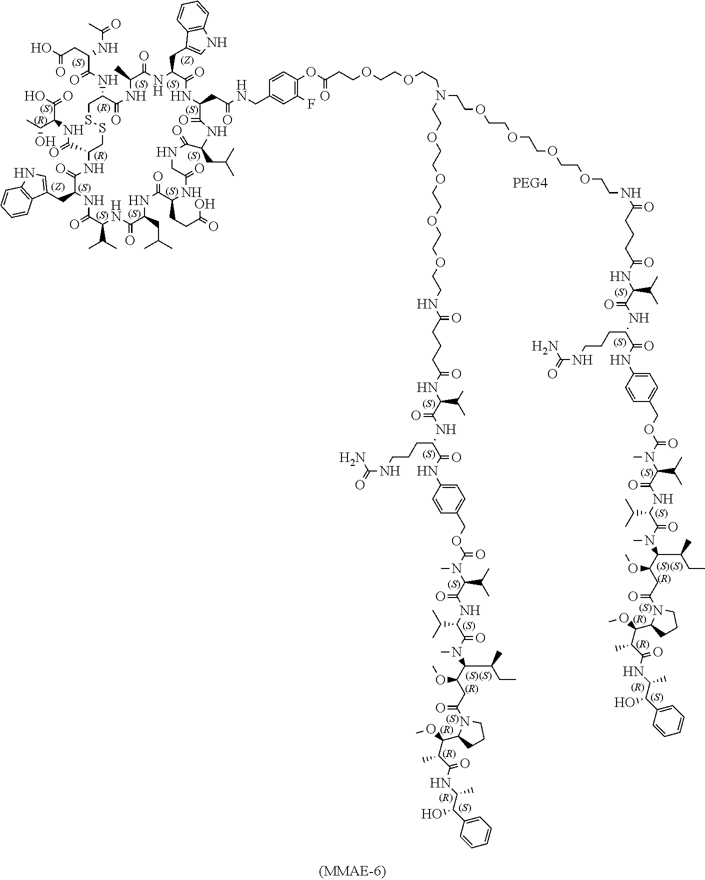

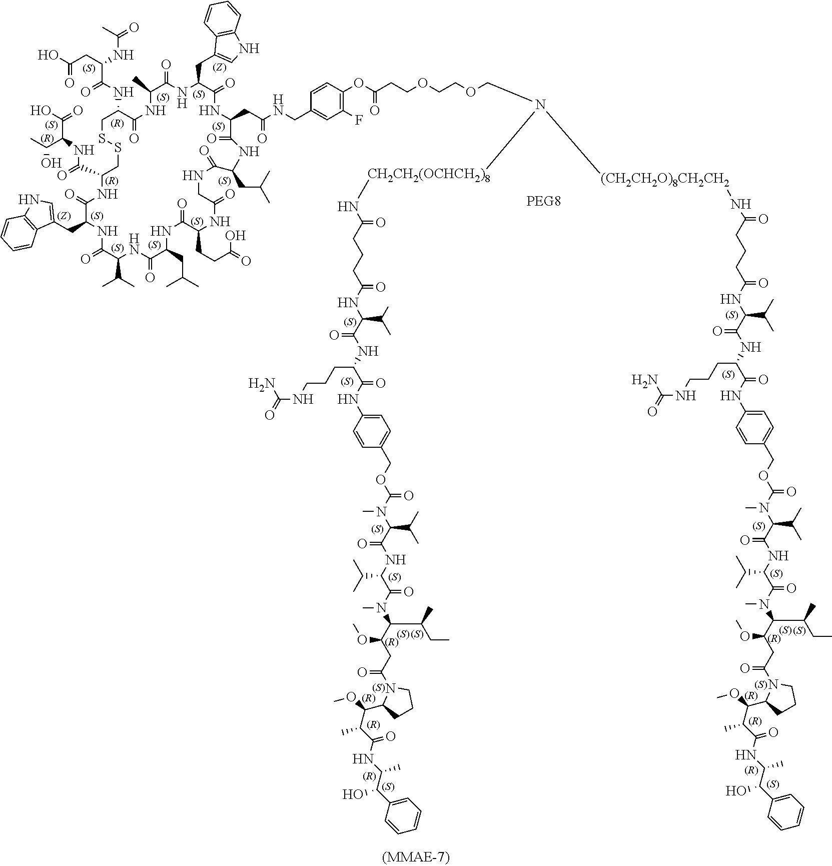

### Example 22. Method for Assessing Drug Antibody Ratio

Among other things, provided technologies can provide increased efficiency (e.g., higher rates and/or yields) and/or selectivity for conjugating a monomethyl auristatin, such as MMAE, to a target agent. Data from certain assessment are provided herein as examples.

In some embodiments, a target agent is a protein agent. In some embodiments, a target agent is an antibody agent. In some embodiments, the present disclosure provides technologies for conjugating moieties of interest to antibodies, e.g., brentuximab, enfortumab, etc.

In some embodiments, reaction partners, e.g., compounds of formula R—I or salts thereof, or more particularly MMAE-1-MMAE-7 as shown in Ex. 8 below, are dissolved in DMSO to 5 mM stock solution.

In some embodiments, reactions are set up with 300 micrograms of antibody. In some embodiments, various conditions, including various buffers, reagent equivalents, reaction time, reaction temperature and reaction concentrations can be utilized.

As an example, one reaction is 300 microliter reaction with 1 mg/mL of antibody in PBS. A reaction partner of the disclosure, such as MMAE-1, (1 microliter of 5 mM stock in DMSO, 2.5 molar equivalents relative to daratumumab) is diluted in 284 microliters of PBS buffer (10 mM phosphate, 150 mM sodium chloride, pH 7.4), then 15 microliters of anti-CD30 antibody, such as brentuximab, (20 mg/mL stock) are added to the reaction mixture followed by incubation at room temperature in the dark. After 4 h reaction buffer was exchanged using Amicon Ultra centrifuge filter (30 KDa MWCO, 0.5 mL volume). First, glycine buffer (100 mM, pH 2.1) is used for buffer exchange to ensure dissociation of target binding moieties after reaction. Then, phosphate buffer saline (pH 7.4) is used for further buffer exchange and storage.

In another example, a reaction is 300 microliter reaction with 1 mg/mL of antibody in borate buffer. Reaction partner (1.2 microliter of 5 mM stock in DMSO, 3.0 molar equivalents relative to MAB) is diluted in 284 microliters of Borate buffer (100 mM borate, pH 8.3), then 15 microliters of daratumumab (20 mg/mL stock) was added to the reaction mixture followed by incubation at room temperature in the dark. After 20 h reaction buffer is exchanged using Amicon Ultra centrifuge filter (30 KDa MWCO, 0.5 mL volume). First, glycine buffer (100 mM, pH 2.1) is used for buffer exchange to ensure dissociation of target binding moieties after reaction. Then, phosphate buffer saline (10 mM phosphate, 150 mM sodium chloride, pH 7.4) is used for further buffer exchange and storage.

Various technologies can be utilized for assessment of reaction results in accordance with the present disclosure.

Provided technologies, among other things, can provide increased conjugation efficiency and selectivity without requiring extra reaction steps. In some embodiments, provided technologies can selectively conjugate desired moieties of interest at selective residue(s) of antibody agents. Among other things, technologies of the present disclosure can provide agents with improved properties and/or activities (e.g., improved purity, homogeneity, etc.) with high efficiency.

In some embodiments, a useful technology is absorbance based DAR analysis. DAR (drug antibody ration, the ratio of moieties of interest and target agent moieties (e.g., antibody agent moieties) can be calculated for various antibody conjugates, e.g., in various reagent screening/assessment methods. Various agents comprising target binding moieties are assessed for conjugation efficiency as reaction partners with targets, e.g. protein agents such as antibody agents, compared to reagents with the same reactive groups but without target binding moieties. In various ratio determination ‘Drug’/moiety of interest is fluorescein isothiocyanate (FITC) dye conjugated to target agents, e.g., antibody agents. DAR molar ratio is defined as a ratio of moles of drug/moiety of interest to moles of target agent/antibody. Molarity is calculated from absorbance of FITC (A₄₈₅) and antibody (A₂₈₀) of conjugated product, and extinction coefficients of FITC and antibody using Beer-Lambert law. Correction coefficient 0.35 is used to correct for absorbance of FITC at 280 nm. Biotek Synergy H1 microplate reader and Take3 microvolume plate are used for absorbance measurements. Concentration of antibody should be at least 3 mg/mL for optimal signal-to-noise in the readings.

### Example 23. Techniques to Determine Antibody Conjugation Sites

### E. Provided Technologies Provide Significantly Improved Selectivity.

Among other things, provided technologies can provide significantly improved selectivity with respect to conjugation sites when target agents have multiple sites available for conjugations. For example, as demonstrated herein, under various conditions various provided technologies selectively conjugate on certain chains of antibody agents, and/or selective residues of antibody agents.

In some embodiments, western blot is utilized to assess antibody conjugation locations (e.g., heavy chain, light chain, etc.). Certain data were presented in the Figures. As shown, technologies of the present disclosure can provide various levels of selectivity. In some embodiments, various technologies provide selectivity for heavy chains over light chains.

In some embodiments, for western blot, samples are first run on NuPage denaturing gel (e.g., Invitrogen, NP0321). Samples were loaded in amount of 50 ng per well. After band separation the gel is transferred on nitrocellulose membrane (Invitrogen, 1B23002) using iBlot. The membrane is blocked with 5% dry milk in PBST buffer (PBS pH 7.4 with 0.1% Tween 20). In some embodiments, for detection of fluorescein conjugated light and heavy chains, primary antibody is mouse anti-fluorescein antibody (EMD Millipore, MAB045) in 1:2500 dilution, and secondary antibody is goat anti-mouse IgG conjugated with HRP (Southern Biotech, 1038-05) in 1:20000 dilution. Detection reagent for antibodies on the nitrocellulose membrane is done using SuperSignal West Femto Chemiluminescent Substrate (Thermo Fisher, 34096). The membrane is imaged on Azure Biosystems c500 for chemiluminescent signal.

In some embodiments, technologies for assessing provided technologies are or comprise mass spectrometry optionally with chromatography technologies (e.g., HPLC, UPLC, etc.). For example, various product agents were assessed by mass spectrometry, e.g., in some embodiments, using Sciex X500 QTOF system equipped with Agilent ZORBAX RRHD (300SB—C8, 2.1×50 mm, 1.8 um) column. In some embodiments, liquid chromatography is utilized together with MS. In one example: mobile phase buffers were A=0.1% Formic acid in water, B=acetonitrile. Protocol conditions were 0-1 min, 2% B; 1-7 min, 2-40% B; 7-7.5 min, 40-80% B; 7.5-9 min, 80% B; 9-9.5 min, 80-2% B; 9.5-10.5 min, 2% B; flow rate is 0.25 mL/min; concentration of the conjugates is 0.1 mg/min; injection volume is 0.01 mL. In some embodiments, BioTool kit is used for intact mass analysis. In some embodiments, mass range is 147,000-155,000 and m/z 2200-3400.

In some embodiments, peptide mapping analysis is utilized for assessment of provided technologies. In some embodiments, conjugated and unconjugated antibody is digested into peptides using trypsin, and peptides comprising conjugation were quantified by ion mass. In some embodiments, trypsin digestion are performed as below:
1. Aliquot 25-50 mcg of total protein sample into a clean protein lo-bind Eppendorf tube.
2. Exchange sample buffer to Smart digest buffer using 7 kDa MWCO gel filtration columns and protocol provided by Thermo Scientific.
3. Add any necessary Smart digest buffer to the buffer exchanged sample to achieve a final volume of 100 mcl.
4. Add 5 mcL of Smart Trypsin solution to the buffer exchanged sample.
5. Digest protein for 15 minutes at 70° C. in a dry bath (Add water to well to ensure proper thermal transfer to sample).
6. Remove sample from bath and allow to cool to room temperature.
7. Add 1 mcL of TCEP Bond Breaker solution to the protein sample.
8. Incubate at room temperature for 30 minutes (away from light).
9. Add 10 mcL of 5% aqueous TFA to the sample to acidify and vortex.
10. Spin down the sample for 3 minutes in a bench top centrifuge at 12,000 rcf.
11. Transfer the sample to a clean autosampler tube, careful to not disturb any undigested protein pellet.

In some embodiments, instrument conditions for analysis is:
LC: Waters Acquity I-Class UPLC
   Mobile phases: A: 0.05% aqueous TFA; B: 0.05% TFA in acetonitrile
   Column: ACQUITY UPLC Peptide BEH C18 Column, 300 Å, 1.7 μm, 2.1 mm×100 mm
   Gradient: Hold 2% B for the 1^{st} minute; 2-65% B over 1-60 minutes
MS: Thermo LTQ Orbitrap Velos Pro MS1, parent ions, resolution of 30000 at 400 Da; range: 300-2000 Da, used a lock mass to ensure accuracy within 5 ppm
Data-dependent method with a 20000 total ion count threshold to trigger fragmentation of the parent ion. Collision energy of 35 eV (standard collision energy for peptide mapping)

In some embodiments, conjugation selectively occurs at K246/K248 of antibody heavy chains. In some embodiments, conjugation sites include K246 of heavy chains. In some embodiments, conjugation sites include K248 of heavy chains. In some embodiments, conjugation sites include K288/K290 of heavy chains. In some embodiments, conjugation sites include K288 of heavy chains. In some embodiments, conjugation sites include K290 of heavy chains. In some embodiments, conjugation sites include K185 of light chains. In some embodiments, conjugation sites include K187 of light chains. In some embodiments, conjugation sites include K414 of heavy chains.

Additional data confirmed that provided technologies can provide efficient and/or selective conjugation to various types of antibody agents (e.g., monoclonal antibody agents, polyclonal antibody agents, pooled antibody agents such as IVIG, IgG1, IgG2, IgG3, and/or IgG4 antibody agents, etc.). Those skilled in the art reading the present disclosure will appreciate that various types of antibodies can also be conjugated with high efficiency and/or selectivity in accordance with the present disclosure (e.g., using compounds and methods that comprise suitable target binding moieties for such antibodies, and various reactive moieties and optionally linker moieties as described herein). A useful protocol for peptide mapping is described below as an example; those skilled in the art that other protocols, including various modifications and variations of the protocol described below, may also be utilized in accordance with the present disclosure:
1. Quantify proteins, e.g., with Pierce 660 reagent.
2. In low-bind Eppendorf tubes dilute 10 μg of sample in 100 μL of Tris 50 mM pH8.0.
3. Reduce proteins by adding 10 mM DTT (dithiothreitol) for 15 minutes at 60° C. in a block heater.
4. Add 15 mM iodoacetamide for alkylation at room temperature for 30 minutes in the dark.
5. Quench the reaction by adding 10 mM DTT.
6. Digest proteins with 0.33 μg of α-chymotrypsin (Sigma) over night at 37° C. in a thermoshaker.
7. Acidify samples with 2 μL of 100% formic acid.
8. Purify peptides on a Strata-X reversed phase SPE (Phenomenex). Peptide were eluted with 60% acetonitrile with 2% formic acid.
9. Dry eluted peptides under nitrogen stream.
10. Reconstitute peptides in 25 μL of mobile phase A.
11. Dilute peptides 1:10 in mobile phase A before injection on LC-MS, e.g., according to the parameters below.

Instrument for analysis as example:
LC: Eksigent microLC200 (Sciex)
Mobile phases: A: 0.2% formic acid and 3% DMSO in water; B: 0.2% formic acid and 3% DMSO in ethanol
Column: Luna Omega PS column 0.3 mm i.d., 3 μm particles, 100 mm (Phenomenex)
Gradient: 2-48% B over 25 minutes at 6 ul/minutes flow rate.
MS: ABSciex TripleTOF 6600+
MS1 (range 350-1250 Da), resolution 35000
DDA method with a 500 cps threshold.

As described herein, provided technologies can provide highly efficient and/or selective (e.g., with respect to conjugation sites) conjugation for various types of antibody agents (e.g., monoclonal antibody agents, polyclonal antibody agents, or pooled antibody agents such as IVIG). Among other things, the present disclosure provides data confirming that technologies of the present disclosure can provide highly efficient and/or selective conjugation of IgG2 and IgG4 antibodies. In some embodiments, reactions were performed in borate buffer pH 8.2, 2.5 M eq of reagent to antibody, 20 h, 25° C. Those skilled in the art reading the present disclosure will appreciate that other types of antibodies can also be conjugated with high efficiency and/or selectivity in accordance with the present disclosure (e.g., using compounds and methods that comprise suitable target binding moieties for such antibodies, and various reactive moieties and optionally linker moieties as described herein).

### Example 24. Provided Product Agents Maintain Properties and Functions of Target Agents

Among other things, provided technologies utilize mild conditions, short pathways (e.g., no separate removal of target binding moieties), etc., and provide conjugation at directed sites and product agents that maintain one or more or all desired properties and/or activities of target agents (e.g., antibody agents). Provided agents comprising antibody agent moieties can maintain interactions with Fc receptors (e.g., FcRn).

Various technologies are useful for assess properties and/or activities of target agents (e.g., antibody agents). For example, in some embodiments, ELISA assay can be utilized to assess binding between provided agents and FcRn receptor. For example, a high binding 96-well plate (e.g., Costar 3922) is coated with neutravidin (Thermo Fisher, 31000) in PBS buffer (pH 7.4), blocked with 5% bovine serum albumin in PBST buffer pH 7.4 (PBS buffer pH 7.4 with 0.05% tween 20), followed by immobilization of Avi-tagged FcRn protein (Acro Biosystems, FCM-H82W4) in PBST buffer pH 6.0. After washing with PBST pH 6.0, antibody (e.g., brentuximab, etc.) and its conjugates are bound to FcRn on the plate in PBST pH 6.0. All bound antibodies and conjugates were detected in PBST pH 6.0 using anti-human F(ab)2 antibody conjugated with HRP. Detection reagent was SuperSignal ELISA Pico Chemiluminescent Substrate (Thermo fisher, 37069) followed by luminescence read on Biotek Synergy H1 microplate reader.

### Example 25. Provided Technologies Provide Efficient Reactions and Removal of Target Binding Moieties

Among other things, the present disclosure provides technologies for removing an agent comprising a target binding moiety (e.g., a reaction product comprising a target binding moiety released after a reaction) from a reaction product (e.g., a product comprising an antibody moiety or a fragment thereof). In some embodiments, a method comprises contacting a composition comprising an agent comprising a target binding moiety and a reaction product wherein the target binding moiety interacts with the reaction product, with an acidic solution. In some embodiments, after contact with an acidic solution, an agent comprising a target binding moiety is separated from a reaction product. In some embodiments, pH of a solution is about 1, 2, 3, or 4. In some embodiments, pH is 1. In some embodiments, pH is 2. In some embodiments, pH is 3. In some embodiments, pH is 4. As confirmed in FIG. 23, agents comprising released target binding moiety from a reaction between 1-44 and daratumumab can be effectively removed, e.g., at pH 2. A protocol is described below as an example.

In some embodiments, mass spectrometry analysis of methanol-precipitated antibody conjugates was utilized for assessment of antibody conjugates from provided technologies. In some embodiments, buffers of different pH were utilized to remove bound leaving group from antibody (e.g., antibody conjugate products) after conjugation. In some embodiments, methanol precipitation was performed as below:
1. Combine one volume of purified antibody conjugate and 3 volumes of methanol.
2. Incubate sample at 4° C. for 1 hour.
3. Centrifuge at 15,500×g for 10 min at 4° C.
4. Recover supernatant and dry in speed vac.
5. Re-suspend in 0.1% aqueous formic acid to 30 uL.

In some embodiments, instrument conditions for analysis was:
LC: ExionLC
Mobile phases: A: 0.1% aqueous formic acid; B: 0.1% formic acid in 95% acetonitrile
Column: Phenomenex Luna C18(2) column (100×2, 3 μm, 100 Å)
Gradient: Hold 5% B for the 1^{st} minute; 5-50% B over 1-7 minutes
MS: Sciex X500B QTOF system
Calibration done with positive calibrant using CDS system. ESI voltage of 5.5 kV, ion source gas 1 and 2 at 40 psi, curtain gas 30 (arbitrary unit), CAD gas 7 (arbitrary unit). Source temperature 350° C., DP 100V, accumulation time 0.25 sec, CE OV. TOF-MS full scan from m/z 300 to m/z 5000 in profile mode.
Sciex OS 1.4 used for acquisition.

### Example 26. Conjugation Method

The desired antibodies (A=brentuximab and B=enfortumab) were buffer exchanged for over 8 dilution volumes (DVs) with 50 mM HEPES buffer, pH 7.5. The target antibody concentration after buffer exchange was >14 mg/mL. A 10 mM stock solution in DMSO was then prepared for each conjugation reagent. Conjugations of the appropriate reagent to the antibody (A or B) was performed using 4 equivalents of reagent at a target antibody concentration of 10 mg/mL in 50 mM HEPES buffer (pH 7.5) with 20% (v/v) DMSO at 25° C. from 1-7 days. The conjugation reactions were analyzed for drug to antibody ratio (DAR) by LC-MS every 24 hrs. When the DAR reached >1.9 (for linear reagents) or >3.8 (for branched reagents), buffer exchange by UFDF over 30 DVs to PBS, pH7.4 was performed to a final concentration range of approximately 5-9 mg/mL. The ADCs were then analyzed for quality under various conditions. The results are listed in Table 5.

TABLE 5
Conjugation Results

| Antibody/ | Start | Agg. | | Yield | Amt. | Conc. | Endo | Resid. | Resid. | Resid. |
|---|---|---|---|---|---|---|---|---|---|---|
| cmp. | (mg) | % | DAR | % | % | mg/mL | (EU/mg) | uABT | Reag. | Payload |
| A-cmp. | 40 | 2.67 | 1.99 | 74.58 | 29.83 | 7.87 | 0.234 | <2.516 | <2.908 | <3.164 |
| 1100 | | | | | | | | | | |
| B-cmp. | 40 | 1.73 | 1.99 | 66.05 | 26.42 | 8.81 | <0.091 | <2.515 | <2.907 | <3.163 |
| 1100 | | | | | | | | | | |
| A-cmp. | 31.4 | 0.73 | 1.98 | 73.34 | 23.03 | 6.45 | 0.471 | 0.834 | <0.309 | <1.524 |
| 1101 | | | | | | | | | | |
| B-cmp. | 40 | 1.12 | 2.04 | 75.13 | 30.05 | 6.94 | <0.086 | <2.516 | <2.907 | <2.907 |
| 1101 | | | | | | | | | | |
| A-cmp. | 40 | 1.29 | 1.99 | 86.49 | 34.59 | 8.28 | <0.097 | <2.516 | <1.184 | <3.164 |
| 1102 | | | | | | | | | | |
| B-cmp. | 42 | 0.97 | 2.00 | 75.5 | 31.71 | 6.97 | <0.095 | <2.515 | <1.183 | <3.163 |
| 1102 | | | | | | | | | | |
| A-cmp. | 42 | 1.26 | 1.97 | 80.12 | 33.65 | 8.03 | 0.139 | <2.516 | 2.456 | <3.164 |
| 1103 | | | | | | | | | | |
| B-cmp. | 42 | 0.83 | 2.01 | 77.19 | 32.42 | 7.11 | <0.098 | <4.191 | 3.601 | <3.163 |
| 1103 | | | | | | | | | | |
| A-cmp. | 40 | 2.76 | 4.01 | 81.80 | 32.72 | 6.54 | <0.183 | <1.480 | 0.880 | <0.700 |
| 1574 | | | | | | | | | | |
| B-cmp. | 40 | 1.23 | 3.92 | 76.22 | 30.49 | 6.63 | <0.181 | <1.479 | <0.699 | <0.699 |
| 1574 | | | | | | | | | | |
| A-cmp. | 49 | 0.61 | 3.68 | 41.47 | 20.32 | 5.70 | <0.140 | <2.516 | <2.908 | <3.164 |
| 1105 | | | | | | | | | | |
| B-cmp. | 38 | 0.60 | 3.62 | 68.21 | 25.92 | 5.89 | <0.102 | <2.515 | <2.907 | <3.163 |
| 1105 | | | | | | | | | | |
| A-cmp. | 38 | 0.70 | 3.87 | 72.95 | 27.72 | 6.32 | 1.165 | <2.516 | <2.908 | <3.164 |
| 1106 | | | | | | | | | | |
| B-cmp. | 38 | 1.68 | 3.85 | 83.18 | 31.61 | 8.93 | <0.112 | <2.515 | <2.907 | <3.163 |
| 1106 | | | | | | | | | | |
| A-cmp. | 40 | 2.56 | 2.01 | 68.65 | 27.46 | 7.04 | <0.085 | <4.194 | <2.908 | <2.908 |
| 1199 | | | | | | | | | | |
| B-cmp. | 40 | 0.74 | 2.04 | 71.53 | 28.60 | 7.15 | 0.260 | <2.515 | <2.907 | <2.907 |
| 1199 | | | | | | | | | | |

Start = Starting amounts of antibody (mg);
Resid. uABT = Residual universal Antibody Binding Terminus (uABT) (% mol/mol of mAb);
Resid. Reag. = Residual Reagent (% mol/mol of total reagent);
Resid. Payload = Residual Payload (% mol/mol of total payload)

### Example 27. Representative Example for Residual Payload Analysis

Residual payload, the amount of unconjugated MATE reagent after loss of directing group (“uABT”), but not conjugated with the antibody following a conjugation reaction, can be quantitated as follows. Solvent Buffer I is prepared by adding 2 g NaCl to the premixed organic solvent (6 mL MeOH and 10 mL CAN). The buffer is mixed and stirred for at least 1 hour, the buffer was allowed to stand for at least 1 hour before use. The supernatant was the saturated sodium chloride solution. Standard Buffer II is prepared by mixing 250 μL of DMSO, 700 μL of PBS, 1000 μL of 6 mg/mL Herceptin in PBS. Add PBS and DMSO are added to the sample to 3 mg/mL (e.g. cmp. 1199 or 1101) or 5 mg/mL with 15% (v/v) DMSO. Then mix 100 uL sample solution with 150 μL solvent I (sample: precipitant 1:1.5 v/v). Vortex solution for 10 minutes at room temperature. Centrifuge solution for 10 minutes at 16,000 rcf at room temperature. Remove the supernatant immediately into a glass vial for analysis. Payload standard stock solution (10 mM) is stored at −80° C. until used. Dilute the reference standard to 1000 μM with DMSO. Add 10 μL of the 1000 μM uABT to 390 μL of buffer 11 for a final concentration of 25 μM. The standard samples are prepared as shown in Table 6.

Take 200 μL of each standard sample and add 300 ul solvent buffer I, to generate the standards for the final standard curve (10 μM, 5 μM, 2 μM 1 μM, 0.5 μM, 0.2 μM, 0.1 μM). Vortex solution for 10 minutes at room temperature. Centrifuge solution for 10 minutes at 16,000 rcf at room temperature. Remove the supernatant immediately into a glass vial for analysis.

TABLE 6
Preparation of Samples for Standard Curve, Residual Payload Analysis

| Standard | Volume of | Volume of | Total | Final Conc. |
|---|---|---|---|---|
| Conc. (μM) | Stock (μL) | buffer II (μL) | Volume (μL) | (μM) |
| 25 | 200 | 200 | 400 | 12.5 |
| 12.5 | 200 | 200 | 400 | 5 |
| 5 | 200 | 200 | 400 | 2.5 |
| 2.5 | 160 | 240 | 400 | 1.25 |
| 1.25 | 200 | 200 | 400 | 0.5 |
| 0.5 | 120 | 80 | 200 | 0.25 |

A standard curve for payload analysis is shown in FIG. 4A. An HPLC trace useful for payload analysis and peak areas for compound 1101 are shown in FIG. 5. The data was collected using a Luna Omega 1.6 m Polar C18 100 A column, where Mobile Phase A is 0.1% TFA in H₂O and Mobile Phase B is 0.1% TFA in ACN.

The HPLC conditions for the standard curve and residual payload analysis are shown in Table 7.

TABLE 7
HPLC Conditions for Residual Payload Analysis

| Column: | Phenomenex, Luna Omega 1.6 μm Polar | | |
|---|---|---|---|
| | C18 100A | | |
| Detection Wavelength: | 280 nm BW 4, Ref = 360 nm BW 4 | | |
| Column Oven Temp.: | 30° C. | | |
| Sampler Temp.: | 6 ± 2° C. | | |
| Flow Rate: | 0.2 mL/min | | |
| Stop Time: | 45 min | | |
| Maximum Pressure: | 600 bar | | |
| Injection Amount: | 100 uL | | |
| Mobile Phases: | A: 0.1% TFA in water | | |
| | B: 0.1% TFA in ACN | | |
| | Time (min) | A (%) | B (%) |
| Gradient Program: | 0.0 | 70 | 30 |
| | 30.0 | 40 | 60 |
| | 31.0 | 10 | 90 |
| | 33.0 | 10 | 90 |
| | 35.0 | 70 | 30 |
| | 45.0 | 70 | 30 |

The residual payload is quantitated via Formulae below.

$$\begin{matrix}\% mol / mol = \frac{\frac{{C}_{free Payload}}{{MW}_{Payload}}}{\frac{{C}_{free Payload}}{{MW}_{Payload}} + DAR \times \frac{{C}_{protein}}{{MW}_{Protein}}} \times 10 \\ {C}_{free Payload} = \frac{\frac{{Area}_{STD}}{Injection {volume}_{STD}}}{\frac{{Area}_{residual}}{Injection {volume}_{residual}}} \times {C}_{STD}\end{matrix}$$

where STD=The standard samples closest to the area of the test sample, Cᵣₑₛidᵤₐₗ=concentration of the free payload (μmol/L), and Cₚᵣₒₜₑᵢₙ=concentration of protein (μmol/L).

### Example 28. Residual Reagent Analysis

Residual reagent analysis is performed is similar to residual payload analysis. Solvent buffer I is prepared by adding 2 g NaCl to the premixed organic solvent of 6 mL MeOH and 10 mL CAN. Buffer I is mixed and stirred for at least 1 hour at least, and the solution to is allowed stand for at least 1 hour before use, the supernatant was the saturated sodium chloride solution. Solvent buffer II is prepared by mixing 250 μL of DMSO, 700 μL of PBS, and 1000 μL of 6 mg/mL Herceptin. Samples are prepared by adding PBS and DMSO to sample to 3 mg/mL with 15% (v/v) DMSO. Then mixing 100 μL of the 3 mg/mL solution with 150 μL solvent I (sample: precipitant 1:1.5 v/v). Vortex solution for 10 minutes at room temperature. The solution is centrifuged for 10 minutes at 16,000 rcf at room temperature. The supernatant is removed immediately into a glass vial for analysis. Reagent Reference Standards are composed of reagent standard stock solution (10 mM). Standards are stored at Stored at −80° C. until used. Reference standards are diluted to 1000 μM with DMSO. 10 μL of the 1000 μM uABT are added to 390 μL of buffer II for a final concentration of 25 μM. Samples for the Reagent standard curve are prepared according to Table 8.

To create the final standard curve Take 200 μL of each standard samples and add 300 ul solvent buffer I for the final standard curve points (10 μM, 5 μM, 2 μM 1 μM, 0.5 μM, 0.2 μM, 0.1 μM). Vortex solution for 10 minutes at room temperature. Centrifuge solution for 10 minutes at 16,000 rcf at room temperature. Remove the supernatant immediately into a glass vial for analysis. The HPLC parameters for the residual reagent analysis are the same as for the residual payload analysis in the preceding example, with the exception of the gradient. The HPLC gradient for the residual reagent analysis is provided in Table 9. A standard curve for residual reagent analysis is shown in FIG. 4B. An HPLC trace for residual reagent analysis and peak areas for compound 1101 are shown in FIG. 6.

TABLE 8
Preparation of Samples for Standard Curve, Residual Reagent Analysis

| Standard | Volume of | Volume of | Total | Final Conc. |
|---|---|---|---|---|
| Conc. (μM) | Stock (μL) | buffer II (μL) | Volume (μL) | (μM) |
| 25 | 200 | 200 | 400 | 12.5 |
| 12.5 | 200 | 200 | 400 | 5 |
| 5 | 200 | 200 | 400 | 2.5 |
| 2.5 | 160 | 240 | 400 | 1.25 |
| 1.25 | 200 | 200 | 400 | 0.5 |
| 0.5 | 120 | 80 | 200 | 0.25 |

TABLE 9
HPLC Conditions for Residual Reagent Analysis

| Mobile Phases: | A: 0.1% TFA in water | | |
|---|---|---|---|
| | B: 0.1% TFA in CAN | | |
| | Time (min) | A (%) | B (%) |
| Gradient Program: | 0.0 | 70 | 30 |
| | 30.0 | 40 | 60 |
| | 31.0 | 10 | 90 |
| | 33.0 | 10 | 90 |
| | 35.0 | 70 | 30 |
| | 45.0 | 70 | 30 |

Residual reagent is calculated using the following Formulae.

$$\begin{matrix}\% mol / mol = \frac{\frac{{C}_{free Reagent}}{{MW}_{Reagent}}}{\frac{{C}_{free Reagent}}{{MW}_{Reagent}} + DAR \times \frac{{C}_{protein}}{{MW}_{Protein}}} \times 10 \\ {C}_{Residual reagent} = \frac{\frac{{Area}_{STD}}{Injection {volume}_{STD}}}{\frac{{Area}_{residual}}{Injection {volume}_{residual}}} \times {C}_{STD}\end{matrix}$$

In the residual reagent equations STD=The standard sample closest to the area of the test sample, C_{residual}=concentration of free reagent (μmol/L), and Cₚᵣₒₜₑᵢₙ=concentration of protein reagent (μmol/L).

### Example 29. Residual uABT Analysis for all Antibody Drug Conjugates (ADS)

Solvent buffers I and II are the same as buffers I and II for the residual reagent analysis. Samples are prepared by adding PBS and DMSO to make the sample at 3 mg/mL with 15% (v/v) DMSO. Then 100 μL of the 3 mg/mL solution is mixed with 150 μL solvent I and vortexed for 10 minutes at room temperature. The solution is centrifuged for 10 minutes at 16,000 rcf at room temperature. The supernatant is removed immediately into a glass vial for analysis. uABT standard stock solution (10 mM) is prepared and stored at −80° C. until used. The reference standard stock solution is diluted to 1400 μM with DMSO. For the reference standards 10 μL of the 1400 μM uABT to 390 μL of buffer II for a final concentration at 35 μM. Table 10 shows the composition of the uABT Analysis standard samples. uABT samples are prepared by combining 200 μL of each standard samples and 300 ul solvent buffer I to provide a final standard curve (14 μM, 7 μM, 3.5 μM 1.75 μM, 0.7 μM, 0.35 μM, 0.21 μM). The samples solution is vortexed for 10 minutes at room temperature and centrifuged for 10 minutes at 16,000 rcf at room temperature. The supernatant is analyzed immediately. HPLC parameters for the residual uABT analysis are the same as for the residual payload analysis given in example 7.

TABLE 10
uABT Standard Samples

| Standard | Volume of | Volume of | Total | Final Conc. |
|---|---|---|---|---|
| Conc. (μM) | Stock (μL) | buffer II (μL) | Volume (μL) | (μM) |
| 35 | 200 | 200 | 400 | 17.5 |
| 17.5 | 200 | 200 | 400 | 8.75 |
| 8.75 | 200 | 200 | 400 | 4.38 |
| 4.38 | 160 | 240 | 400 | 1.75 |
| 1.75 | 200 | 200 | 400 | 0.88 |
| 0.88 | 120 | 80 | 200 | 0.53 |

Residual uABT is calculated using the following Formulae.

$$\begin{matrix}\% mol / mol = \frac{{C}_{Residual uABT}}{{C}_{protein}} \times 10 \\ {C}_{Residual uABT} = \frac{\frac{{Area}_{STD}}{Injection {volume}_{STD}}}{\frac{{Area}_{residual}}{Injection {volume}_{residual}}} \times {C}_{STD}\end{matrix}$$

In the above uABT formulae, STD is the standard sample closest to the area of the test sample, C_{residual} is the concentration of free uABT (μmol/L) and Cₚᵣₒₜₑᵢₙ is the concentration of protein (μmol/L). A standard curve for residual uABT analysis is shown in FIG. 4C. An HPLC trace for residual uABT analysis and peak areas are shown in FIG. 7.

### Example 30. DAR Analysis Methods

The HPLC and MS parameters for the DAR analysis are given in Tables 11 and 12 respectively

TABLE 11
HPLC Parameters for DAR Analysis

| Column: | Agilent, PLRP-S, 150 * 2.1 mm, 8 μm | | |
|---|---|---|---|
| Detection Wavelength: | 280 nm BW 4 | | |
| Column Oven Temp.: | 80° C. | | |
| Sampler Temp.: | 6 ± 2° C. | | |
| Flow Rate: | 0.5 mL/min | | |
| Stop Time: | 8 min | | |
| Maximum Pressure: | 600 bar | | |
| Injection Amount: | 2 μg | | |
| Mobile Phases: | A: 0.05% TFA, 0.05% FA in water | | |
| | B: 0.05% TFA, 0.05% FA in ACN | | |
| | Time (min) | A (%) | B (%) |
| Gradient Program: | 0.0 | 75 | 25 |
| | 0.7 | 70 | 30 |
| | 4.0 | 40 | 60 |
| | 4.1 | 10 | 90 |
| | 5.5 | 10 | 90 |
| | 5.6 | 75 | 25 |
| | 8.0 | 75 | 25 |

TABLE 12
MS Parameters for DAR Analysis

| lon Source | Dual ESI | |
|---|---|---|
| Ion Polarity | Positive | |
| Gas Temp | 350° | C. |
| Drying Gas | 13 | L/min |
| Nebulizer | 45 | psig |
| Vcap | 5000 | V |
| Mass Min Range | 500 | m/z |
| Mass Max Range | 8000 | m/z |

The following formula is used for DAR analysis.

$$DAR = \frac{1 \times {Abundance}_{{DAR}_{+}} \times 2 \times {Abundance}_{DAR 2} + … + n \times {Abundance}_{DARn}}{{Abundance}_{{DAR}_{+}} + … + Abundance DARn}$$

While a number of embodiments have been described, it is apparent that our basic examples may be altered to provide other embodiments that utilize technologies (e.g., compounds, agents, compositions, methods, etc.) of the present disclosure.

1. A compound having the structure of formula R-I:
LG-RG-L^{RM}-MOI (R-I)
or a salt thereof, wherein:
LG is R^{LG}-L^{LG};
R^{LG} is 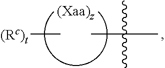
R^{c}-(Xaa)z-, a nucleic acid moiety, or a small molecule moiety;
each Xaa is independently a residue of an amino acid or an amino acid analog;
t is 0-50;
z is 1-50;
each R^{c} is independently -L^{a}-R′;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
L^{LG} is -L^{LG1}-, L^{LG1}-L^{LG2}, -L^{LG1}-L^{LG2}-L^{LG3}- or -L^{LG1}-L^{LG2}-L^{LG3}-L^{LG4}-;
RG is -L^{RG1}-L^{RG2}-, -L^{LG4}-L^{RG1}-L^{RG2}-, -L^{LG3}-L^{LG4}-L^{RG1}-L^{RG2}-, -L^{LG2}-L^{LG3}-L^{LG4}-L^{RG1}-L^{RG2}-;
each of L^{LG1}, L^{LG2}, L^{LG3}, L^{LG4}, L^{RG1}, L^{RG2}, and L^{RM} is independently L;
each L is independently a covalent bond, or a bivalent optionally substituted, linear or branched C₁₋₁₀₀ group comprising one or more aliphatic moieties, aryl moieties, heteroaliphatic moieties each independently having 1-20 heteroatoms, heteroaromatic moieties each independently having 1-20 heteroatoms, or any combinations of any one or more of such moieties, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or —[(—O—C(R′)₂—C(R′)₂—)ₙ]—, wherein n is 1-20;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms; and
MOI is a moiety of interest comprising monomethyl auristatin E (MMAE).

2. The compound or salt of claim 1, wherein LG is or comprises a target binding moiety that binds to a target agent, wherein the target agent is an antibody agent.

3. The compound or salt of claim 1 or 2, wherein LG is or comprises a target binding moiety that binds to the Fc region of an antibody agent.

4. The compound or salt of any preceding claim wherein LG is or comprises a target binding moiety that binds to a target agent, wherein the target agent is an antibody agent that is or comprises enfortumab, brentuximab, or trastuzumab.

5. The compound or salt of any preceding claim, wherein LG is or comprises a group selected from any of A-1 to A-50 in Table A-1.

6. The compound or salt of any preceding claim, wherein R^{LG} is or comprises DCAWXLGELVWCT (SEQ ID NO:18), wherein the two cysteine residues optionally form a disulfide bond, and X is an amino acid residue.

7. The compound or salt of any preceding claim, wherein the compound comprises one or more groups selected from:

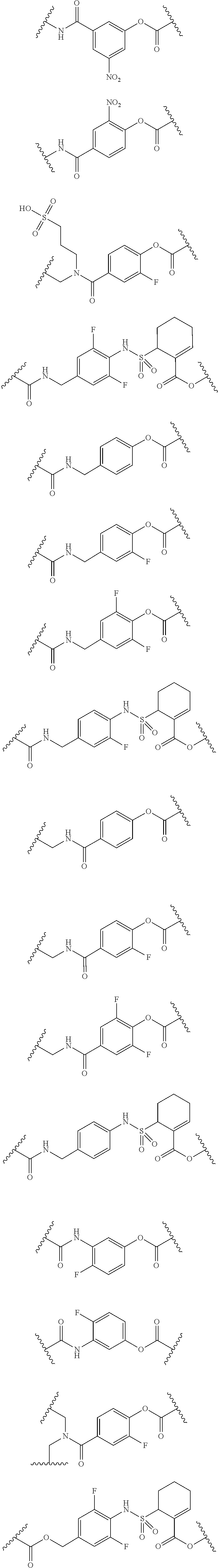

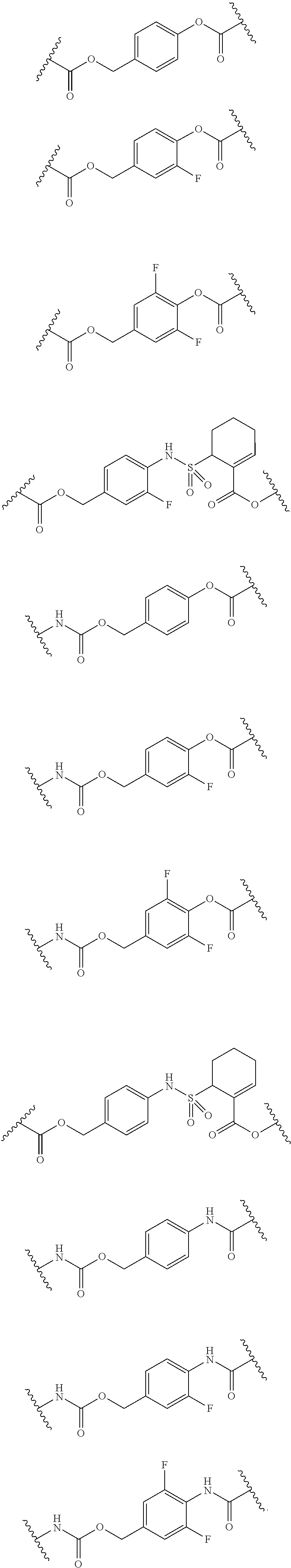

8. The compound or salt of any preceding claim wherein L^{RM} is or comprises —(CH₂CH₂O)n- where n is independently selected at each occurrence from integers 2, 3, 4, 5, 6, 7, and 8.

9. The compound or salt of any preceding claim wherein L^{RM} is or comprises —(CH₂CH₂O)n-(CH₂)n-NHC(O)—(CH₂)n-, —[(CH₂CH₂O)n-(CH₂)n-NHC(O)]m-(CH₂)n-, and —(CH₂CH₂O)n-(CH₂)n-N((CH₂CH₂O)n-(CH₂)n-)((CH₂CH₂O)n-(CH₂)n-) where m is independently selected at each occurrence from integers 1, 2, 3, and 4.

10. A method of preparing an agent having the structure of P-I:
P-L^{PM}-MOI (P—I)
or a salt thereof, wherein:
P is a target agent moiety;
L^{PM} is a linker; and
MOI is a moiety of interest that is or comprises monomethyl auristatin E (MMAE) comprising steps of:
1) contacting the target agent with a reaction partner having the structure of formula R-I:
LG-RG-L^{RM}-MOI (R-I)
or a salt thereof, wherein:
LG is a group comprising a target binding moiety that binds to a target agent,
RG is a reactive group;
L^{RM} is a linker; and
MOI is the moiety of interest that is or comprises MMAE; and
2) forming an agent having the structure of formula P—I; or
a method of preparing an agent having the structure of P-II:
P—N-L^{PM}-MOI (P-II)
wherein:
P—N is a protein agent moiety comprising a lysine residue;
L^{PM} is a linker; and
MOI is a moiety of interest that is or comprises monomethyl auristatin E (MMAE);
the method comprising:
contacting P—N with a reaction partner having a structure of formula R-I:
LG-RG-L^{RM}-MOI (R-I)
or a salt thereof, wherein:
LG is a group comprising a protein-binding moiety that binds to P—N,
RG is a reactive group;
L^{RM} is a linker; and
MOI is the moiety of interest that is or comprises MMAE.

11. The method of claim 10, wherein a target agent is or comprises an antibody agent.

12. The method of claim 10, wherein the antibody agent is or comprises an anti-CD30 monoclonal antibody, such as brentuximab or an anti-nectin-4-monoclonal antibody, such as enfortumab.

13. The method of claim 11 or 12, wherein the moiety of interest is selectively attached to the antibody agent at K246 or K248 of an IgG1 heavy chain or a corresponding location.

14. The method of claim 11 or 12, wherein the moiety of interest is selectively attached to the antibody agent at K251 or K253 of an IgG2 heavy chain or a corresponding location.

15. The method of claim 11 or 12, wherein the moiety of interest is selectively attached to the antibody agent at K239 or K241 of an IgG4 heavy chain or a corresponding location.

16. The method of any one of claims 10 to 12, wherein the contacting and forming steps are performed in one chemical reaction.

17. A composition providing a plurality of agents each of which independently comprise:
an antibody agent moiety,
a moiety of interest that is or comprises monomethyl auristatin E (MMAE), and
optionally a linker moiety linking the antibody agent moiety and the moiety of interest; wherein the antibody agent moieties of the of the plurality of agents comprise a common amino acid sequence or can bind to a common antigen, and agents of the plurality share a common modification independently at at least one common amino acid residue of the antibody agent moieties; and
wherein about 1%-100% of all agents that comprise an antibody agent moiety that comprise the common amino acid sequence or can bind to the common antigen and the moiety of interest are agents of the plurality.

18. The composition of claim 17, wherein antibody agent moieties of agents of the plurality can bind to a common antigen.

19. The composition of claim 18, wherein a common amino acid residue is K246 or K248 of an IgG1 antibody heavy chain or an amino acid residue corresponding thereto.

20. The composition of claim 18, wherein a common amino acid residue is K251 or K253 of an IgG2 antibody heavy chain or an amino acid residue corresponding thereto.

21. The composition of claim 18, wherein a common amino acid residue is K239 or K241 of an IgG4 antibody heavy chain or an amino acid residue corresponding thereto.

22. The composition of claim 18, wherein each agent of the plurality does not contain —S-Cy-, wherein -Cy- is optionally substituted 5-membered monocyclic ring, does not contain —S—S— which is not formed by cysteine residues and does not contain —SH or salt form thereof that is not of a cysteine residue.

23. The composition of claim 18, wherein each agent of the plurality does not contain —S—CH₂—CH₂—.

24. The composition of claim 18, comprising one or more groups selected from

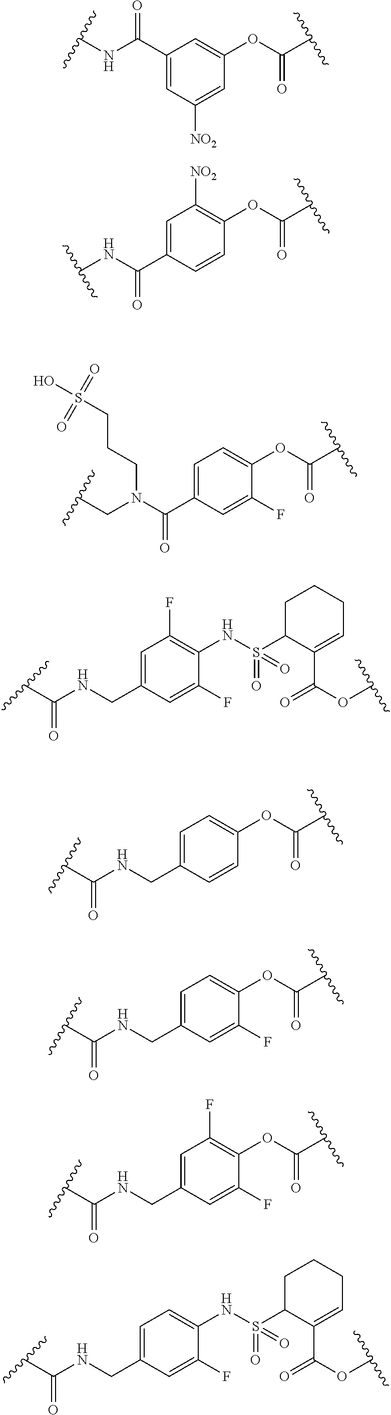

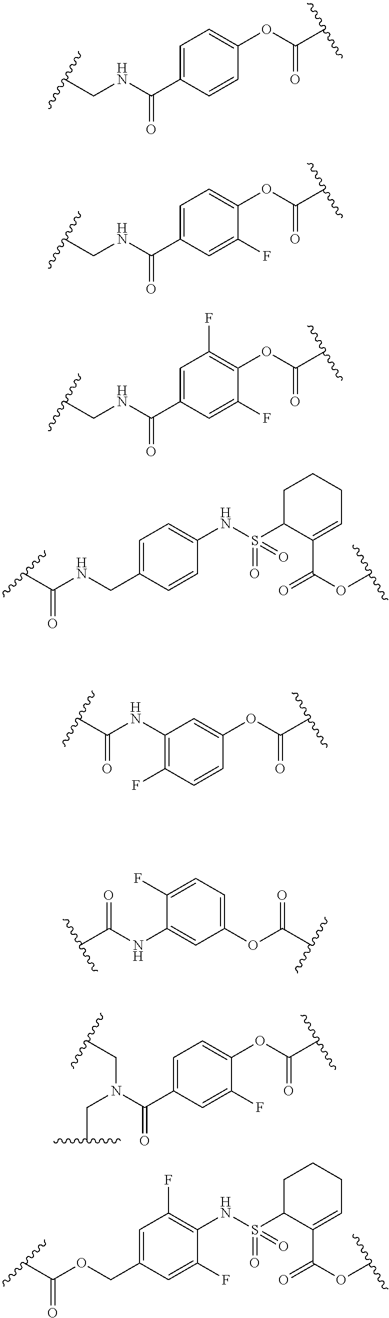

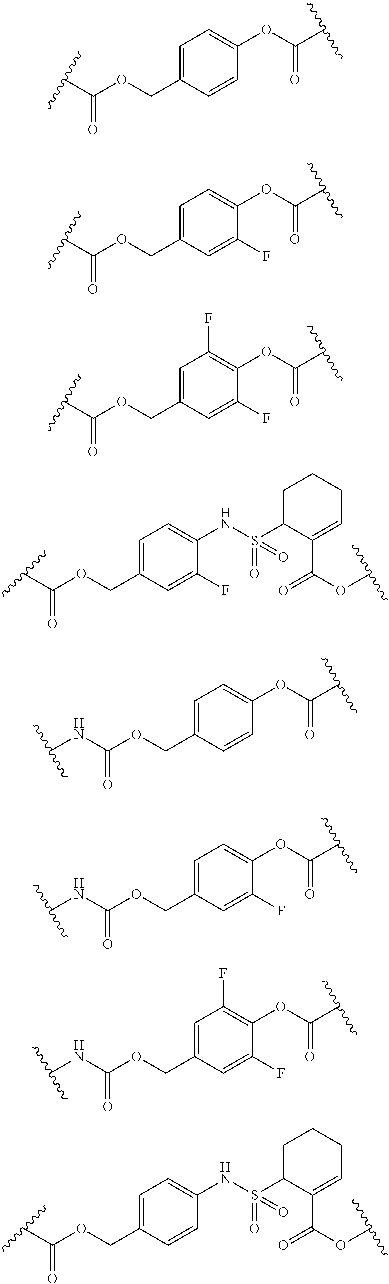

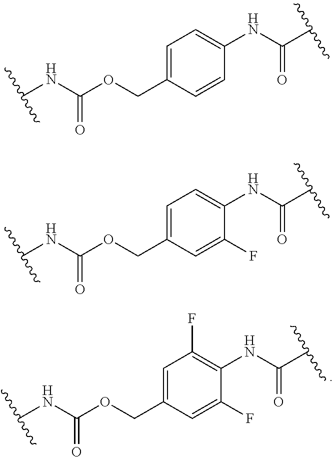 [SMILES: *NC(=O)OCc1ccc(NC(*)=O)cc1 ; *NC(=O)OCc1ccc(NC(*)=O)c(F)c1 ; *NC(=O)OCc1cc(F)c(NC(*)=O)c(F)c1]

25. A compound of claim 1, or a salt thereof, where R^{LG} is is a polypeptide 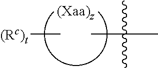
or R^{c}-(Xaa)z- comprising an amino acid residue of at least one of the following compounds: 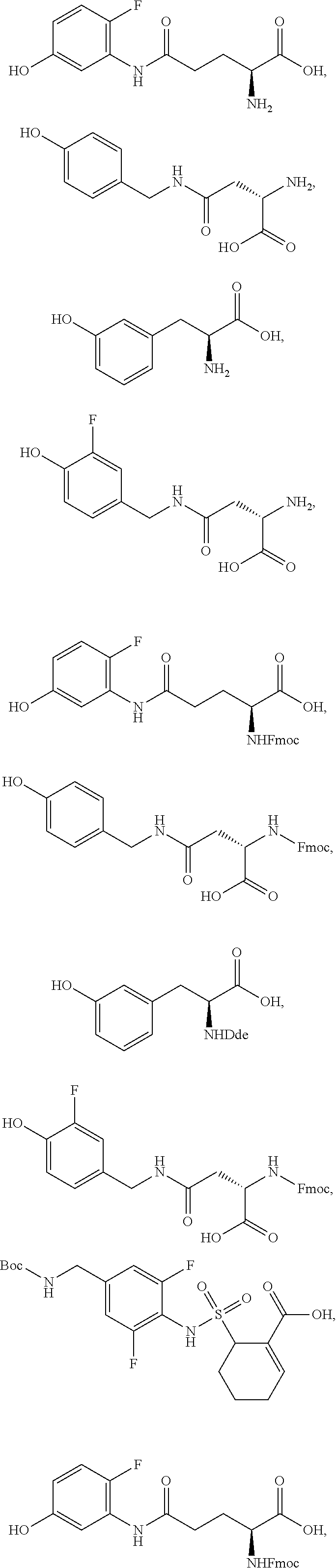
or a salt thereof.

26. A compound or salt of claim 1, wherein the compound is selected from

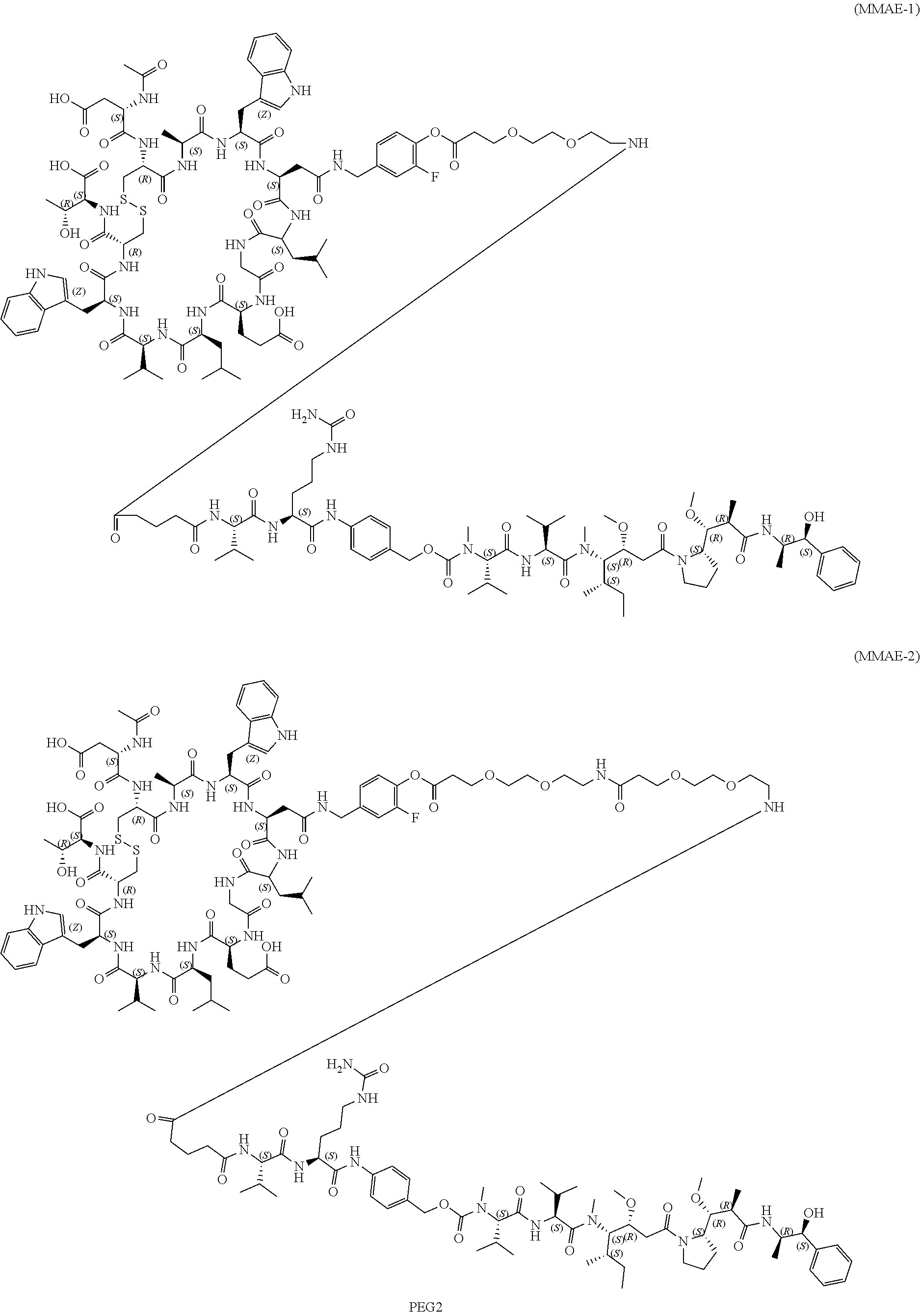

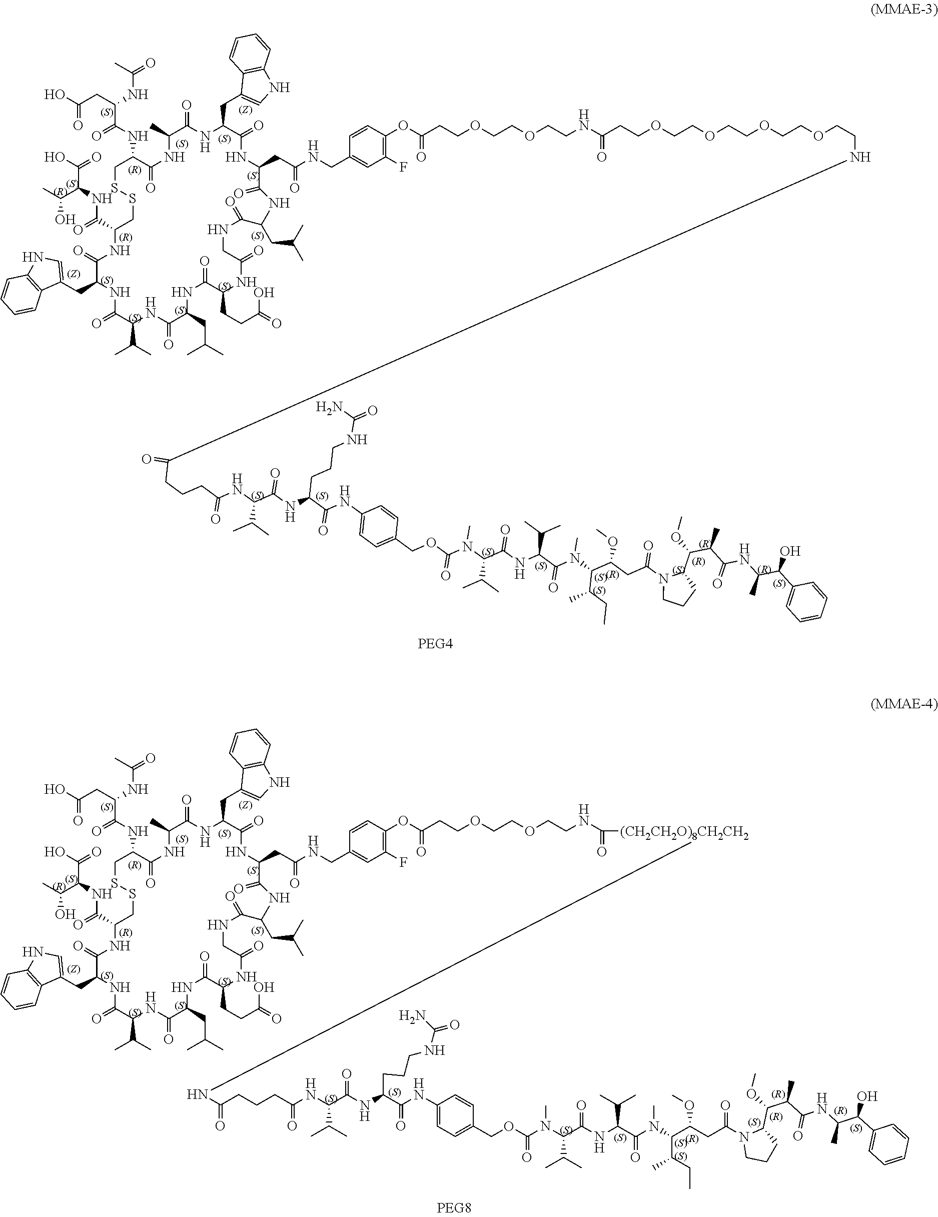

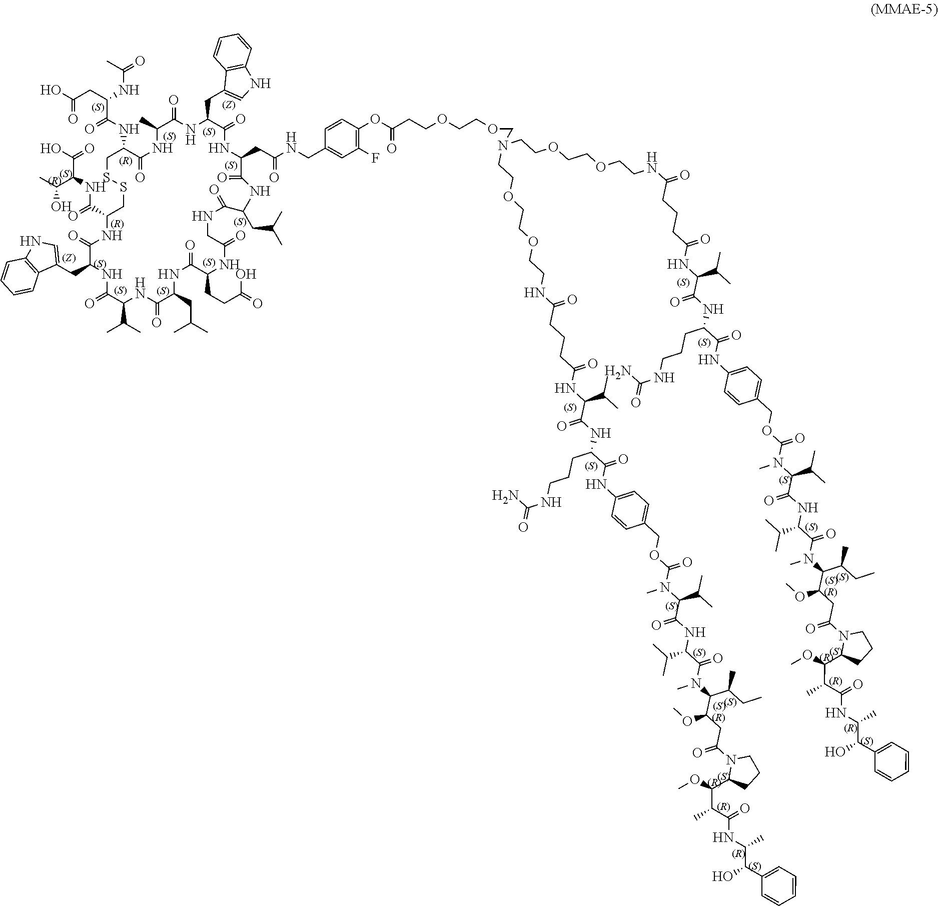

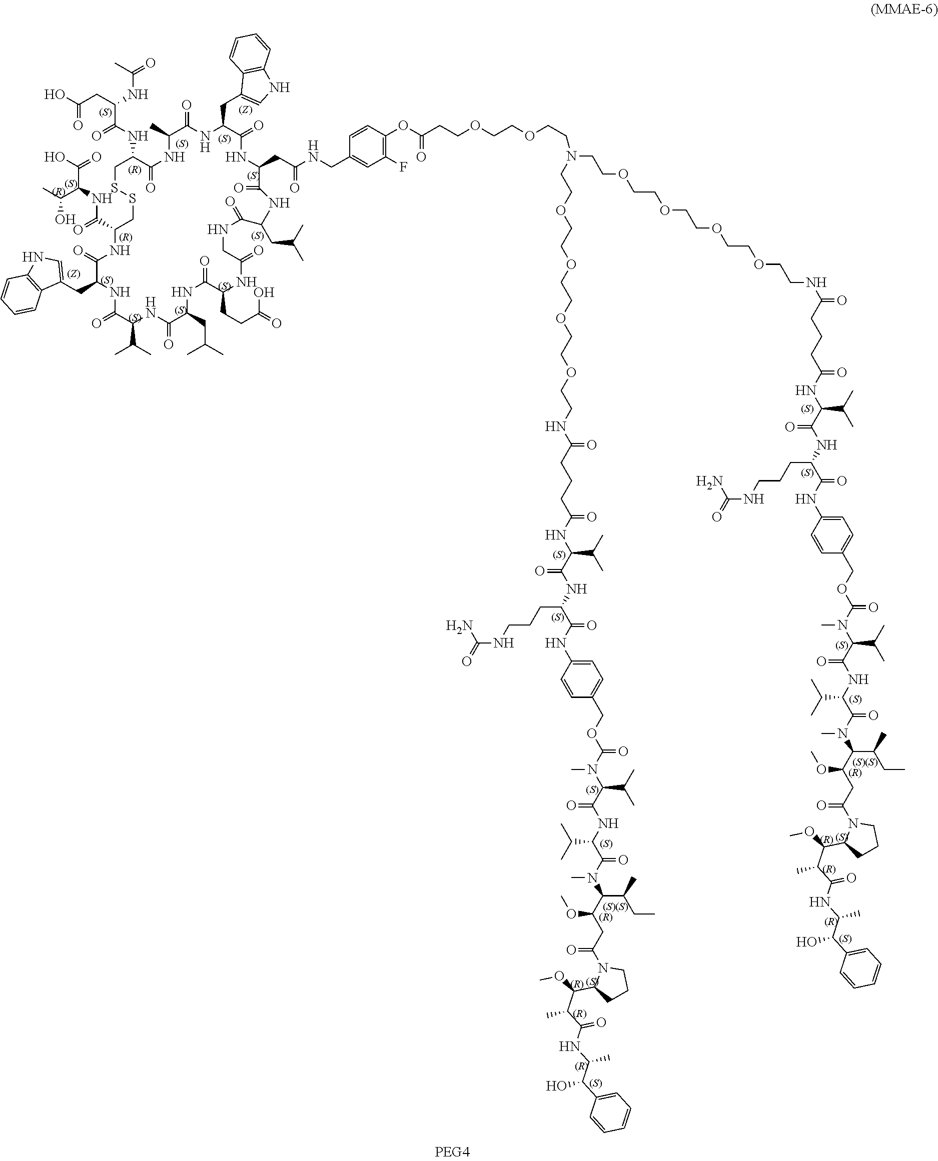

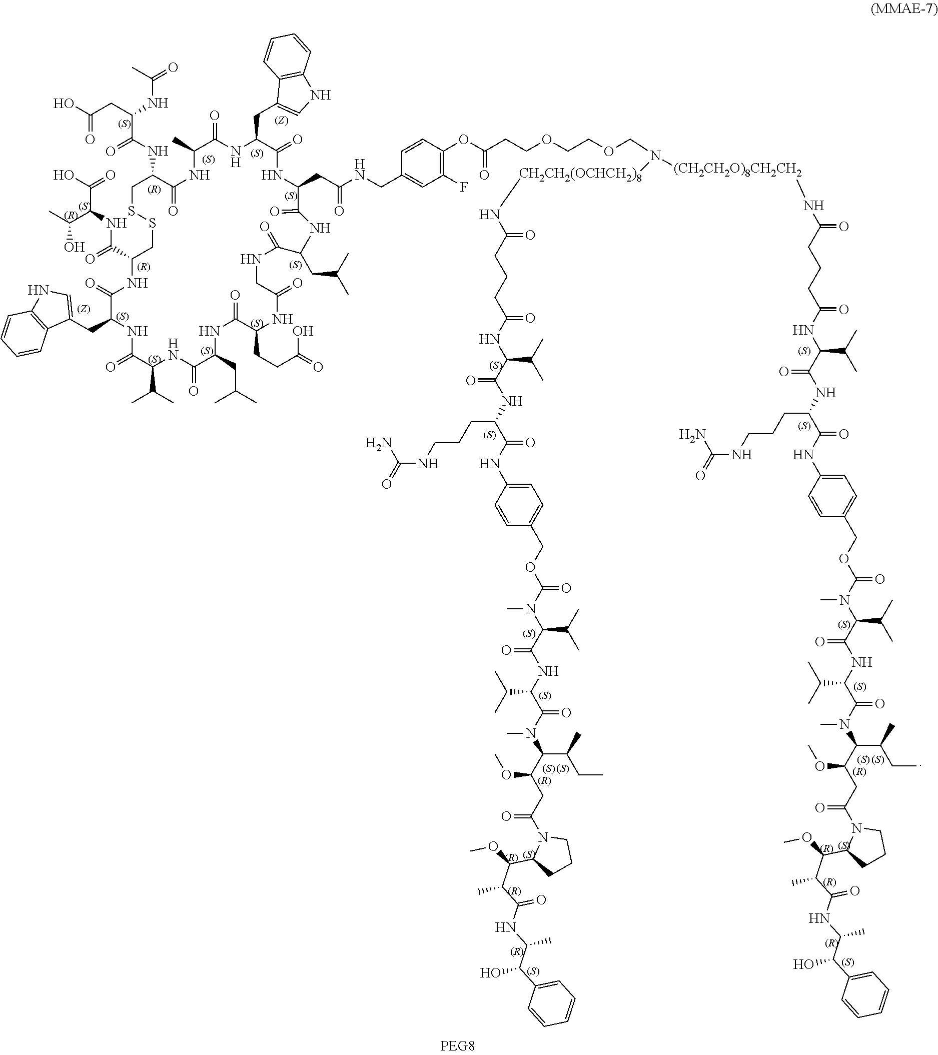

27. A compound having the structure of formula R-I:
LG-RG-L^{RM}-MOI (R-I)
or a salt thereof, wherein:
LG is a group comprising a target binding moiety that binds to a target agent,
RG is a reactive group;
L^{RM} is a linker; and
MOI is a moiety of interest comprising MMAE,
wherein the target agent is an antibody comprising an IgG heavy chain comprising K246 or K248, and
wherein the target binding moiety is configured to bind the antibody so as to bring the reactive group in proximity with K246 or K248 of the IgG heavy chain to enable a reaction between K246 or K248 and the reactive group that results in attachment of a moiety comprising L^{RM}-MOI to K246 or K248 and expulsion of the group containing a target binding moiety from the compound.

28. A compound having the structure of formula R-I:
LG-RG-L^{RM}-MOI (R-I)
or a salt thereof, wherein:
LG is a group comprising a target binding moiety that binds to a target agent,
RG is a reactive group;
L^{RM} is a linker; and
MOI is a moiety of interest comprising monomethyl auristatin E (MMAE).

29. A composition comprising:
a first compound having the structure of formula (P-II):
P—N-L^{PM}-MOI (P-II)
wherein:
P—N is a protein agent moiety comprising a lysine residue;
L^{PM} is a linker; and
MOI is a moiety of interest comprising monomethyl auristatin E (MMAE); and
a second compound having the structure:
LG-OH (LG-I)
wherein LG is a group comprising a target binding moiety that binds to a target agent.

30. The composition of claim 1, further comprising:
a third compound having the formula (R-I):
LG-RG-L^{RM}-MOI (R-I)
wherein:
LG is a group comprising a target binding moiety that binds to a target agent, which is identical to LG in formula (LG-I);
RG is a reactive group;
L^{RM} is a linker, which is identical to in formula (P-II); and
MOI is a moiety of interest comprising monomethyl auristatin E (MMAE);
a fourth compound having the formula (R—III):
HO-RG-L^{RM}-MOI (R—III)
or a combination thereof.


SEQUENCE LISTING (71 sequences)

SEQ ID NO: 1 - protein, 6 aa, Artificial Sequence
  Synthetic
HWRGWA

SEQ ID NO: 2 - protein, 4 aa, Artificial Sequence
  Synthetic
WGRR

SEQ ID NO: 3 - protein, 4 aa, Artificial Sequence
  Synthetic
RRGW

SEQ ID NO: 4 - protein, 8 aa, Artificial Sequence
  Synthetic
NKFRGKYK

SEQ ID NO: 5 - protein, 8 aa, Artificial Sequence
  Synthetic
NRFRGKYK

SEQ ID NO: 6 - protein, 7 aa, Artificial Sequence
  Synthetic
NARKFYK

SEQ ID NO: 7 - protein, 8 aa, Artificial Sequence
  Synthetic
NARKFYKG

SEQ ID NO: 8 - protein, 6 aa, Artificial sequence
  synthetic
HWRGWV

SEQ ID NO: 9 - protein, 7 aa, Artificial SEquence
  Synthetic
KHFRNKD

SEQ ID NO: 10 - protein, 7 aa, Artificial SEquence
  Synthetic
RHRFNKD

SEQ ID NO: 11 - protein, 4 aa, Artificial Sequence
  Synthetic
RTYK

SEQ ID NO: 12 - protein, 4 aa, Artificial Sequence
  Synthetic
ELVW

SEQ ID NO: 13 - protein, 5 aa, Artificial SEquence
  Synthetic
GELVW

SEQ ID NO: 14 - protein, 9 aa, Artificial Seqeunce
  Synthetic
AWHLGELVW

SEQ ID NO: 15 - protein, 9 aa, Artificial sequence
  Synthetic
AWDLGELVW

SEQ ID NO: 16 - protein, 9 aa, Artificial sequence
  Synthetic
  SITE (3)..(3): X is any amino acid
AWXLGELVW

SEQ ID NO: 17 - protein, 13 aa, Artificial sequence
  Synthetic
  disulfid (2)..(12)
DCAWHLGELVWCT

SEQ ID NO: 18 - protein, 13 aa, Artificial sequence
  Synthetic
  disulfid (2)..(12)
  SITE (5)..(5): X is any amino acid
DCAWXLGELVWCT

SEQ ID NO: 19 - protein, 13 aa, Artificial SEquence
  Synthetic
  disulfid (2)..(12)
DCAWDLGELVWCT

SEQ ID NO: 20 - protein, 13 aa, Artificial Sequence
  Synthetic
  SITE (2)..(2): D proline
  SITE (4)..(4): D proline
  SITE (7)..(7): X is any amino acid
DPLPAWXLGELVW

SEQ ID NO: 21 - protein, 13 aa, Artificial SEquence
  Synthetic
  SITE (2)..(2): D proline
  SITE (4)..(4): D proline
DPLPAWDLGELVW

SEQ ID NO: 22 - protein, 13 aa, Artificial SEquence
  Synthetic
  SITE (2)..(2): D proline
  SITE (4)..(4): D proline
DPLPAWHLGELVW

SEQ ID NO: 23 - protein, 17 aa, Artificial SEqeunce
  synthetic
  SITE (2)..(2): D proline
  SITE (4)..(4): D proline
  disulfid (5)..(14)
  SITE (9)..(9): X is any amino acid
DPLPDCAWXLGELVWCT

SEQ ID NO: 24 - protein, 17 aa, Artificial SEquence
  Synthetic
  SITE (2)..(2): D proline
  SITE (4)..(4): D proline
  disulfid (6)..(16)
DPLPDCAWHLGELVWCT

SEQ ID NO: 25 - protein, 17 aa, Artificial sequence
  Synthetic
  SITE (2)..(2): D proline
  SITE (4)..(4): D proline
  Disulfid (6)..(16): D proline
DPLPDCAWDLGELVWCT

SEQ ID NO: 26 - protein, 15 aa, Artificial Seqeunce
  Synthetic
  disulfid (1)..(15)
  disulfid (3)..(13)
  SITE (6)..(6): X is an amino acid
CDCAWXLGELVWCTC

SEQ ID NO: 27 - protein, 15 aa, artificial sequence
  synthetic
  disulfid (1)..(15)
  disulfid (3)..(13)
CDCAWHLGELVWCTC

SEQ ID NO: 28 - protein, 15 aa, artificial sequence
  synthetic
  disulfid (1)..(15)
  disulfid (3)..(13)
CDCAWDLGELVWCTC

SEQ ID NO: 29 - protein, 33 aa, Artificial sequence
  synthetic
FNMQQQRRFYEALHDPNLNEEQRNAKIKSIRDD

SEQ ID NO: 30 - protein, 34 aa, artificial sequence
  synthetic
  disulfid (5)..(34)
FNMQCQRRFYEALHDPNLNEEQRNAKIKSIRDDC

SEQ ID NO: 31 - protein, 17 aa, Artificial sequence
  synthetic
  disulfid (4)..(14)
RGNCAYHRGQLVWCTYH

SEQ ID NO: 32 - protein, 17 aa, artificial sequence
  synthetic
  disulfid (4)..(14)
RGNCAYHKGQLVWCTYH

SEQ ID NO: 33 - protein, 17 aa, artificial sequence
  synthetic
  disulfid (4)..(14)
RGNCKYHRGQLVWCTYH

SEQ ID NO: 34 - protein, 17 aa, artificial sequence
  synthetic
  disulfid (4)..(14)
RGNCAWHRGKLVWCTYH

SEQ ID NO: 35 - protein, 17 aa, artificial sequence
  synthetic
  disulfid (4)..(14)
RGNCKWHRGELVWCTYH

SEQ ID NO: 36 - protein, 17 aa, artificial sequence
  synthetic
  disulfid (4)..(14)
RGNCKWHRGQLVWCTYH

SEQ ID NO: 37 - protein, 17 aa, artificial sequence
  synthetic
  disulfid (4)..(14)
RGNCKYHLGELVWCTYH

SEQ ID NO: 38 - protein, 17 aa, artificial sequence
  synthetic
  disulfid (4)..(14)
RGNCKYHLGQLVWCTYH

SEQ ID NO: 39 - protein, 13 aa, artificial sequence
  synthetic
  disulfid (2)..(12)
DCKWHLGELVWCT

SEQ ID NO: 40 - protein, 13 aa, artificial sequence
  synthetic
  disulfid (2)..(12)
DCKYHLGELVWCT

SEQ ID NO: 41 - protein, 13 aa, artificial sequence
  synthetic
  disulfid (2)..(12)
DCKWHRGELVWCT

SEQ ID NO: 42 - protein, 13 aa, artificial sequence
  synthetic
  disulfid (2)..(12)
DCKWHLGQLVWCT

SEQ ID NO: 43 - protein, 13 aa, artificial sequence
  synthetic
  disulfid (2)..(12)
DCKYHRGELVWCT

SEQ ID NO: 44 - protein, 13 aa, artificial sequence
  synthetic
  disulfid (2)..(12)
DCKYHLGQLVWCT

SEQ ID NO: 45 - protein, 13 aa, artificial sequence
  synthetic
  disulfid (2)..(12)
DCKWHRGQLVWCT

SEQ ID NO: 46 - protein, 13 aa, artificial sequence
  synthetic
  disulfid (2)..(12)
DCKYHRGQLVWCT

SEQ ID NO: 47 - protein, 34 aa, artificial sequence
  synthetic
  disulfid (5)..(34)
FNKQCQRRFYEALHDPNLNEEQRNARIRSIRDDC

SEQ ID NO: 48 - protein, 34 aa, artificial sequence
  synthetic
  disulfid (5)..(34)
FNMQCQRRFYEALHDPNLNEEQRNARIRSIKDDC

SEQ ID NO: 49 - protein, 34 aa, artificial sequence
  synthetic
  disulfid (5)..(34)
FNMQCQRRFYEALHDPNLNKEQRNARIRSIRDDC

SEQ ID NO: 50 - protein, 34 aa, artificial sequence
  synthetic
  disulfid (5)..(34)
FNMQCQRRFYEALHDPNLNEEQRNARIRSIRDDC

SEQ ID NO: 51 - protein, 17 aa, artificial sequence
  synthetic
  disulfid (4)..(14)
RGNCAWHLGQLVWCKYH

SEQ ID NO: 52 - protein, 17 aa, artificial sequence
  synthetic
  disulfid (4)..(14)
RGNCAWHLGELVWCKYH

SEQ ID NO: 53 - protein, 17 aa, artificial sequence
  synthetic
  disulfid (4)..(14)
RGNCAYHLGQLVWCTKH

SEQ ID NO: 54 - protein, 17 aa, artificial sequence
  synthetic
  disulfid (4)..(14)
RGNCAYHLGQLVWCTYK

SEQ ID NO: 55 - protein, 17 aa, artificial sequence
  synthetic
  disulfid (4)..(14)
RGNCAYHRGQLVWCTKH

SEQ ID NO: 56 - protein, 34 aa, artificial sequence
  synthetic
  disulfid (5)..(34)
KNMQCQRRFYEALHDPNLNEEQRNARIRSIRDDC

SEQ ID NO: 57 - protein, 34 aa, artificial sequence
  synthetic
  disulfid (5)..(34)
FNMQCQKRFYEALHDPNLNEEQRNARIRSIRDDC

SEQ ID NO: 58 - protein, 34 aa, artificial sequence
  synthetic
  disulfid (5)..(34)
FNMQCQRRFYEAKHDPNLNEEQRNARIRSIRDDC

SEQ ID NO: 59 - protein, 34 aa, artificial sequence
  synthetic
  disulfid (5)..(34)
FNMQCQRRFYEALHDPNLNEEQRKARIRSIRDDC

SEQ ID NO: 60 - protein, 34 aa, artificial sequence
  synthetic
  disulfid (5)..(34)
FNMQCKRRFYEALHDPNLNEEQRNARIRSIRDDC

SEQ ID NO: 61 - protein, 34 aa, artificial sequence
  synthetic
  disulfid (5)..(34)
FNMQCQRRFYEALHDPNLNEEQRNARIRSIRKDC

SEQ ID NO: 62 - protein, 17 aa, artificial sequence
  synthetic
  Acetylation (1)..(1)
  Disulfid (4)..(14): X is any K or R
  SITE (8)..(8): X is any K or R
GPDCAYHXGELVWCTFH

SEQ ID NO: 63 - protein, 11 aa, artificial sequence
  synthetic
  disulfid (1)..(11)
  SITE (2)..(2): X is any amino acid
  SITE (5)..(7): X is any amino acid
CXYHXXXLVWC

SEQ ID NO: 64 - protein, 12 aa, artificial sequence
  synthetic
  SITE (1)..(1): X is any amino acid
  disulfid (2)..(12)
  SITE (3)..(3): X is any amino acid
  SITE (6)..(8): X is any amino acid
XCXYHXXXLVWC

SEQ ID NO: 65 - protein, 12 aa, artificial sequence
  synthetic
  disulfid (1)..(11)
  SITE (2)..(2): X is any amino acid
  SITE (5)..(7): X is any amino acid
  SITE (12)..(12): X is any amino acid
CXYHXXXLVWCX

SEQ ID NO: 66 - protein, 17 aa, artificial sequence
  synthetic
  site (1)..(3): X is any amino acid and up to three may be absent
  disulfid (4)..(14)
  site (5)..(5): X is any amino acid
  site (8)..(10): X is any amino acid
  site (15)..(17): X is any amino acid and up to three may be absent
XXXCXYHXXXLVWCXXX

SEQ ID NO: 67 - protein, 15 aa, artificial sequence
  Synthetic
  site (1)..(1): X is any amino acid
  disulfid (2)..(12)
  site (3)..(3): X is any amino acid
  site (6)..(8): X is any amino acid
  site (13)..(15): X is any amino acid
XCXYHXXXLVWCXXX

SEQ ID NO: 68 - protein, 17 aa, artificial sequence
  synthetic
  site (1)..(3): X is any amino acid and up to two of them may be absent
  disulfid (4)..(14)
  site (5)..(6): X is any amino acid
  site (8)..(8): X is any amino acid
  site (10)..(10): X is any amino acid
  site (15)..(17): X is any amino acid and up to two of them may be absent
XXXCXXHXGXLVWCXXX

SEQ ID NO: 69 - protein, 15 aa, artificial sequence
  synthetic
  disulfid (2)..(12)
  site (2)..(4): X is any amino acid
  site (6)..(6): X is any amino acid
  site (8)..(8): X is any amino acid
  site (13)..(15): X is any amino acid
DCXXHXGXLVWCXXX

SEQ ID NO: 70 - protein, 13 aa, artificial sequence
  synthetic
  disulfid (2)..(12)
  site (3)..(4): X is any amino acid
  site (6)..(6): X is any amino acid
  site (8)..(8): X is any amino acid
DCXXHXGXLVWCT

SEQ ID NO: 71 - protein, 17 aa, artificial sequence
  synthetic
  disulfid (4)..(14)
  site (5)..(6): X is any amino acid
  site (8)..(8): X is any amino acid
  site (10)..(10): X is any amino acid
  site (15)..(17): X is any amino acid
RGNCXXHXGXLVWCXXX